(19) United States
(12) Patent Application Publication
(10) Pub. No.: US 2023/0277297 A1
(43) Pub. Date: Sep. 7, 2023

(54) DEVICES FOR AORTIC REPAIR, AND ASSOCIATED SYSTEMS AND METHODS

(71) Applicant: Mavericks Endo, Inc., Menlo Park, CA (US)

(72) Inventors: G Ray Martin, Redwood City, CA (US); Steven Kim, Los Altos, CA (US); Hanson Gifford, Woodside, CA (US); Katie Miyashiro, San Francisco, CA (US); Doug Sutton, Pacifica, CA (US); Anuja Harshadray Patel, Pacifica, CA (US); Richard Roy Newhauser, JR., Belmont, CA (US)

(21) Appl. No.: 18/179,254

(22) Filed: Mar. 6, 2023

Related U.S. Application Data
(60) Provisional application No. 63/316,896, filed on Mar. 4, 2022.
(60) Provisional application No. 63/425,600, filed on Nov. 15, 2022.
(60) Provisional application No. 63/437,916, filed on Jan. 9, 2023.

(51) Int. Cl. A61F 2/07

(52) U.S. Cl. CPC A61F 2/07; A61F 2002/061

(57) ABSTRACT

Aortic repair devices and associated systems and methods are disclosed herein. An aortic repair device configured in accordance with embodiments of the present technology can include a base member configured to be implanted in an aorta proximate to a diseased portion of the aorta, such as an aneurysm or dissection. The base member can direct blood flow past the diseased portion of the aorta to perfuse a portion of the aorta distal of the diseased portion and/or various branch vessels. In some embodiments, one or more spanning members or other implants can be coupled (e.g., docked) to the base member during the same or a separate intravascular procedure to provide additional flow paths past the diseased portion.

### CROSS-REFERENCE TO RELATED APPLICATIONS

This application claims the benefit of U.S. Provisional Patent Application No. 63/316,896, filed Mar. 4, 2022, and titled “DEVICES FOR AORTIC REPAIR, AND ASSOCIATED SYSTEMS AND METHODS,” U.S. Provisional Patent Application No. 63/425,600, filed Nov. 15, 2022, and titled “DEVICES FOR AORTIC REPAIR, AND ASSOCIATED SYSTEMS AND METHODS,” and U.S. Provisional Patent Application No. 63/437,916, filed Jan. 9, 2023, and titled “DEVICES FOR AORTIC REPAIR, AND ASSOCIATED SYSTEMS AND METHODS,” each of which is incorporated herein by reference in its entirety.

### TECHNICAL FIELD

The present technology is directed to devices, systems, and methods for repairing a diseased aorta, and more particularly to devices configured to be implanted at least partially within the proximal aorta to treat aneurysms and dissections in the ascending aorta and/or the aortic arch.

### BACKGROUND

Aneurysms, dissections, penetrating ulcers, intramural hematomas, and/or transections may occur in blood vessels, and most typically occur in the aorta and peripheral arteries. A diseased region of the aorta may extend into areas having vessel bifurcations or segments of the aorta from which smaller “branch” arteries extend.

The diseased region of the aorta and other vessels can be bypassed with a stent graft placed inside the vessel to span the diseased region. The stent graft can effectively seal off the diseased region from further exposure to blood flow, preventing the aneurysm, dissection, or other type of diseased region from worsening. However, the use of stent grafts to internally bypass a diseased region of a vessel is not without challenges. In particular, care must be taken so that the stent graft does not cover or occlude critical branch vessels, yet the stent graft must adequately seal against the healthy regions of the vessel wall and remain open to provide a flow conduit for blood to flow past the diseased region.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIGS. 1A and 1B are side views of a diseased aorta and surrounding anatomy in which an aortic repair device can be implanted in accordance with embodiments of the present technology.

FIGS. 2A-2D are side cross-sectional views of a diseased aorta in which an aortic repair device can be implanted in accordance with embodiments of the present technology.

FIGS. 3A and 3B are a side view and a perspective side view, respectively, of an aortic repair device in accordance with embodiments of the present technology. FIG. 3C is a cross-sectional view of the aortic repair device of FIGS. 3A and 3B taken along the line 3C-3C in FIG. 3A in accordance with embodiments of the present technology. FIG. 3D is a side view of the aortic repair device of FIGS. 3A-3C implanted within an aorta in accordance with embodiments of the present technology.

FIG. 4A a side view of an aortic repair device implanted within an aorta in accordance with embodiments of the present technology.

FIG. 4B is a side view of an aortic repair device implanted within an aorta in accordance with additional embodiments of the present technology.

FIG. 5A is a side view of an aortic repair device 500 in accordance with embodiments of the present technology. FIGS. 5B and 5C are cross-sectional views of the aortic repair device of FIG. 5A taken along the line 5B-5B in FIG. 5A in accordance with embodiments of the present technology.

FIG. 6A is a side view of an aortic repair device in accordance with embodiments of the present technology. FIG. 6B is a cross-sectional view of the aortic repair device of FIG. 6A taken along the line 6B-6B in FIG. 6A in accordance with embodiments of the present technology.

FIGS. 7A and 7B are a distally-facing perspective end view and a distally-facing end view, respectively, of an aortic repair device in accordance with embodiments of the present technology. FIG. 7C is a distally-facing end view of the aortic repair device of FIGS. 7A and 7B expanded within an aorta in accordance with embodiments of the present technology.

FIGS. 8A-8E are a side view, a perspective side view, a proximally-facing perspective view, a proximally-facing perspective end view, and a distally-facing perspective end view, respectively, of an aortic repair device in accordance with embodiments of the present technology.

FIGS. 8F and 8G are cross-sectional views of the aortic repair device of FIGS. 8A-8E taken along the line 8F-8F in FIG. 8B. FIG. 8H is a cross-sectional view of the aortic repair device of FIGS. 8A-8G taken along the line 8F-8F in FIG. 8B with a spanning member deployed within a lumen of the aortic repair device in accordance with embodiments of the present technology.

FIGS. 9A and 9B are cross-sectional views of the aortic repair device of FIG. 8H during a first delivery stage and a second delivery stage in accordance with embodiments of the present technology.

FIGS. 10A and 10B are a distally-facing end view and a distally-facing perspective end view, respectively, of an aortic repair device in accordance with embodiments of the present technology. FIGS. 10C and 10D are a distally-facing end view and a distally-facing perspective end view, respectively, of the aortic repair device of FIGS. 10A and 10B with a spanning member deployed within a primary lumen of the aortic repair device in accordance with embodiments of the present technology.

FIG. 11A is a cross-sectional view of an aortic repair device in accordance with embodiments of the present technology. FIG. 11B is a cross-sectional side view of a stent of a spanning member of the aortic repair device of FIG. 11A in accordance with embodiments of the present technology.

FIG. 12A is a cross-sectional view of an aortic repair device in accordance with embodiments of the present technology. FIG. 12B is a cross-sectional side view of a stent of a spanning member of the aortic repair device of FIG. 12A in accordance with embodiments of the present technology.

FIGS. 13A-13C are a perspective side view, a distally-facing perspective end view, and another distally-facing perspective end view, respectively, of a spanning member in accordance with embodiments of the present technology.

FIGS. 14A and 14B are perspective side views of a spanning member in accordance with embodiments of the present technology. FIGS. 14C and 14D are a distally-facing perspective end view and an enlarged distally-facing perspective end view, respectively, of an aortic repair device including the spanning member of FIGS. 14A and 14B deployed within a base member in accordance with embodiments of the present technology.

FIGS. 15A and 15B are a side view and a distally-facing end view, respectively, of a spanning member in accordance with embodiments of the present technology. FIG. 15C is a distally-facing end view of an aortic repair device including the spanning member of FIGS. 15A and 15B in accordance with embodiments of the present technology. FIGS. 15D and 15E are side views of the spanning member of FIGS. 15A-15C when subject to blood flow and having a graft material attached to an inside and an outside of a plurality of stents, respectively, in accordance with embodiments of the present technology.

FIGS. 16A and 16B are a perspective side view and a perspective end view, respectively, of a spanning member in accordance with embodiments of the present technology.

FIGS. 17A-17C are a perspective side view, a side cross-sectional view, and a perspective end view, respectively, of a spanning member in accordance with embodiments of the present technology.

FIG. 18A is an end view of a spanning member in a constrained configuration and an expanded configuration in accordance with embodiments of the present technology. FIG. 18B is a distally-facing end view of an aortic repair device 1800 including the spanning member of FIG. 18A in accordance with embodiments of the present technology.

FIG. 19 is a distally-facing end view of an aortic repair device in accordance with embodiments of the present technology.

FIGS. 20A and 20B are an enlarged perspective side view and a distally-facing perspective end view, respectively, of a spanning member in accordance with embodiments of the present technology.

FIG. 21 is a perspective side view of a spanning member configured in accordance with embodiments of the present technology.

FIGS. 22A and 22B are perspective end views of an aortic repair device configured in accordance with embodiments of the present technology.

FIG. 23 is a side view of a stent for use with the aortic repair device of FIGS. 22A and 22B in accordance with embodiments of the present technology.

FIG. 24 is a side view of an aorta illustrating aspects of aortic repair device delivery procedures.

FIGS. 25A-25E are perspective side views of various aortic repair devices configured in accordance with embodiments of the present technology.

FIGS. 26A and 26B are enlarged side views of a portion of an aortic repair device in a first position and a second position, respectively, in accordance with embodiments of the present technology.

FIGS. 27A and 27B are a side perspective view and a perspective end view, respectively, of an aortic repair device configured in accordance with embodiments of the present technology. FIG. 27C is a perspective view of a first stent component of the aortic repair device of FIGS. 27A and 27B in accordance with embodiments of the present technology. FIG. 27D shows the aortic repair device of FIGS. 27A and 27B implanted within an aorta and conforming to the wall of the ascending aorta proximate the aortic arch in accordance with embodiments of the present technology.

FIGS. 28A and 28B are a perspective view and a perspective end view, respectively, of an aortic repair device configured in accordance with embodiments of the present technology.

FIGS. 29A and 29B are perspective end views of an aortic repair device configured in accordance with embodiments of the present technology.

FIGS. 30A and 30B are perspective end views of an aortic repair device configured in accordance with embodiments of the present technology. FIGS. 30C and 30D are a perspective end view and an end view, respectively, of a secondary lumen stent component of the aortic repair device of FIGS. 30A and 30B in accordance with embodiments of the present technology. FIGS. 30E and 30F are perspective end views of the aortic repair device of FIGS. 30A and 30B with a spanning member deployed within a primary lumen of the aortic repair device in accordance with embodiments of the present technology.

FIGS. 31A and 31B are perspective end views of an aortic repair device configured in accordance with embodiments of the present technology. FIGS. 31C and 31D are a perspective end view and an end view, respectively, of a secondary lumen stent of the aortic repair device of FIGS. 31A and 31B in accordance with embodiments of the present technology. FIGS. 31E-31G are perspective end views of the aortic repair device of FIGS. 31A and 31B with a spanning member deployed within a primary lumen of the aortic repair device in accordance with embodiments of the present technology. FIGS. 31H-31L are perspective end views of the aortic repair device of FIGS. 31A and 31B illustrating increasing radial compression of the aortic repair device in accordance with embodiments of the present technology.

FIG. 32A is a perspective end view of an aortic repair device deployed within a lumen in accordance with embodiments of the present technology. FIG. 32B is an end view of a secondary lumen stent component of the aortic repair device of FIG. 32A in accordance with embodiments of the present technology. FIGS. 32C and 32D are perspective end views of the aortic repair device of FIG. 32A with a spanning member deployed within a primary lumen of the aortic repair device in accordance with embodiments of the present technology.

FIG. 33 is a cross-sectional view of an aortic repair device configured in accordance with embodiments of the present technology.

FIGS. 34A and 34B are perspective side views of an aortic repair device in a longitudinally elongated position and a longitudinally compressed position, respectively, in accordance with embodiments of the present technology.

FIGS. 35A and 35B are perspective side views of an aortic repair device in a longitudinally elongated position and a longitudinally compressed position, respectively, in accordance with embodiments of the present technology.

FIGS. 36A and 36B are perspective side views of an aortic repair device in a longitudinally elongated position from different circumferential perspectives in accordance with embodiments of the present technology. FIG. 36C is a perspective side view of the aortic repair device in a longitudinally compressed position in accordance with embodiments of the present technology. FIG. 36D is a planar view of a first stent and a second stent of the aortic repair device of FIGS. 36A-36C in accordance with embodiments of the present technology.

FIG. 37A is a perspective side view of an aortic repair device configured in accordance with embodiments of the present technology. FIG. 37B is a planar view of a first stent and a second stent of the aortic repair device of FIG. 37A configured in accordance with embodiments of the present technology.

FIG. 38 is a planar view of a first stent and a second stent of an aortic repair device in accordance with additional embodiments of the present technology.

FIGS. 39A and 39B are a side view and a perspective side view, respectively, of an aortic repair device in a longitudinally elongated position in accordance with embodiments of the present technology. FIG. 39C is a perspective side view of the aortic repair device of FIGS. 39A and 39B in a longitudinally compressed position in accordance with embodiments of the present technology.

FIGS. 40A and 40B are a side view and a perspective side view, respectively, of an aortic repair device in a longitudinally elongated position in accordance with embodiments of the present technology. FIG. 40C is a perspective side view of the aortic repair device of FIGS. 40A and 40B in a longitudinally compressed position in accordance with embodiments of the present technology. FIG. 40D is a side view of the aortic repair device of FIGS. 39A-39C or 40A-40C implanted within an aorta in accordance with embodiments of the present technology.

FIGS. 41A and 41B are side views of a spanning member configured in accordance with embodiments of the present technology. FIG. 41C is an enlarged view of a portion of the spanning member shown in FIG. 41B in accordance with embodiments of the present technology.

FIGS. 41D-41F are enlarged views of a proximal end portion or a distal end portion of a component of an aortic repair device in accordance with embodiments of the present technology.

FIG. 42A is an anterior view of an aorta and surrounding anatomy in which an aortic repair device can be implanted in accordance with embodiments of the present technology. FIGS. 42B-42D are side views of various aortic repair devices in accordance with embodiments of the present technology.

FIGS. 43A-43C are a side view, a perspective side view, and a distally-facing perspective end view, respectively, of an aortic repair device in accordance with embodiments of the present technology. FIG. 43D is a side view of the aortic repair device of FIGS. 43A-43C implanted within an aorta in accordance with embodiments of the present technology.

FIGS. 44A-44C are a side view, a perspective side view, and distally-facing perspective end view, respectively, of a spanning member in accordance with embodiments of the present technology. FIGS. 44D and 44E are a perspective side view and a side view, respectively, of an aortic repair device including the spanning member of FIGS. 44A-44C coupled to a base member of FIGS. 43A-43D in accordance with embodiments of the present technology.

FIG. 45A is an enlarged side view of a spanning member in accordance with embodiments of the present technology. FIG. 45B is a side view of an aortic repair device including the spanning member of FIG. 45A coupled to the base member of FIGS. 43A-43D in accordance with embodiments of the present technology. FIG. 45C is a proximally-facing perspective view of a primary leg of the base member of FIG. 45B in accordance with embodiments of the present technology. FIG. 45D is an enlarged perspective side view of a proximal end portion of the spanning member of FIG. 45A in accordance with embodiments of the present technology. FIG. 45E is a proximally-facing perspective of the spanning member of FIG. 45D coupled to the primary leg of the base member of FIG. 45C in accordance with embodiments of the present technology.

FIG. 46 is a perspective side view of a base member of an aortic repair device in accordance with embodiments of the present technology.

FIGS. 47A and 47B are a distally-facing perspective view and a proximally-facing perspective end view, respectively, of an aortic repair device in accordance with embodiments of the present technology. FIGS. 47C and 47D are a perspective side view and a distally-facing perspective end view, respectively, of the base member of the aortic repair device of FIGS. 47A and 47B in accordance with embodiments of the present technology. FIGS. 47E-47G are cross-sectional views of the base member of the aortic repair device of FIGS. 47C and 47D in accordance with embodiments of the present technology.

FIGS. 48A-48D are an isometric side view, a perspective side view, an enlarged perspective side view, and a distally-facing perspective end view, respectively, of an aortic repair device in accordance with embodiments of the present technology. FIGS. 48E-48H are cross-sectional views of the aortic repair device as indicated in FIG. 48A in accordance with embodiments of the present technology.

FIG. 49 is an isometric side view of a spanning member configured to be coupled to a base member of the aortic repair device of FIGS. 48A-48H in accordance with embodiments of the present technology.

FIG. 50 is a side view of an aortic repair device comprising a pair of the base members of FIGS. 48A-48H and the spanning member of FIG. 49 implanted within an aorta in accordance with embodiments of the present technology.

FIGS. 51A-51D are a side view, a perspective side view, a perspective end view, and a distally-facing perspective end view, respectively, of an aortic repair device in accordance with embodiments of the present technology. FIGS. 51E and 51F are a distally-facing end view and a side view, respectively, of the aortic repair device of FIGS. 51A-51C in accordance with additional embodiments of the present technology.

FIGS. 52A-52C are side views of the aortic repair device of FIG. 51D during different stages of implantation within an aorta in accordance with embodiments of the present technology.

FIGS. 53A and 53B are an enlarged side view and a perspective side view, respectively, of a septum and a stent of the aortic repair device of FIGS. 51A-51C in accordance with embodiments of the present technology.

FIGS. 54A-54C are an enlarged side view, a perspective top view, and a perspective side view, respectively, of a septum and a stent of the aortic repair device of FIGS. 51A-51C in accordance with embodiments of the present technology.

FIGS. 55A and 55B are a cross-sectional view and a perspective end view, respectively, of a base member of the aortic repair device of FIGS. 51A-51C in accordance with additional embodiments of the present technology. FIGS. 55C and 55D are perspective end views of the base member of FIGS. 55A and 55B including a first spanning member, a second spanning member, and a third spanning member, coupled to the base member in accordance with embodiments of the present technology.

FIG. 56A is an enlarged isometric end view of an aortic repair device in accordance with embodiments of the present technology. FIGS. 56B and 56C are side views of the aortic repair device of FIG. 56A during different stages of implantation within an aorta in accordance with embodiments of the present technology.

FIG. 57 is a side view of an aortic repair device implanted within an aorta in accordance with embodiments of the present technology.

FIG. 58A is a side view of an aortic repair device implanted within an aorta in accordance with additional embodiments of the present technology.

FIG. 58B is a side view of the aortic repair device of FIG. 58A implanted within an aorta in a different configuration in accordance with embodiments of the present technology.

FIG. 58C is a side view of the aortic repair device of FIG. 58B implanted within an aorta and including an additional extending member in accordance with embodiments of the present technology.

FIG. 59 is a side view of an aortic repair device configured in accordance with embodiments of the present technology.

FIG. 60A is a side view of an aortic repair device implanted within an aorta in accordance with additional embodiments of the present technology. FIGS. 60B and 60C are cross-sectional views of the aortic repair device of FIG. 60A taken along the line 60B-60B in accordance with embodiments of the present technology.

FIGS. 61A-61F are side views of various stages of a procedure to implant an aortic repair device within the aorta in accordance with embodiments of the present technology.

FIGS. 62A and 62B provide a schematic view of the aortic repair device of FIGS. 61A-61F implanted within the aorta in accordance with embodiments of the present technology.

FIGS. 63A and 63B provide a schematic view of the aortic repair device of FIGS. 61A-61F implanted within the aorta in accordance with additional embodiments of the present technology.

FIGS. 64A and 64B provide a schematic view of the aortic repair device of FIGS. 61A-61F implanted within the aorta in accordance with additional embodiments of the present technology.

FIG. 65A is a side view of an aortic repair device implanted within an ascending aorta in accordance with embodiments of the present technology.

FIG. 65B is a side view of the aortic repair device of FIG. 65A implanted within the descending aorta in accordance with embodiments of the present technology.

FIG. 66A is a side view of an aortic repair device implanted within an aorta in accordance with additional embodiments of the present technology. FIG. 66B is a cross-sectional view of the aortic repair device of FIG. 66A taken along the line 66B-66B in FIG. 66A in accordance with additional embodiments of the present technology.

FIG. 67A is a side view of an aortic repair device implanted within an aorta in accordance with additional embodiments of the present technology. FIG. 67B is a cross-sectional view of the aortic repair device of FIG. 67A taken along the line 67B-67B in FIG. 67A in accordance with additional embodiments of the present technology.

FIG. 68A is a side view of an aortic repair device implanted within an aorta in accordance with additional embodiments of the present technology. FIG. 68B is a cross-sectional view of the aortic repair device of FIG. 68A taken along the line 68B-68B in FIG. 68A in accordance with additional embodiments of the present technology.

FIG. 69A is a side view of an aortic repair device implanted within an aorta in accordance with additional embodiments of the present technology. FIG. 69B is a cross-sectional view of the aortic repair device of FIG. 69A taken along the line 69B-69B in FIG. 69A in accordance with additional embodiments of the present technology.

FIG. 70 is a side view of an aortic repair device implanted within an aorta in accordance with embodiments of the present technology.

FIG. 71 is a side view of an aortic repair device implanted within an aorta in accordance with embodiments of the present technology.

FIGS. 72A and 72B are enlarged isometric side views of base member of an aortic repair device in accordance with embodiments of the present technology.

FIGS. 73A and 73B are end views illustrating the sequential collapse of an anchor of the base member of FIGS. 72A and 72B for loading into a delivery catheter system in accordance with embodiments of the present technology.

FIGS. 74A and 74B are enlarged isometric side views of a base member of the aortic repair device of FIG. 72B in accordance with embodiments of the present technology.

FIGS. 75A and 75B are a side view and an enlarged isometric side view, respectively, of an aortic repair device in accordance with embodiments of the present technology. FIG. 75C is an isometric side view of a stent of the aortic repair device of FIGS. 75A and 75B in accordance with embodiments of the present technology. FIG. 75D is a side view of the aortic repair device of FIGS. 75A-75C implanted within an aorta in accordance with embodiments of the present technology.

FIGS. 76A-76C are side views of an aortic repair device during different stages of implantation within an aorta in accordance with embodiments of the present technology.

FIGS. 77A and 77B are side views of an aortic repair device during different stages of implantation within an aorta in accordance with embodiments of the present technology.

FIGS. 78A and 78B are side views of an aortic repair device during different stages of implantation within an aorta in accordance with embodiments of the present technology.

FIGS. 79A and 79B are side views of an aortic repair device implanted within an aorta in accordance with embodiments of the present technology.

FIG. 80 is a side view of an aortic repair device implanted within an aorta in accordance with embodiments of the present technology.

FIG. 81A is a side view of an aortic repair device implanted within an aorta in accordance with embodiments of the present technology. FIG. 81B is an enlarged isometric side view of a portion of the aortic repair device of FIG. 81A in accordance with embodiments of the present technology.

FIGS. 82A and 82B are side views of a branch perfusion member and a spanning member, respectively, in accordance with embodiments of the present technology. FIG. 82C is a side view of a portion of a modularly assembled aortic repair device including the branch perfusion member and the spanning member of FIGS. 82A and 82B in accordance with embodiments of the present technology.

FIGS. 83A and 83B are a perspective side view and a distally-facing perspective end view, respectively, of a spanning member of an aortic repair device in accordance with embodiments of the present technology.

FIG. 84 is a perspective side view of a base member of an aortic repair device in accordance with embodiments of the present technology.

FIGS. 85A and 85B are a perspective side view and a distally-facing perspective end view, respectively, of a base member of an aortic repair device in accordance with embodiments of the present technology.

FIG. 86 is a side view of an aortic repair device in accordance with embodiments of the present technology.

FIGS. 87A and 87B are side views of a spanning member in a first position and a second position, respectively, in accordance with embodiments of the present technology.

FIG. 87C is an enlarged side view of a portion of the spanning member of FIGS. 87A and 87B in accordance with additional embodiments of the present technology.

FIGS. 88A and 88B are perspective side views of a spanning member of an aortic repair device in a first position and a second position, respectively, in accordance with embodiments of the present technology.

FIG. 88C is an enlarged side view of a portion of the spanning member of FIGS. 88A and 88B illustrating two different potential attachments of elastic fibers of a tension member in accordance with embodiments of the present technology.

FIGS. 89A and 89B are perspective side views of a base member of an aortic repair device in a first position and a second position, respectively, in accordance with embodiments of the present technology.

FIGS. 90A and 90B are enlarged views of a pair of stents of a component of an aortic repair device in a free state and a compacted state, respectively, in accordance with embodiments of the present technology.

FIGS. 90C and 90D are enlarged views of the stents of FIGS. 90A and 90B in the free state and the compacted state, respectively, after attachment of one or more tensioning sutures to the stents in accordance with embodiments of the present technology.

FIGS. 90E and 90F are schematic sides views of the aortic repair device of FIGS. 90C and 90D in the free state and the compacted state, respectively, in accordance with embodiments of the present technology.

FIGS. 91A and 91B are enlarged views of the stents of FIGS. 90A-90D in the free state and the compacted state, respectively, in accordance with additional embodiments of the present technology.

### DETAILED DESCRIPTION

The present technology is directed to devices for treating a diseased aorta of a patient, such as a human patient, and associated systems and methods. In some embodiments, for example, an aortic repair device includes a base member configured to be implanted at least partially in a proximal region of an aorta proximate to a diseased portion of the aorta, such as an aneurysm or dissection. The base member can provide a conduit for directing blood flow past the diseased portion of the aorta to perfuse a portion of the aorta distal of the diseased portion and/or various branch vessels. In some embodiments, one or more spanning members or other implants can be coupled (e.g., docked) to the base member during the same or a separate intravascular procedure to provide additional flow conduits past the diseased portion.

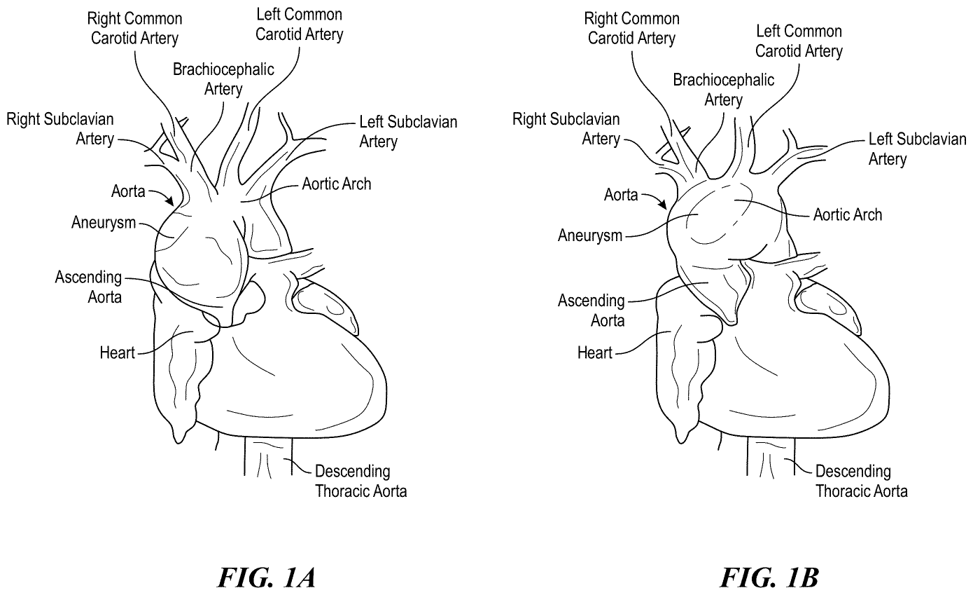

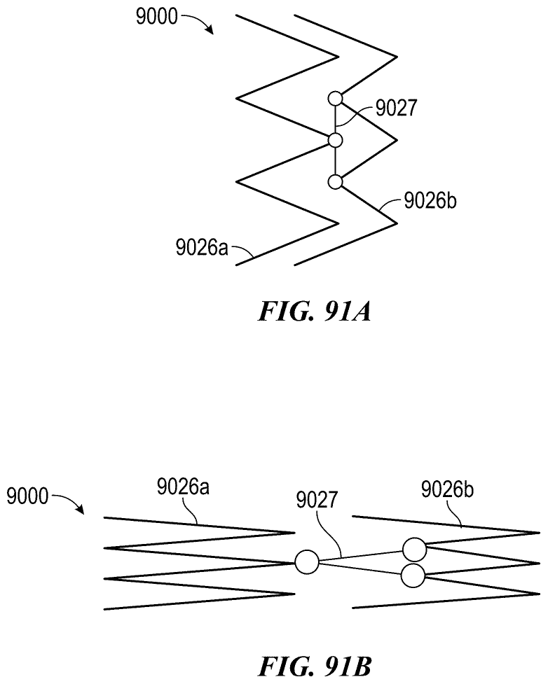

Specific details of several embodiments of the present technology are described herein with reference to FIGS. 1-91B. The present technology, however, can be practiced without some of these specific details. In some instances, well-known structures and techniques often associated with catheter-based delivery systems, implantable repair devices, and the like, have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

When used with reference to an aortic repair device, the term “distal” can reference a portion of the aortic repair device positioned and/or configured to be positioned farther from the heart and downstream in the path of blood flow, while the term “proximal” can reference a portion of the aortic repair device positioned and/or configured to be positioned closer to the heart and upstream in the path of blood flow. In contrast, when used with reference to a catheter subsystem or delivery procedure, the term “distal” can reference a portion of the catheter system farther from an operator and/or handle while the term “proximal” can reference a portion of the catheter system closer to the operator and/or handle. Accordingly, as set forth below, often a proximal portion of an aortic repair device is farther from the operator and/or handle of a catheter system used to deliver the aortic repair device than a distal portion of the aortic repair device. Likewise, often a distal portion of the aortic repair device is closer to the operator and/or handle of the catheter system than the proximal portion.

Also, as used herein, the designations “rearward,” “forward,” “upward,” “downward,” etc., are not meant to limit the referenced component to use in a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### I. OVERVIEW

FIGS. 1A and 1B are side views of a diseased aorta and surrounding anatomy in which an aortic repair device can be implanted in accordance with embodiments of the present technology. Referring to FIGS. 1A and 1B together, the aorta is the largest vessel in the human body and carries oxygenated blood away from the left ventricle and the aortic valve of the heart for circulation to all parts of the body. The aorta is divided into different segments including the ascending aorta (which extends from the left ventricle), the aortic arch, and the descending thoracic aorta. The ascending aorta and the aortic arch can together be referred to as the “proximal aorta.” The aorta includes branches into several supra-aortic arteries including the brachiocephalic artery, the left common carotid artery, and the left subclavian artery—each of which extends from the aortic arch. The brachiocephalic artery is the first branch of aortic arch and feeds blood flow to the right common carotid artery and the right subclavian artery for supply to the right arm, head, and neck. It is also known as the innominate artery or the brachiocephalic trunk. The left common carotid artery is the second branch of the aortic arch and feeds blood flow to the left head and neck. The left subclavian artery is the third branch of the aortic arch and feeds blood flow to the left arm.

The diseased aorta includes an aneurysm in the ascending aorta in FIG. 1A and an aneurysm in the aortic arch in FIG. 1B. Aortic aneurysms are enlargements (e.g., dilations) of the aorta that weaken the aorta and increase the likelihood of rupture. Most people with aortic aneurysms do not have symptoms until the aorta ruptures, and the mortality rate after an aortic aneurysm rupture can exceed 90%. Often, aortic aneurysms are detected during routine medical testing such as chest X-rays, computed tomography (CT) imaging, ultrasound imaging of the heart, magnetic resonance imaging (MRI), and the like.

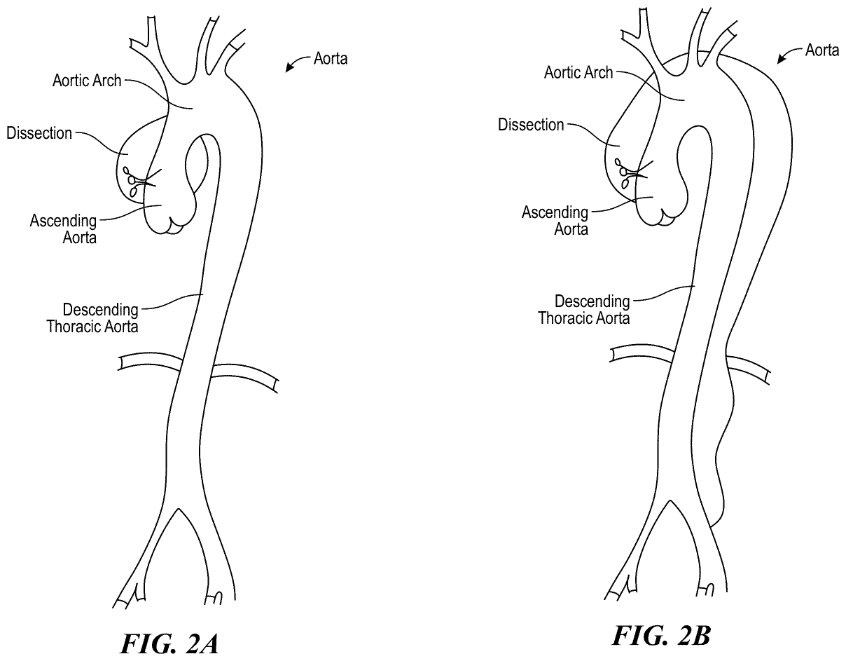

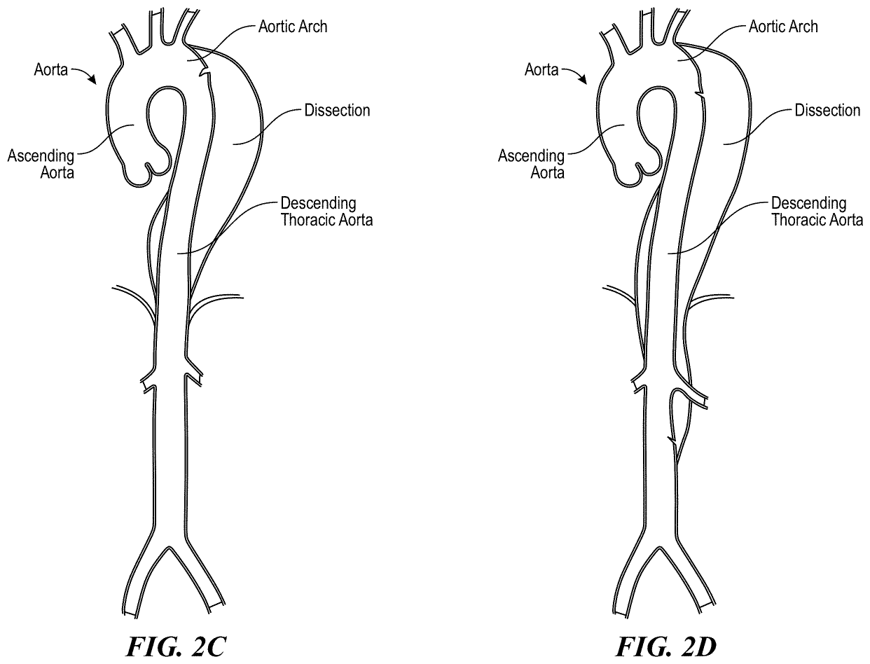

FIGS. 2A-D are side cross-sectional views of a diseased aorta in which an aortic repair device can be implanted in accordance with embodiments of the present technology. The diseased aorta includes a smaller type A aortic dissection in FIG. 2A, and a larger Type A aortic dissection in FIG. 2B. The diseased aorta includes a smaller type B aortic dissection in FIG. 2C, and a larger Type B aortic dissection in FIG. 2D. Aortic dissections are tears of the intimal layer of the aorta that cause blood to dissect the intimal layer and block flow. Type A aortic dissections originate in the proximal aorta (e.g., the ascending aorta) and can progressively extend from the ascending aorta, along the aortic arch, and/or down along the descending aorta as shown in FIG. 2B. Type B aortic dissections originate in the descending aorta and can progressively extend down along the descending aorta as shown in FIG. 2D. Aortic dissections can be acute or chronic, with acute aortic dissections frequently causing a sudden onset of severe pain in the chest, back, and/or abdomen. Most acute aortic dissections require emergency surgery, and present about a 1% risk of death every hour for the first two days after dissection-meaning that urgent diagnosis and surgery are critical for patient treatment.

Referring to FIGS. 1A-2D together, aspects of the present technology are directed to aortic repair devices that can be implanted within the aorta to treat an aortic aneurysm, a Type A aortic dissection, a Type B aortic dissection, and/or other types of diseased states. In some embodiments, an aortic repair device can span across the origin of an aneurysm or dissection and provide one or more flow conduits for diverting blood flow away from and/or past the diseased portion. In some embodiments, an aortic repair device can span between and provide one or more flow conduits for directing blood flow between the aorta and one or more branching arteries.

### II. SELECTED EMBODIMENTS OF AORTIC REPAIR DEVICES INCLUDING AN INTERNAL SEPTUM AND/OR INTERNAL ATTACHMENT MECHANISM

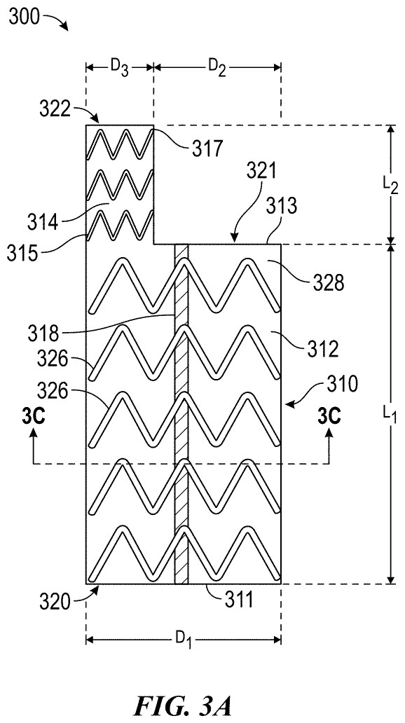

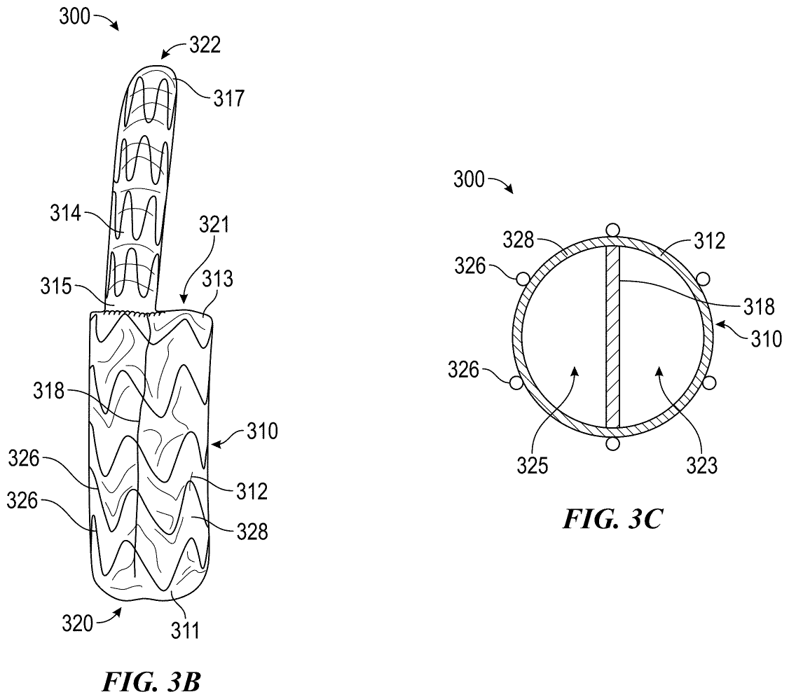

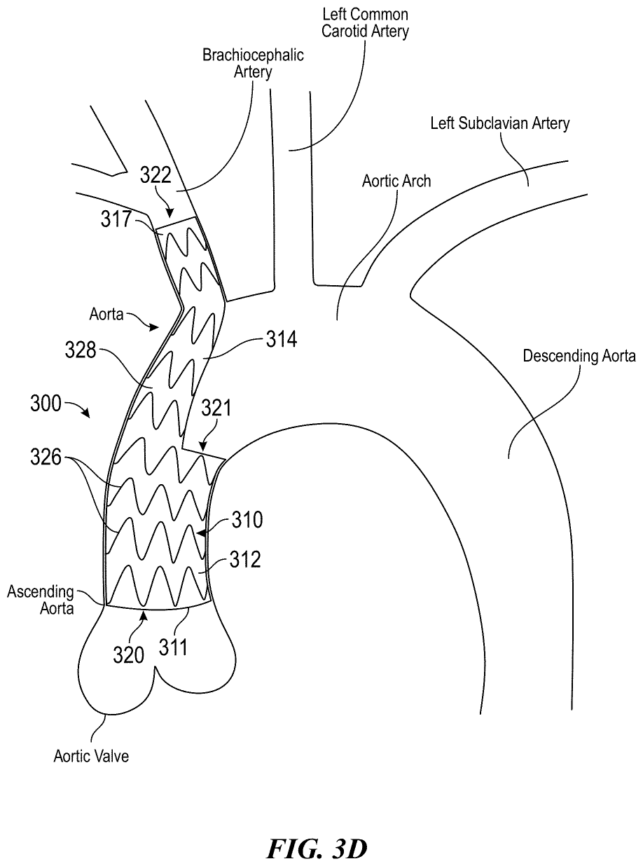

FIGS. 3A and 3B are a side view and a perspective side view, respectively, of an aortic repair device 300 (which can also be referred to as an aortic prosthesis, an aortic treatment device, an aortic implant, and/or the like) in accordance with embodiments of the present technology. Referring to FIGS. 3A and 3B together, in the illustrated embodiment the aortic repair device 300 comprises a base member 310 configured to be implanted in a diseased aorta. The base member 310 includes a tubular body 312 (e.g., a main body) and a tubular leg 314 extending distally from the body 312. More specifically, the body 312 includes a proximal (e.g., first, leading) end portion 311 defining a proximal (e.g., first, leading) terminus of the base member 310 and a distal (e.g., second, trailing) end portion 313. The leg 314 includes a proximal (e.g., first, leading) end portion 315 coupled to and/or integrally extending form the distal end portion 313 of the body 312 and a distal (e.g., second, trailing) end portion 317 defining a distal (e.g., second, trailing) terminus of the base member 310. In the illustrated embodiment, the body 312 has a first length L₁ (FIG. 3A) and the leg 314 as a second length L₂ (FIG. 3A) less than the first length L₁. In some embodiments, the second length L₂ is the same as or greater than the first length L₁. In some embodiments, the first length L₁ can be between about 20-120 millimeters and the second length L₂ can be between about 20-120 millimeters.

FIG. 3C is a cross-sectional view of the aortic repair device 300 of FIGS. 3A and 3B taken along the line 3C-3C in FIG. 3A in accordance with embodiments of the present technology. Referring to FIGS. 3A-3C together, the base member 310 can be generally hollow and define one or more lumens (e.g., flow conduits) therethrough. Accordingly, in the illustrated embodiment the base member 310 includes (i) a proximal opening 320 (e.g., a fluid opening, a first opening, a first body fluid opening, an inlet, and/or the like) at the proximal end portion 311 of the body 312 and having a first diameter D₁ (FIG. 3A), (ii) a distal body opening 321 (e.g., a fluid opening, a second opening, a second body fluid opening, a distal body outlet, and/or the like) at the distal end portion 313 of the body 312 and having a second diameter D₂ (FIG. 3A), and (iii) a distal leg opening 322 (e.g., a fluid opening, a third opening, a leg fluid opening, a distal leg outlet, and/or the like) the distal end portion 317 of the leg 314 and having a third diameter D₃ (FIG. 3A). The first diameter D₁ is larger than the second and third diameters D₂, D₃ and can be sized to generally match or be larger than (e.g., oversized relative to) the diameter of a patient's aorta (e.g., between about 20-60 millimeters, between about 26-54 millimeters, about 40 millimeters). The third diameter D₃ can be sized to generally match or be larger than a branch vessel of the patient's aorta, such as the brachiocephalic artery (e.g., between about 10-22 millimeters, about 16 millimeters). In some embodiments, the second diameter D₂ can be larger than the third diameter D₃.

In some embodiments, the base member 310 further includes a septum 318 (e.g., a flow divider) positioned within the body 312 and dividing the body 312 into a primary lumen 323 (FIG. 3C; e.g., a first lumen) and a branch or secondary lumen 325 (FIG. 3C; e.g., a second lumen). The body 312 is shown as partially transparent in FIG. 3A so that the septum 318 is visible, and the septum is partially obscured in FIG. 3B. In the illustrated embodiment, the septum 318 (i) extends entirely through the body 312 between the proximal and distal end portions 311, 313 and (ii) is centered within the body 312 such that the septum 318 extends along the first diameter D₁ and the primary and secondary lumens 323, 325 have the same size (e.g., cross-sectional area, volume) within the body 312. As described in greater detail below, in other embodiments the septum 318 can (i) extend only partially through the body 312 and/or (ii) can be offset within the body 312 such that the primary and secondary lumens 323, 325 have different sizes. In yet other embodiments described below, the body 312 can include multiple septa that divide the body into more than two lumens.

The primary lumen 323 can extend from and define a flow path (e.g., conduit) between the proximal opening 320 and the body opening 321. Similarly, the secondary lumen 325 can extend from and define a flow path between the proximal opening 320 and the leg opening 322. As shown in FIG. 3C, the primary lumen 323 and the secondary lumen 325 can each have a D-like cross-sectional shape within the body 312 as the septum 318 bifurcates the lumen defined by the body 312. The secondary lumen 325 can have a circular cross-sectional shape within the leg 314.

In some embodiments, the base member 310 is an expandable stent graft comprising one or more stents 326 and a graft material 328. The stents 326 can comprise one or multiple interconnected struts and can also be referred to as a stent structure. The body 312 and the leg 314 can be integrally formed as part of the same stent graft, or can be separate components that are releasably or permanently coupled together. The graft material 328 can comprise fabric, woven polyester, polytetrafluoroethylene, polyurethane, silicone, and/or other suitable materials known in the art of stent grafts, and is configured to inhibit or even prevent blood flow therethrough. In some embodiments, the septum 318 comprises the same material as the graft material 328 or another type of graft material. Accordingly, the graft material 328 and the septum 318 can define and enclose the primary lumen 323 and the secondary lumen 325 and are configured to maintain blood flowing along the flow paths defined thereby. As described in further detail below, in some embodiments the septum 318 can comprise a non-graft material such as a metal mesh that is permeable to blood. In some such embodiments, the aortic repair device 300 can include one or more sealing features positioned proximate the distal end portion 313 of the body 312.

The stents 326 can extend circumferentially to define the tubular shape of the body 312 and the leg 314 and can be interconnected or separate. In some embodiments, the stents 326 have the illustrated V-pattern shape (e.g., including alternating proximal and distal apices). The stents 326 can be coupled to an outer surface of the graft material 328 as shown in FIGS. 3A-3C via stitching and/or suitable techniques, and/or can be coupled to an inner surface of the graft material 328. The stents 326 can be configured to self-expand and, accordingly, can be formed from a shape memory material, such as nickel-titanium alloy (nitinol). In other embodiments, the shape of the stents 326, the number of the stents 326, and/or the arrangement of the stents 326 can be varied.

FIG. 3D is a side view of the aortic repair device 300 implanted within an aorta in accordance with embodiments of the present technology. In some embodiments, the aorta can include an aneurysm, dissection, and/or other diseased portion as described in detail above with reference to FIGS. 1A-2B. In the illustrated embodiment, the body 312 is implanted within the proximal aorta (e.g., the ascending aorta and/or the aortic arch) with the proximal end portion 311 positioned proximate to the aortic valve, and the leg 314 extends from the body 312 to the brachiocephalic artery where the distal end portion 317 is positioned. Referring to FIGS. 3A-3D together, the stents 326 can expand the graft material 328 into contact with the inner wall of the aorta and/or the brachiocephalic artery to provide a seal between the vessel and the base member 310. More particularly, the body 312 can sealingly contact the inner wall of the proximal aorta such that all or substantially all blood flow through the aorta enters the proximal opening 320 and flows through either the primary lumen 323 or the secondary lumen 325. As shown in FIG. 3D, the base member 310 can direct the blood flow (i) through the primary lumen 323 and out of the body opening 321 into the aorta to perfuse the aorta and (ii) through the secondary lumen 325 and out of the leg opening 322 into the brachiocephalic artery to perfuse the brachiocephalic artery.

In some aspects of the present technology, the base member 310 can be positioned against/adjacent to a diseased portion of the aorta, such as the origin of a dissection and/or aneurysm, to block blood flow into the diseased portion by diverting the blood flow through the base member 310 and past the diseased portion. In additional aspects of the present technology, the entire outer surface of the body 312 has a tubular shape configured to engage and seal with the inner wall of the aorta. That is, the body 312 can provide a relatively long sealing region along all or substantially all of the first length L₁.

The base member 310 can be delivered to the aorta in a collapsed configuration within a catheter system (not shown). The catheter system can access the aorta via any suitable intravascular path-such as an aortic approach, a transfemoral approach, a transcarotid approach, a transsubclavian approach, and so on.

In some embodiments, as described in detail below, other implantable devices can be modularly coupled to the base member 310 during the same or a different surgical/interventional procedure. For example, the length L₂ of the leg 314 can be relatively short such that the distal end portion 317 of the leg 314 is positioned in the aorta, and a separate tubular stent graft or other implant can be coupled to the leg 314 and extend into the brachiocephalic artery or another branch vessel. Likewise, a separate tubular sent graft or other implant can be coupled to the body 312 and extend farther through the aorta and/or to a branch vessel.

As described in detail below, in some embodiments the base member 310 can be implanted within the descending aorta in a reversed or flipped orientation. Accordingly, in a flipped orientation, the “distal” portions of the base member 310 are instead “proximal” portions of the base member 310 and vice versa. Therefore, one of ordinary skill in the art will understand that the use of the terms “proximal” and “distal” can be based on the orientation of the base member 310 or can refer to the corresponding structure of the base member 310 (i.e., the “distal” and “proximal” portions can refer to the corresponding structure of the base member 310 described in detail herein regardless of the actual orientation of the base member 310 within the aorta). Likewise, the terms “distal” and “proximal” can be substituted with the terms “first” and “second,” “leading” and “trailing,” and/or the like.

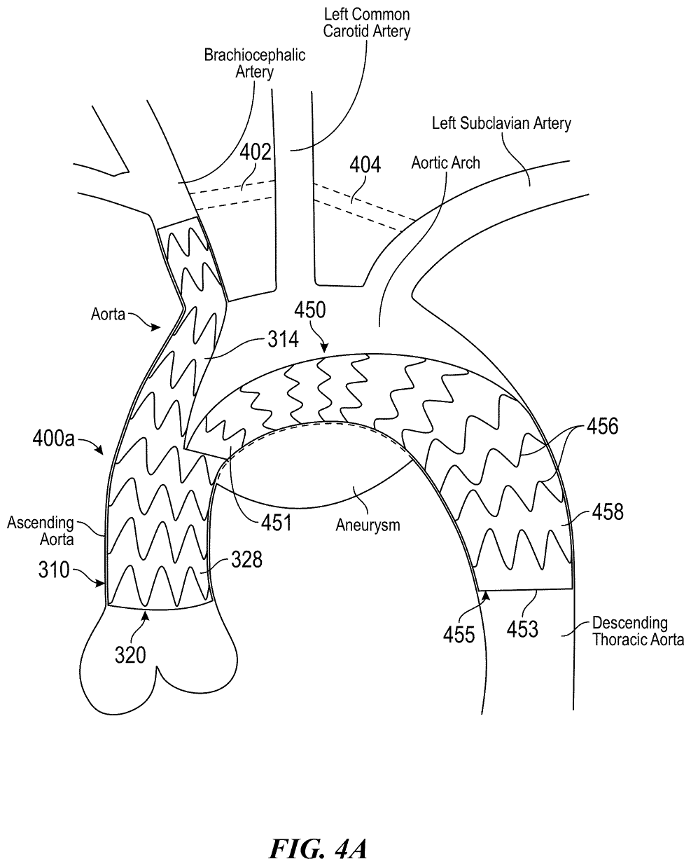

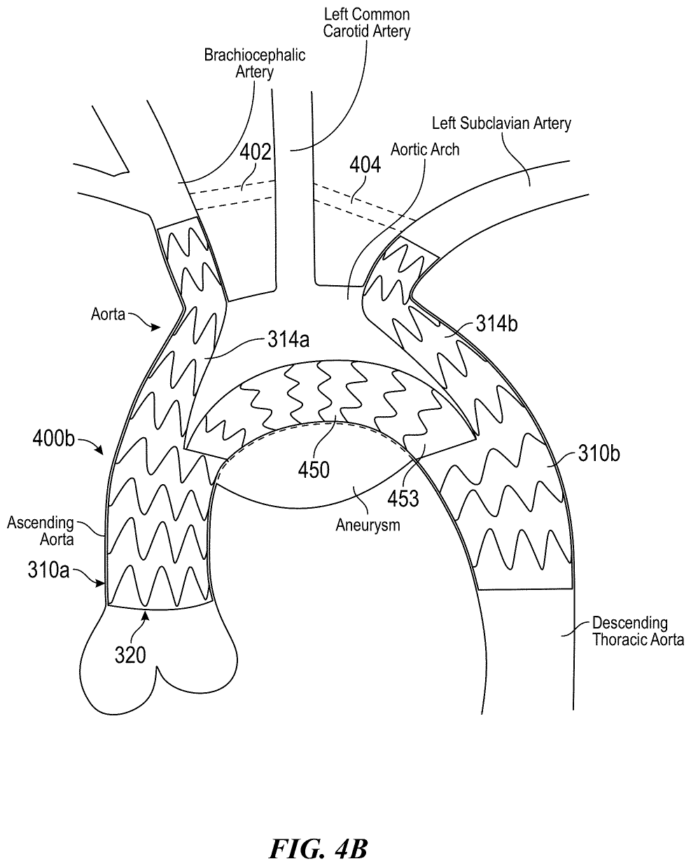

FIG. 4A is a side view of an aortic repair device 400a implanted within an aorta in accordance with embodiments of the present technology. In the illustrated embodiment, the aortic repair device 400a includes the base member 310 described in detail above with reference to FIGS. 3A-3D, and a separate spanning member 450 coupled to (e.g., attached to, docked to) the base member 310. The spanning member 450 can have a tubular shape defining a lumen and can include features generally similar to those of the base member 310. For example, in some embodiments the spanning member 450 is an expandable stent graft comprising one or more struts or stents 456 and a graft material 458 coupled to the stents 456.

In the illustrated embodiment, the spanning member 450 includes a proximal end portion 451 (e.g., a first end portion, a leading end portion, and/or the like; partially obscured by the base member 310) defining a proximal terminus of the spanning member 450 and a distal end portion 453 (e.g., .g., a second end portion, a trailing end portion, and/or the like) defining a distal terminus of the spanning member 450. The spanning member 450 can be generally hollow and define one or more lumens (e.g., flow conduits) therethrough. Accordingly, in the illustrated embodiment the spanning member 450 includes a proximal opening (e.g., a fluid opening, a first spanning fluid opening, a leading spanning fluid opening, and/or the like) obscured by the base member 310) and a distal opening 455 (e.g., a fluid opening, a second spanning fluid opening, a trailing spanning fluid opening, and/or the like).

Referring to FIGS. 3A-4A together, the proximal end portion 451 of the spanning member 450 is at least partially positioned within the body 312 within the primary lumen 323 and can sealingly engage the body 312 within the primary lumen 323 to define a continuous blood flow path from the proximal opening 320 of the base member 310, through the primary lumen 323, and through the lumen of the spanning member 450 to the distal opening 455. That is, the graft material 458 of the spanning member 450 can sealingly engage the septum 318 and the graft material 328 of the base member 310 within the primary lumen 323 such that blood flow is routed through the primary lumen 323 to the lumen of the spanning member 450. In some aspects of the present technology, because the septum 318 extends the entire length of the body 312, the septum 318 provides a docking or sealing region for engaging the spanning member 450 along the entire length of the primary lumen 323. In some embodiments, at least a portion of the spanning member 450 (e.g., the distal end portion 453) sealingly engages the aorta within the aortic arch and/or the descending thoracic aorta. Accordingly, the aortic repair device 400 can direct blood flow through the secondary lumen 325 to the brachiocephalic artery and through the primary lumen 323 and the spanning member 450 to the descending thoracic aorta.

Accordingly, in some aspects of the present technology the aortic repair device 400 can divert blood flow past a diseased portion of the aorta, such an aneurysm in the aortic arch shown in FIG. 4. In some embodiments, the spanning member 450 can be delivered to the aorta in a collapsed configuration within a catheter system (not shown) in the same or a separate procedure as the base member 310. For example, often a patient may initially only need treatment within the ascending aorta and/or the aortic arch to treat an initial dissection or aneurysm but, at a later time (e.g., months or years later), may require additional treatment of the aortic arch and/or the descending thoracic aorta as the diseased state progresses. Accordingly, the base member 310 can be implanted during an initial procedure and the spanning member 450 can be implanted during a later procedure and modularly coupled to the base member 310 to provide further treatment of the aortic arch and/or the descending thoracic aorta (e.g., by bypassing blood flow past the aneurysm shown in FIG. 4).

Referring to FIG. 4A, the spanning member 450 bypasses the left common carotid artery and the left subclavian artery and thus may partially or fully occlude those vessels. In some embodiments, a bypass 402 between the perfused brachiocephalic artery and/or a bypass 404 between the left common carotid artery and the left subclavian artery (and/or between the brachiocephalic artery and the left subclavian artery) can be surgically created to perfuse those vessels. In other embodiments described in detail below, the aortic repair device 400 can include additional implantable devices (e.g., stent grafts) coupled to the spanning member 450 and/or the base member 310 that are configured (e.g., sized, shaped, positioned) to perfuse different branch vessels. Similarly, in some embodiments the spanning member 450 does not sealingly engage the aorta such that blood can flow in retrograde to the left subclavian artery and the left common carotid artery to perfuse those vessels.

FIG. 4B is a side view of an aortic repair device 400b implanted within an aorta in accordance with embodiments of the present technology. In the illustrated embodiment, the aortic repair device 400a includes the base member 310 described in detail above with reference to FIGS. 3A-3D (identified as first base member 310a) and the spanning member 450 coupled to (e.g., attached to, docked to) the first base member 310a. In the illustrated embodiment, the aortic repair device 400b further includes a second base member 310b (which can be identical or substantially identical to the first base member 310a) implanted at least partially within the descending thoracic aorta. The spanning member 450 spans from the base member 310a and is coupled to the second base member 310b. Specifically, with reference to FIGS. 3A-4B together, the distal end portion 453 of the spanning member 450 can be positioned within the body 312 within the primary lumen 323 of the second base member 310b and can sealingly engage the body 312 within the primary lumen 323 to define a continuous blood flow path from the spanning member 450 through the primary lumen 323, and out of the proximal opening 320 of the second base member 310b. The leg 314b of the second base member 310b can extend into a second branch artery (e.g., the left subclavian artery) for perfusing the second branch artery. For example, the second base member 310b can receive retrograde blood flow through the trailing opening 320 for perfusing the second branch artery. Alternatively or additionally, the secondary lumen 325 of the second base member 310b can be perfused via the primary lumen 323 where the septum 318 extends only partially from the leading end portion 313 toward the trailing end portion 311. That is, blood flow from the spanning member 450 can flow into the primary lumen 323 within the body 312 and around the septum 318 into the secondary lumen 325 where the septum 318 terminates within the body 312. In some aspects of the present technology, the aortic repair device 400b can provide for a full arch treatment in which (i) the leg 314a of the first base member 310a directs blood flow to a first branch artery (e.g., the brachiocephalic artery), (ii) the leg 314b of the second base member 310b directs blood flow to a second branch artery (e.g., the left subclavian artery), (iii) a third branch artery (e.g., the left common carotid artery) is perfused via bypass 402 and/or 404, and (iv) the primary lumens of the first and second base members 310a-b and the spanning member 450 collectively direct blood flow to the descending thoracic aorta.

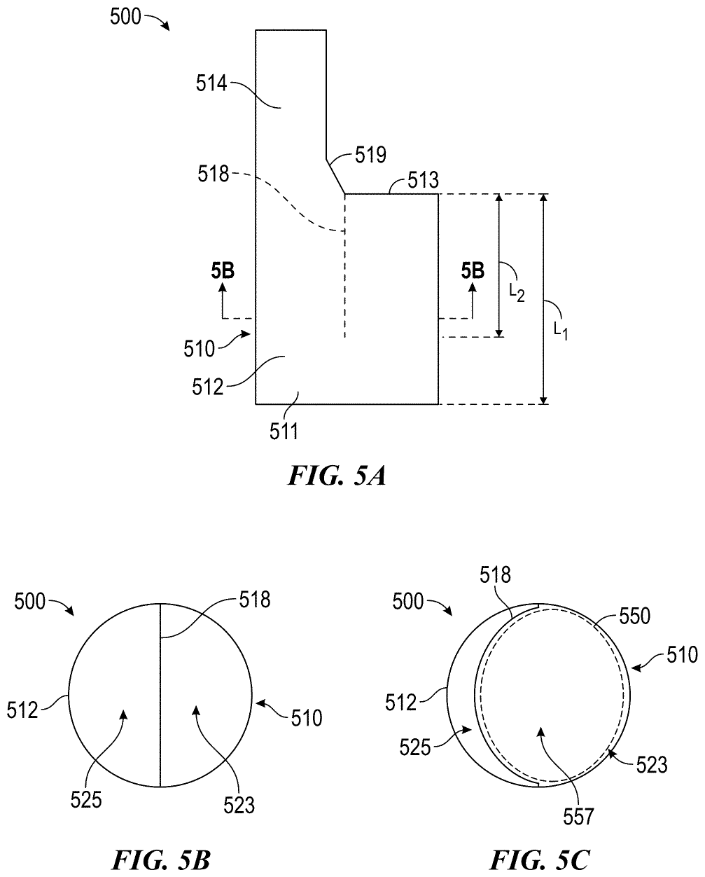

FIGS. 5A-8H illustrate additional embodiments of aortic repair devices including a base member and/or a spanning member in accordance with embodiments of the present technology. The various aortic repair devices can include some features that are at least generally similar in structure and function, or identical in structure and function, to one another and/or to the corresponding features of the aortic repair devices 300 and 400 described in detail above with reference to FIGS. 3A-4, and can operate in a generally similar or identical manner to one another and/or to the aortic repair device 300 and 400. Moreover, one or more of the different features of the aortic repair devices of the present technology can be combined and/or omitted.

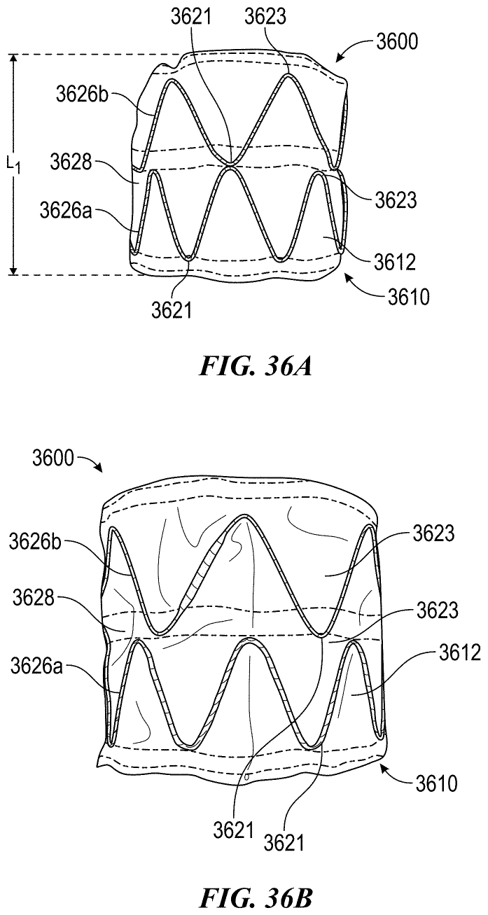

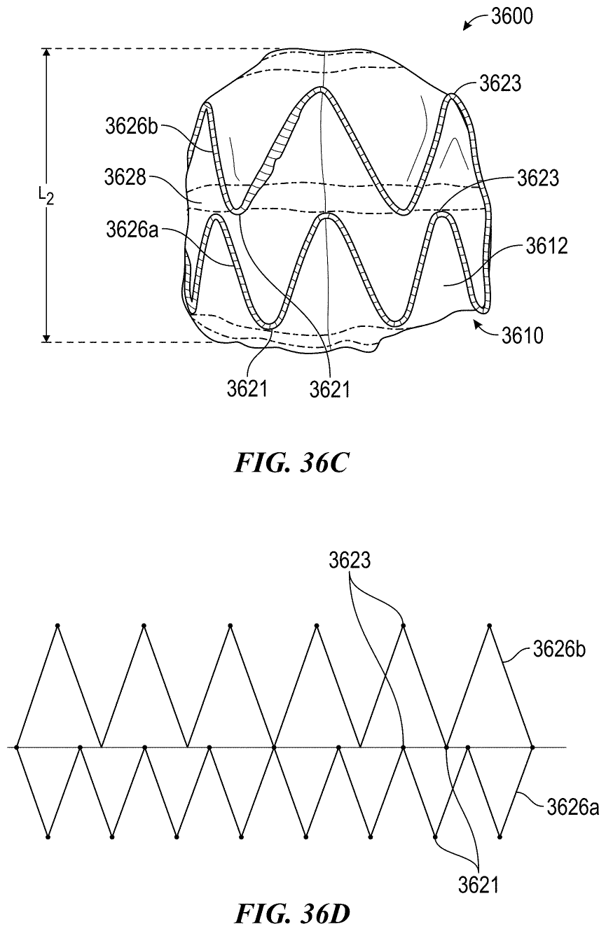

FIG. 5A is a side view of an aortic repair device 500 in accordance with embodiments of the present technology. In the illustrated embodiment, the aortic repair device 500 includes a base member 510 having a body 512 and a leg 514. The body 512 includes a proximal end portion 511, a distal end portion 513, and a septum 518 extending from the distal end portion 513 only partially toward the proximal end portion 511. That is, the septum 518 extends only partially through the body 512 and has a second length L₂ less than a first length L₁ of the body 512. In some aspects of the present technology, the septum 518 reduces a volume (e.g., a total volume and/or a maximum volume per unit length anywhere along a length of the aortic repair device 500) of the base member 510 as compared to a septum extending fully through the body 512 (e.g., with second length L₂ equal to the first length L₁). In some aspects of the present technology, reducing the volume of the base member 510 can reduce the delivery profile of the aortic repair device 500—potentially allowing the aortic repair device 500 to be loaded into and delivered through a relatively smaller catheter and/or delivered along a narrower intravascular delivery pathway. However, the shorter septum 518 can reduce the available area for sealingly engaging (e.g., docking) a spanning member, such as a spanning member 550 shown in FIG. 5C. In the illustrated embodiment, the leg 514 further includes an angled portion 519 extending from the body 512 that reduces the diameter of a secondary lumen defined by the leg 514. Further, because the septum 518 extends only partially through the body 512, the body 512 can define a sealing region near the proximal end portion 511 of the body 512 that can receive an extending member, as described in further detail below with reference to FIG. 36C. Such an extending member can be coupled to the proximal end portion 511 of the body 512 to provide further sealing and/or anchoring within the aorta.

FIGS. 5B and 5C are cross-sectional views of the aortic repair device 500 of FIG. 5A taken along the line 5B-5B in FIG. 5A in accordance with embodiments of the present technology. Referring to FIG. 5B, the body 512 has a generally circular cross-sectional shape and the septum 518 is generally centered within the body 512 such that the septum 518 divides (e.g., bifurcates) the body 512 into a primary lumen 523 and a secondary lumen 525 having the same size. FIG. 5C illustrates the aortic repair device 500 with a spanning member 550 coupled to the base member 510 within the primary lumen 523 and defining a spanning lumen 557. In the illustrated embodiment, an outer surface of the spanning member 550 at least partially sealingly engages the septum 518 and an inner surface of the body 512 such that blood flow is substantially routed through the spanning lumen 557.

In some embodiments, the spanning member 550 deflects the septum 518 away from a center of the body 512 when expanded within the body 512 as shown in FIG. 5C. This deflection can reduce the cross-sectional size of the secondary lumen 525 and a corresponding volumetric flow rate provided by the secondary lumen 525, while increasing the cross-sectional size of and volumetric flow rate provided by the primary lumen 523. In some aspects of the present technology, this change in size of the primary and secondary lumens 523, 525 is advantageous because the primary lumen 523 is positioned to perfuse the aorta and all its branching vessels, while the secondary lumen 525 is positioned to perfuse a branch vessel requiring less blood flow than the aorta. However, in some embodiments the shape of the secondary lumen 525 (e.g., a thin crescent shape) when the spanning member 550 is deployed can restrict blood flow through the secondary lumen 525. Accordingly, as described in detail below, in some embodiments the body 512 can be shaped differently and/or the septum 518 can be positioned differently to facilitate a more uniform blood flow through the primary and secondary lumens 523, 525 and/or a blood flow proportionate to the physiologic needs of a patient through the primary and secondary lumens 523, 525 (e.g., with the aorta receiving more blood flow then branch vessels).

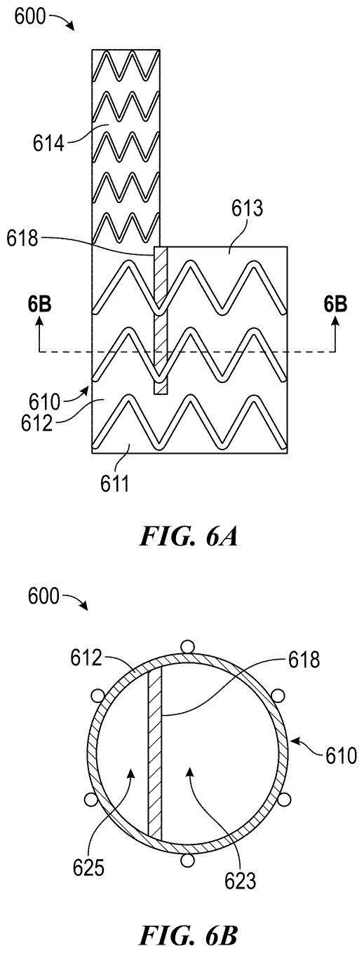

FIG. 6A is a side view of an aortic repair device 600 in accordance with embodiments of the present technology. FIG. 6B is a cross-sectional view of the aortic repair device 600 of FIG. 6A taken along the line 6B-6B in FIG. 6A in accordance with embodiments of the present technology. Referring to FIG. 6A, the aortic repair device 600 includes a base member 610 having a body 612 and a leg 614. The body 612 includes a proximal end portion 611, a distal end portion 613, and a septum 618 extending from the distal end portion 613 only partially toward the proximal end portion 611. That is, the septum 618 can terminate a distance from the proximal end portion 611. Referring to FIG. 6B, the body 612 has a generally circular cross-sectional shape and the septum 618 is offset within the body 612 such that the septum 618 divides the body 612 into a primary lumen 623 and a secondary lumen 625 having a smaller size and corresponding volumetric flow rate than the primary lumen 623. In some embodiments, the septum 618 is configured not to deflect or to only slightly deflect when a spanning member is deployed within the primary lumen 623.

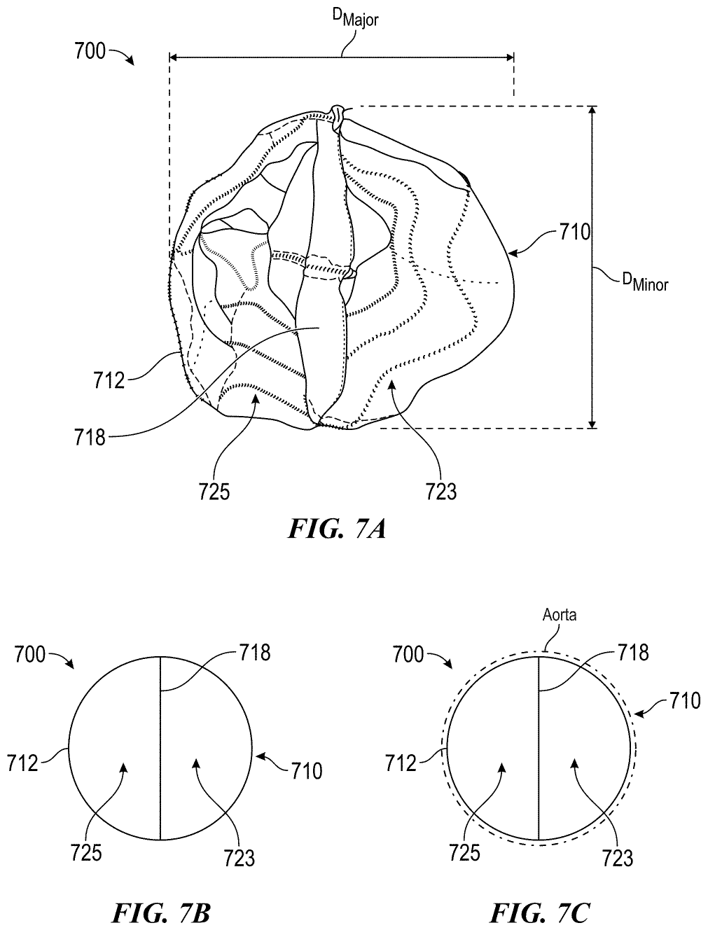

FIGS. 7A and 7B are a distally-facing perspective end view and a distally-facing end view, respectively, of an aortic repair device 700 in accordance with embodiments of the present technology. Referring to FIGS. 7A and 7B together, the aortic repair device 700 includes a base member 710 having a body 712 with a septum 718 extending fully or partially therethrough and defining a primary lumen 723 and a secondary lumen 725. In the illustrated embodiment, when the aortic repair device 700 is expanded without radial constraint (e.g., outside of an aorta), the body 712 has an oval cross-sectional shape including a minor diameter D_{Minor} (FIG. 7A) and a major diameter D_{Major} (FIG. 7A). The septum 718 can extend generally along the minor diameter D_{Minor} and can have length longer than the minor diameter D_{Minor} such that the septum 718 is loose or floppy (e.g., not fully tensioned) when the base member 710 is expanded outside the aorta. In some embodiments, the septum 718 can have a length that generally equal to the minor diameter D_{Minor} such that the septum 718 is taut (e.g., has minimal floppiness).

FIG. 7C is a distally-facing end view of the aortic repair device 700 expanded within an aorta in accordance with embodiments of the present technology. Referring to FIGS. 7A-7C together, in some embodiments the body 712 can be oversized relative to the aorta along at least the major diameter D_{Major} such that, when the base member 710 is expanded within the aorta, the aorta compresses and shortens the body 712 along the major diameter D_{Major}. In some embodiments, the minor diameter D_{Minor} can be selected to match the size of the aorta or be oversized relative to the aorta such that the minor diameter D_{Minor} remains generally the same when implanted within the aorta. In other embodiments, the minor diameter D_{Minor} can be selected to be smaller than the size of the aorta such that compression of the body 712 along the major diameter D_{Major} correspondingly lengthens the body 712 along the minor diameter D_{Minor} to increase the tension in the septum 718. That is, in such embodiments, the body 712 can assume a generally circular cross-sectional shape in the aorta with the major diameter D_{Major} decreased and the minor diameter D_{Minor} increased relative to its expanded state outside the aorta. In some aspects of the present technology, the increased tension in the septum 718 can inhibit substantial deflection of the septum 718 by a spanning member (not shown) when the spanning member is deployed within the primary lumen 723 (e.g., deflection of the type shown in FIG. 5C). This can preserve the size of the flow paths through the primary and secondary lumens 723, 725.

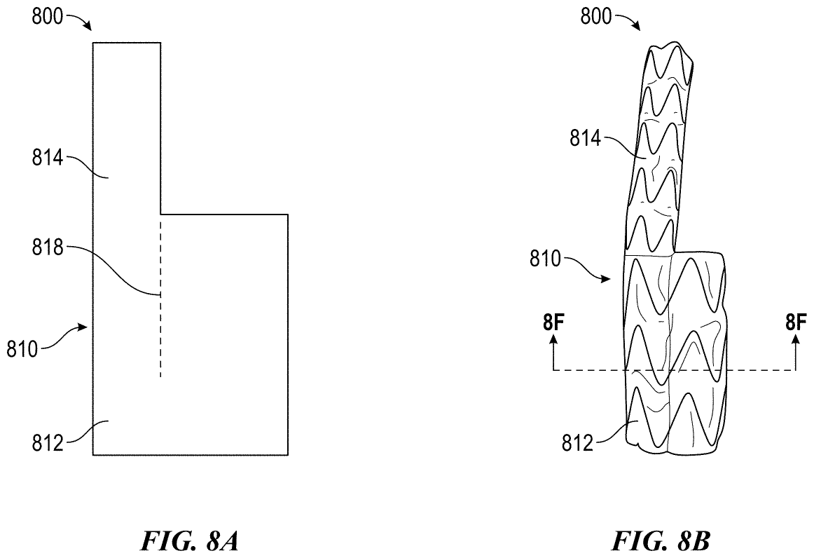

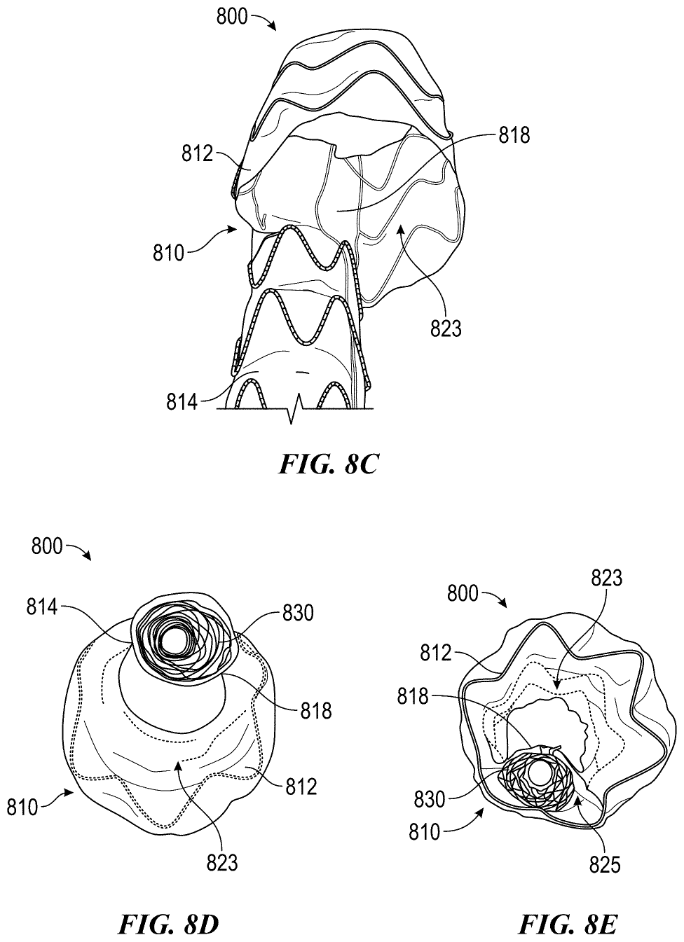

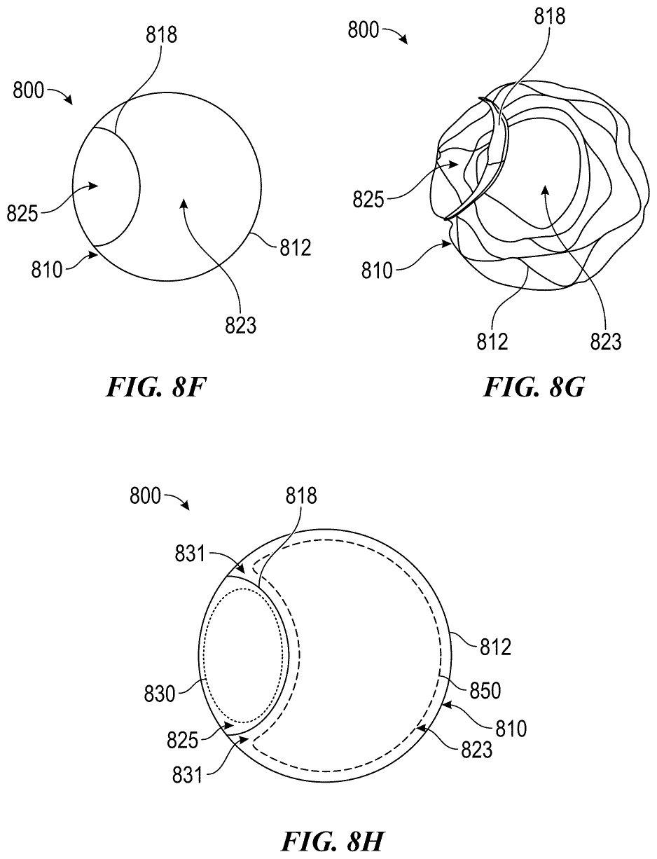

FIGS. 8A-8E are a side view, a perspective side view, a proximally-facing perspective view, a proximally-facing perspective end view, and a distally-facing perspective end view, respectively, of an aortic repair device 800 in accordance with embodiments of the present technology. FIGS. 8F and 8G are cross-sectional views of the aortic repair device 800 taken along the line 8F-8F in FIG. 8B in accordance with embodiments of the present technology. Referring to FIGS. 8A-8G together, the aortic repair device 800 includes a base member 810 having a body 812 with a septum 818 extending fully or partially therethrough. The septum 818 is laterally offset (e.g., as described in detail above with reference to FIGS. 6A and 6B) within the body 812 such that the septum 818 divides the body 812 into a primary lumen 823 and a secondary lumen 825 having a smaller size than the primary lumen 823. The septum 818 can be sized (e.g., have a length) such that, when the base member 810 is expanded, the septum 818 has a curved shape and the secondary lumen 825 has a corresponding curved cross-sectional shape (e.g., a generally circular, generally oval).

Referring to FIGS. 8D and 8E together, the aortic repair device 800 further includes a stent graft or stent 830 positioned within the secondary lumen 825 at least partially within the body 812. The stent 830 can be delivered and deployed within the secondary lumen 825 during the same procedure and/or from the same catheter system used to implant the base member 810, and/or can be delivered to the secondary lumen 825 in a separate procedure and/or from a different catheter system. In some embodiments, the stent 830 is delivered from a separate catheter advanced through a branch lumen in which the outlet of the leg 814 is positioned, such as the brachiocephalic artery. The stent 830 can comprise an expandable mesh of interconnected stents or a similar structure and can have a tubular shape with a generally circular cross-sectional shape. When positioned within the secondary lumen 825, the stent 830 can expand against the septum 818 and the inner surface of the body 812 to expand the secondary lumen 825. Accordingly, when a spanning member is deployed within the primary lumen 823, the stent 830 can help maintain the shape of the secondary lumen 825 preserving the cross-sectional area and flow rate thereof.

For example, FIG. 8H is a cross-sectional view of the aortic repair device taken along the line 8F-8F in FIG. 8B with a spanning member 850 deployed within the primary lumen 823 in accordance with embodiments of the present technology. As shown in FIG. 8H, the spanning member 850 and the stent 830 can conform to the shape of one another. More specially, the stent 830 is configured to exert a greater radial-outward force than the spanning member 850 such that the stent 830 inhibits the septum 818 from substantially deflecting and narrowing the secondary lumen 825, thereby maintaining a good flow path through the secondary lumen 825. As shown in FIG. 8H, the spanning member 850 can slightly compress the stent 830 such that the stent 830 has a generally oval cross-sectional shape, and the spanning member 850 can conform and seal around the septum 818 and the stent 830.

With continued reference to FIG. 8H, in some embodiments the spanning member 850 may not conform fully to the septum 818 and the stent 830 such that one or more gaps 831 exist between the spanning member 850 and the septum 818 and/or the body 812. The gaps 831 may exist along regions of the septum 818 and the body 812 defining sharp corners, narrowed regions, and the like. Such gaps 831 can provide leak paths for blood flow through the aortic repair device 800.

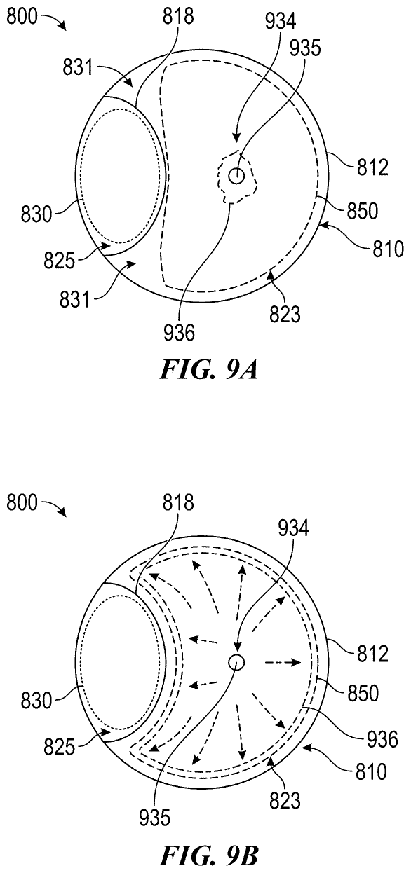

FIGS. 9A-20B illustrate additional embodiments of aortic repair devices including features for inhibiting (e.g., filling, plugging, preventing) gaps, such as the gaps 831, in accordance with embodiments of the present technology. The various aortic repair devices can include some features that are at least generally similar in structure and function, or identical in structure and function, to one another and/or to the corresponding features of the aortic repair devices described in detail above with reference to FIGS. 3A-8H, and can operate in a generally similar or identical manner to one another and/or to the aortic repair devices described above.

FIGS. 9A and 9B, for example, are cross-sectional views of the aortic repair device 800 of FIG. 8H (e.g., end cross-sectional views along a plane perpendicular to a longitudinal axis of the primary lumen 823) during a first delivery stage and a second delivery stage in accordance with embodiments of the present technology. Referring to FIG. 9A, after implanting the aortic repair device 800 within an aorta, a balloon catheter assembly 934 can be advanced to within the spanning member 850. The balloon catheter assembly 934 can include an inflation source 935 and a balloon 936 fluidly coupled to the inflation source 935. The inflation source 935 can include one or more inflation lumens configured to route a fluid into the balloon 936 to inflate/dilate the balloon 936. FIG. 9A shows the balloon 936 before inflation and FIG. 9B shows the balloon 936 after inflation. Referring to FIG. 9B, in some embodiments the balloon 936 has a cross-sectional shape that generally matches the profile of the primary lumen 823, to which the spanning member 950 should conform and seal to. Accordingly, inflating the balloon 936 can help drive the spanning member 950 to conform and seal against the entire circumference of the primary lumen 823—including within the difficult to seal gaps 831. After inflating the balloon 936 to drive the spanning member 950 to conform and seal to the body 812 within the primary lumen 823, the balloon 936 can be deflated and the balloon catheter assembly 934 can be removed from the aortic repair device 800 and the patient.

In some embodiments, the balloon 936 can have a crescent, D-shaped, half circle, quarter circle, or other cross-sectional shape that matches or generally matches the profile (e.g., circumference, perimeter) of the primary lumen 823. In some embodiments, different portions of the balloon 936 can have different compliances to facilitate the non-circular cross-sectional shape. In some embodiments, the inflation source 935 can inflate the balloon 936 according to a predetermined sequence, rate, pressure, and/or the like. In some embodiments, one or more additional balloon catheter assemblies can be inserted into the spanning member 850 and/or the stent 830. For example, the balloon 936 can be a modest semi-compliant balloon having a generally circular cross-sectional shape and can be inflated in the spanning member 950 to the point of partially compressing the stent 830 so that the spanning member 850 and the balloon 936 reach and fill the gaps 831. Then, a non-compliant balloon (not shown) can be inserted into and slowly inflated within the stent 830. As the non-compliant balloon inflates and fills the secondary lumen 825, the non-compliant balloon can slowly deflate the balloon 936 thereby giving up space for expansion but maintaining support in the gaps 831. In some embodiments, pressure in the balloon 936 can be maintained by reducing the inflation pressure of the balloon 936 using the inflation source 935 as the volume of the balloon 936 is compressed and reduced.

In some embodiments, the balloon 936 can have a generally cylindrical shape and can be inflated to fully compress the stent 830. After fully compressing the stent 830, the balloon 936 can be deflated to permit the stent 830 to expand against the septum 818 and the spanning member 850 to recreate the flow path through the secondary lumen 825. In some aspects of the present technology, fully compressing the stent 830 and the septum 818 while positioning the spanning member 850 can facilitate good conformance of the spanning member 850 with the septum 818 reducing or eliminating the gaps 831. In some embodiments, a balloon can be inserted into and expanded within the stent 830 to promote its expansion against the spanning member 850. In other embodiments, the spanning member 850 can be deployed first and the balloon 936 expanded before the stent 830 is deployed within the compressed secondary lumen 825 and allowed to expand.

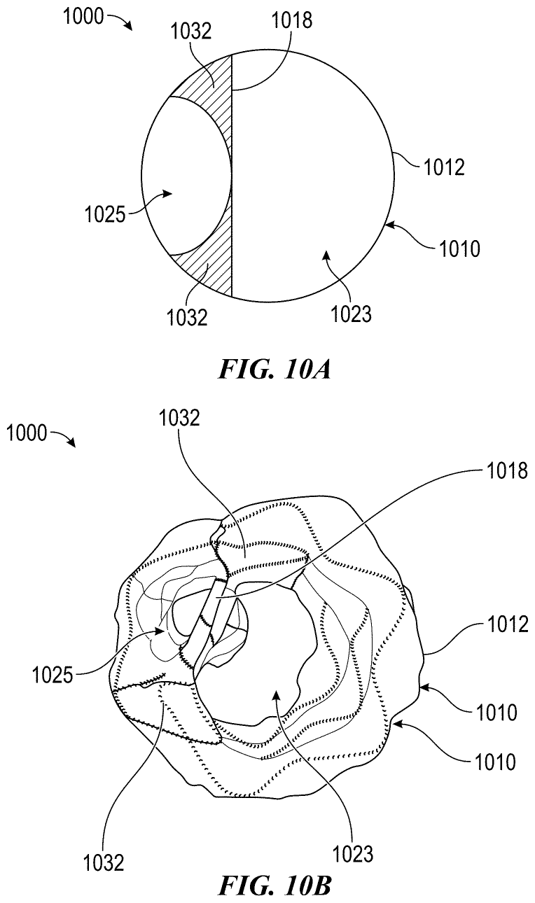

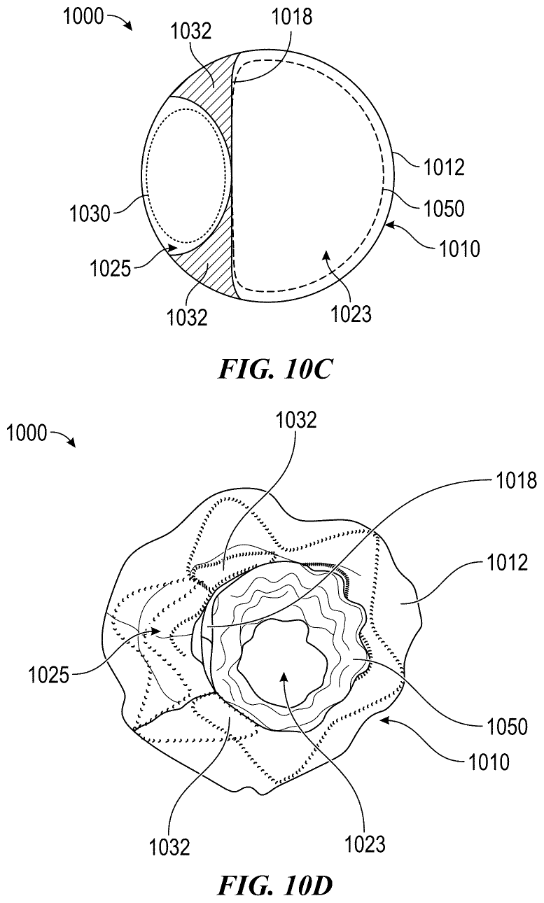

FIGS. 10A and 10B are a distally-facing end view and a perspective end view, respectively, of an aortic repair device 1000 in accordance with embodiments of the present technology. Referring to FIGS. 10A and 10B together, in the illustrated embodiment the aortic repair device 1000 includes a base member 1010 having a body 1012 with a septum 1018 extending fully or partially therethrough. The septum 1018 is laterally offset within the body 1012 such that the septum 1018 divides the body 1012 into a primary lumen 1023 and a secondary lumen 1025 having a smaller size than the primary lumen 1023. In the illustrated embodiment, the base member 1010 further includes one or more filler portions 1032 (e.g., gussets, caps) coupled to the septum 1018 between the primary and secondary lumens 1023, 1025. The filler portions 1032 can comprise fabric and/or materials impermeable to blood flow.

FIGS. 10C and 10D are a distally-facing end view and a perspective end view, respectively, of the aortic repair device 1000 with a spanning member 1050 deployed within the primary lumen 1023 in accordance with embodiments of the present technology. FIG. 10C further illustrates an optional stent 1030 deployed within the secondary lumen 1025. Referring to FIGS. 10C and 10D together, in the illustrated embodiment the filler portions 1032 help define a relatively uniform oval-like cross-sectional shape of the primary lumen 1023. That is, the filler portions 1032 can fill any sharp corners, narrowed, regions and the like (e.g., in the areas around the gaps 831 shown in FIG. 8H) which may be difficult for the spanning member 1050 to conform to. Accordingly, in some aspects of the present technology, the filler portions 1032 can help improve the conformance and sealing of the spanning member 1050 within the body 1012—thereby reducing or eliminating leaks through the aortic repair device 1000 when the device is deployed within an aorta.

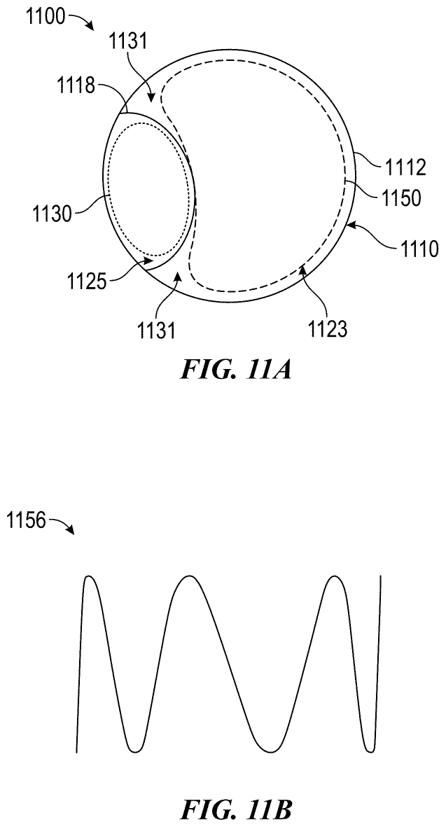

FIG. 11A is a cross-sectional view of an aortic repair device 1100 in accordance with embodiments of the present technology. In the illustrated embodiment, the aortic repair device 1100 includes a base member 1110 having a body 1112 with a septum 1118 extending fully or partially therethrough. The septum 1118 is laterally offset within the body 1112 such that the septum 1118 divides the body 1112 into a primary lumen 1123 and a secondary lumen 1125 having a smaller size than the primary lumen 1123. In the illustrated embodiment, a spanning member 1150 is deployed within the primary lumen 1123 and a stent 1130 is deployed within the secondary lumen 1125. As shown, the spanning member 1150 may not fully conform and seal to the perimeter of the primary lumen 1123 such that one or more gaps 1131 exist.

FIG. 11B is a cross-sectional side view of a stent 1156 of the spanning member 1150 in accordance with embodiments of the present technology. The spanning member 1150 can comprise one or more of the stents 1156 attached to a graft material (not shown; e.g., as described in detail above with reference to FIG. 4). In some embodiments, the stent 1156 has a ring-like shape having a periodic or V-shaped pattern. In the illustrated embodiment, the stent 1156 has a relatively large amplitude and low frequency. In some aspects of the present technology, the relatively large amplitude and low frequency stents 1156 can make the spanning member 1150 relatively easy to manufacture and/or load into a catheter for delivery. However, the spanning member 1150 may not be very conformable as shown in FIG. 11A-causing the gaps 1131.

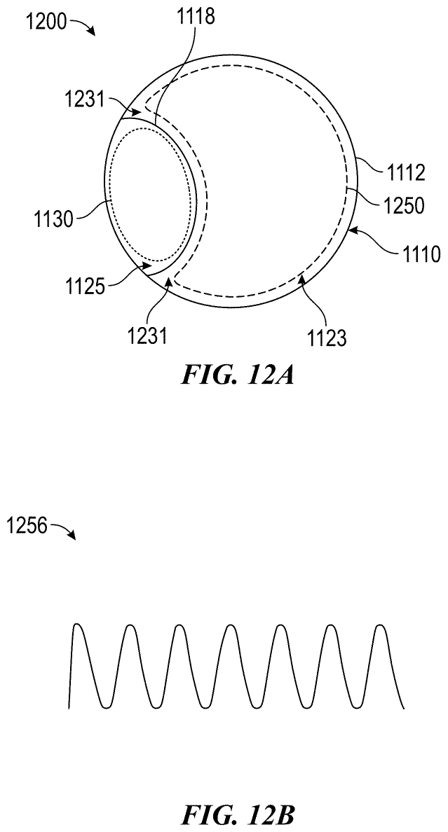

FIG. 12A is a cross-sectional view of an aortic repair device 1200 in accordance with embodiments of the present technology. In the illustrated embodiment, the aortic repair device 1200 includes the base member 1110 and the stent 1130 described in detail with reference to FIG. 11A, but includes a different spanning member 1250. FIG. 12B is a cross-sectional side view of a stent 1256 of the spanning member 1250 in accordance with embodiments of the present technology. In the illustrated embodiment, the stent 1256 has a relatively small amplitude and high frequency (e.g., as compared to the stent 1156 described in detail with reference to FIG. 11B). In some aspects of the present technology, the relatively small amplitude and high frequency stents 1256 can make the spanning member 1250 relatively more difficult to manufacture (e.g., by requiring additional attachments to a graft material) and/or more challenging to load into a catheter for delivery. However, the spanning member 1250 can be more circumferentially conformable as shown in FIG. 12A eliminating or reducing the presence of gaps 1231.

In general, the stents of a spanning member in accordance with the present technology can be selected to balance the radial force (e.g., force apposition) of the spanning member with conformability (e.g., contour adaptability) by modifying one or more of an amplitude, frequency (e.g., number of apices), wall thickness, and/or wire diameter/strut geometry and dimension of the stents. For example, the wall thickness of the spanning member can be reduced to improve the conformability while reducing the radial force. In some embodiments, the manner in which the spanning member lays up on a mating surface can be improved by “scallop” cutting the unsupported V-space at the end portions of the spanning member resulting in somewhat of a sawtooth shape. Further, one or both end portions of the spanning member may be flared outward for improved apposition. Moreover, the spanning member can include a series of stent rings with different properties (e.g., first stent ring at the end can be softer with smaller wire diameter and/or wall thickness and scallops or flaring; a second stent ring can be stiffer with bigger wire diameter and/or wall thickness, shorter amplitude, etc.; third stent ring can be a standard/typical design used in conventional stent grafts; and so on).

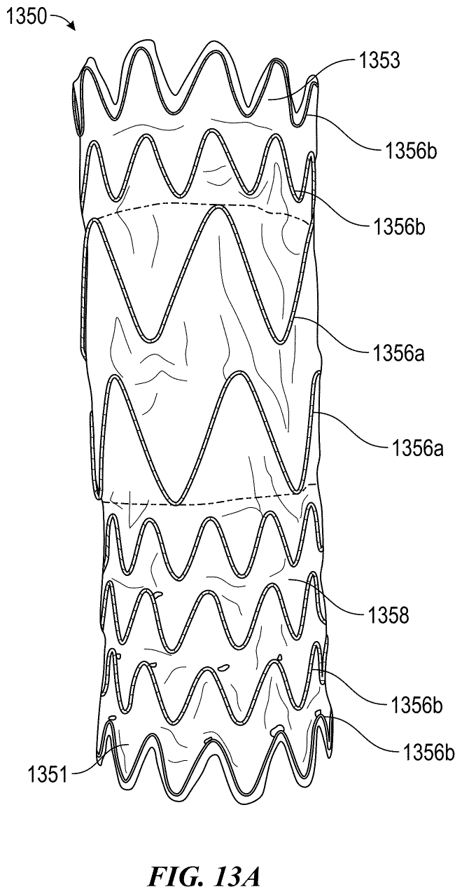

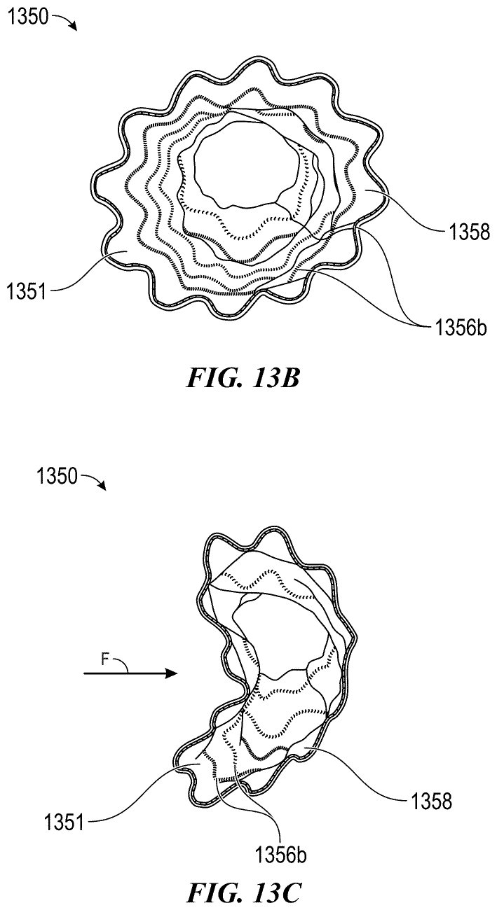

For example, FIGS. 13A-13C are a perspective side view, a distally-facing perspective end view, and another distally-facing perspective end view, respectively, of a spanning member 1350 in accordance with embodiments of the present technology. Referring to FIG. 13A, the spanning member 1350 includes a proximal end portion 1351 configured to expand within and conform to a base member and a distal end portion 1353 configured to expand within and conform to a vessel, such as the aorta (e.g., the aortic arch, the descending thoracic aorta). The spanning member 1350 includes a plurality of stents 1356 (e.g., including individually identified first stents 1356a and second stents 1356b) attached to a graft material 1358. In the illustrated embodiment, the first stents 1356a have a greater amplitude and lower frequency than the second stents 1356b. In some embodiments, the first stents 1356a can have a greater wire diameter than the second stents 1356b.

The second stents 1356b can be positioned at, proximate, and/or adjacent to the proximal and distal end portions 1351, 1353 to provide for increased conformance at the proximal and distal end portions 1351, 1353. FIG. 13C, for example, illustrates the relatively high circumferential conformance of the spanning member 1350 at the proximal end portion 1351 when an outside force indicated by arrow F acts on the spanning member 1350 (e.g., as applied by a stent deployed within a secondary lumen of a base member). The first stents 1356a can be positioned within a middle region of the spanning member 1350. In some embodiments, the spanning member 1350 includes more of the second stents 1356b near the proximal end portion 1351 such that the proximal end portion 1351 is relatively conformable for docking to and sealing with a base member.

Referring to FIGS. 13A-13C together, in some embodiments a most proximal one of the second stents 1356b and/or a most distal one of the second stents 1356b have a larger diameter than the other ones of the stents 1356 such that the spanning member 1350 is flared at one or both of the proximal and distal end portions 1351, 1353. In the illustrated embodiment, the graft material 1358 can be cut to track the pattern of the most proximal one of the second stents 1356b and/or the most distal one of the second stents 1356b such that the graft material 1358 has a scalloped or V-pattern at the one or both of the proximal and distal end portions 1351, 1353. In some aspects of the present technology, such flaring and/or scalloping of the proximal and distal end portions 1351, 1353 can improve the conformance and sealing properties of the spanning member 1350.

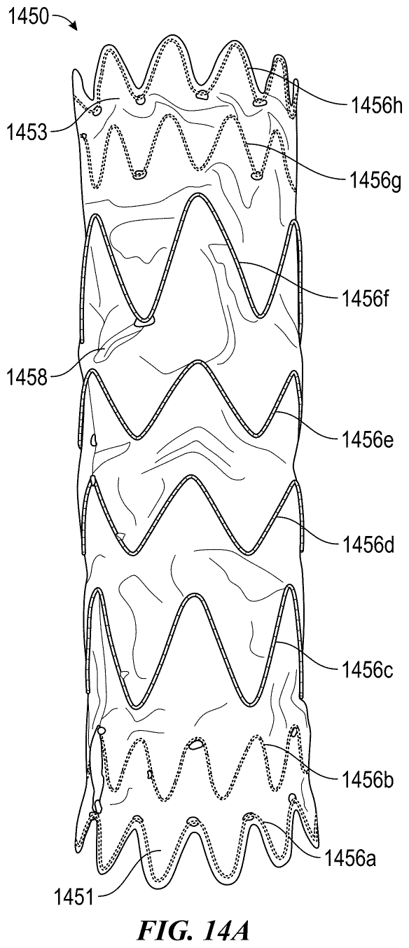

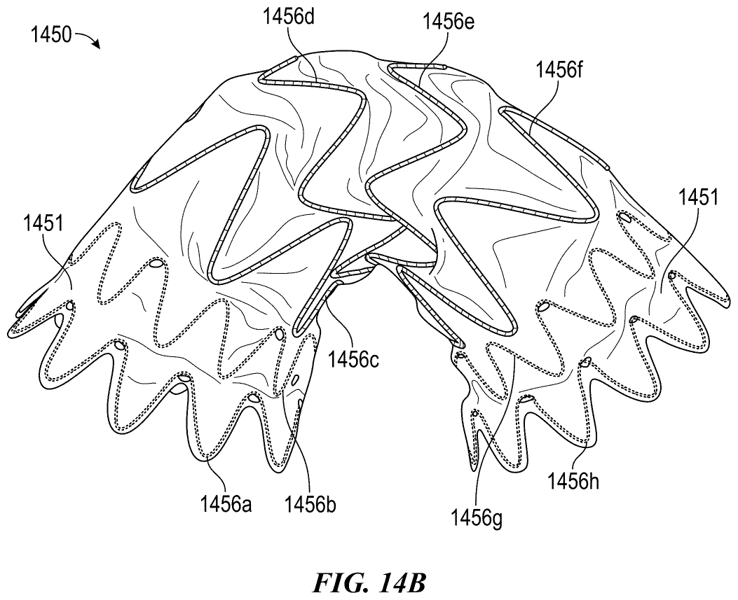

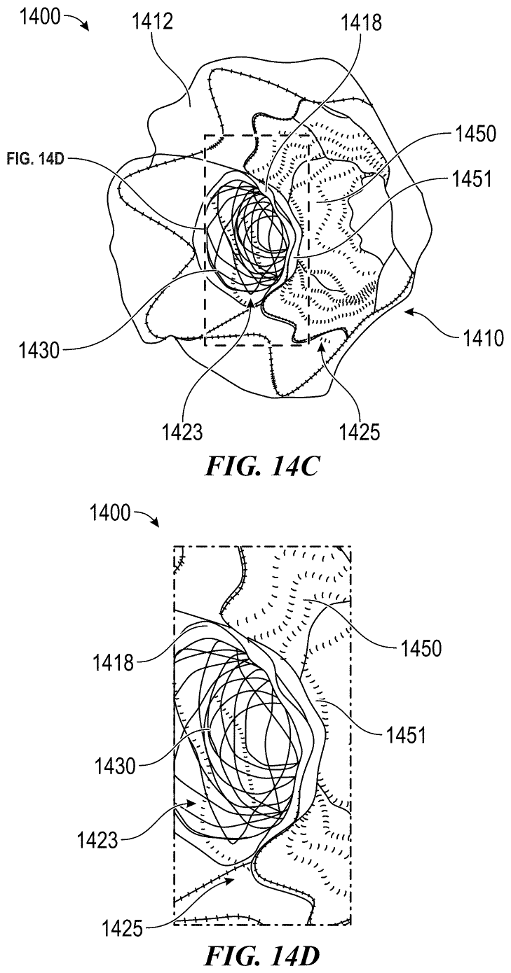

FIGS. 14A and 14B are perspective side views of a spanning member 1450 in accordance with embodiments of the present technology. Referring to FIGS. 14A and 14B together, the spanning member 1450 includes a proximal end portion 1451 configured to expand within and conform to a base member and a distal end portion 1453 configured to expand within and conform to a vessel, such as the aorta (e.g., the aortic arch, the descending thoracic aorta). The spanning member 1450 includes a plurality of stents 1456 (e.g., including individually identified first through eight stents 1456a-h, respectively) attached to a graft material 1458.

In the illustrated embodiment, the first, second, seventh, and eighth stents 1456a, b, g, h are positioned at the proximal and distal end portions 1451, 1453 and have a smaller amplitude and larger frequency than the third through sixth stents 1456c-f positioned therebetween. The third through sixth stents 1456c-f can have the same or generally the same frequency, and the third and sixth stents 1456c, f can have a larger amplitude than the fourth and fifth stents 1456d, e positioned therebetween. The first stent 1456a can have a first diameter of, for example, about 44 millimeters. The second through seventh stents 1456b-g can have a second diameter less than the first diameter of, for example, about 40 millimeters. The eight stent 1456h can have a third diameter greater than the second diameter and less than the first diameter of, for example, about 42 millimeters. Accordingly, the proximal end portion 1451 can be flared relative to the rest of the spanning member 1450 to improve conformance and sealing to a base member. Likewise, the distal end portion 1453 can also be flared relative to the adjacent ones of stents 1456 (e.g., but flared less than the proximal end portion 1451).

In some embodiments, the first, second, seventh, and eighth stents 1456a, b, g, h can each have a relatively small first wire diameter of, for example, about 0.14 inch to provide a high level of radial conformance. The third through sixth stents 1456c-f therebetween can each have a greater second wire diameter to provide a relatively greater radial-outward force. For example, in some embodiments the third through sixth stents 1456c-f have a wire diameter of about 0.025 inch, 0.018 inch, 0.016 inch, and 0.022 inch, respectively. Further, in some embodiments the third through sixth stents 1456c-f can be spaced apart from one another at a distance greater than a distance between the first, second, seventh, and eighth stents 1456a, b, g, h.

FIGS. 14C and 14D are a distally-facing perspective end view and an enlarged distally-facing perspective end view, respectively, of an aortic repair device 1400 including the spanning member 1450 deployed within a base member 1410 in accordance with embodiments of the present technology. Referring to FIGS. 14C and 14D together, the base member 1410 includes a body 1412 with a septum 1418 extending fully or partially therethrough. The septum 1418 is laterally offset within the body 1412 such that the septum 1418 divides the body 1412 into a primary lumen 1423 and a secondary lumen 1425 having a smaller size than the primary lumen 1423. A stent 1430 is deployed within the secondary lumen 1425. As shown, the relatively conformable proximal portion 1451 of the spanning member 1450 can conform and seal around the septum 1418 and the stent 1430 without leaving substantial gaps (e.g., leak paths).

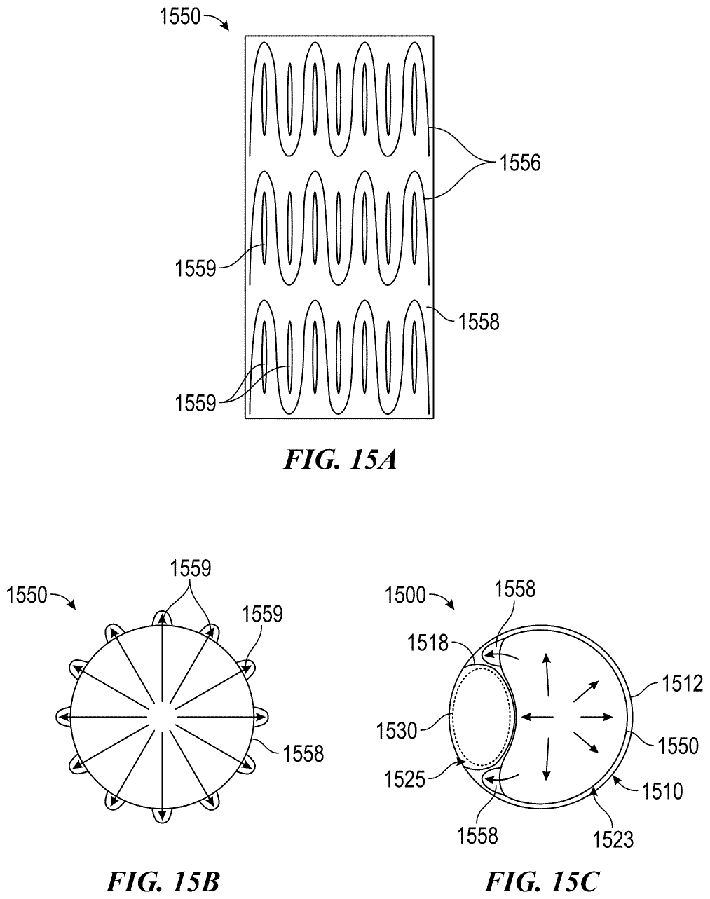

FIGS. 15A and 15B are a side view and a distally-facing end view, respectively, of a spanning member 1550 in accordance with embodiments of the present technology. FIG. 15C is a distally-facing end view of an aortic repair device 1500 including the spanning member 1550 of FIGS. 15A and 15B in accordance with embodiments of the present technology. Referring to FIG. 15C, the aortic repair device 1500 can include a base member 1510 having a body 1512 with a septum 1518 extending fully or partially therethrough. The septum 1518 is laterally offset within the body 1512 such that the septum 1518 divides the body 1512 into a primary lumen 1523 and a secondary lumen 1525 having a smaller size than the primary lumen 1523. A stent 1530 is deployed within the secondary lumen 1525.

Referring to FIGS. 15A-15C together, the spanning member 1550 can comprise a plurality of stents 1556 attached to a graft material 1558. In the illustrated embodiment, the graft material 1558 includes filler portions 1559, which can comprise pockets, loose portions, and/or baggy portions of the graft material 1558. The filler portions 1559 can extend radially outward from the stents 1556 and be circumferentially and/or longitudinally disposed around the spanning member 1550. In some embodiments, the filler portions 1559 can protrude outward in response to blood pressure within a vessel as shown by arrows in FIGS. 15B and 15C. Referring to FIG. 15C, in some aspects of the present technology the filler portions 1559 can help the spanning member 1550 conform and seal around the circumference of the primary lumen 1523 without leaving substantial gaps (e.g., leak paths). In some embodiments, the filler portions 1559 are asymmetrically positioned around the spanning member 1550 (e.g., along only a portion of the perimeter thereof) and the spanning member 1550 can be aligned within the body 1512 during a surgical procedure such that the filler portions 1559 engage and surround the septum 1518.

The graft material 1558 can be attached radially outside the stents 1556, or radially inside the stents 1556 such that the filler portions 1559 extend/extrude through the stents 1556. FIGS. 15D and 15E are side views of the spanning member 1550 when subject to blood flow (e.g., when implanted within a vessel (not shown)) and having the graft material 1558 attached to an inside and an outside of the stents 1556 respectively, in accordance with embodiments of the present technology. As shown in FIG. 15D, blood flow through/past the spanning member 1550 can cause the filler portions 1559 to extend radially past the stents 1556 in the areas therebetween when the graft material 1558 is attached inside the stents 1556. As shown in FIG. 15E, blood flow through/past the spanning member 1550 can cause the filler portions 1559 to billow outward around the attachment points of the graft material 1558 to the stents 1556 when the graft material 1558 is attached outside the stents 1556.

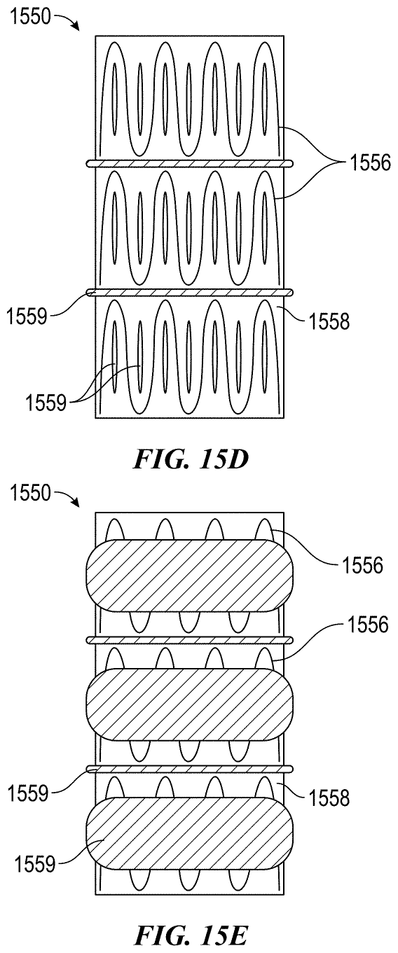

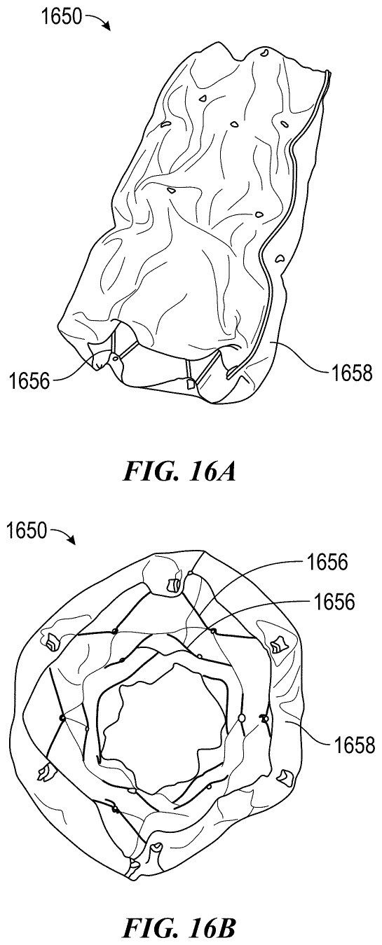

FIGS. 16A and 16B are a perspective side view and a perspective end view, respectively, of a spanning member 1650 in accordance with embodiments of the present technology. Referring to FIGS. 16A and 16B together, the spanning member 1650 includes a graft material 1658 attached to an outside of a plurality of stents 1656. The graft material 1658 can be oversized relative to the stents 1656 such that the graft material 1658 is loose or baggy and can thus help fill and seal any gaps when the spanning member 1650 is deployed within a base member. For example, the graft material 1658 can have a diameter (e.g., about 50 millimeters) larger than a diameter (e.g., about 40 millimeters) of the stents 1656.

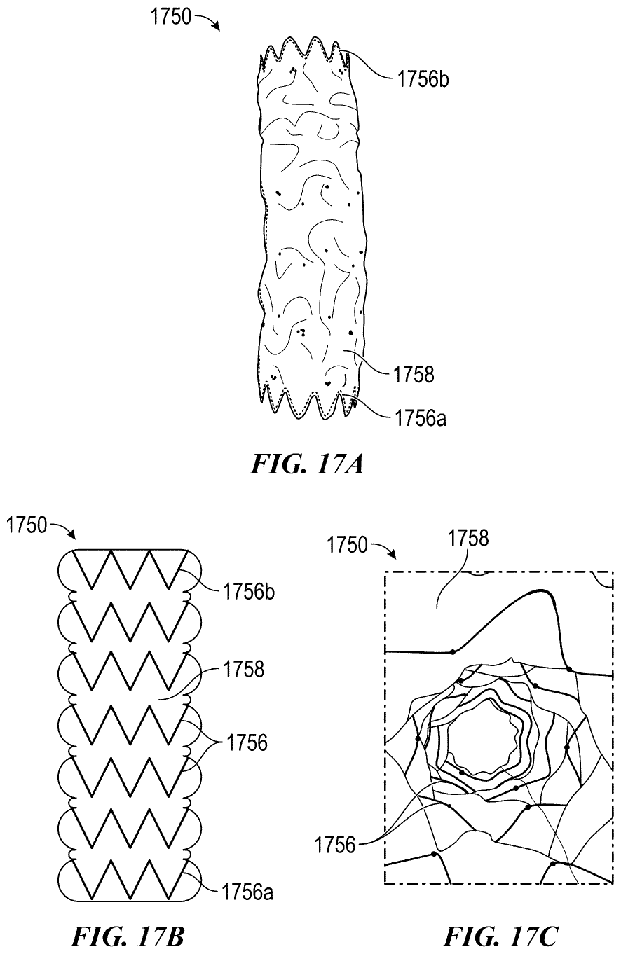

FIGS. 17A-17C are a perspective side view, a side cross-sectional view, and a perspective end view, respectively, of a spanning member 1750 in accordance with embodiments of the present technology. Referring to FIGS. 17A-17C together, the spanning member 1750 includes a graft material 1758 attached to a plurality of stents 1756 (e.g., including an individually identified proximal-most stent 1756a and distal-most stent 1756b). The graft material 1758 can be oversized relative to the stents 1756 such that the graft material 1758 is loose or baggy and can thus help fill and seal any gaps when the spanning member 1750 is deployed within a base member. In the illustrated embodiment, the proximal-most stent and distal most-stents 1756a, b are high frequency and low amplitude stents attached to the outside of the graft material 1758 and can help improve the conformance and of the spanning member 1750. The graft material 1758 can be attached to an outside of the stents 1756 other than the proximal-most stent and distal most-stents 1756a, b.

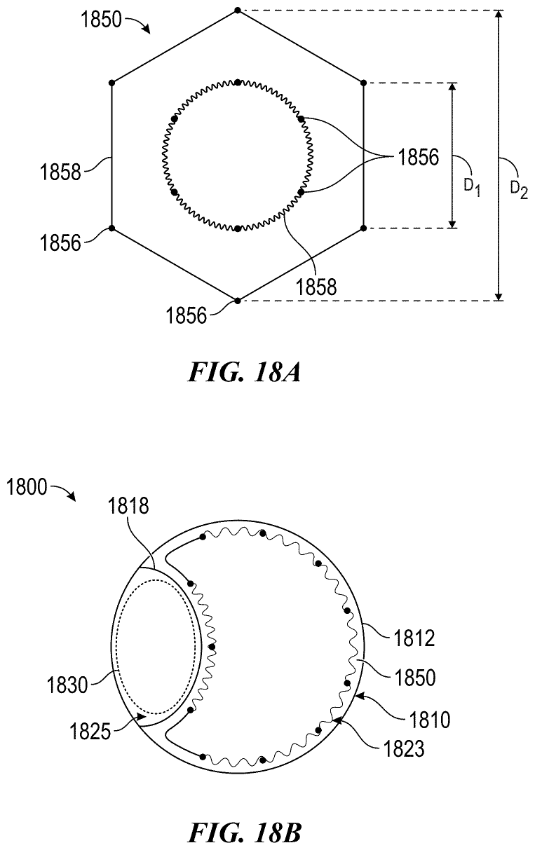

FIG. 18A is an end view of a spanning member 1850 in a constrained (e.g., first) configuration and an expanded (e.g., second) configuration in accordance with embodiments of the present technology. FIG. 18B is a distally-facing end view of an aortic repair device 1800 including the spanning member 1850 of FIG. 18A in accordance with embodiments of the present technology. Referring to FIG. 18B, the aortic repair device 1800 can include a base member 1810 having a body 1812 with a septum 1818 extending fully or partially therethrough. The septum 1818 is laterally offset within the body 1812 such that the septum 1818 divides the body 1812 into a primary lumen 1823 and a secondary lumen 1825 having a smaller size than the primary lumen 1823. A stent 1830 is deployed within the secondary lumen 1825.

Referring to FIG. 18A, the spanning member 1850 includes a graft material 1858 attached to a plurality of struts 1856 of a stent. The spanning member 1850 has a first diameter D₁ in the constrained configuration and a second diameter D₂ greater than the first diameter D₁ in the expanded configuration. The graft material 1858 is configured to be relatively loose or baggy in the constrained configuration, and can be taut in the expanded configuration. In some embodiments the second diameter D₂ is between about 1.2-2.5 (e.g., between about 1.3-2.0) times larger than the first diameter D₁. Referring to FIGS. 18A and 18B together, in some embodiments the diameter D₁ is selected to be equal or about equal to the diameter of a target vessel (e.g., aorta) in which the spanning member 1850 is to be implanted such that the spanning member 1850 assumes the constrained configuration when deployed within the base member 1810. Accordingly, in some aspects of the present technology the loose graft material 1858 can help fill help the spanning member 1850 conform and seal around the circumference of the primary lumen 1823 without leaving substantial gaps (e.g., leak paths) through the body 1812.

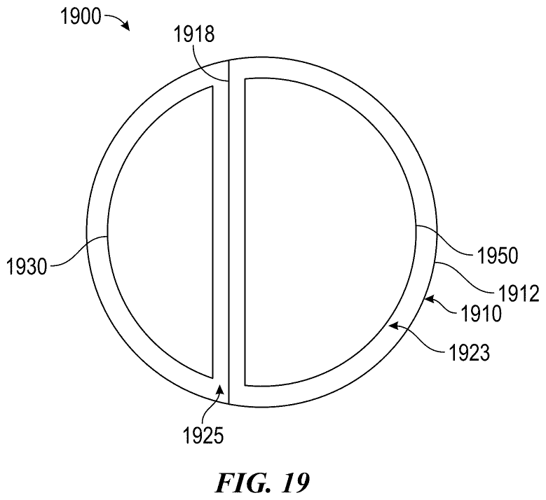

FIG. 19 is a distally-facing end view of an aortic repair device 1900 in accordance with embodiments of the present technology. In the illustrated embodiment, the aortic repair device 1900 includes a base member 1910 having a body 1912 with a septum 1918 extending fully or partially therethrough. The septum 1918 is laterally offset within the body 1912 such that the septum 1918 divides the body 1912 into a primary lumen 1923 and a secondary lumen 1925 having a smaller size than the primary lumen 1923. A stent 1930 is deployed within the secondary lumen 1925, and a spanning member 1950 is deployed within the primary lumen 1923. In the illustrated embodiment, both the stent 1930 and the spanning member 1950 have a D-like cross-sectional shape that together form a generally circular shape. The matching D-like cross-sectional shapes can help improve the conformance and seal of (i) the spanning member 1950 within the primary lumen 1923 and (ii) the stent 1930 within the secondary lumen 1925, thereby inhibiting leaks through the body 1912.

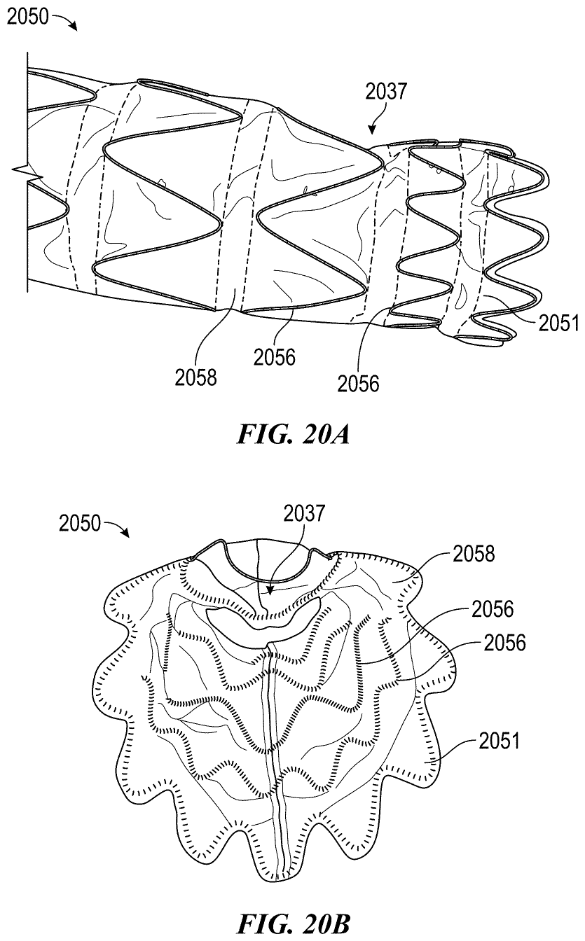

FIGS. 20A and 20B are an enlarged perspective side view and a distally-facing perspective end view, respectively, of a spanning member 2050 in accordance with embodiments of the present technology. Referring to FIGS. 20A and 20B together, the spanning member 2050 includes a graft material 2058 attached to a plurality of stents 2056. The spanning member 2050 can further include a proximal end portion 2051 configured to be positioned within a lumen of a base member, such as the base member 810 described in detail above with reference to FIGS. 8A-8H. In the illustrated embodiment, the stents 2056 near the proximal end portion 2051 are configured (e.g., shaped and sized) to form an indent 2037 such that the proximal end portion 2051 of the spanning member 2050 has, for example, a crescent or kidney-bean cross-sectional shape. With additional reference to FIGS. 8A-8H, when the spanning member 2050 is deployed within the primary lumen 823 of the base member 810, the indent 2037 can be positioned adjacent the stent 830 and the septum 818, and is shaped to generally match the shape of the stent 830 and the septum 818 such that the spanning member 850 conforms and seals to the circumference of the primary lumen 823.

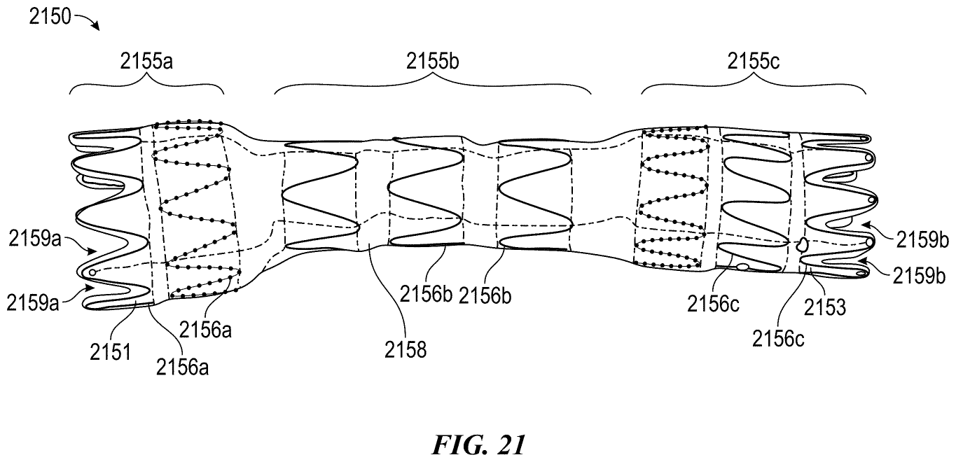

FIG. 21 is a perspective side view of a spanning member 2150 in accordance with embodiments of the present technology. In the illustrated embodiment, the spanning member 2150 includes a proximal end portion 2151 configured to expand within and conform to a base member and a distal end portion 2153 configured to expand within and conform to a vessel, such as the aorta (e.g., the aortic arch, the descending thoracic aorta), and/or another base member. The spanning member 2150 includes a plurality of stents 2156 (e.g., including individually identified first stents 2156a, second stents 2156b, and third stents 2156c) attached to a graft material 2158. The first stents 2156a define a first (e.g., proximal) region 2155a of the spanning member 2150, the second stents 2156b define a second (e.g., middle) region 2155b of the spanning member 2150, and the third stents 2156c define a third (e.g., distal) region 2155c of the spanning member 2150. In some embodiments, the second stents 2156b can have a reduced diameter compared to the first stents 2156a and/or the third stents 2156c such that second region 2155b is narrower than the first region 2155a and/or the third region 2155c. In the illustrated embodiment, the graft material 2158 (i) does not extend proximally past the most proximal one of the first stents 2156a such that the spanning member 2150 has proximal openings 2159a that track the contours of the most proximal one of the first stents 2156a (e.g., arranged in a scalloped pattern) and (ii) does not extend distally past the most distal one of the third stents 2156c such that the spanning member 2150 has distal openings 2159b that track the contours of the most distal one of the third stents 2156c. That is, the most proximal one of the first stents 2156a can be bare and exposed proximally from the graft material 2158 and the most distal one of the third stents 2156c can be bare and exposed distally from the graft material 2158. In some aspects of the present technology, omitting the graft material 2158 in this manner to form the proximal openings 2159a and the distal openings 2159b can improve the ability to position the spanning member 2150 in one or more base members by reducing a risk of unintentional sealing if the spanning member 2150 is too long or positioned too far with the base member.

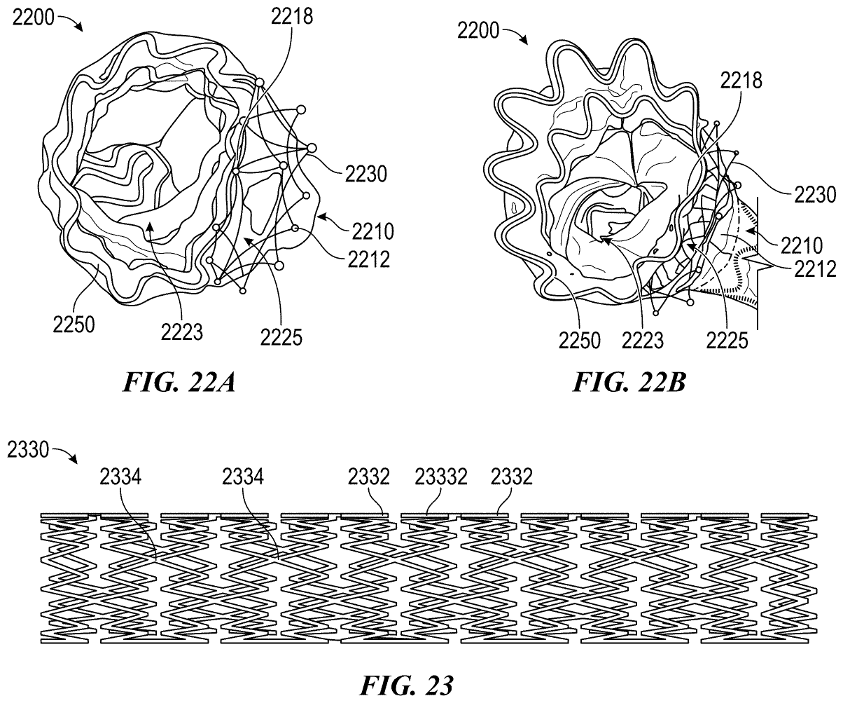

FIGS. 22A and 22B are perspective end views of an aortic repair device 2200 in accordance with embodiments of the present technology. In the illustrated embodiment, the aortic repair device 2200 includes a base member 2210 having a body 2212 with a septum 2218 extending fully or partially therethrough and dividing the body 2212 into a primary lumen 2223 and a secondary lumen 2225 having a smaller size than the primary lumen 2223. In the illustrated embodiment, a spanning member 2250 is deployed within the primary lumen 2223 and a stent 2230 is deployed within the secondary lumen 2225.

As best seen in FIG. 22B, if the spanning member 2250 has a significantly greater radial strength than the stent 2230, the spanning member 2250 can compress, flatten, and/or crush the stent 2230 such that the secondary lumen 2225 is unduly constricted and narrowed. Accordingly, in some embodiments the stent 2230 can be configured to have a radial strength that inhibits undue compression by the spanning member 2250.

FIG. 23, for example, is a side view of a stent 2330 for use with the aortic repair device 2200 of FIGS. 22A and 22B having increased radial strength (e.g., radial resistive force) in accordance with embodiments of the present technology. In the illustrated embodiment, the stent 2330 has a plurality of first struts 2332 having a zigzag or undulating shape (e.g., including a plurality of V-shaped portions) or a diamond shape (e.g., including a plurality of diamond-shaped portions) that extend in a circumferential direction and that are interconnected by longitudinally extending second struts 2334. The stent 2330 can be made to have increased radial strength by (i) increasing the thickness of the first struts 2332 and/or the second struts 2334 in a radial direction by, for example, cutting (e.g., laser cutting) the stent 2330 from a tube having a thicker wall, (ii) increasing the width of the first struts 2332 and/or the second struts 2334 in a longitudinal and/or circumferential direction, (iii) increasing the number of the second struts 2334 (e.g., by including second struts 2334 between all or most of the apices of adjacent ones of the first struts 2332), and/or (iv) increasing a “V-angle” of the first struts 2332 (e.g., by including fewer of the V-shaped portions or diamond-shaped portions around the circumference of the first struts 2332 and/or shorter V-shaped portions or diamond-shaped portions). Referring to FIGS. 22A-23 together, the increased radial strength can inhibit or even prevent the spanning member 2550 from overly compressing the stent 2330 and narrowing the secondary lumen 2225.

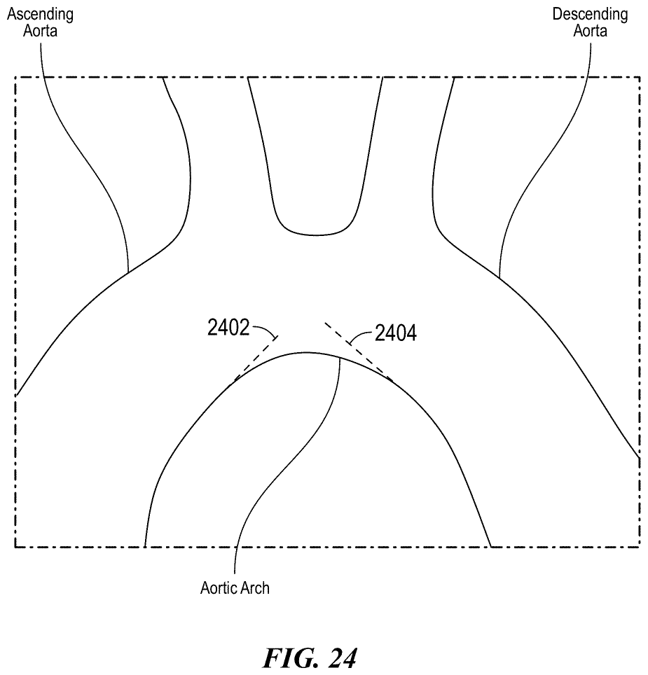

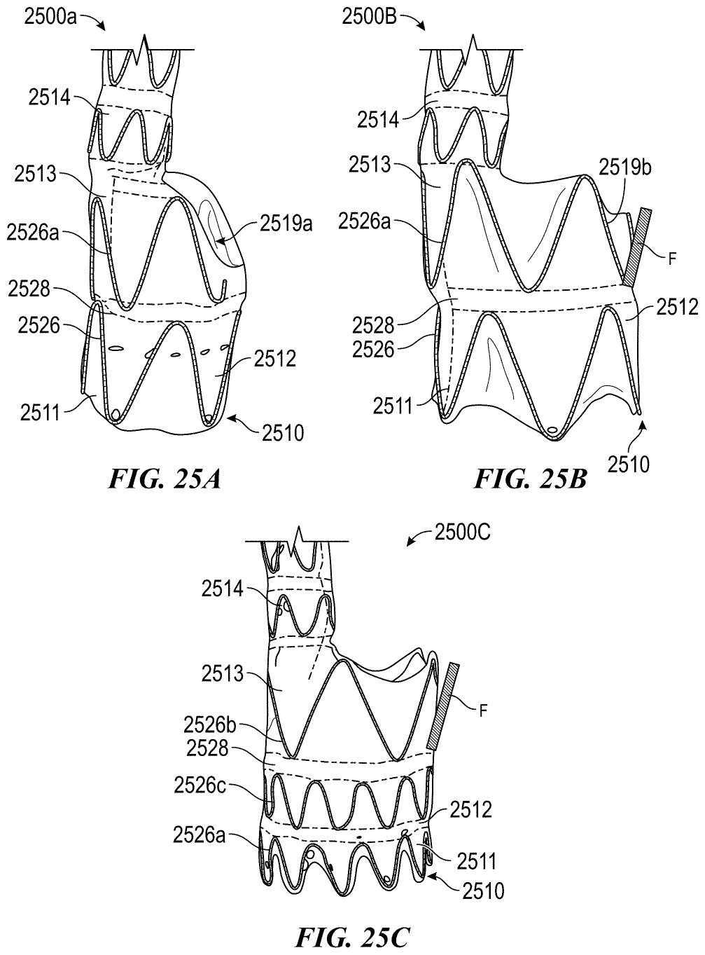

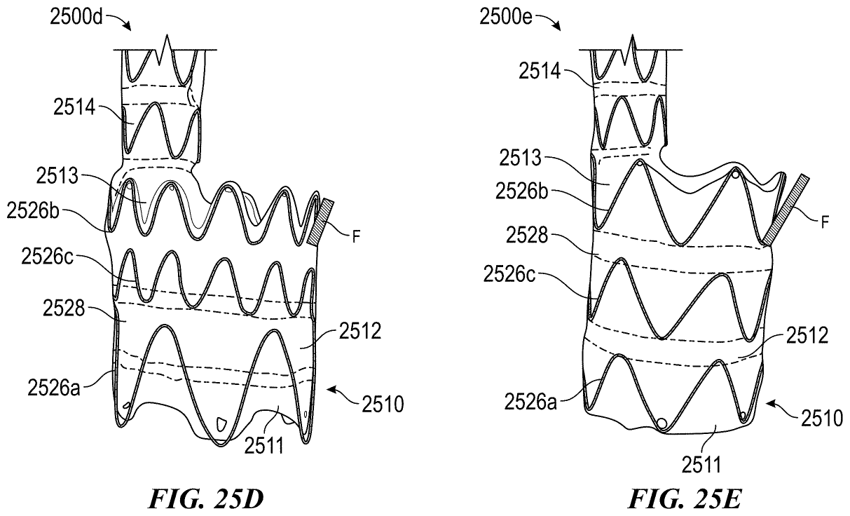

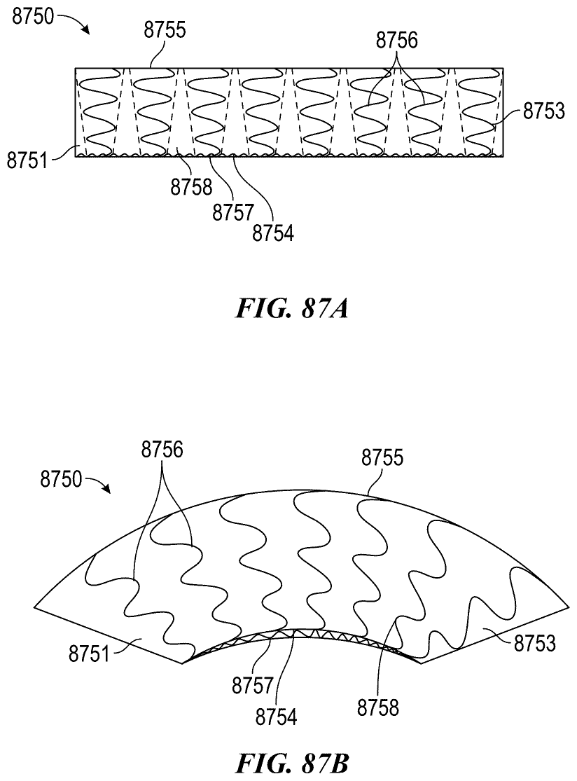

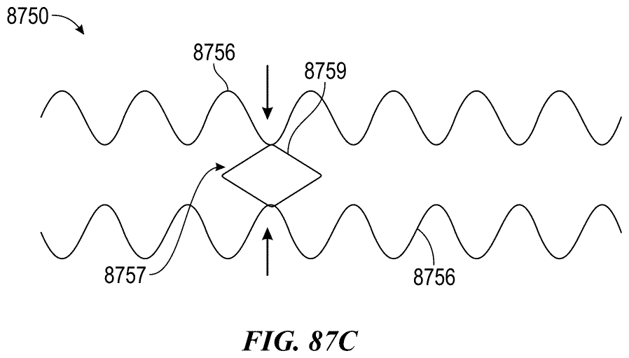

FIG. 24 is a side view of an aorta illustrating aspects of aortic repair device delivery procedures. As shown in FIG. 24, a first line 2402 illustrates where a tubular body of a base member implanted in the ascending aorta may lift off from and not conform to the aorta proximate the aortic arch. Likewise, a second line 2404 in FIG. 24 illustrates where a tubular body of a base member implanted in the descending aorta may lift off from and not conform to the aorta proximate the aortic arch. In such instances, the seal length of the base member may decrease. In some embodiments, any of the aortic repair devices described herein can include features configured to promote conformance of the aortic repair device to the aorta (e.g., along relatively curved portions of the aorta, such as the aortic arch) to inhibit or even prevent the aortic repair device from lifting off the wall of the aorta. For example, FIGS. 25A-27D illustrate additional embodiments of aortic repair devices including features that enhance conformance of base members to arched vessel walls in accordance with embodiments of the present technology, and FIGS. 87A-87C illustrate additional embodiments of aortic repair devices including features that enhance conformance of spanning members to arched vessel walls in accordance with embodiments of the present technology. The various aortic repair devices can include some features that are at least generally similar in structure and function, or identical in structure and function, to one another and/or to the corresponding features of the aortic repair devices described in detail above with reference to FIGS. 3A-23, and can operate in a generally similar or identical manner to one another and/or to the aortic repair devices described above.

FIGS. 25A-25E, for example, are perspective side views of various aortic repair devices 2500a-2500e, respectively, in accordance with embodiments of the present technology. In the illustrated embodiments, the aortic repair devices 2500a-2500e each include a base member 2510. having (i) a body 2512 including a proximal end portion 2511 and a distal end portion 2513 and (ii) a leg 2514 extending from the distal end portion 2513. The base members 2510 each comprise one or more stents 2526 coupled to a graft material 2528. In some embodiments, the body 2512 of any one of the aortic repair devices 2500a-e can be flared outwardly at the proximal end portion 2511 and/or the distal end portion 2513. For example, the distal end portion 2513 can be flared outwardly along and/or generally along a line F shown in each of FIGS. 25B-25E to improve the conformance and/or seal of the body 2512 within the aorta and, in particular, around a curved portion of the aorta (e.g., proximate the aortic arch).

Referring to FIGS. 25A and 25B together, the stents 2526 along the body 2512 include an individually identified mid stent 2526a positioned at and/or near the distal end portion 2513 between the body 2512 and the leg 2514 and having a V-pattern shape (e.g., including alternating proximal and distal apices). In the embodiment illustrated in FIG. 25A, one of the V-shaped portions of the mid stent 2526a and the graft material thereon is removed opposite the leg 2514 to form an opening or cut-out 2519a (e.g., having a scalloped or scoop shape). In the embodiment illustrated in FIG. 25B, the mid stent 2526a has a wedge portion 2519b opposite the leg 2514 and having a reduced amplitude relative to the rest of the mid stent 2526a. In some aspects of the present technology, the cut-out 2519a and/or the wedge portion 2519b can improve the conformance and seal of the body 2512 within the aorta and, in particular, around a curved portion of the aorta (e.g., proximate the aortic arch).

Referring to FIGS. 25C-25D together, the stents 2526 along the body 2512 include an individually identified proximal stent 2526a at and/or near the proximal end portion 2511, a distal stent 2526b at and/or near the distal end portion 2513, and a mid stent 2526c between the proximal stent 2526a and the distal stent 2526b. The stents 2526a-c each have a periodic V-pattern shape (e.g., including alternating proximal and distal apices). In the embodiment illustrated in FIG. 25C, the distal stent 2526b has larger amplitude (e.g., length in a direction between the apices and between the proximal end portion 2511 and the distal end portion 2513 of the body 2512) and smaller frequency than those of the proximal stent 2526a and the mid stent 2526c. Accordingly, the proximal end portion 2511 (e.g., entry) of the body 2512 can have a relatively high frequency. In the embodiment illustrated in FIG. 25D, the proximal stent 2526a has larger amplitude (e.g., length in a direction between the apices and between the proximal end portion 2511 and the distal end portion 2513 of the body 2512) and smaller frequency than those of the distal stent 2526b and the mid stent 2526c. Accordingly, the distal end portion 2513 (e.g., exit) of the body 2512 can have a relatively high frequency. In the embodiment illustrated in FIG. 25E, the stents 2526a-c each have the same amplitude and frequency. In some aspects of the present technology, the arrangement of the stents 2526a-c in one or more of FIGS. 25C-25E can improve the conformance and seal of the body 2512 within the aorta and, in particular, around a curved portion of the aorta (e.g., proximate the aortic arch).

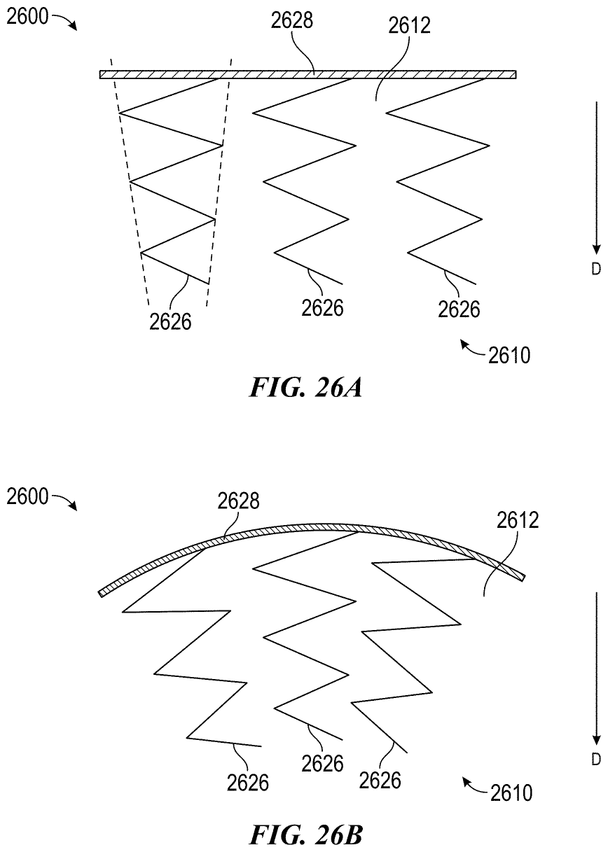

FIGS. 26A and 26B are enlarged side views of a portion of an aortic repair device 2600 in a first position (e.g., a non-deployed position) and a second position (e.g., a deployed position), respectively, in accordance with embodiments of the present technology. Referring to FIGS. 26A and 26B together, the aortic repair device 2600 includes a base member 2610 having a body 2612 comprising one or more stents 2626 coupled to a graft material 2628. Similar to the embodiment shown in FIG. 25B, in the illustrated embodiment the stents 2626 have a V-pattern shape (e.g., including alternating proximal and distal apices) that decreases in amplitude in a direction D. The direction D can be in a direction toward the interior wall of the aortic arch having the lesser curvature (e.g., smaller radius of curvature, shorter length). In some aspects of the present technology, the decreasing amplitude of the stents 2626 can improve the conformance and seal of the body 2612 within the aorta and, in particular, around a curved portion of the aorta (e.g., proximate the aortic arch). In some embodiments, the graft material 2628 and/or the stents 2626 can be pre-curved to have the second position shown in FIG. 26B.

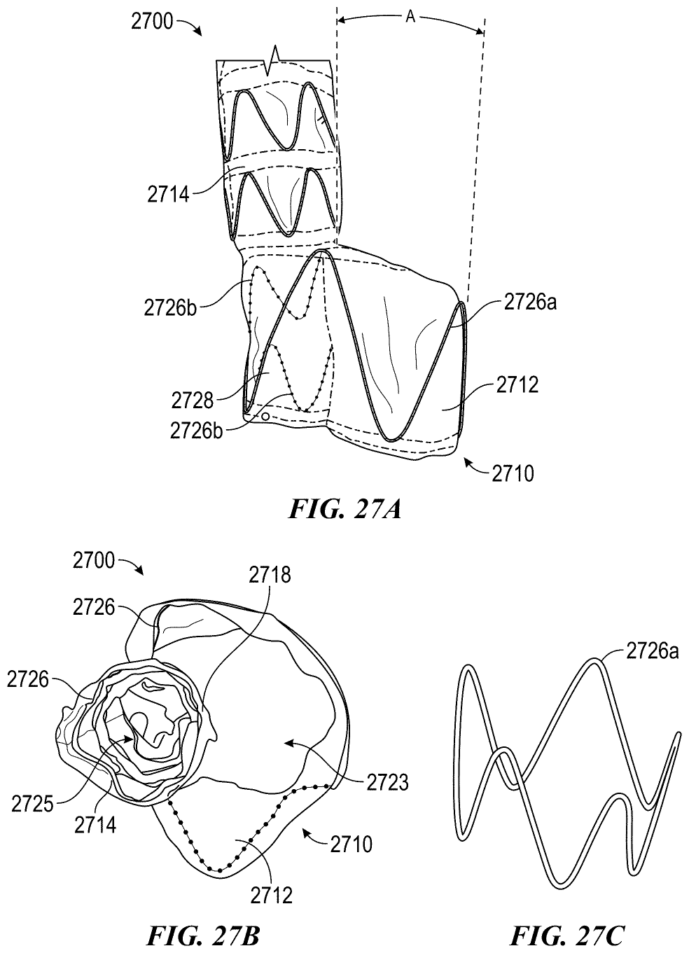

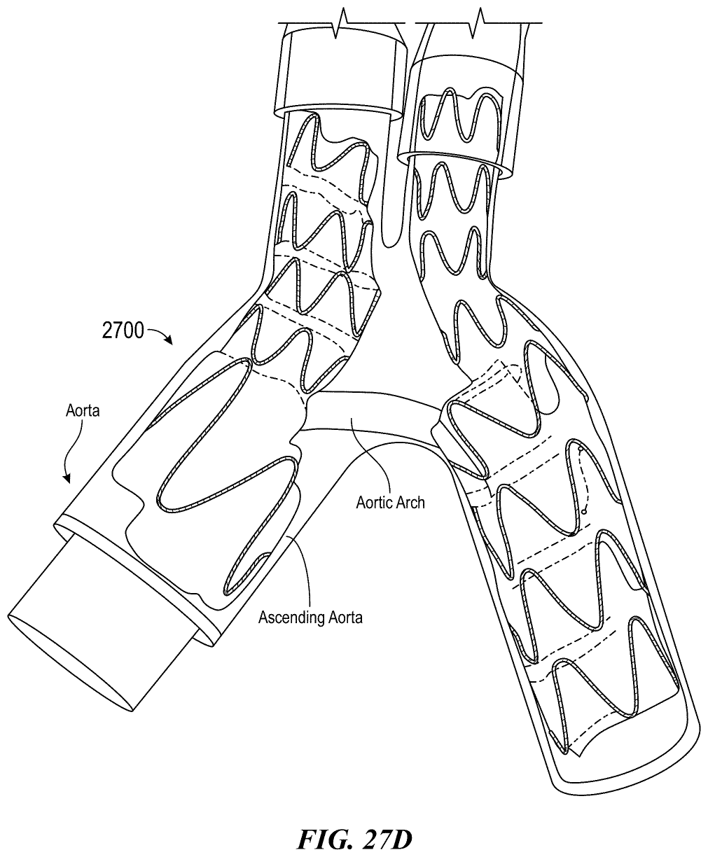

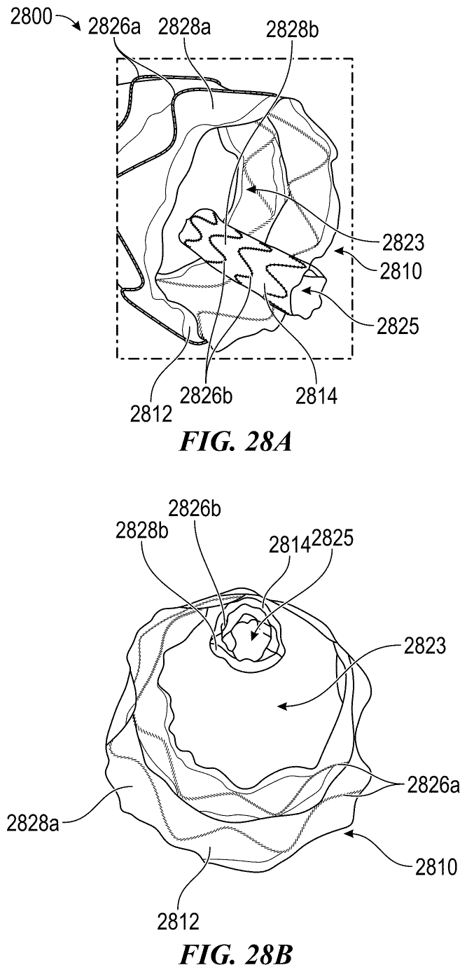

FIGS. 27A and 27B are an enlarged side perspective view and a perspective end view, respectively, of an aortic repair device 2700 in accordance with embodiments of the present technology. Referring to FIGS. 27A and 27B together the aortic repair device 2700 includes a base member 2710 having a body 2712, a leg 2714 extending from the body 2712, and a septum 2718 (FIG. 27B) extending fully or partially therethrough and dividing the body 2212 into a primary lumen 2723 (FIG. 27B) and a secondary lumen 2725 (FIG. 27B) having a smaller size than the primary lumen 2223. The base member 2710 comprises one or more stents 2726 coupled to a graft material 2728. More specifically, referring to FIG. 27A, the stents 2726 along the body 2712 can include a first stent 2726a extending entirely circumferentially about the body 2712 and second stents 2726b extending entirely or partially circumferentially about the secondary lumen 2725 (FIG. 27B). As described in greater detail below with reference to FIGS. 28A-33, the second stents 2726b can support and preserve the secondary lumen 2725 (FIG. 27B) when a spanning member is deployed within the primary lumen 2723 (FIG. 27B).

FIG. 27C is a perspective view of the first stent 2726a in accordance with embodiments of the present technology. Referring to FIGS. 27A and 27C together, and similar to the aortic repair device 2500b described in detail above with reference to FIG. 25B, the first stent 2726a can have a V-pattern shape (e.g., including alternating proximal and distal apices) that varies (e.g., decreases) in amplitude in a direction away from the leg 2714. In some embodiments, the portion of the first stent 2726a positioned farthest from the leg 2714 can also be angled outward away from the leg 2714 at an angle A (FIG. 27A). In some aspects of the present technology, the decreasing amplitude and/or the outward angle A of the first stent 2726a can improve the conformance and seal of the body 2712 within the aorta and, in particular, around a curved portion of the aorta (e.g., proximate the aortic arch). For example, FIG. 27D shows the aortic repair device 2700 implanted within an aorta and conforming to the wall of the ascending aorta proximate the aortic arch in accordance with embodiments of the present technology.

FIGS. 87A and 87B are side views of a spanning member 8750 of an aortic repair device in a first position (e.g., a straight position, an extended position) and a second position (e.g., a curved position, a relaxed position), respectively, in accordance with embodiments of the present technology. Referring to FIGS. 87A and 87B together, in the illustrated embodiment the spanning member 8750 comprises a plurality of stents 8756 attached to a graft material 8758. The spanning member 8750 further has a leading (e.g., proximal) end portion 8751, a trailing (e.g., distal) end portion 8753, a first side portion 8754 configured to conform to a portion of an interior wall of the aortic arch having a lesser curvature (e.g., a smaller radius of curvature, shorter length), and a second side portion 8755 opposite to the first side portion 8754 and configured to conform to a portion of the interior wall of the aortic arch having a greater curvature (e.g., greater radius of curvature, longer length). The first side portion 8754 can include features that cause the spanning member to passively shorten such that the spanning member 8750 takes on an arch-like shape upon deployment (FIG. 87B). In some embodiments, for example, the stents 8756 have a V-pattern shape (e.g., including alternating proximal and distal apices) that decreases in amplitude in a direction from the second side portion 8755 toward the first side portion 8754. In some aspects of the present technology, the decreasing amplitude of the stents 8756 can improve the conformance and seal of the spanning member 8750 within the aorta and, in particular, around a curved portion of the aorta (e.g., proximate the aortic arch) in a similar manner as described in detail above with reference to FIGS. 26A and 26B.

In some embodiments, the spanning member 8750 can include a tension member 8757 (e.g., a shortening member) positioned along at least a segment of the first side portion 8754. The tension member 8757 can be an elongate member that spans fully or partially between the leading end portion 8751 and the trailing end portion 8753, and can be configured to passively shorten the first side portion 8754 of the spanning member 8750 to transition the spanning member 8750 from the straight first position shown in FIG. 87A to the curved second position shown in FIG. 87B. The tension member 8757 can be (i) an elastic band, cord, or other element, (ii) a spring (e.g., a flat spring, a zig-zag nitinol tension flat spring, a flattened coil spring), (iii) a strip of stretchy material (e.g., urethane, thermoplastic elastomer (TPE)), (iv) a stent-like element, and/or (v) the like. The tension member 8757 can be secured inside the graft material 8758 (e.g., to minimize wear of the tension member 8757) or outside the graft material 8758 (e.g., to minimize potential embolization within the spanning member 8750). In some embodiments, the tension member 8757 is covered with a polymer or other material and/or inserted into a seam/channel formed in the graft material 8758. In some embodiments, the tension member 8757 can comprise a plurality of individual springs or other shortening members positioned between individual ones of the stents 8756. FIG. 87C, for example, is an enlarged side view of a portion of the spanning member 8750 in accordance with additional embodiments of the present technology. In the illustrated embodiment, the tension member 8757 comprises one or more springs 8759 positioned between individual ones of the stents 8756.

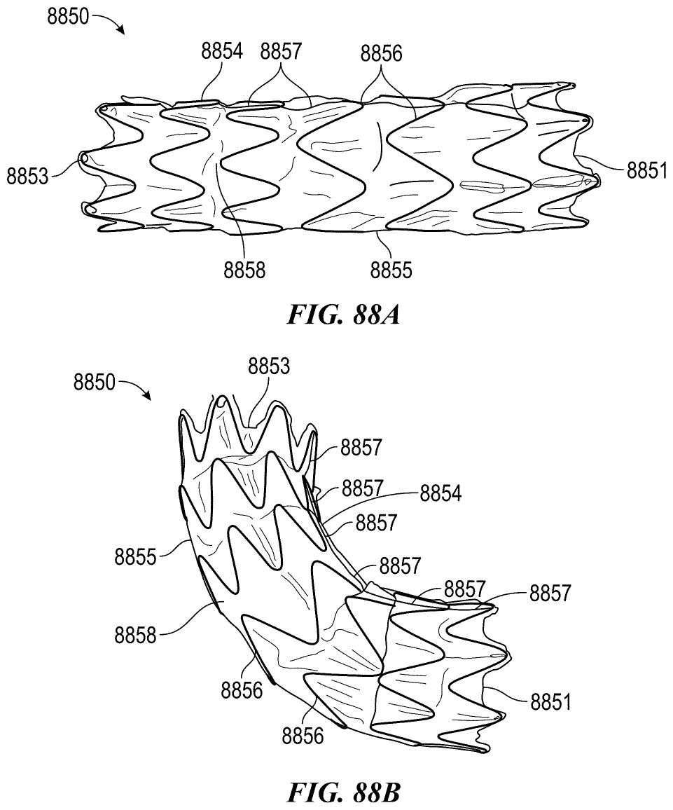

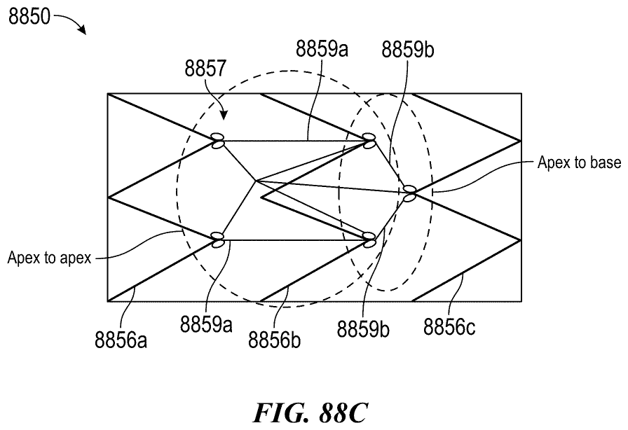

FIGS. 88A and 88B are perspective side views of a spanning member 8800 of an aortic repair device in a first position (e.g., a straight position, an extended position) and a second position (e.g., a curved position, a relaxed position), respectively, in accordance with embodiments of the present technology. Referring to FIGS. 88A and 88B together, in the illustrated embodiment the spanning member 8850 comprises a plurality of stents 8856 attached to a graft material 8858. The spanning member 8850 further has a leading (e.g., proximal) end portion 8851, a trailing (e.g., distal) end portion 8853, a first side portion 8854 configured to conform to a portion of an interior wall of the aortic arch having a lesser curvature (e.g., a smaller radius of curvature, shorter length), and a second side portion 8855 opposite to the first side portion 8854 and configured to conform to a portion of the interior wall of the aortic arch having a greater curvature (e.g., greater radius of curvature, longer length).

The first side portion 8854 includes a tension member 8857 comprising a plurality of individual elastic (e.g., Lycra) fibers that span between adjacent ones of the stents 8856 and that passively shorten the first side portion 8854 when the spanning member 8850 is unconstrained such that the spanning member 8850 assumes an arch-like shape upon deployment (FIG. 88B). For example, in the illustrated embodiment the tension member 8857 can comprise six of the elastic fibers (also referred to as “elastic components”)—one positioned between each adjacent pair of the seven illustrated stents 8856. In some embodiments, more of the elastic fibers can be positioned between an adjacent pair of the stents 8856. The elastic fibers of the tension member 8857 can be sewn onto the exterior of the graft material 8858 at the apices (or another portion) of the stents 8856 such that the elastic members draw the stents 8856 toward one another along the first side portion 8854. The tension member 8857 can be stretched to the straight first position (FIG. 88A) for loading into a delivery catheter in a cylindrical configuration. Upon deployment, the tension member 8857 can cause the spanning member 8850 to assume the curved second position (FIG. 88B).

In some embodiments, the tension member 8857 draws some or all of the stents 8856 toward and at least partially over/under an adjacent one of the stents 8856. Accordingly, the first side portion 8854 can have a series of steps (e.g., rises and falls) there along. In some embodiments, such overlap can resemble a “shingle” pattern in which the overlap of adjacent stents 8856 happens in a consistent pattern. For example, for adjacent ones of the stents 8856, the tension member 8857 can consistently pull the leading one of the stents 8856 toward and/or over the trailing one of the stents 8856. Such a consistent pattern of overlap (“shingling”) can facilitate consistent blood flow through the spanning member 8850.

The elastic fibers of the tension member 8857 can be secured between adjacent ones of the stents 8856 in various arrangements. FIG. 88C, for example, is an enlarged side view of a portion of the spanning member 8850 illustrating two different potential attachments of elastic fibers of the tension member 8857 in accordance with embodiments of the present technology. In the illustrated embodiment, a pair of first elastic fibers 8859a of the tension member 8857 are secured via knots to adjacent apices of an adjacent pair of the stents 8856 (e.g., in an apex-to-apex arrangement between an individually identified first stent 8856a and second stent 8856b). Further, a pair of second elastic fibers 8859b of the tension member 8857 are secured via knots to the apices of an one of an adjacent pair of the stents 8856 and to the same base of the other one of the adjacent pair of the stents 8856 (e.g., in an apex-to-base arrangement between the second stent 8856b and an individually identified third stent 8856b). In other embodiments, the elastic fibers of the tension member 8857 can be secured to the stents 8856 in other manners.

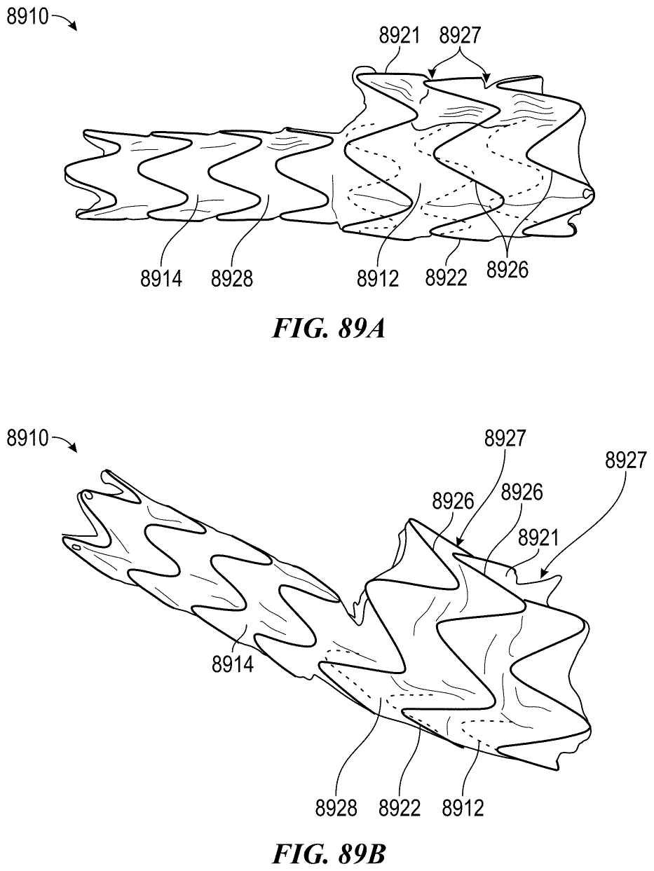

In various embodiments, one or more tension members can be incorporated into other components of the aortic repair devices described herein. For example, FIGS. 89A and 89B are perspective side views of a base member 8910 of an aortic repair device in a first position (e.g., a straight position, an extended position) and a second position (e.g., a curved position, a relaxed position), respectively, in accordance with embodiments of the present technology. Referring to FIGS. 89A and 89B together, the base member 8910 has a body 8912 configured to be implanted in the aorta and a leg 8914 extending from the body 8912 and configured to be implanted in a branch vessel. The base member 8910 comprises a plurality of stents 8926 attached to a graft material 8928. The body 8912 further includes a first side portion 8921 configured to conform to a portion of an interior wall of the aortic arch having a lesser curvature (e.g., a smaller radius of curvature, shorter length), and a second side portion 8922 opposite to the first side portion 8921 and configured to conform to a portion of the interior wall of the aortic arch having a greater curvature (e.g., greater radius of curvature, longer length).

In the illustrated embodiment, the first side portion 8921 of the body 8912 includes a tension member 8927 comprising a plurality of individual elastic (e.g., Lycra) fibers that span between adjacent ones of the stents 8926 along the body 8912 and that passively shorten the first side portion 8921 when the base member 8910 is unconstrained such that the body 8912 takes on an arch-like shape upon deployment (FIG. 89B). For example, in the illustrated embodiment the tension member 8927 can comprise two of the elastic fibers-one positioned between each adjacent pair of the three illustrated stents 8926 along the body 8912. The elastic fibers of the tension member 8957 can be sewn onto the exterior of the graft material 8928 at the apices (or another portion) of the stents 8926 such that the elastic members draw the stents 8926 toward one another along the first side portion 8921. In some embodiments, the elastic fibers of the tension member 8957 can be secured to the stents 8926 in the same or a similar manner to that described in detail above with reference to FIG. 88C (e.g., in an apex-to apex, apex-to-base, and/or other arrangement). The tension member 8927 can be stretched to the straight first position (FIG. 89A) for loading into a delivery catheter in a cylindrical configuration. Upon deployment, the body 8912 of the base member 8910 can assume the curved second position (FIG. 88B). In some embodiments, in the curved second position (FIG. 89B), the tension member 8927 pulls some or all of the stents 8926 toward and at least partially over/under an adjacent one of the stents 8926 such that the first side portion 8921 has as stepped “shingle” pattern, as described in detail above with reference to FIG. 88B.

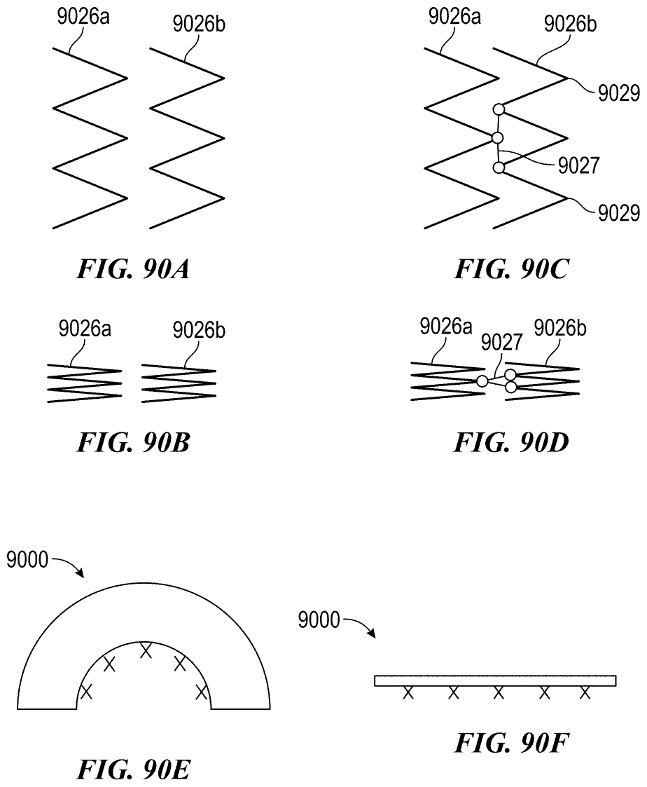

In some embodiments, a tension member for use in an aortic repair device can comprise non-elastic fibers, sutures, and/or structures that are arranged to passively shorten a portion of the aortic repair device. For example, FIGS. 90A and 90B are enlarged views of a pair of stents 9026 (including an individually identified first stent 9026a and second stent 9026b) of a component of an aortic repair device 9000—such as any of the base members and/or spanning members described herein—in a free state and a compacted state, respectively, in accordance with embodiments of the present technology. In the illustrated embodiment, the stents 9026 are identical and positioned in phase with one another (e.g., with their apices aligned). FIGS. 90C and 90D are enlarged views of the stents 9026 in the free state and the compacted state, respectively, after attachment of one or more tensioning sutures 9027 to the stents 9026 in accordance with embodiments of the present technology. The tensioning suture 9027 can comprise a typical surgical suture material and can be non-elastic. In the illustrated embodiment, the tensioning suture 9027 connects a single apex of the first stent 9026a to a pair of bases of the second stent 9026b to reduce the distance between the stents 9026 in the free state. Accordingly, in the free state, the tensioning suture 9027 can selectively shorten a portion (e.g., a side portion) of the aortic repair device 9000 proximate the tensioning suture 9027 to, for example, better conform the aortic repair device 9000 to a curvature within an aorta. In the compacted state, the tensioning suture 9027 can move (e.g., fold, pivot about the apex of the first stent 9026a) such that the aortic repair device 9000 can return to an elongate cylindrical state. In some embodiments, the tensioning suture 9027 can connect the stents 9026 in a different manner. For example, the tensioning suture 9027 can connect one or more apices of the first stent 9026a to one or more remote apices 9029 of the second stent 9026b to decrease the distance between the stents 9026 in the free state and increase nesting/shingling therebetween. FIGS. 90E and 90F are schematic sides views of the aortic repair device 9000 in the free state (e.g., bent state) and the compacted state (e.g., straight state), respectively, in accordance with embodiments of the present technology.

In some embodiments, the stents 9026 can have different configurations (e.g., shapes, sizes, patterns) to change the amount of nesting therebetween or otherwise affect the shortening of the aortic repair device 9000. For example, FIGS. 91A and 91B are enlarged views of the stents 9026 in the free state and the compacted state, respectively, in which the second stent 9026b has an alternating stent apex height in accordance with additional embodiments of the present technology. In particular, the second stent 9026b can have a smaller amplitude at the apices/bases where the tensioning suture 9027 connects to further increase the amount of nesting between the stents 9026 (and corresponding shortening and arching of the aortic repair device 9000).

FIGS. 28A-33 illustrate additional embodiments of aortic repair devices including a base member having integrated features for supporting a septum and a secondary leg lumen to, for example, inhibit compression of the secondary leg lumen and maintain blood flow through the secondary leg lumen when a spanning member is deployed within the base member in accordance with embodiments of the present technology. Such features can replace the need for a sperate support stent to be implanted within the base member. The various aortic repair devices can include some features that are at least generally similar in structure and function, or identical in structure and function, to one another and/or to the corresponding features of the aortic repair devices described in detail above with reference to FIGS. 3A-27D, and can operate in a generally similar or identical manner to one another and/or to the aortic repair devices described above.

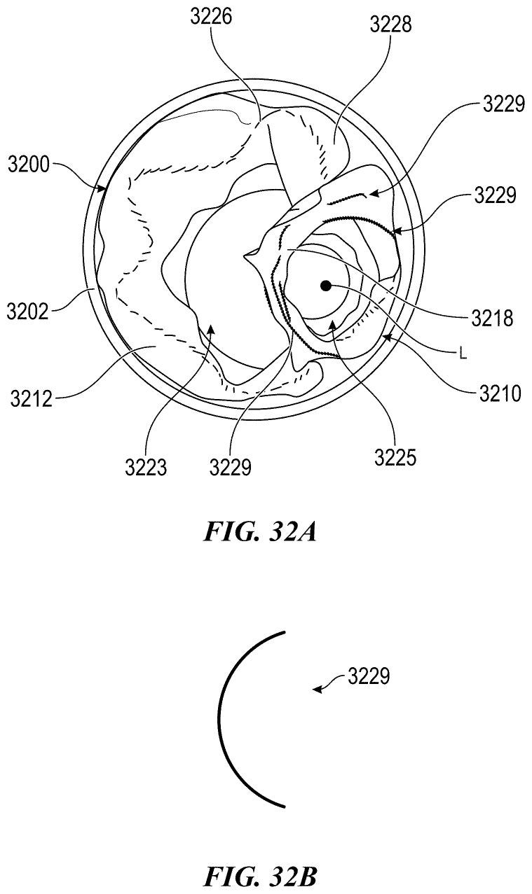

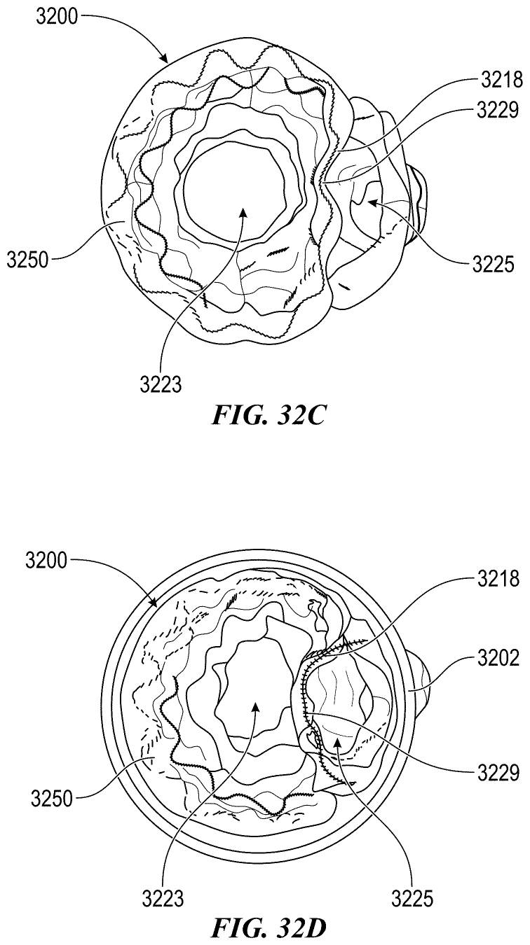

FIGS. 28A and 28B, for example, are a perspective view and a perspective end view, respectively, of an aortic repair device 2800 in accordance with embodiments of the present technology. Referring to FIGS. 28A and 28B together, the aortic repair device 2800 includes a base member 2810 having a (i) body 2812 defining a primary lumen 2823 and (ii) a leg 2814 defining a secondary lumen 2825. In the illustrated embodiment, the body 2812 and the leg 2814 are separate stent grafts secured together via, for example, stitching. More specifically, the body 2812 includes body stents 2826a coupled to a body graft material 2828a and the leg 2814 includes leg stents 2826b coupled to a leg graft material 2828b. The body and leg stents 2826a-b can be formed from the same material and have a similar shape (e.g., including a circumferential V-pattern) and the body and leg graft materials 2828a-b can be formed from the same material (e.g., fabric). The leg graft material 2828b can function as a septum and define the secondary lumen 2825. In some embodiments, the leg stents 2826b extend entirely circumferentially about the secondary lumen 2825 while, in other embodiments (e.g., as described in greater below with reference to FIGS. 32A-32D), the leg stents 2826b can extend only partially circumferentially about the secondary lumen 2825.

The leg stents 2826b can provide increased radial support for the leg graft material 2828b compared to an unsupported septum such as, for example, the septum 818 shown in FIGS. 8A-8H when the stent 830 is not deployed within the secondary lumen 825. Accordingly, in some aspects of the present technology, the leg stents 2826b can help preserve the cross-sectional flow area of the secondary lumen 2825 when a spanning member is deployed within the primary lumen 2823. Moreover, the leg stents 2826b are integral with the base member 2810 such that a separate step or procedure is not needed to implant the leg stents 2826b therein (e.g., as compared to the deployment of the stent 830 described in detail above with reference to FIGS. 8A-8H).

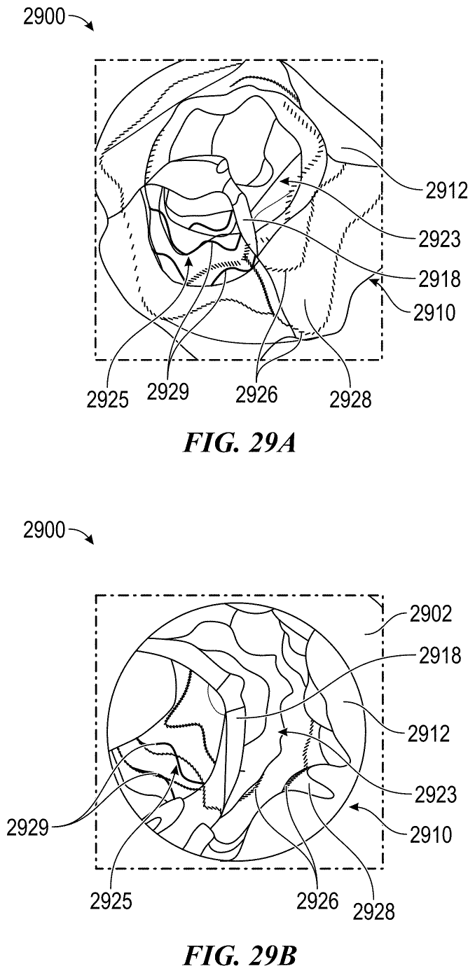

FIGS. 29A and 29B are perspective end views of an aortic repair device 2900 in accordance with embodiments of the present technology. In FIG. 29B, the aortic repair device 2900 is deployed within a lumen 2902, such as a portion of an aorta. Referring to FIGS. 29A and 29B together, the aortic repair device 2900 includes a base member 2910 having a body 2912 with a septum 2918 extending fully or partially therethrough and dividing the body 2912 into a primary lumen 2923 and a secondary lumen 2925 having a smaller size than the primary lumen 2923. In the illustrated embodiment, the body 2912 includes body stents 2926 coupled to a graft material 2928. In the illustrated embodiment, the base member 2910 further includes one or more secondary lumen stents 2929 positioned within the secondary lumen 2925. In some embodiments, the secondary lumen stents 2929 extend entirely circumferentially about the secondary lumen 2925 and are coupled to the graft material 2928 and/or the septum 2918 via, for example, stitching. The secondary lumen stents 2929 can be self-expanding and can have a V-pattern or other shape. In some embodiments, the secondary lumen stents 2929 have a circular cross-sectional shape. In other embodiments, the secondary lumen stents 2929 have a non-circular cross-sectional shape, such as an oval or other shape. In some embodiments, as shown in FIG. 29B, the secondary lumen stents 2929 bow outward toward the primary lumen 2223 when the aortic repair device 2900 is compressed within the lumen 2902.

The secondary lumen stents 2929 can provide increased radial support for the septum 2918 compared to an unsupported septum such as, for example, the septum 818 shown in FIGS. 8A-8H when the stent 830 is not deployed within the secondary lumen 825. Accordingly, in some aspects of the present technology, the secondary lumen stents 2929 can help preserve the cross-sectional flow area of the secondary lumen 2925 when a spanning member is deployed within the primary lumen 2923. For example, the secondary lumen stents 2929 can resist and/or match the radial force of the spanning member deployed within the primary lumen 2923 to inhibit displacement of the septum 2918 and maintain the cross-sectional flow area of the secondary lumen 2925. The outward bowing of the secondary lumen stents 2929 can further resist compression from the spanning member. In some embodiments, the secondary lumen stents 2929 can be stiff enough to inhibit compression of the secondary lumen 2925 while also being compliant enough to conform with the spanning member sufficiently to inhibit significant gutters or openings between the spanning member and the septum 2918. In additional aspects of the present technology, the secondary lumen stents 2929 are integral with the base member 2910 such that a separate step or procedure is not needed to implant the secondary lumen stents 2929 therein (e.g., as compared to the deployment of the stent 830 described in detail above with reference to FIGS. 8A-8H).

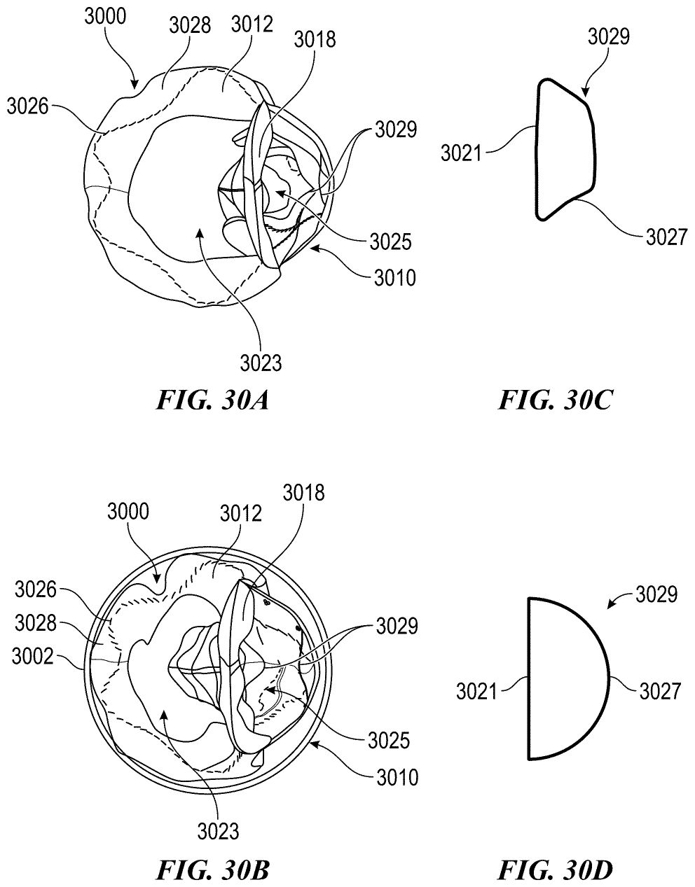

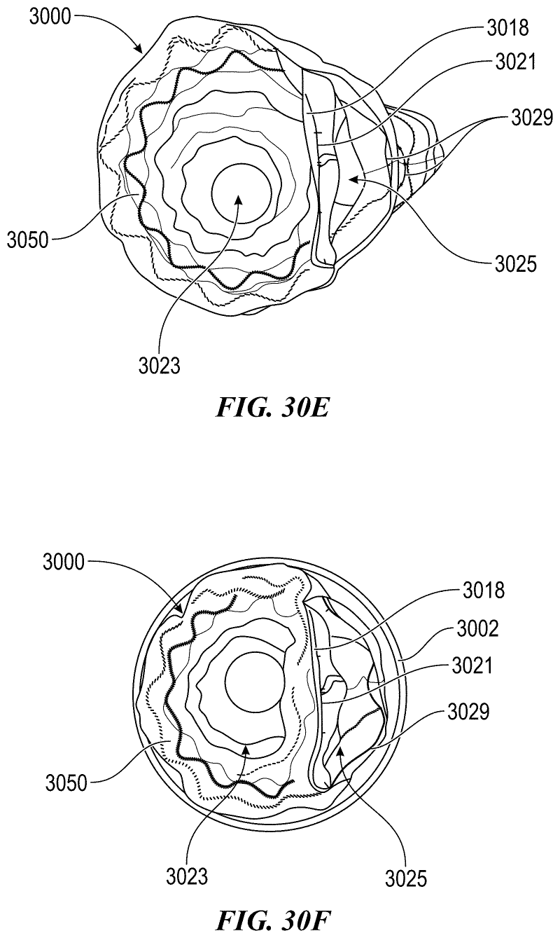

FIGS. 30A and 30B are perspective end views of an aortic repair device 3000 in accordance with embodiments of the present technology. In FIG. 30B, the aortic repair device 3000 is deployed within a lumen 3002, such as a portion of an aorta. Referring to FIGS. 30A and 30B together, the aortic repair device 3000 includes a base member 3010 having a body 3012 with a septum 3018 extending fully or partially therethrough and dividing the body 3012 into a primary lumen 3023 and a secondary lumen 3025 having a smaller size than the primary lumen 3023. In the illustrated embodiment, the body 3012 includes body stents 3026 coupled to a graft material 3028. The base member 3010 can further include one or more secondary lumen stents 3029 positioned within the secondary lumen 3025. In some embodiments, the secondary lumen stents 3029 extend entirely circumferentially about the secondary lumen 3025 and are coupled to the graft material 3028 and/or the septum 3018 via, for example, stitching.

FIGS. 30C and 30D are a perspective end view and an end view, respectively, of one of the secondary lumen stents 3029 in accordance with embodiments of the present technology. Referring to FIGS. 30C and 30D together, the secondary lumen stents 3029 can have a D-like cross-sectional shape including a generally flat portion 3021 and a generally curved portion 3027. The secondary lumen stents 3029 can further include a V-pattern extending circumferentially about the flat portion 3021 and the curved portion 3027 as shown in FIG. 30C. Referring to FIGS. 30A-30D together, the flat portions 3021 of the secondary lumen stents 3029 can be positioned adjacent and/or coupled to the septum 3018 and the curved portions 3027 of the secondary lumen stents 3029 can be positioned adjacent and/or coupled to the graft material 3028 of the body 3012. Accordingly, in the unconstrained and constrained positions shown in FIGS. 30A and 30B, respectively, the septum 3018 can extend generally linearly between the primary lumen 3023 and the secondary lumen 3025.

Similar to the embodiments described in detail above with reference to FIGS. 28A-29C, the secondary lumen stents 3029 can provide increased radial support for the septum 3018 compared to an unsupported septum such as, for example, the septum 818 shown in FIGS. 8A-8H when the stent 830 is not deployed within the secondary lumen 825. Accordingly, in some aspects of the present technology, the secondary lumen stents 3029 can help preserve the cross-sectional flow area of the secondary lumen 3025 when a spanning member is deployed within the primary lumen 3023. For example, FIGS. 30E and 30F are perspective end views of the aortic repair device 3000 with a spanning member 3050 deployed within the primary lumen 3023. In FIG. 30F, the aortic repair device 3000 is deployed within the lumen 3002. Referring to FIGS. 30E and 30F together, the secondary lumen stents 3029 resist and/or match the radial force of the spanning member 3050 to inhibit displacement of the septum 3018 and maintain the cross-sectional flow area of the secondary lumen 3025. In some embodiments, the secondary lumen stents 3029 can be stiff enough to inhibit compression of the secondary lumen 3025 while also being compliant enough to conform with the spanning member 3050 sufficiently to inhibit significant gutters or openings between the spanning member 3050 and the septum 3018. Moreover, the flat portions 3021 of the secondary lumen stents 3029 can maintain the septum 3018 in a generally flat or linear configuration such that the spanning member 3050 can conform thereto with minimal gutters or openings between the septum 3018 and the spanning member 3050. Referring to FIGS. 30A-30F together, in additional aspects of the present technology the secondary lumen stents 3029 are integral with the base member 3010 such that a separate step or procedure is not needed to implant the secondary lumen stents 3029 therein (e.g., as compared to the deployment of the stent 830 described in detail above with reference to FIGS. 8A-8H).

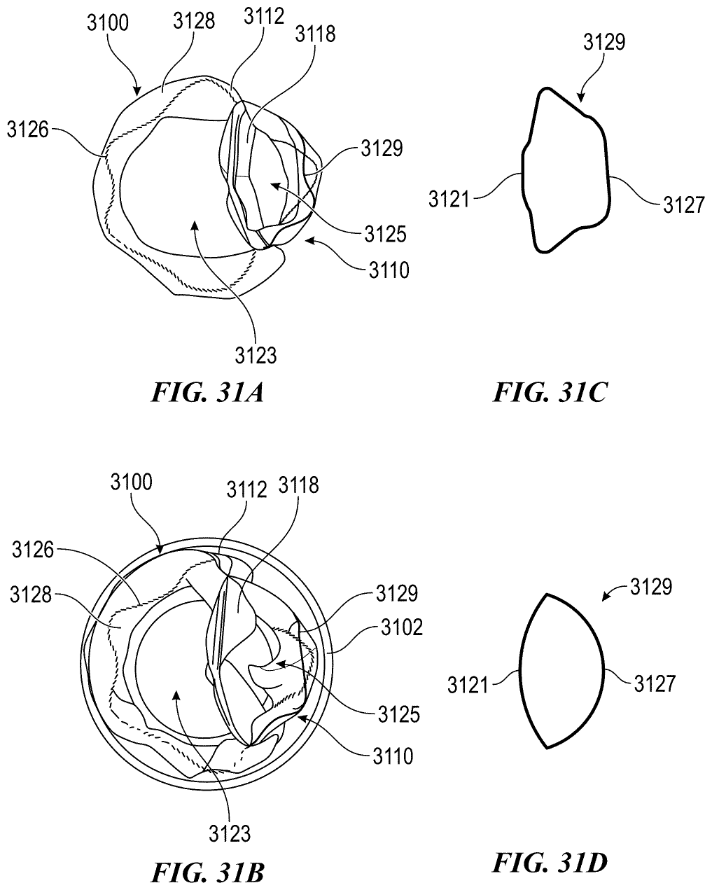

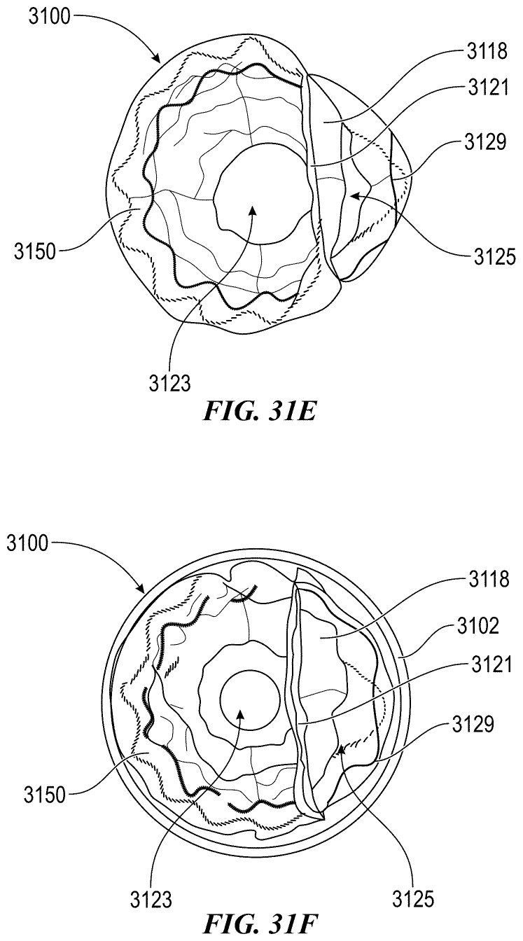

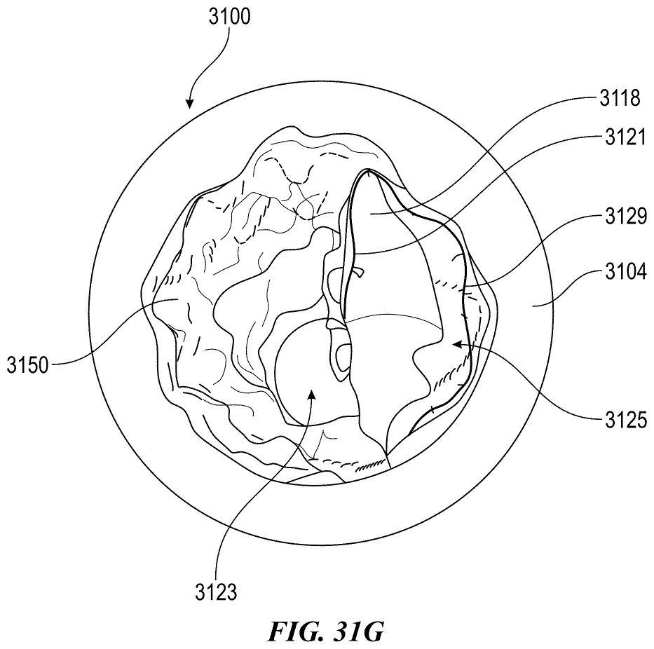

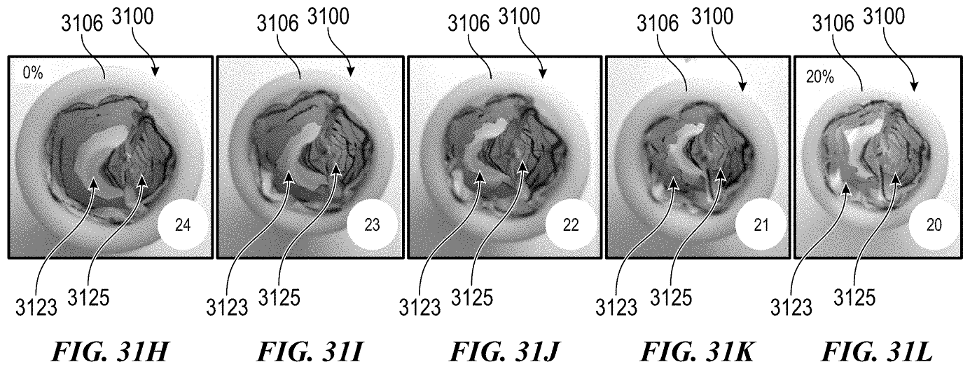

FIGS. 31A and 31B are perspective end views of an aortic repair device 3100 in accordance with embodiments of the present technology. In FIG. 31B, the aortic repair device 3100 is deployed within a first lumen 3102, such as a portion of an aorta. Referring to FIGS. 31A and 31B together, the aortic repair device 3100 includes a base member 3110 having a body 3112 with a septum 3118 extending fully or partially therethrough and dividing the body 3112 into a primary lumen 3123 and a secondary lumen 3125 having a smaller size than the primary lumen 3123. In the illustrated embodiment, the body 3112 includes body stents 3126 coupled to a graft material 3128. In the illustrated embodiment, the base member 3110 further includes one or more secondary lumen stents 3129 positioned within the secondary lumen 3125. In some embodiments, the secondary lumen stents 3129 extend entirely circumferentially about the secondary lumen 3125 and are coupled to the graft material 3128 and/or the septum 3118 via, for example, stitching.

FIGS. 31C and 31D are a perspective end view and an end view, respectively, of one of the secondary lumen stents 3129 in accordance with embodiments of the present technology. Referring to FIGS. 31C and 31D together, the secondary lumen stents 3129 can have a bowed-D-like cross-sectional shape including a curved first portion 3121 and a curved second portion 3127. The curved first portion 3121 can have a radius of curvature that is greater than a radius of curvature of the curved second portion 3127. That is, the curved first portion 3121 can be relatively flatter than the curved second portion 3127. The curved first portion 3121 can also be referred to as a spacer, a bowed portion, and/or the like. The secondary lumen stents 3129 can further include a V-pattern extending circumferentially about the curved first portion 3121 and the curved second portion 3129 as shown in FIG. 31C. Referring to FIGS. 31A-31D together, the curved first portions 3121 of the secondary lumen stents 3129 can be positioned adjacent and/or coupled to the septum 3118 and the curved second portions 3127 of the secondary lumen stents 3129 can be positioned adjacent and/or coupled to the graft material 3128 of the body 3112. Accordingly, in the unconstrained and constrained positions shown in FIGS. 31A and 31B, respectively, the septum 3118 can be curved outward (e.g., bowed outward) between the primary lumen 3123 and the secondary lumen 3125 along the curved first portions 3121.

Similar to the embodiments described in detail above with reference to FIGS. 28A-30F, the secondary lumen stents 3129 can provide increased radial support for the septum 3118 compared to an unsupported septum such as, for example, the septum 818 shown in FIGS. 8A-8H when the stent 830 is not deployed within the secondary lumen 825. Accordingly, in some aspects of the present technology, the secondary lumen stents 3129 can help preserve the cross-sectional flow area of the secondary lumen 3125 when a spanning member is deployed within the primary lumen 3123. For example, FIGS. 31E-31G are perspective end views of the aortic repair device 3100 with a spanning member 3150 deployed within the primary lumen 3123 in accordance with embodiments of the present technology. In FIG. 31E, the aortic repair device 3100 is unconstrained, in FIG. 31F the aortic repair device 3100 is deployed within the first lumen 3102 having a first diameter (e.g., 31 millimeters), and in FIG. 31G the aortic repair device 3100 is deployed within a second lumen 3104 having a second diameter (e.g., 29 millimeters) smaller than the first diameter. Referring to FIGS. 31E-31G together, the secondary lumen stents 3129 resist and/or match the radial force of the spanning member 3150 to inhibit displacement of the septum 3118 and maintain the cross-sectional flow area of the secondary lumen 3125. In some embodiments, the secondary lumen stents 3129 can be stiff enough to inhibit compression of the secondary lumen 3125 while also being compliant enough to conform with the spanning member 3150 sufficiently to inhibit significant gutters or openings between the spanning member 3150 and the septum 3118. Referring to FIGS. 31A-31G together, in additional aspects of the present technology the secondary lumen stents 3129 are integral with the base member 3110 such that a separate step or procedure is not needed to implant the secondary lumen stents 3129 therein (e.g., as compared to the deployment of the stent 831 described in detail above with reference to FIGS. 8A-8H).

As shown in FIG. 31G, when the lumen 3104 is smaller and further limits overall radial expansion of the aortic repair device 3100, the curved first portions 3121 of the secondary lumen stents 3129 can bow and/or flex further outward (e.g., curve more, become more convex) to maintain the cross-sectional flow area of the secondary lumen 3125. In some embodiments, the structure that defines the first lumen 3123 can be relatively more compressible (e.g., less compression-resistant) than the structure that defines the second lumen 3125 such that a ratio of the cross-sectional flow area of the first lumen 3123 to the cross-sectional flow area of the secondary lumen 3125 decreases when the aortic repair device 3100 is radially compressed, regardless of whether the spanning member 3150 is deployed within the primary lumen 3123. For example, FIGS. 31H-31L are perspective end views of the aortic repair device 3100 illustrating increasing radial compression of the aortic repair device 3100 in accordance with embodiments of the present technology. Specifically, referring to FIGS. 31H-31L together, the aortic repair device 3100 is deployed within a lumen 3106, such as a portion of an aorta, having a progressively decreasing diameter from FIG. 31H to FIG. 31L. In the illustrated embodiment, the diameter (e.g., 20 mm) of the lumen 3106 is 20% less in FIG. 31L than the diameter (e.g., 24 mm) of the lumen 3106 in FIG. 31H and progressively decreases therebetween in FIGS. 31I-31K (e.g., from 23 mm, to 22 mm, to 21 mm, respectively). In some embodiments, the aortic repair device 3100 can have a diameter (e.g., 24 mm) that matches the diameter of the lumen 3106 shown in FIG. 31H such that the aortic repair device 3100 is not oversized relative to the lumen 3106. The structure defining the secondary lumen 3125 is relatively less compressible than the first lumen 3123 such that the ratio of the cross-sectional flow area of the secondary lumen 3125 to the cross-sectional flow area of the primary lumen 3123 increases from FIG. 31H to FIG. 31L as radial expansion of the aortic repair device 3100 is further limited by the lumen 3106 (e.g., the inner diameter of the aorta). Thus, in some aspects of the present technology the secondary lumen 3125 is well preserved through a range of oversizing of the aortic repair device 3100 relative to the lumen 3106.

FIG. 32A is a perspective end view of an aortic repair device 3200 deployed within a lumen 3202 in accordance with embodiments of the present technology. In the illustrated embodiment, the aortic repair device 3200 includes a base member 3210 having a body 3212 with a septum 3218 extending fully or partially therethrough and dividing the body 3212 into a primary lumen 3223 and a secondary lumen 3225 having a smaller size than the primary lumen 3223. In the illustrated embodiment, the body 3212 includes body stents 3226 coupled to a graft material 3228. In the illustrated embodiment, the base member 3210 further includes one or more secondary lumen stents 3229 positioned within the secondary lumen 3225. In some embodiments, the secondary lumen stents 3229 extend only partially circumferentially about the secondary lumen 3225 and are coupled to the graft material 3228 and/or the septum 3218 via, for example, stitching. More specifically, FIG. 32B is an end view of one of the secondary lumen stents 3229 in accordance with embodiments of the present technology. In the illustrated embodiment, the secondary lumen stents 3229 can extend only partially about a longitudinal axis L (FIG. 32A) of the secondary lumen 3225 and can have a curved shape, such as semi-circular or bowed shape. The secondary lumen stents 3229 can further include a V-pattern extending circumferentially there along. Referring to FIGS. 32A and 32B together, the secondary lumen stents 3229 can be positioned adjacent and/or coupled to the septum 3218. Accordingly, in an unconstrained position (not shown) and the constrained position shown in FIG. 32A, the septum 3218 can be curved outward (e.g., bowed outward) between the primary lumen 3223 and the secondary lumen 3225 along secondary lumen stents 3229. In some aspects of the present technology, the secondary lumen stents 3229 can reduce the overall delivery profile of the base member 3210 by omitting the secondary lumen stents 3229 adjacent to the body stents 3226 (e.g., where the body stents 3226 provide an outward sealing force to the body 3212).

Similar to the embodiments described in detail above with reference to FIGS. 28A-31G, the secondary lumen stents 3229 can provide increased radial support for the septum 3218 compared to an unsupported septum such as, for example, the septum 818 shown in FIGS. 8A-8H when the stent 830 is not deployed within the secondary lumen 825. Accordingly, in some aspects of the present technology, the secondary lumen stents 3229 can help preserve the cross-sectional flow area of the secondary lumen 3225 when a spanning member is deployed within the primary lumen 3223. For example, FIGS. 32C and 32D are perspective end views of the aortic repair device 3200 with a spanning member 3250 deployed within the primary lumen 3223. In FIG. 32C, the aortic repair device 3200 is unconstrained, and in FIG. 32D the aortic repair device 3200 is deployed within the lumen 3202. Referring to FIGS. 32C and 32D together, the secondary lumen stents 3229 resist and/or match the radial force of the spanning member 3250 to inhibit displacement of the septum 3218 and maintain the cross-sectional flow area of the secondary lumen 3225. In some embodiments, the secondary lumen stents 3229 can be stiff enough to inhibit compression of the secondary lumen 3225 while also being compliant enough to conform with the spanning member 3250 sufficiently to inhibit significant gutters or openings between the spanning member 3250 and the septum 3218. Referring to FIGS. 32A-32D together, in additional aspects of the present technology the secondary lumen stents 3229 are integral with the base member 3210 such that a separate step or procedure is not needed to implant the secondary lumen stents 3229 therein (e.g., as compared to the deployment of the stent 832 described in detail above with reference to FIGS. 8A-8H).

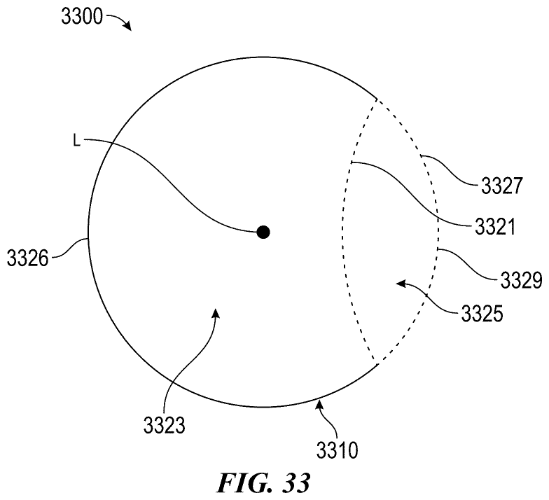

FIG. 33 is a cross-sectional view of an aortic repair device 3300 in accordance with embodiments of the present technology. In the illustrated embodiment, the aortic repair device 3300 includes a base member 3310 including one or more body stents 3326 defining a primary lumen 3323 and one or more secondary lumen stents 3329 defining a secondary lumen 3325. In the illustrated embodiment, the secondary lumen stents have a bowed-D like cross-sectional shape including a curved first portion 3321 and a curved second portion 3327. The curved first portions 3321 of the secondary lumen stents 3329 can be positioned adjacent and/or coupled to a septum of the base member 3310 and the curved second portions 3327 of the secondary lumen stents 3329 can be positioned adjacent and/or coupled to a graft material of a body of the base member 3310. In some aspects of the present technology, the secondary lumen stents 3329 resist and/or match the radial force of a spanning member deployed within the primary lumen 3323 to inhibit displacement of the septum and maintain the cross-sectional flow area of the secondary lumen 3325.

In the illustrated embodiment, the body stents 3326 can extend only partially about a longitudinal axis L of the primary lumen 3323 and can have a curved shape, such as a circular shape. The body stents 3326 and the curved second portions 3327 of the secondary lumen stents 3329 can together define a constant or nearly constant circumferential stent structure about the base member 3310 to, for example, provide an outward sealing force when the base member 3310 is implanted within an aorta. Moreover, in some aspects of the present technology the arrangement of the body stents 3326 and the secondary lumen stents 3329 can reduce the overall delivery profile of the base member 3310 by omitting a portion of the body stents 3326 adjacent the curved second portions 3327 of the secondary lumen stents 3329 (e.g., such that the separate stents do not overlap).

Referring to FIGS. 28A-33 together, any of the secondary lumen stents can be made stronger (e.g., to have an increased radial force, an increased crush resistance, and/or the like) by increasing the wire diameter of the secondary lumen stents and/or by heat treating (e.g., shape setting) the wires for a longer duration and/or at a higher temperature. Increasing the strength of the secondary lumen stents can help improve the ability of the secondary lumen stents to preserve the cross-sectional flow area of the secondary lumen during radial compression and/or when a spanning member is deployed within the primary lumen.

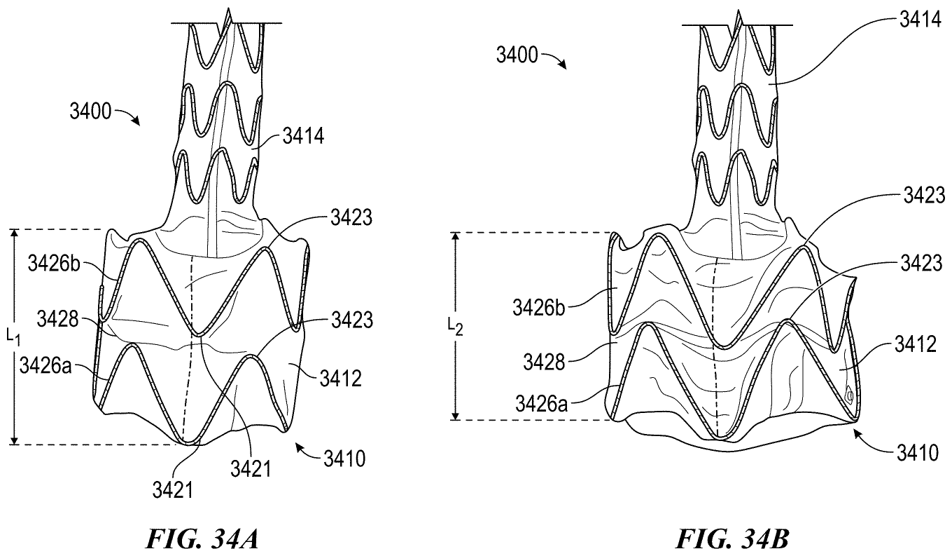

FIGS. 34A-40C illustrate additional embodiments of aortic repair devices including a base member having stents configured to inhibit or permit longitudinal compression of the base member in accordance with embodiments of the present technology. The various aortic repair devices can include some features that are at least generally similar in structure and function, or identical in structure and function, to one another and/or to the corresponding features of the aortic repair devices described in detail above with reference to FIGS. 3A-33, and can operate in a generally similar or identical manner to one another and/or to the aortic repair devices described above.

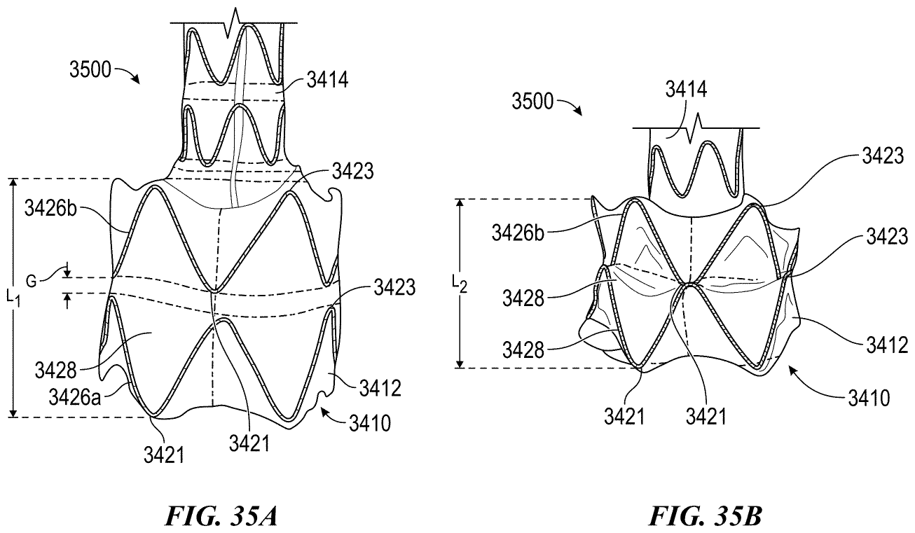

For example, FIGS. 34A and 34B are perspective side views of an aortic repair device 3400 in a longitudinally elongated position and a longitudinally compressed position, respectively, in accordance with embodiments of the present technology. Similarly, FIGS. 35A and 35B are perspective side views of an aortic repair device 3500 in a longitudinally elongated position and a longitudinally compressed position, respectively, in accordance with embodiments of the present technology. The aortic repair device 3400 and the aortic repair device 3500 can include several features that are generally similar or identical. For example, referring to FIGS. 34A-35B together, both the aortic repair device 3400 and the aortic repair device 3500 include a base member 3410 having a body 3412, a leg 3414 extending from the body 3412, and a septum (obscured in FIGS. 34A-35B) extending fully or partially through the body 3412 and dividing the body 3412 into a primary lumen and a secondary lumen. The base member 3410 comprises one or more stents 3426 coupled to a graft material 3428. More specifically, the stents 3426 along the body 3412 can include a first stent 3426a (e.g., a proximal stent) and a second stent 3426b (e.g., a distal stent) each having a periodic V-pattern shape including alternating proximal apices 3421 and distal apices 3423. In some embodiments, the first stent 3426a and the second stent 3426b are identical—for example, each having a same size and a same number of the proximal apices 3421 and the distal apices 3423.

Referring to FIGS. 34A and 34B together, the proximal apices 3421 of the first stent 3426a are aligned or generally aligned circumferentially with the proximal apices 3421 of the second stent 3426b. Likewise, the distal apices 3423 of the first stent 3426a are aligned or generally aligned circumferentially with the distal apices 3423 of the second stent 3426b. That is, the first stent 3426a and the second stent 3426b can be arranged in phase or substantially in phase. As shown in FIG. 34B, this arrangement of the stents 3426a-b can permit the body 3412 to longitudinally compress when, for example, the aortic repair device 3400 is implanted within the aorta and subject to forces from blood flow through the aorta. For example, the distal apices 3423 of the first stent 3426a can move toward the distal apices 3423 of the second stent 3426b while folding or otherwise compressing the graft material 3428 therebetween. In some embodiments, the longitudinal compression (e.g., a difference between a length L₁ of the body 3412 in the longitudinally elongated position shown in FIG. 34A and a length L₂ of the body 3412 in the longitudinally compressed position shown in FIG. 34B) can be about 40%, less than about 40%, about 20%, less than about 20%, etc.

Referring to FIGS. 35A and 35B together, the proximal apices 3421 of the first stent 3426a are aligned or generally aligned circumferentially with the distal apices 3423 of the second stent 3426b. Likewise, the distal apices 3423 of the first stent 3426a are aligned or generally aligned circumferentially with the proximal apices 3421 of the second stent 3426b. That is, the first stent 3426a and the second stent 3426b can be arranged out of phase (e.g., 180 degrees out of phase) or substantially out of phase. As shown in FIG. 35A, in the longitudinally elongated position, the distal apices 3423 of the first stent 3426a can be spaced apart from the corresponding ones of the proximal apices 3421 of the second stent 3426b by a gap G. The gap G can provide the body 3412 with a high flexibility as the body 3412 can flex along the gap G where the stents 3426a-b are omitted. As shown in FIG. 35B, this arrangement of the stents 3426a-b can permit the body 3412 to longitudinally compress when, for example, the aortic repair device 3400 is implanted within the aorta and subject to forces from blood flow through the aorta. For example, the distal apices 3423 of the first stent 3426a can move toward and under and/or over the proximal apices 3421 of the second stent 3426b (e.g., reducing or eliminating the gap G) while folding or otherwise compressing the graft material 3428 therebetween. In some embodiments, the longitudinal compression (e.g., a difference between a length L₁ of the body 3412 in the longitudinally elongated position shown in FIG. 35A and a length L₂ of the body 3412 in the longitudinally compressed position shown in FIG. 35B) can be about 20%.

FIGS. 36A and 36B are perspective side views of an aortic repair device 3600 in a longitudinally elongated position from different circumferential perspectives in accordance with embodiments of the present technology. FIG. 36C is a perspective side view of the aortic repair device 3600 in a longitudinally compressed position in accordance with embodiments of the present technology. Referring to FIGS. 36A-36C, the aortic repair device 3600 can include a base member 3610 having a body 3612, a leg (obscured in FIGS. 36A-36C) extending from the body 3612, and a septum (obscured in FIGS. 36A-36C) extending fully or partially through the body 3612 and dividing the body 3612 into a primary lumen and a secondary lumen. The base member 3610 comprises one or more stents 3626 coupled to a graft material 3628. More specifically, the stents 3626 along the body 3612 can include a first stent 3626a (e.g., a proximal stent) and a second stent 3626b (e.g., a distal stent) each having a periodic V-pattern shape including alternating proximal apices 3621 and distal apices 3623. In the illustrated embodiment, the first stent 3626a and the second stent 3626b each have a different number of the proximal apices 3621 and the distal apices 3623 (e.g., a different frequency).

More specifically, FIG. 36D is a planar view of the first stent 3626a and the second stent 3626b in accordance with embodiments of the present technology. In the illustrated embodiment, the first stent 3626a includes eight V-shaped portions such that the first stent 3626a has eight of the proximal apices 3621 and eight of the distal apices 3623, and the second stent 3626b includes six V-shaped portions such that the second stent 3626b has six of the proximal apices 3621 and six of the distal apices 3623. In other embodiments, the stents 3626a-b can have different numbers of the proximal apices 3621 and/or the distal apices 3623. In the illustrated embodiment, the first stent 3426a and the second stent 3426b each have a regular pattern such that the V-shaped portions thereof are equally spaced apart from another. Accordingly, a first subset of the distal apices 3623 of the first stent 3626a can be circumferentially aligned with the proximal apices 3621 of the second stent 3626b, while a second subset of the distal apices 3623 of the first stent 3626a can be circumferentially offset from the proximal apices 3621 of the second stent 3626b. In the illustrated embodiment, the first subset includes two of the distal apices 3623 of the first stent 3626a and the second subset includes six of the distal apices 3623 of the first stent 3626a. (FIG. 36D illustrates a wraparound view such that the distal apices 3623 of the first stent 3626a at the right and left ends of the first stent 3636a represent the same one of the distal apices 3623 of the first stent 3626a.) For example, the distal apices 3623 of the first stent 3626a positioned approximately 180 degrees apart (e.g., on opposite sides of the body 3612 shown in FIGS. 36A-36C) are circumferentially aligned with corresponding ones of the proximal apices 3621 of the second stent 3626b. In other embodiments, the number of the distal apices 3623 of the first stent 3626a in the first subset and the second subset can differ based on, for example, the total number of apices of the stents 3626a-b. Further, in the illustrated embodiment the aligned ones of the distal apices 3623 of the first stent 3626a and the proximal apices 3621 of the second stent 3626b can abut each other such that they are not separated by a gap.

Referring to FIGS. 36A-36D together, the circumferentially aligned ones of the distal apices 3623 of the first stent 3626a and the proximal apices 3621 of the second stent 3626b can inhibit longitudinal compression (e.g., provide an increased column force) when, for example, the aortic repair device 3600 is implanted within the aorta and subject to forces from blood flow through the aorta. For example, the longitudinal compression (e.g., a difference between a length L₁ of the body 3612 in the longitudinally elongated position shown in FIG. 36A and a length L₂ of the body 3612 in the longitudinally compressed position shown in FIG. 36C) can be about 10% or less than 10%. In some aspects of the present technology, such inhibiting of longitudinal compression can help maintain the seal between the body 3612 and the aorta when aortic repair device 3600 is implanted therein. Further, the circumferentially offset ones of the distal apices 3623 of the first stent 3626a and the proximal apices 3621 of the second stent 3626b can provide the body 3612 with a relatively high flexibility.

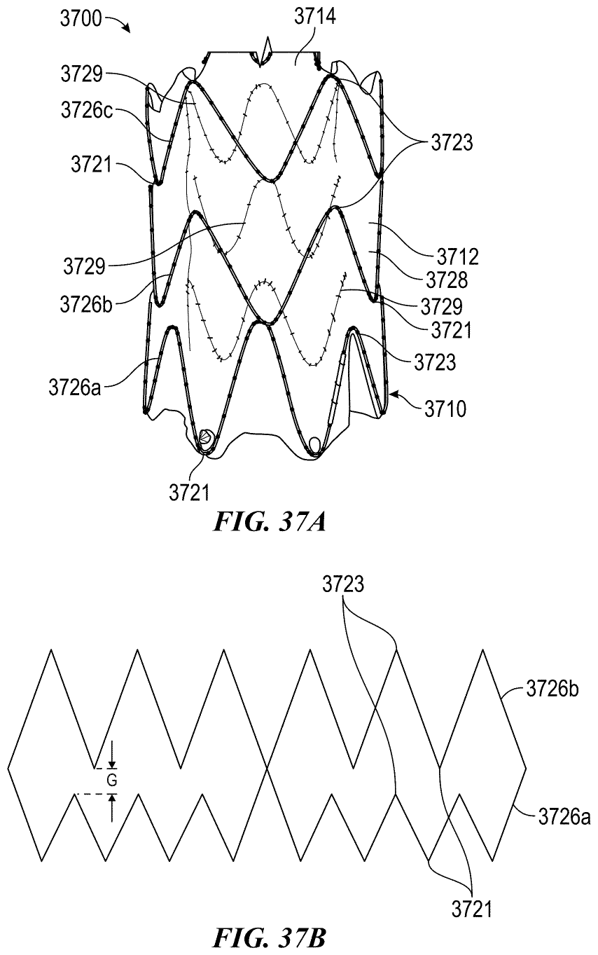

For any of the aortic repair devices disclosed herein, the phase, amplitude, frequency, and/or spacing of different stents (also referred to as stent components, such as stent rings) of an aortic repair device can be selected to at least partially provide for a desired axial stiffness and/or flexibility profile. Additionally or alternatively, the phase, amplitude, and/or frequency of individual stents can differ along their circumference to at least partially provide the desired axial stiffness and/or flexibility profile. FIG. 37A, for example, is a perspective side view of an aortic repair device 3700 in accordance with embodiments of the present technology. In the illustrated embodiment, the aortic repair device 3700 can include a base member 3710 having a body 3712, a leg 3714 (partially obscured in FIG. 37A) extending from the body 3712, and a septum (obscured in FIG. 37A) extending fully or partially through the body 3712 and dividing the body 3712 into a primary lumen and a secondary lumen. The base member 3710 comprises one or more stents 3726 coupled to a graft material 3728. More specifically, the stents 3726 along the body 3712 can include a first stent 3726a (e.g., a proximal stent), a second stent 3726b (e.g., a mid stent), and a third stent 3726c (e.g., a distal stent) each having a periodic V-pattern shape including alternating proximal apices 3721 and distal apices 3723. In the illustrated embodiment, the first stent 3726a and the second stent 3726b each have a different number of the proximal apices 3721 and the distal apices 3723 (e.g., a different frequency), and are arranged such that a subset (e.g., two) of the distal apices 3723 of the first stent 3726a are circumferentially aligned with corresponding ones of the proximal apices 3721 of the second stent 3726b (e.g., as described in detail above with reference to FIGS. 36A-36D). The second stent 3726b and the third stent 3726c are substantially identical and are arranged in phase (e.g., as described in detail above with reference to FIGS. 34A and 34B).

More specifically, FIG. 37B is a planar view of the first stent 3726a and the second stent 3726b in accordance with embodiments of the present technology. In the illustrated embodiment, the first stent 3726a has a varying amplitude such that (i) the V-shaped portions of the first stent 3726a have a greater amplitude at the ones of the distal apices 3723 circumferentially aligned with the proximal apices 3721 of the second stent 3726b and (ii) a gap G exists between the circumferentially offset ones of the distal apices 3723 of the first stent 3726a and the proximal apices 3721 of the second stent 3726b. Referring to FIGS. 37A and 37B together, in some aspects of the present technology this arrangement of the first and second stents 3726a-b (i) positions more of the flexible graft material 3728 between the stents 3726a-b (e.g., unsupported by the stents 3726a-b) where the distal apices 3723 of the first stent 3726a are circumferentially offset from the proximal apices 3721 of the second stent 3726b to increase the flexibility therebetween and (ii) abuts the distal apices 3723 of the first stent 3726a with the circumferentially aligned ones of the proximal apices 3721 of the second stent 3726b to provide axial stiffness where the distal apices 3723 of the first stent 3726a are aligned with the proximal apices 3721 of the second stent 3726b.

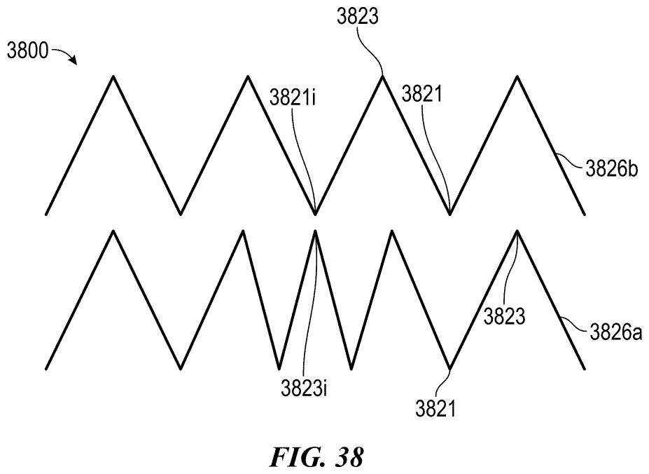

FIG. 38 is a planar view of a first stent 3826a (e.g., a proximal stent) and a second stent 3826b (e.g., a distal stent) of an aortic repair device 3800 in accordance with additional embodiments of the present technology. The first and second stents 3826a-b each have a periodic V-pattern shape including alternating proximal apices 3821 and distal apices 3823. In the illustrated embodiment, the first stent 3726a has an irregular frequency (e.g., spacing between the proximal apices 3821 and/or the distal apices 3823) and the second stent 3726b has a regular frequency such that one of the distal apices 3823 of the first stent 3826a (identified as an aligned distal apex 3823i) is circumferentially aligned with a corresponding one of the proximal apices 3821 of the second stent 3826b (identified as an aligned proximal apex 3821i). That is, the shift in spacing between the proximal apices 3821 and/or the distal apices 3823 of the first stent 3826a (e.g., a shift in the width of the V-shaped portions thereof) can align the aligned distal apex 3823i in close proximity with the aligned proximal apex 3821i. In some aspects of the present technology, the arrangement of the aligned distal apex 3823i and the aligned proximal apex 3821i can increase the axial (e.g., columnar) stiffness of the aortic repair device 3800. In other embodiments, the frequency of the first stent 3826a and/or the second stent 3826b can be varied in other manners to align one or more of the distal apices 3823 of the first stent 3826a with one or more of the proximal apices 3821 of the second stent 3826b to provide a desired amount of axial stiffness.

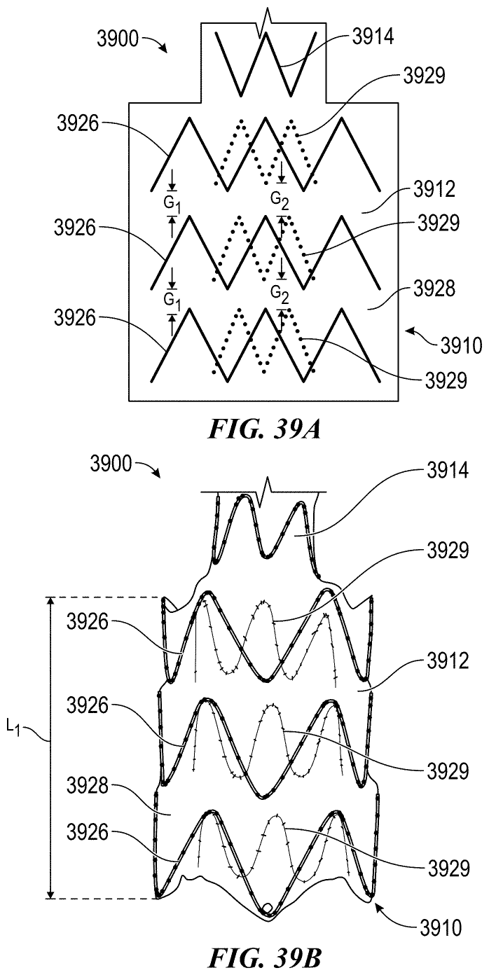

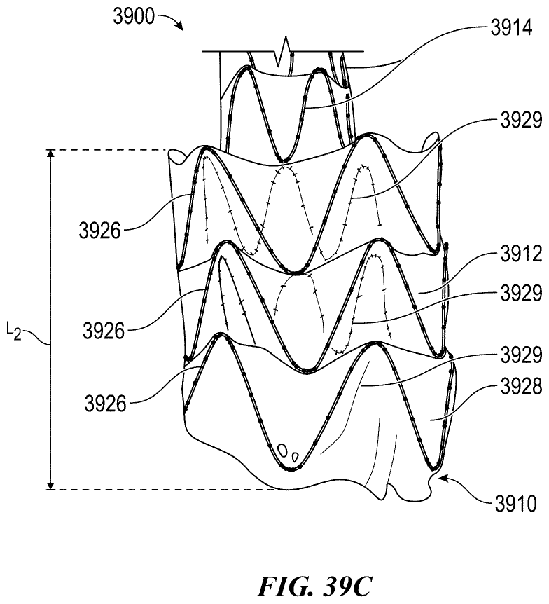

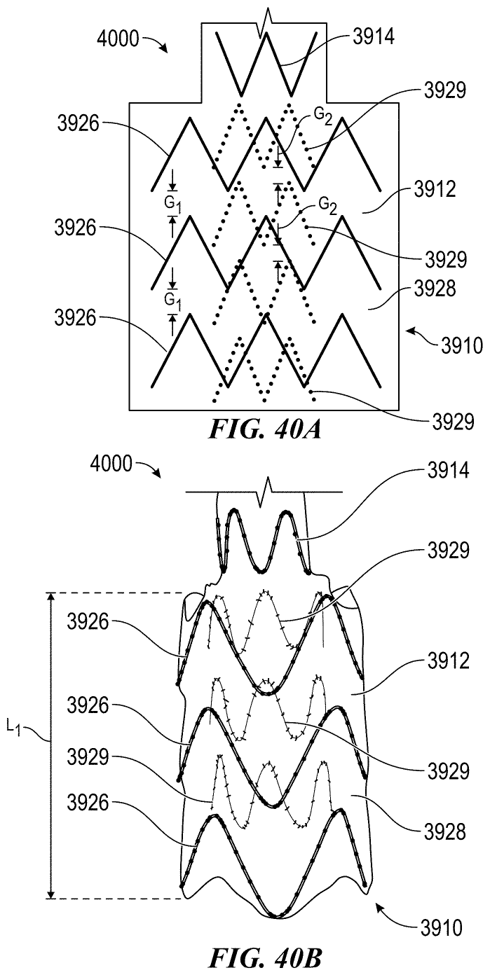

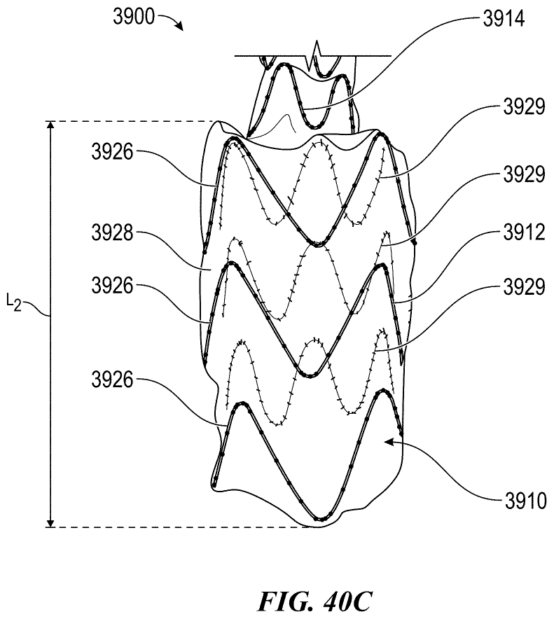

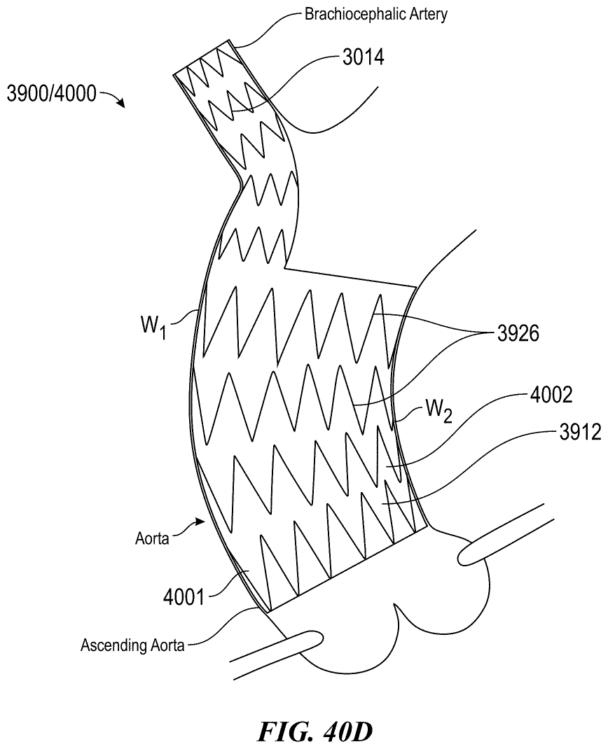

FIGS. 34A-38 illustrate various aortic repair devices having configurations of stents that extend around the entire perimeter/circumference of a base member that inhibit or permit longitudinal compression of the base member. In some embodiments, the axial stiffness and/or flexibility profile of an aortic repair device can further be selected based on the configuration of one or more secondary lumen stents (e.g., as described in detail above with reference to FIGS. 28A-33). For example, FIGS. 39A and 39B are a side view and a perspective side view, respectively, of an aortic repair device 3900 in a first position, and FIG. 39C is a perspective side view of the aortic repair device 3900 of FIG. 39B in a second position in which the aortic repair device 3900 is longitudinally compressed in accordance with embodiments of the present technology. Similarly, FIGS. 40A and 40B are a side view and a perspective side view, respectively, of an aortic repair device 4000 in a first position, and FIG. 40C is a perspective side view of the aortic repair device 4000 in a second position in which the aortic repair device 3900 is longitudinally compressed in accordance with embodiments of the present technology.

The aortic repair device 3900 and the aortic repair device 4000 can include several features that are generally similar or identical. For example, referring to FIGS. 39A-40C together, both the aortic repair device 3900 and the aortic repair device 4000 include a base member 3910 having a body 3912, a leg 3914 extending from the body 3912 (partially obscured in FIGS. 39A-40C), and a septum (obscured in FIGS. 39A-40C) extending fully or partially through the body 3912 and dividing the body 3912 into a primary lumen and a secondary lumen. The base member 3910 comprises one or more body stents 3926 (which can also be referred to as “aortic stents”) coupled to a graft material 3928. In the illustrated embodiments, the body stents 3926 are (i) generally identical (e.g., each having a periodic V-pattern shape including alternating proximal apices and distal apices and having the same amplitude and frequency), (ii) arranged in phase with another, and (iii) separated from adjacent ones of the body stents 3926 by a gap G₁ (FIGS. 39A and 40A). The base member 3910 further comprises one or more secondary lumen stents 3929 (partially obscured in FIGS. 39B, 39C, 40B, and 40C) positioned within the secondary lumen defined within the body 3912. In the illustrated embodiment, the secondary lumen stents 3929 are (i) generally identical (e.g., each having a periodic V-pattern shape including alternating proximal apices and distal apices and having the same amplitude and frequency), (ii) arranged in phase with another, and (iii) separated from adjacent ones of the secondary lumen stents 3929 by a gap G₂ (FIGS. 39A and 40A). In some embodiments, the body stents 3926 have generally the same amplitude as the secondary lumen stents 3929 but have a lower frequency than the secondary lumen stents 3929.

Referring to FIGS. 39A-39C together, in the illustrated embodiment the secondary lumen stents 3929 substantially overlap (e.g., are axially aligned with) corresponding ones of the body stents 3926. Accordingly, the gaps G₁ and G₂ between the body stents 3926 and the secondary lumen stents 3929, respectively, partially or fully overlap such that the graft material 3928 in the overlap is not supported by the body stents 3926 or the secondary lumen stents 3929 and can easily fold and compress to reduce or eliminate the gaps G₁ and G₂ between adjacent body stents 3926 and secondary lumen stents 3929. As shown in FIG. 39C, this arrangement of the body stents 3926 and the secondary lumen stents 3929 can permit the body 3912 to longitudinally compress when, for example, the aortic repair device 3900 is implanted within the aorta and subject to forces from blood flow through the aorta. In some embodiments, the longitudinal compression (e.g., a difference between a length L₁ of the body 3912 in the first position shown in FIG. 39B and a length L₂ of the body 3912 in the longitudinally compressed second position shown in FIG. 39C) can be about 40%, less than about 40%, about 20%, less than about 20%, etc.

Referring to FIGS. 40A-40C together, in the illustrated embodiment the secondary lumen stents 3929 are shifted relative to the body stents 3926 such that the secondary lumen stents 3929 extend along (e.g., substantially overlap, are axially aligned with) corresponding ones of the gaps G₁ between the body stents 3926. Similarly, the body stents 3926 extend along corresponding ones of the gaps G₂ between the secondary lumen stents 3929. Accordingly, the graft material 3928 along the body 3912 has no or few axial positions in which it is not supported by one of the body stents 3926 and/or the secondary lumens stents 3929. As shown in FIG. 40C, this arrangement of the body stents 3926 and the secondary lumen stents 3929 can substantially inhibit longitudinal compression of the body 3912 when, for example, the aortic repair device 3900 is implanted within the aorta and subject to forces from blood flow through the aorta. In some embodiments, the longitudinal compression (e.g., a difference between a length L₁ of the body 3912 in the first position shown in FIG. 39B and a length L₂ of the body 3912 in the longitudinally compressed second position shown in FIG. 39C) can be about 20%, less than about 20%, about 10%, less than about 10%, etc.

Referring again to FIGS. 39A-40C together, the secondary lumen stents 3929 can combine with the body stents 3926 to provide a selected axial stiffness around the circumference of the implantable device 3900/4000. Because the secondary lumen stents 3929 are positioned in the secondary lumen of the body 3912, the stiffness provided by the secondary lumen stents 3929 only affects the portion (e.g., side) of the body 3912 adjacent the secondary lumen of the body 3912. Accordingly, in some embodiments the portion of the body 3912 adjacent the secondary lumen of the body 3912 and the secondary lumen stents 3929 therein can have a greater axial stiffness than a portion of the body 3912 adjacent the primary lumen. In some aspects of the present technology, the relatively less stiff portion of the body 3912 adjacent the primary lumen is configured to be positioned along the interior wall of the aortic arch having the lesser curvature.

More specifically, for example, FIG. 40D is a side view of the aortic repair device 3900 or the aortic repair device 4000 implanted within an aorta in accordance with embodiments of the present technology. In the illustrated embodiment, the aortic repair device 3900/4000 is implanted in the ascending aorta such that the leg 3914 extends into a branch artery, such as the brachiocephalic artery. Accordingly, the leg 3914 and a first side portion 4001 of the body 3912 (e.g., a side adjacent to the secondary lumen extending through the body 3912) can be positioned against and/or adjacent to a first wall portion W₁ of the aorta, and a second side portion 4002 (e.g., a side portion adjacent to the primary lumen extending through the body 3912) can be positioned against and/or adjacent to a second wall portion W₂ of the aorta having a lesser curvature (e.g., lesser radius of curvature) and shorter length than the first wall portion W₁. In some embodiments, as described in detail above, the aortic repair device 3900/4000 can have a greater axial stiffness (e.g., minimal compression) at the first side portion 4001 of the body 3912 adjacent the first wall portion W₁ than at the second side portion 4002 of the body 3912 adjacent the second wall portion W₂. For example, the secondary lumen stents 3929 (39A-40C) can cooperate with the body stents 3926 to resist longitudinal compression at the first side 4001, but are not present at the second side portion 4002 such that the second side portion 4002 is relatively more flexible. Accordingly, in some aspects of the present technology, the first side portion 4001 is in tension after deployment while the relatively more flexible second side portion 4002 can axially compress and/or nest along the second wall portion W₂.

In other embodiments, the body stents 3926 and/or the secondary lumen stents 3929 can have other configurations (e.g., having different frequencies, amplitudes, shapes) and/or can be arranged differently relative to another (e.g., with little or no gap therebetween, out of phase with one another). Referring to FIG. 37A, for example, one or more secondary lumen stents 3729 (partially obscured) can extend fully or partially between (i) the first body stent 3726a having a variable frequency and the second body stent 3726b to provide increased axial stiffness therebetween and/or (ii) the second body stent 3726b and the third body stent 3726c to provide increased axial stiffness therebetween.

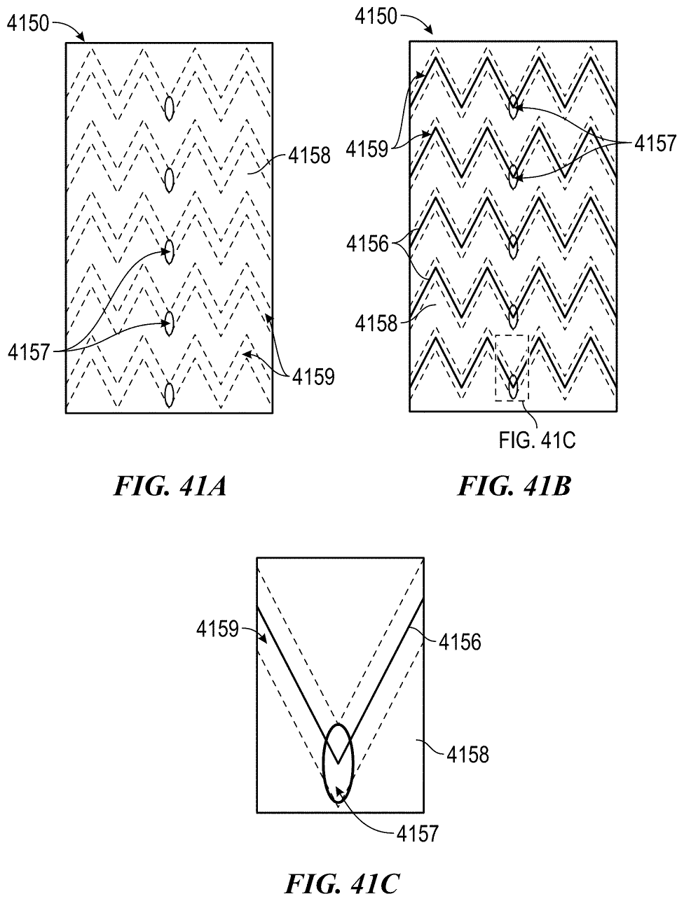

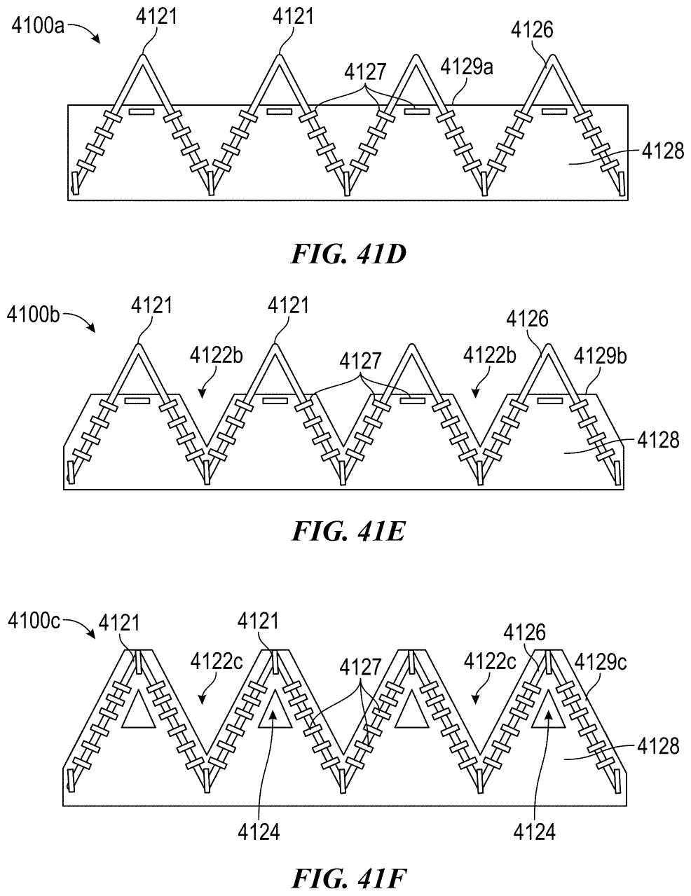

In any of the embodiments described herein, the graft material of an aortic repair device can be sewn or otherwise attached to the stents of the aortic repair device. In other embodiments, the stents can be inserted through corresponding channels or pockets in the graft material to secure the stents to the graft material. For example, FIGS. 41A and 41B are side views of a spanning member 4150 in accordance with embodiments of the present technology. FIG. 41C is an enlarged view of a portion of the spanning member 4150 shown in FIG. 41B in accordance with embodiments of the present technology. Referring to FIGS. 41A-41C together, the spanning member 4150 includes a graft material 4158 having pockets or channels 4159 configured (e.g., shaped and sized) to receive corresponding ones of a plurality of stents 4156 through small access opening or ports 4157. The stents 4156 are removed from the channels 4159 in FIG. 41A, and inserted into the channels 4159 in FIGS. 41B and 41C. The graft material 4158 can be formed by weaving or another suitable method that enables the creation of the channels 4159 in the wall thereof. The stents 4156 can then be inserted into the channels 4159 to have a desired shape. For example, the channels 4159 can have a zig-zag pattern, a Z-pattern, a V-pattern, a straight pattern, a sinusoidal helix pattern, etc., such that the stents 4156 conform to the shape of the channels 4159. The stents 4156 can be wires that are inserted (e.g., pulled via a tether and/or pushed) through the access openings 4157 into and through the channels 4159 and can have atraumatic (e.g., ball) ends and/or can be crimped after insertion into the channels 4159. Any of the components of the aortic repair devices described herein (e.g., spanning members, base members, etc.) can be formed in such a manner.

In some embodiments, graft material can be omitted or removed from a distal end portion and/or a proximal end of any of the components of the aortic repair devices described herein (e.g., spanning members, base members, etc.). For example, FIGS. 41D-41F are enlarged views of a proximal end portion or a distal end portion of a component—such as any of the base members and/or spanning members described herein—of various aortic repair devices 4100a-4100c, respectively, in accordance with embodiments of the present technology. Referring to FIGS. 41D-41F together, the aortic repair devices 4100a-c each include a stent 4126 having apices 4121 and a graft material 4128 secured to the stent 4126 via stitching 4127. The apices 4121 can be apices located at a distal-most end portion and/or proximal-most end portion of the component.

Referring to FIG. 41D, the graft material 4128 can extend only partially along the stent 4126 such that the apices 4121 are exposed from the graft material 4128. In the illustrated embodiment, an edge 4129a of the graft material 4128 is linear. This configuration defines apertures between the individual apices 4121 and an edge 4129a of the graft material 4128 and leaves a portion of the space between adjacent apices 4121 open, both of which allow blood to flow therethrough.

Referring to FIG. 41E, the graft material 4128 can extend only partially along the stent 4126 and follow the curvature of the stent 4126 between the apices 4121, leaving the apices 4121 and the spaces therebetween exposed from the graft material 4128. Accordingly, in the illustrated embodiment the graft material 4128 can define an edge 4129b that generally tracks (e.g., follows) the struts of the stent 4126 between the apices 4121 to form inlets 4122b (also referred to as openings, recesses, or serrations) at the edge 4129b of the graft material 4128. The inlets 4122b can be formed along the edge 4129b by removing all or a portion of the graft material 4128 between the apices 4121 (e.g., via trimming) before and/or after attachment to the stent 4126.

Referring to FIG. 41F, the graft material 4128 can extend fully to the apices 4121 of the stent 4126, and the graft material 4128 may include first openings 4122c (e.g., serrations, inlets) between the apices 4121 and second openings 4124 within (e.g., under) the individual apices 4121. The first and second openings 4122c, 4124 can be formed by removing some of the graft material 4128 from between and/or within the apices, respectively. Accordingly, in the illustrated embodiment the graft material 4128 defines an edge 4129c that generally tracks (e.g., follows) the curvature of the struts of the stent 4126 along the perimeter of the stent 4126, and also provides openings between the struts.

As described above, the arrangements of the graft material 4128 and the stent 4126 shown in FIGS. 41D-41F provide openings, inlets, and/or bare stent regions at one or both ends of a spanning member and/or base member. This allows for lateral flow through the end regions of the spanning/base member, which can aid in deployment as it increases the viable landing zone for the device and reduces the precision necessary for aligning the end region with the target site. For example, the spanning/base member can overshoot the target site such that the end region is exposed within the vessel and/or the end region may partially or fully overlay a branching lumen (e.g., a vessel or other stent component) without obstructing blood flow. This can be of particular use when deploying within a curved vessel (e.g., the aortic arch) where the curvature may make alignment of stents more difficult than in a substantially linear vessel.

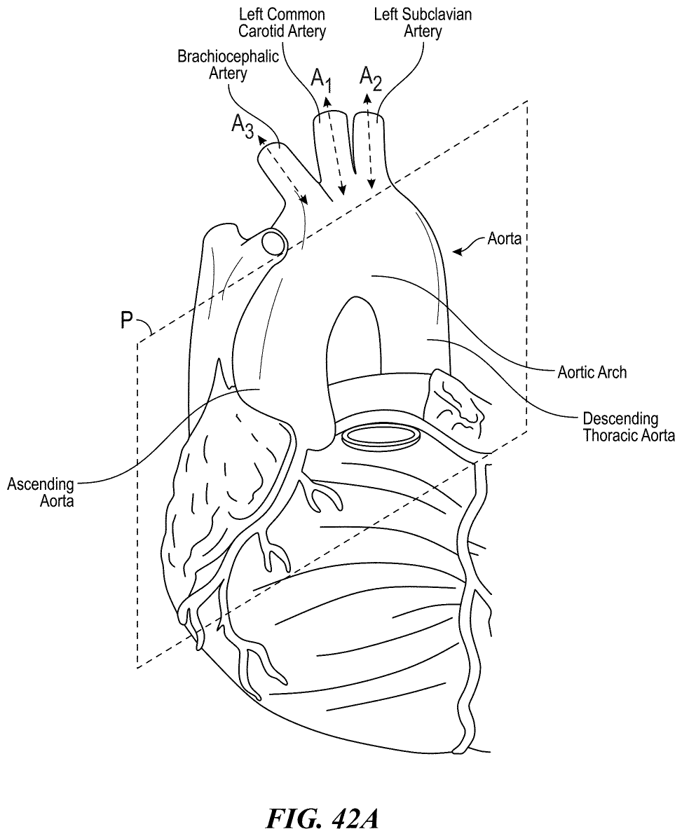

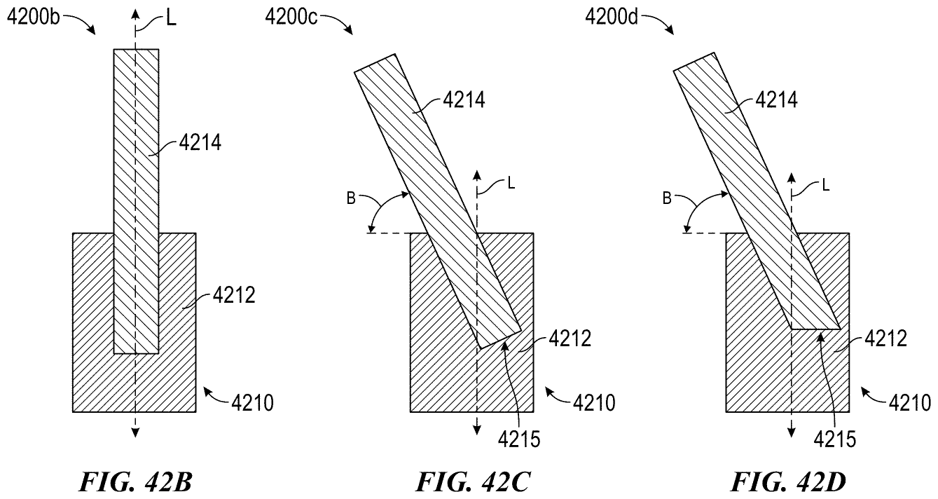

In some embodiments described herein, the leg and/or another portions of an aortic repair device can be angled or otherwise oriented to match the anatomy of the aorta. For example, FIG. 42A is an anterior view of an aorta and surrounding anatomy in which an aortic repair device can be implanted in accordance with embodiments of the present technology. As shown in FIG. 42A, the aorta (e.g., the ascending aorta, the aortic arch, and/or an upper portion of the descending thoracic aorta) can generally lay in a plane P. The left common carotid artery and the left subclavian artery can extend along axes A₁ and A₂, respectively, that are angled relative to (e.g., not parallel to) the plane P, and the brachiocephalic artery can extend along an axis A₃ that is farther angled relative to the plane P than the axes A₁ and A₂. That is, the brachiocephalic artery can be positioned more out of the plane P of the aorta than the left common carotid artery and the left subclavian artery. Accordingly, an aortic repair device in accordance with embodiments of the present technology can have a leg arranged to accommodate the angled takeoff to the brachiocephalic artery, the left common carotid artery, and/or the left subclavian artery.

More specifically, for example, FIGS. 42B-42D are side views of aortic repair devices 4200b-4200d, respectively, in accordance with embodiments of the present technology. Referring to FIGS. 42B-42D together, the aortic repair devices 4200b-d can each include a base member 4210 having a body 4212 configured to be implanted in the aorta, a leg 4214 extending from the body 4212 and configured to be implanted in a branch vessel (e.g., the brachiocephalic artery, the left common carotid artery, and/or the left subclavian artery), and a septum (obscured in FIGS. 42B-42D) extending fully or partially through the body 4212 and dividing the body 4212 into a primary lumen and a secondary lumen.

Referring to FIG. 42B, the leg 4214 can extend generally parallel to a longitudinal axis L of the body 4212. Referring to FIGS. 42C and 42D together, the leg 4214 can be angled relative to the longitudinal axis L of the body 4212 by an angle B. Referring to FIGS. 42A, 42C, and 42D together, the angle B can be selected to correspond to or substantially match the angle at which one of the brachiocephalic artery, the left common carotid artery, and/or the left subclavian artery are angled relative to the plane P. Accordingly, in some aspects of the present technology the leg 4214 of the aortic repair devices 4200c-d can be offset from the longitudinal axis L of the body 4212 to better mate with the angled anatomy of a branch vessel relative to the aorta in which the body 4212 is implanted. This can reduce the degree of bending of the leg 4214 relative to the body 4212 and/or reduce kinking of the leg 4214 when the aortic repair devices 4200c-d are implanted into anatomy with offset branching arteries. Referring to FIG. 42C, in some embodiments a proximal opening 4215 to the leg 4214 is also angled relative to the longitudinal axis L of the body 4212. Referring to FIG. 42D, in some embodiments the proximal opening 4215 is beveled such that the proximal opening 4215 extends orthogonal to the longitudinal axis L to, for example, maintain the cross-sectional flow area of the proximal opening 4215.

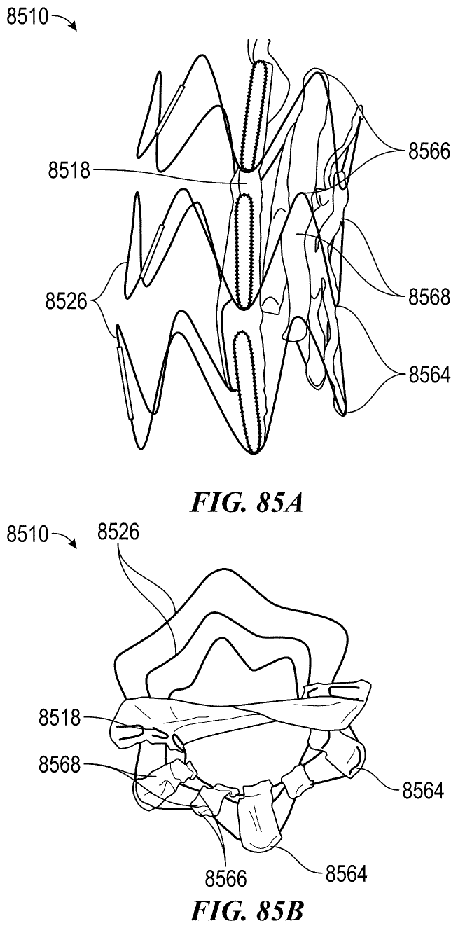

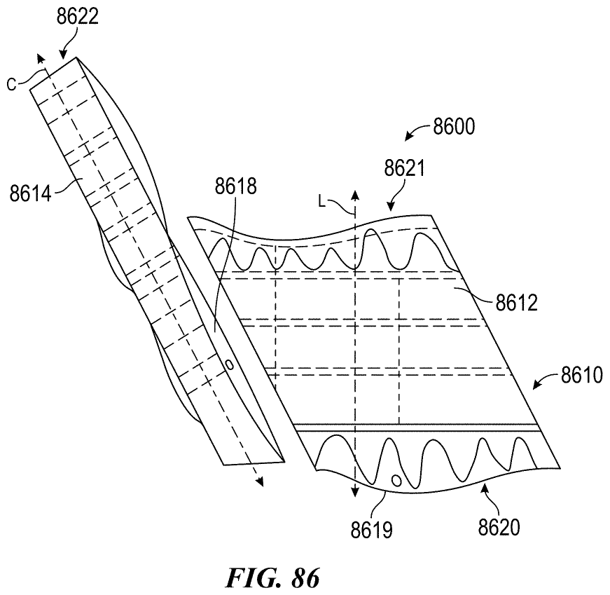

FIG. 86 is a side view of an aortic repair device 8600 in accordance with embodiments of the present technology. In the illustrated embodiment, the aortic repair device 8600 includes a base member 8610 having a body 8612 configured to be implanted in the aorta and a leg 8614 extending from the body 8612 and configured to be implanted in a branch vessel (e.g., the brachiocephalic artery, the left common carotid artery, and/or the left subclavian artery). In FIG. 86, the aortic repair device 8600 is shown as partially exploded, with the leg 8614 separated from the primary lumen of the body 8612 for clarity. When the aortic repair device 8600 is assembled, a septum 8618 extends fully or partially through the body 8612 to divide the body 8612 into a primary lumen that provides for fluid flow through the portion of the body 8612 not taken up by the leg 8614 and a secondary lumen that provides for fluid flow through the leg 8614.

In the illustrated embodiment, the leg 8614 has a central axis C extending orthogonal to a trailing (e.g., distal) opening 8622 thereof, and the body 8612 has a central axis L extending orthogonal to a leading (e.g., proximal) opening 8620 and a trailing (e.g., distal) primary lumen opening 8621 thereof. In some embodiments, the central axis C of the leg 8614 is non-parallel (e.g., angled, offset) to the central axis L of the body 8612. This offset in the central axis of the body 8612 and the leg 8614 can cause the leg 8614 to curve, curl, or spiral (e.g., partially spiral) around the arch of the aortic lumen when the aortic repair device 8600 is implanted within an aorta. The angled leg 8614 can allow the aortic repair device 8600 to beneficially absorb force and/or flow proximate to the leading portion of the leg 8614 (e.g., a D-shaped channel of the leg 8614 formed by one or more D-shaped stents), which is closer to a lesser curve of the aorta, and then transitions to the trailing portion of the leg 8614 as it curls and exits the aorta. In some embodiments, the aortic repair device 8600 can include one or more radiopaque markers 8619 positioned, for example, at the proximal end of the body 8612 (e.g., on an outer curve of the body 8612).

### III. SELECTED EMBODIMENTS OF AORTIC REPAIR DEVICES INCLUDING MULTIPLE LEGS

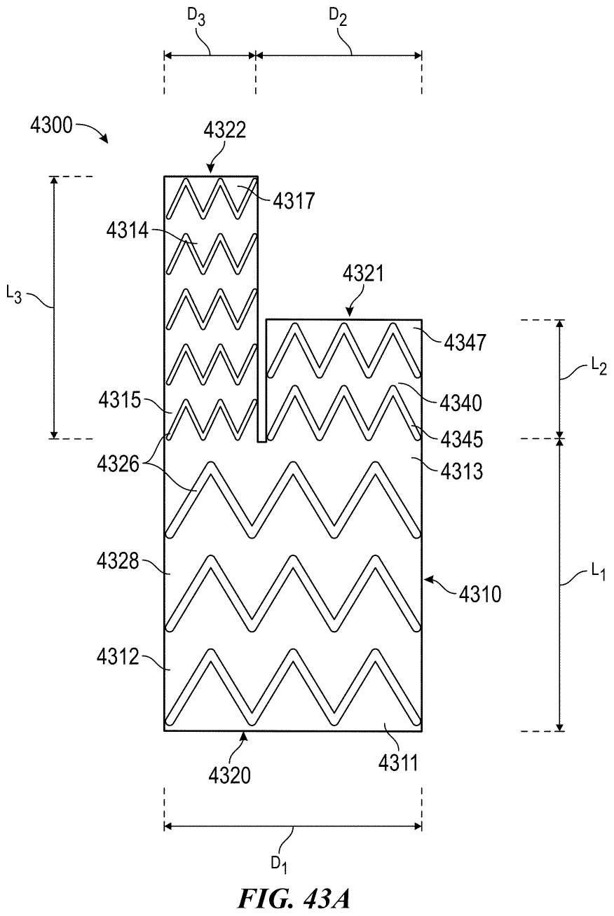

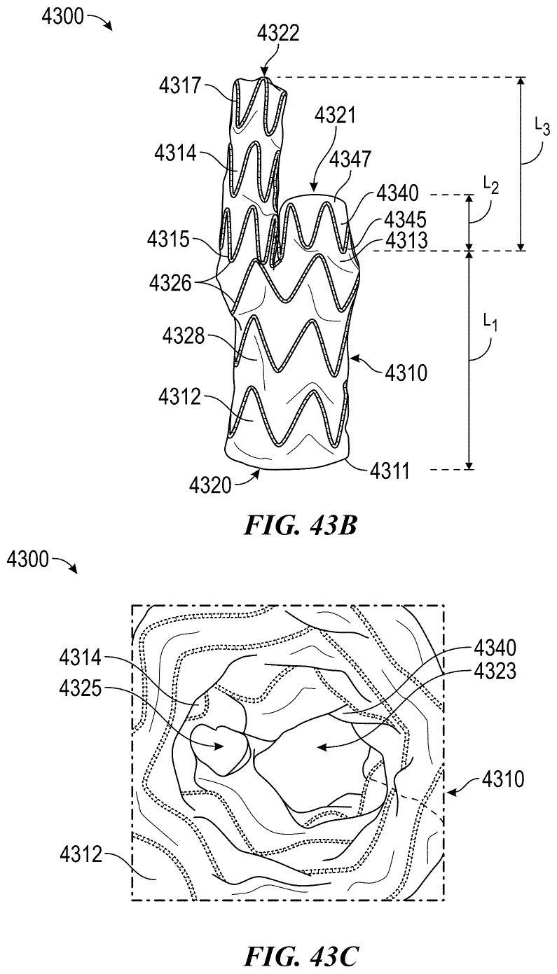

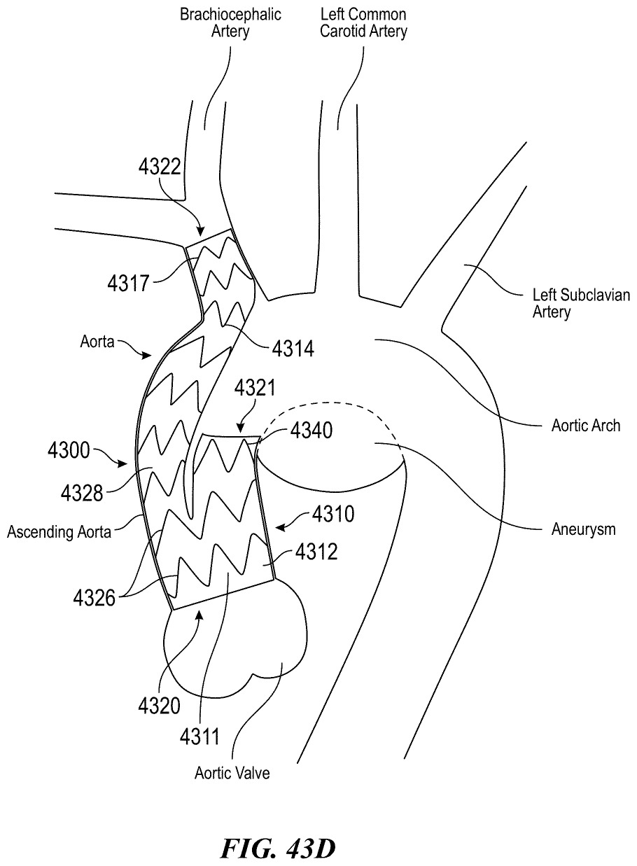

FIGS. 43A and 43B are a side view and a perspective side view, respectively, of an aortic repair device 4300 in accordance with embodiments of the present technology. The aortic repair device 4300 can include some features that are at least generally similar in structure and function, or identical in structure and function, to the corresponding features of any of the aortic repair devices described in detail above with reference to FIGS. 3A-42D, and can operate in a generally similar or identical manner to the aortic repair devices of FIGS. 3A-42D. For example, in the illustrated embodiment the aortic repair device 4300 includes a base member 4310 configured to be implanted in a diseased aorta and comprising, for example, a stent graft including a graft material 4328 attached to a plurality of stents 4326.

In the illustrated embodiment, the base member 4310 includes (i) a tubular body 4312, (ii) a tubular primary leg 4340 extending distally from the body 4312, and (iii) a tubular secondary leg 4314 extending distally from the body 4312. More specifically, the body 4312 includes a proximal end portion 4311 defining a proximal terminus of the base member 4310 and a distal end portion 4313. The primary leg 4340 includes a proximal end portion 4345 coupled to the distal end portion 4313 of the body 4312 and a distal end portion 4347. The secondary leg 4314 includes a proximal end portion 4315 coupled to the distal end portion 4313 of the body 4312 and a distal end portion 4327 defining a distal terminus of the base member 4310. In the illustrated embodiment, the body 4312 has a first length L₁, the primary leg 4340 has a second length L₂ less than the first length L₁, and the secondary leg 4324 has a third length L₃ greater than the second length L₂. In other embodiments, the first through third lengths L₁-L₃ can be different. For example, the third length L₃ can be shorter than the length L₁ and/or shorter than the length L₂. In some embodiments, the first length L₁ can be between about 20-80 millimeters, the second length L₂ can be between about 20-40 millimeters, and the third length L₃ can be between about 30-100 millimeters.

The base member 4310 can be generally hollow and define one or more lumens therethrough. Accordingly, in the illustrated embodiment the base member 4310 includes (i) a proximal opening 4320 (e.g., a first opening, an inlet) at the proximal end portion 4311 of the body 4310 and having a first diameter D₁ (FIG. 43A), (ii) a distal primary leg opening 4321 (e.g., a second opening, a distal body outlet) at the distal end portion 4347 of the primary leg 4340 and having a second diameter D₂ (FIG. 43A), and (iii) a distal secondary leg opening 4322 (e.g., a third opening, a distal leg outlet) at the distal end portion 4317 of the secondary leg 4314 and having a third diameter D₃ (FIG. 43A). The first diameter D₁ is larger than the second and third diameters D₂, D₃ and can be sized to generally match or be larger than (e.g., oversized relative to) the diameter of a patient's aorta (e.g., between about 20-60 millimeters, between about 26-54 millimeters, about 40 millimeters). The third diameter D₃ can be sized to generally match or be larger than a branch vessel of the patient's aorta, such as the brachiocephalic artery (e.g., between about 10-30 millimeters, about 18 millimeters). In some embodiments, the second diameter D₂ can be larger than the third diameter D₃ (e.g., between about 20-40 millimeters, about 28 millimeters).

FIG. 43C is a distally-facing perspective end view of the aortic repair device 4300 of FIGS. 43A and 43B in accordance with embodiments of the present technology. Referring to FIG. 43C, the body 4312 and the primary leg 4340 together define a primary lumen 4323 through the base member 4310, and the body 4312 and the secondary leg 4314 together define a secondary lumen 4325 through the base member 4310. Referring to FIGS. 43A-43C together, the primary lumen 4323 can extend from and define a flow path between the proximal opening 4320 and the primary leg opening 4321. Similarly, the secondary lumen 4325 can extend from and define a flow path between the proximal opening 4320 and the secondary leg opening 4322. As shown in FIG. 43C, the primary lumen 4323 and the secondary lumen 4325 can each have a generally circular cross-sectional shape within the body 4312 and within the primary and secondary legs 4340, 4314, respectively.

FIG. 43D is a side view of the aortic repair device 4300 implanted within an aorta in accordance with embodiments of the present technology. In some embodiments, the aorta can include an aneurysm, dissection, and/or other diseased portion as described in detail above with reference to FIGS. 1A-2B. In the illustrated embodiment, the body 4312 is implanted within the proximal aorta (e.g., the ascending aorta and/or the aortic arch) with the proximal end portion 4311 positioned proximate to the aortic valve, and the secondary leg 4314 extending from the body 4312 to the brachiocephalic artery where the distal end portion 4317 is positioned. Referring to FIGS. 43A-43D together, the stents 4326 can expand the graft material 4328 into contact with the inner wall of the aorta and/or the brachiocephalic artery to provide a seal between the vessels and the base member 4310. More particularly, the body 4312 can sealingly contact the inner wall of the proximal aorta such that all or substantially all blood flow through the aorta enters the proximal opening 4320 and flows through either the primary lumen 4323 or the secondary lumen 4325. As shown in FIG. 43D, the base member 4310 can direct the blood flow (i) through the primary lumen 4323 and out of the primary leg opening 4321 into the aorta to perfuse the aorta and (ii) through the secondary lumen 4325 and out of the secondary leg opening 4322 into the brachiocephalic artery to perfuse the brachiocephalic artery.

The base member 4310 can be delivered to the aorta in a collapsed configuration within a catheter system (not shown). The catheter system can access the aorta via any suitable intravascular path-such as an aortic approach, a transfemoral approach, a transcarotid approach, a left common carotid artery (LCCA) approach, a left subclavian artery (LSA) approach, and so on.

In some aspects of the present technology, the base member 4310 can be positioned against/adjacent a diseased portion of the aorta, such as the origin of a dissection and/or aneurysm, to block blood flow into the diseased portion by diverting the blood flow through the base member 4310 and past the diseased portion. In additional aspects of the present technology, the outer surface of the body 4310 has a tubular shape configured to engage and seal with the inner wall of the aorta—but the base member 4310 may not seal against the aorta along the lengths of the primary and secondary legs 4340, 4314. Accordingly, the base member 4310 may provide a relatively shorter sealing region compared to, for example, the base member 310 described in detail above with reference to FIGS. 3A-3D. However, the primary lumen 4323 can have a generally circular cross-sectional shape within the primary leg 4340. This can facilitate the coupling of other implantable devices, such a spanning member, to the base member 4310.

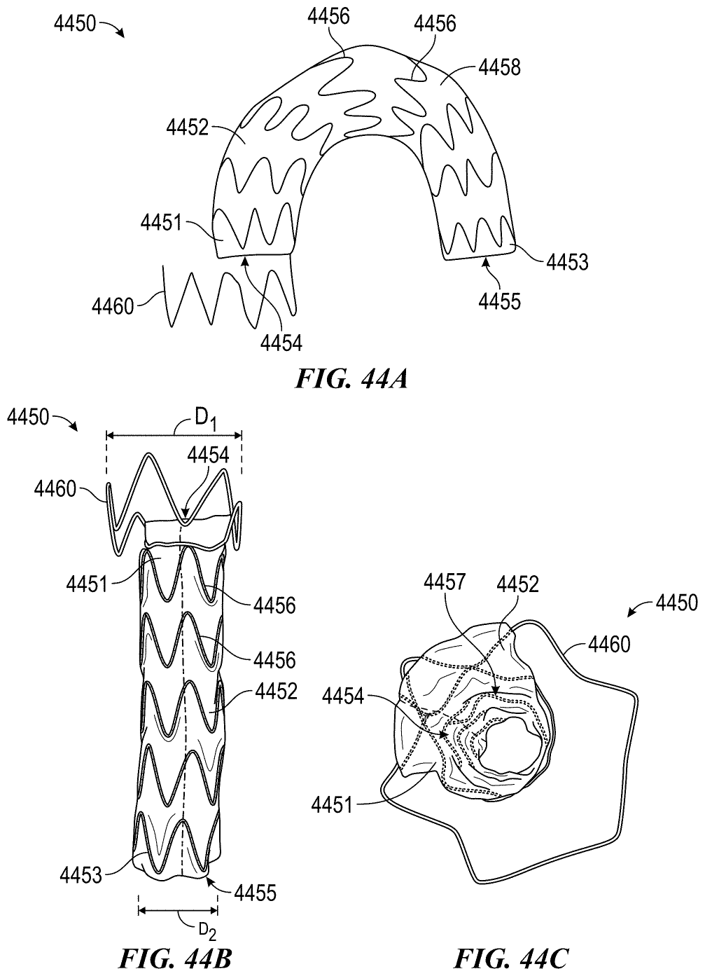

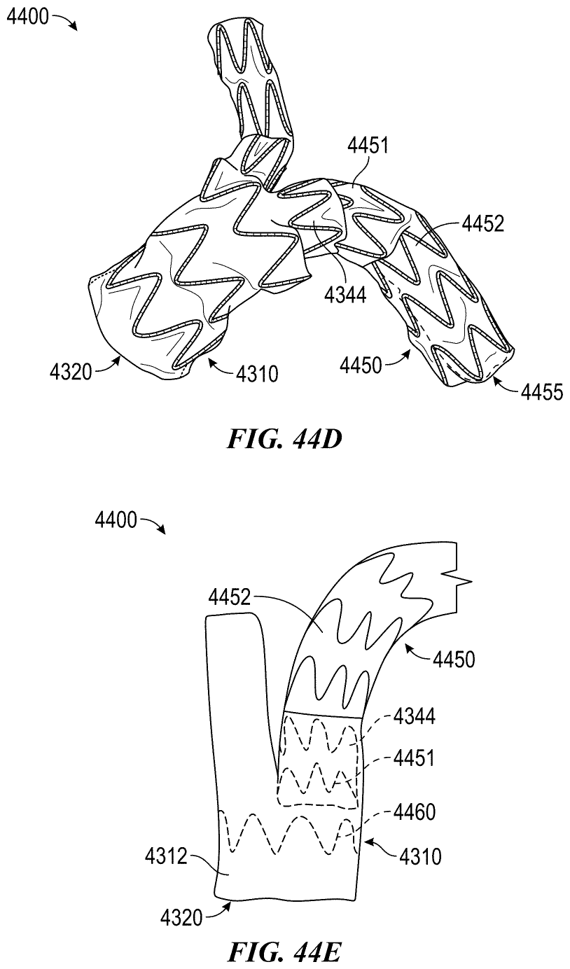

FIGS. 44A-44C, for example, are a side view, a perspective side view, and distally-facing perspective end view, respectively, of a spanning member 4450 in accordance with embodiments of the present technology. Referring to FIGS. 44A-44C together, the spanning member 4450 can include some features that are at least generally similar in structure and function, or identical in structure and function, to the corresponding features of any of the spanning members described in detail above with reference to FIGS. 3A-38B, and can operate in a generally similar or identical manner to the spanning members of FIGS. 3A-38B.

For example, in the illustrated embodiment the spanning member 4450 includes a body 4452 having a tubular shape defining a spanning lumen 4457 and extending between a proximal end portion 4451 and a distal end portion 4453. Accordingly, in the illustrated embodiment the body 4452 includes a proximal opening 4454 and a distal opening 4455. The spanning member 4450 can comprise an expandable stent graft comprising one or more struts or stents 4456 and a graft material 4458 coupled to the stents 4456.

In the illustrated embodiment, the spanning member 4450 further includes an anchor 4460 at and/or coupled to the proximal end portion 4451 of the body 4452. The anchor 4460 can be a bare stent or other expandable element and, when the spanning member 4450 is expanded and unconstrained (e.g., outside of an aorta), can have a first diameter D₁ (FIG. 44B) that is larger than a second diameter D₂ (FIG. 44B) of the body 4452. In the illustrated embodiment, the anchor 4460 is a bare stent having a generally circular cross-sectional shape and a V-pattern. In some embodiments, the anchor 4460 can be laterally offset relative to the body 4452 such that, for example, a longitudinal axis concentric with the anchor 4460 is offset from a longitudinal axis concentric with the body 4452.

FIGS. 44D and 44E are a perspective side view and a side view, respectively, of an aortic repair device 4400 including the spanning member 4450 coupled to (e.g., docked to) the base member 4310 of FIGS. 43A-43D in accordance with embodiments of the present technology. Referring to FIGS. 44D and 44E together, the anchor 4460 and the proximal end portion 4451 of the body 4452 are at least partially positioned within the base member 4310. More specifically, (i) the anchor 4460 can be expanded into contact with an inner surface of the body 4312 to anchor and secure the body 4452 of the spanning member 4450 relative to the base member 4310 and (ii) the proximal end portion 4451 of the body 4452 can be expanded into contact with and sealingly engage an inner surface of the primary leg 4344. In the illustrated embodiment, the anchor 4460 is offset relative to the body 4452 of the spanning member 4450 such that the anchor 4460 engages substantially the entire circumference of the body 4312.

Referring to FIGS. 43A-44E together, the aortic repair device 4400 can define a continuous blood flow path from the proximal opening 4320 of the base member 4310, through the primary lumen 4325, and through the spanning lumen 4457 to the distal opening 4455. In some aspects of the present technology, increasing the second length L₂ (FIG. 43A) of the primary leg 4344 can increase the available area for sealing between the spanning member 4450 and the base member 4310 thereby increasing the potential robustness of the seal therebetween. However, increasing the second length L₂ requires decreasing the length L₁ of the body 4312—thereby decreasing the available area for sealing between the body 4312 and the aorta—or increasing the total length of the body 4312 and the primary leg 4344. Accordingly, in some aspects of the present technology the second length L₂ is selected to (i) be just long enough to ensure a robust seal between the spanning member 4350 and the primary leg 4344 and to (ii) correspondingly maximize the first length L₁ to ensure a robust seal between the body 4312 and the aorta. In some embodiments, the second length L₂ is about 1 centimeter.

As described in detail above with reference to, for example, FIG. 4, the spanning member 4450 can divert blood flow past a diseased portion of the aorta, such as an aneurysm in the aortic arch (FIG. 43D) when the aortic repair device 4400 is implanted within a patient. In some embodiments, the spanning member 4450 can be delivered to the aorta in a collapsed configuration within a catheter system (not shown) in the same or a separate procedure as the base member 4310. For example, often a patient may initially only need treatment within the ascending aorta and/or aortic arch to treat an initial dissection or aneurysm but, at a later time (e.g., months or years later), may require additional treatment of the aortic arch and/or descending thoracic aorta as the diseased state progresses. Accordingly, the base member 4310 can be implanted during an initial procedure and the spanning member 4450 can be implanted during a later procedure and modularly coupled to the base member 4310 to provide further treatment of the aortic arch and/or descending thoracic aorta (e.g., by bypassing blood flow past the aneurysm shown in FIG. 43D).

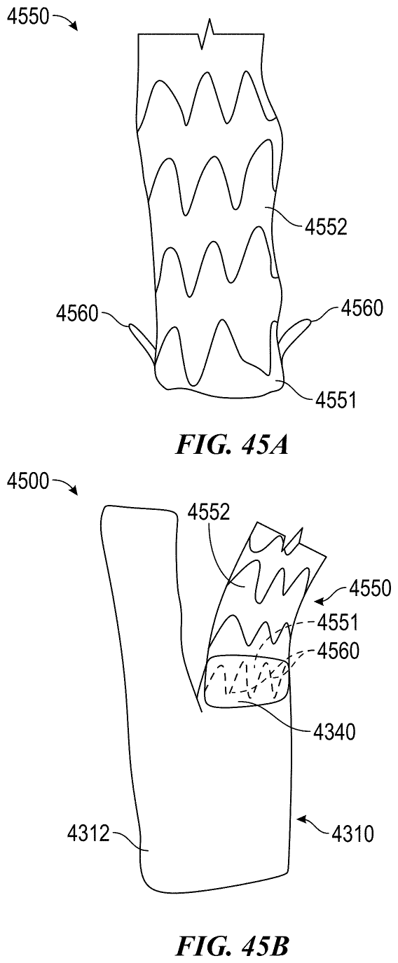

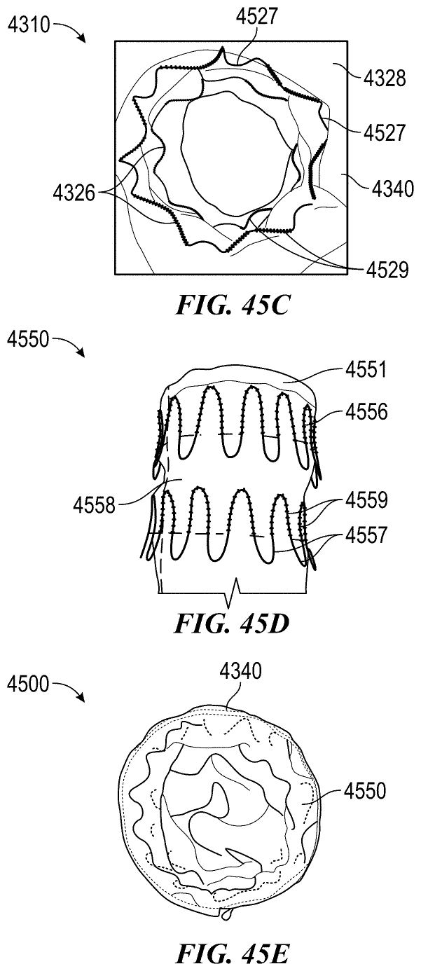

FIG. 45A is an enlarged side view of a spanning member 4550 in accordance with embodiments of the present technology. The spanning member 4550 can include some features that are at least generally similar in structure and function, or identical in structure and function, to the corresponding features of the spanning member 4450 described in detail above with reference to FIGS. 44A-44E, and can operate in a generally similar or identical manner to the spanning member 4450. For example, the spanning member 4550 includes a tubular body 4552 (e.g., a stent graft) defining a lumen and having a proximal end portion 4551. In the illustrated embodiment, the spanning member 4550 further includes multiple anchoring or fixation elements 4560 at and/or coupled to the proximal end portion 4551. The fixation elements 4560 can be hooks, wires, loops, and/or other elements.

FIG. 45B is a side view of an aortic repair device 4500 including the spanning member 4550 coupled to (e.g., docked to) the base member 4310 of FIGS. 43A-43D in accordance with embodiments of the present technology. In the illustrated embodiment, the fixation elements 4560 secure and seal the proximal end portion 4551 of the body 4552 to the inner surface of the primary leg 4340 of the base member 4310. In other embodiments, additional fixation elements can alternatively or additionally be positioned on the inner surface of the primary leg 4340. For example, such fixation elements can comprise a portion of one or more stents used to form the base member 4310 and that project inward into the lumen of the primary leg 4340.

FIG. 45C is a proximally-facing perspective view of the primary leg 4340 of the base member 4310 of FIG. 45B in accordance with embodiments of the present technology. FIG. 45D is an enlarged perspective side view of the proximal end portion 4551 of the spanning member 4550 of FIG. 45A in accordance with embodiments of the present technology. FIG. 45E is a proximally-facing perspective of the spanning member 4550 coupled to the primary leg 4340 of the base member 4310 in accordance with embodiments of the present technology. As shown in FIG. 45C, some or all of the stents 4326 within the primary leg 4340 can include (i) first portions 4529 fixed to (e.g., via sutures) and apposing the graft material 4328 and (ii) second portions 4527 (e.g., exposed portions, non-sewn portions) that are not fixed to the graft material 4328 and that extend radially inward from an interior surface of the graft material 4328 (e.g., are tilted or lifted off the interior surface of the graft material 4328). As shown in FIG. 45D, the spanning member 4550 can include one or more stents 4556 and a graft material 4558. Some or all of the stents 4556 can similarly include (i) first portions 4559 fixed to (e.g., via sutures) and apposing the graft material 4558 and (ii) second portions 4557 (e.g., exposed portions, non-sewn portions) that are not fixed to the graft material 4558 and that extend radially outward from an exterior surface of the graft material 4558 (e.g., are tilted or lifted off the exterior surface of the graft material 4558).

When the spanning member 4550 is coupled to the primary leg 4340 as shown in FIG. 45E, the second portions 4527 of the primary leg 4340 can oppose, interlock, and/or engage with the second portions 4557 of the spanning member 4550 to inhibit migration of the spanning member 4550 relative to the base member 4310. In some embodiments, the coupling of the spanning member 4550 to the primary leg 4340 of the base member 4310 can be modulated by adjusting (i) an extent that the second portions 4527 of the stents 4326 of the base member 4310 project inward toward the spanning member 4550, (ii) an extent that the second portions 4557 of the stents 4556 of the spanning member 4550 project outward toward the primary leg 4340, (iii) a frequency/periodicity of the second portions 4527 of the stents 4326 of the primary leg 4340, and/or (iv) a frequency/periodicity of the second portions 4557 of the stents 4556 of the spanning member 4550 such that some region(s) of the free second portions 4527, 4557 will likely interface when the spanning member 4550 is coupled to the primary leg 4340.

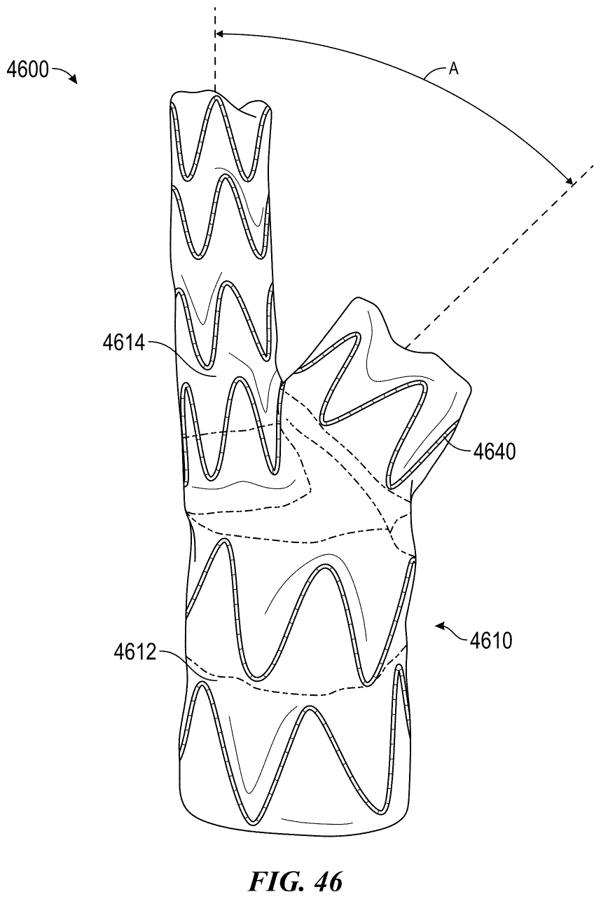

FIG. 46 is a perspective side view of a base member 4610 of an aortic repair device 4600 in accordance with embodiments of the present technology. The base member 4610 can include some features that are at least generally similar in structure and function, or identical in structure and function, to the corresponding features of the base member 3910 described in detail above with reference to FIGS. 39A-39D, and can operate in a generally similar or identical manner to the base member 3910. For example, the base member 4610 includes a proximal body 4612, a primary leg 4640 extending distally from the body 4612, and a secondary leg 4614 extending distally from the body 4612. In the illustrated embodiment, however, the primary leg 4660 extends at an angle A relative to the body 4612 and the secondary leg 4614 of, for example, between about 30°-60° (e.g., about 45°). In some aspects of the present technology, the angled primary leg 4640 can be angled toward/along the ascending aorta or another portion of the aorta thereby making the primary leg 4640 easier to couple a spanning member or other implant to. More specifically, the angled primary leg 4640 can point toward/along the natural inner curvature of the ascending aorta. IV. Selected Embodiments of Aortic Repair Devices Having Reduced Delivery Profiles

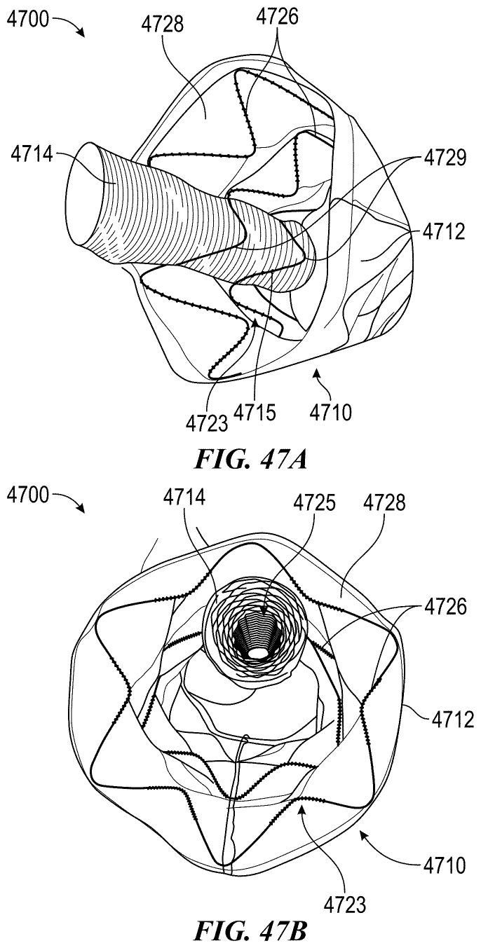

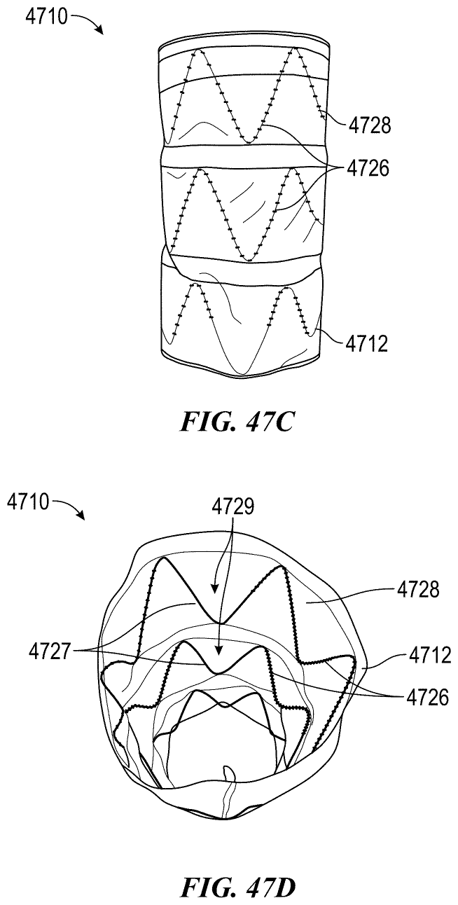

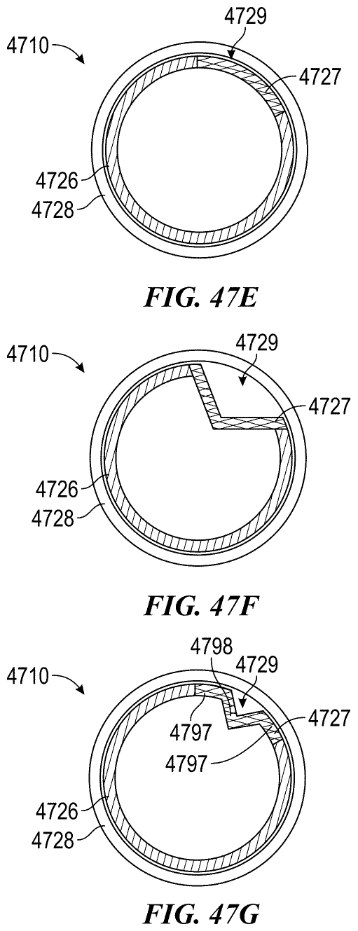

In some aspects of the present technology, graft material is the primary element of an aortic repair device that contributes to the volume of the device and its ability to be packaged into a catheter. Accordingly, decreasing the amount of graft material within the overall aortic repair device and/or efficiently distributing the graft material on components of the aortic repair device delivered separately (e.g., a base member, a spanning member, etc.) can reduce the delivery profile of the aortic repair device-potentially allowing the aortic repair device to be loaded into and delivered through a relatively smaller catheter and/or delivered along a narrower intravascular delivery pathway. FIGS. 47A-56C illustrate different examples of aortic repair devices having reduced delivery profiles. The aortic repair devices described in detail with reference to FIGS. 47A-56C can include some features that are at least generally similar in structure and function, or identical in structure and function, to one another and/or to the corresponding features of any of the aortic repair devices described in detail above with reference to FIGS. 3A-46, and can operate in a generally similar or identical manner to one another and/or to the aortic repair devices of FIGS. 3A-46.

FIGS. 47A and 47B are a distally-facing perspective view and a distally-facing perspective end view, respectively, of an aortic repair device 4700 in accordance with embodiments of the present technology. Referring to FIGS. 47A and 47B together, the aortic repair device 4700 includes a base member 4710 having a body 4712 defining a primary lumen 4723. The aortic repair device 4700 further includes a separate leg member 4714 coupled to the body 4712 and defining a secondary lumen 4725. The leg member 4714 can be stent graft having a similar construction to the base member 4710.

FIGS. 47C and 47D are a perspective side view and a proximally-facing perspective end view, respectively, of the base member 4710 of FIGS. 47A and 47B in accordance with embodiments of the present technology. Referring to FIGS. 47A-47D together, the base member 4710 comprises a graft material 4728 attached to a plurality of stents 4726. In the illustrated embodiment, an exposed portion 4727 (e.g., a V-segment) of one or more of the stents 4726 is not attached to the graft material 4728 and the exposed portions 4727 are at least partially aligned to define a channel 4729. A proximal portion 4715 of the leg member 4714 is inserted into the channel 4729 and sealing engages an inner surface of the body 4712. In some embodiments, the exposed portions 4727 of the stents 4726 exert a radial force against the leg member 4714 that keeps the leg member 4714 in sealing engagement with the inner surface of the body 4712. That is, the exposed portions 4727 of the stents 4726 can act as an internal attachment mechanism for the leg member 4714. Accordingly, in the illustrated embodiment the leg member 4714 is a separate component from the base member 4710 and the base member 4710 does not include a septum therein.

FIGS. 47E-47G are cross-sectional views of the base member 4710 taken along one of the stents 4726 in accordance with embodiments of the present technology. As shown in FIG. 47E, in some embodiments the exposed portions 4727 of the stents 4726 fully contact/appose an interior surface of the graft material 4728 before the leg member 4714 (FIGS. 47A and 47B) is inserted through the channel 4729 between the exposed portions 4727 and the graft material 4728. As shown in FIG. 47F, in some embodiments the exposed portions 4727 extend radially inward from the interior surface of the graft material 4728 (e.g., are “tilted” off the interior surface of the graft material 4728) before the leg member 4714 (FIGS. 47A and 47B) is inserted through the channel 4729 between the exposed portions 4727 and the graft material 4728. As shown in FIG. 47G, in some embodiments the exposed portions 4727 include a contact portion 4797 that apposes the graft material 4728 and an apex portion 4798 that extends radially inward from the interior surface of the graft material 4728 (e.g., is lifted off the interior surface of the graft material 4728) before the leg member 4714 (FIGS. 47A and 47B) is inserted through the channel 4729 between the exposed portions 4727 and the graft material 4728.

In some embodiments, the leg member 4714 can be implanted during the same or a separate procedure as the base member 4710. For example, the base member 4710 can be implanted during an initial procedure (e.g., an index procedure) such that the stents 4726 drive the graft material 4728 to seal against the aorta and divert flow past a diseased portion of the aorta. Then, if the diseased state progresses, the leg member 4714 can be implanted to divert flow past the progressed diseased state alone or in combination with one or more implantable devices. In the embodiments shown in FIGS. 47F and 47G, the exposed portions 4727 of the stents 4726 can provide a path for cannulation of a guidewire used to implant the leg portion 4714 because the exposed portions 4727 do not fully appose the graft material 4728.

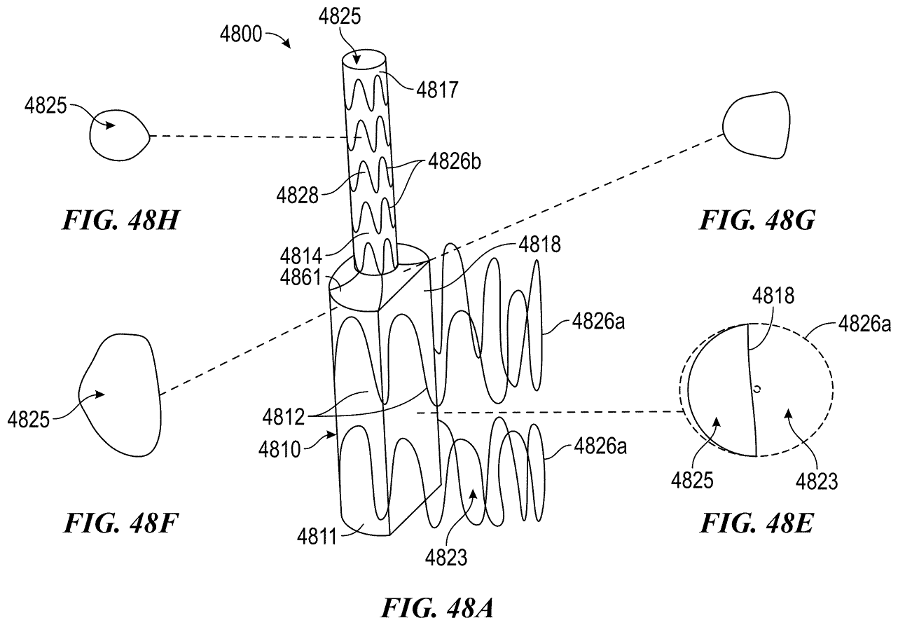

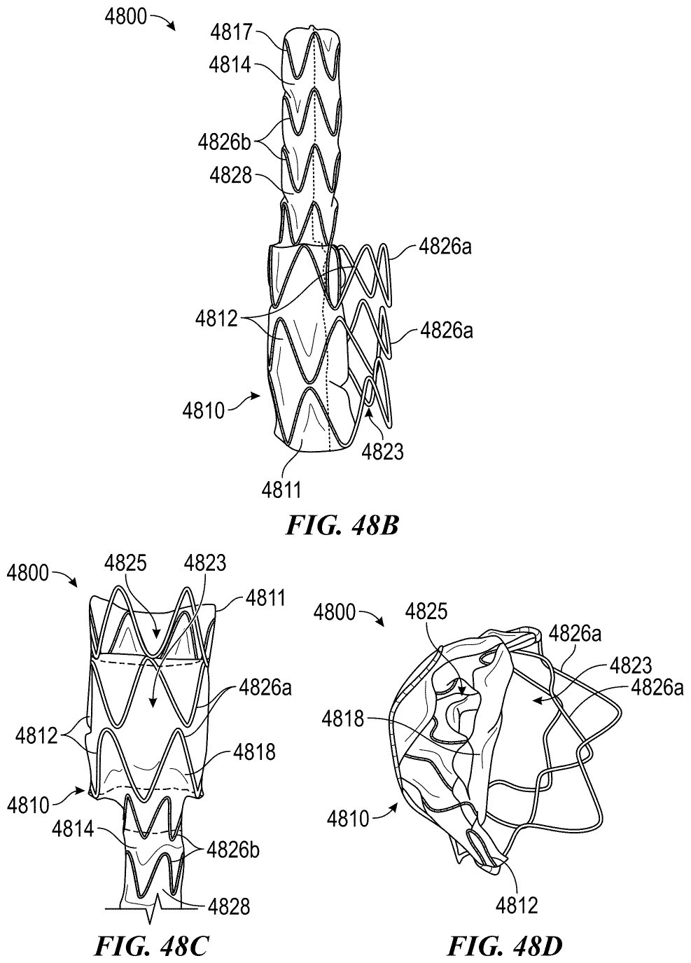

FIGS. 48A-48D are an isometric side view, a perspective side view, an enlarged perspective side view, and a distally-facing perspective end view, respectively, of an aortic repair device 4800 in accordance with embodiments of the present technology. FIGS. 48E-48H are cross-sectional views of the aortic repair device 4800 as indicated in FIG. 48A in accordance with embodiments of the present technology. Referring to FIGS. 48A-48D together, in the illustrated embodiment the aortic repair device 4800 includes a base member 4810 configured to be implanted in a diseased aorta. The base member 4810 can include a body 4812 and a leg 4814 extending distally from the body 4812. The body 4812 can define a proximal end portion 4811 of the base member 4810 and the leg 4814 can define a distal end portion 4817 of the base member 4810. The base member 4810 can comprise a stent graft including a graft material 4828 attached to a plurality of stents 4826 (including individually identified proximal stents 4826a and distal stents 4826b). In the illustrated embodiment, the proximal stents 4826a have a generally circular or ring-like shape, and the graft material 4828 only extends around a portion of the proximal stents 4826a to divide the body 4812 into (i) a primary lumen 4823 that is not enclosed by the graft material 4828 and (ii) a secondary lumen 4825 that is enclosed by the graft material 4828. In some embodiments, the graft material 4828 includes a separate or integral septum 4818 dividing the body 4812 into the primary and secondary lumens 4823, 4825. The septum 4818 can extend fully or partially through the body 4812 and can be centered or offset within the body 4812 such that the primary and secondary lumens 4823, 4825 have the same or different sizes. Accordingly, the primary and secondary lumens 4823, 4825 can each have a generally D-like cross sectional shape within the body 4812 as shown in FIGS. 48E and 48F.

The secondary lumen 4825 can extend to and through the leg 4814. In the illustrated embodiment, the distal stents 4826b also have a generally circular or ring-like shape, and the graft material 4828 can extend entirely around the distal stents 4826b to partially define the secondary lumen 4825. The secondary lumen 4825 can have a generally circular cross-sectional shape within the leg 4814 as shown in FIG. 48H. In some embodiments, the base member 4810 includes a transition region 4861 (FIG. 48A) between the leg 4814 and the body 4812, and the secondary lumen 4825 extends through the transition region 4861 where it has a cross-sectional shape as shown in FIG. 48B that transitions between the D-like cross-sectional shape within the body 4812 and the circular cross-sectional shape within the leg 4814.

The base member 4810 and a corresponding spanning member can be delivered to the aorta in a collapsed configuration within the same or separate catheter systems. The catheter system can access the aorta via any suitable intravascular path-such as an aortic approach, a transfemoral approach, a transcarotid approach, a left common carotid artery (LCCA) approach, a left subclavian artery (LSA) approach, and so on. In some aspects of the present technology, omitting the graft material around the primary lumen 4823 can reduce the overall delivery profile of the base member 4810—potentially allowing the base member 4810 to be loaded into and delivered through a relatively smaller catheter and/or delivered along a narrower intravascular delivery pathway.

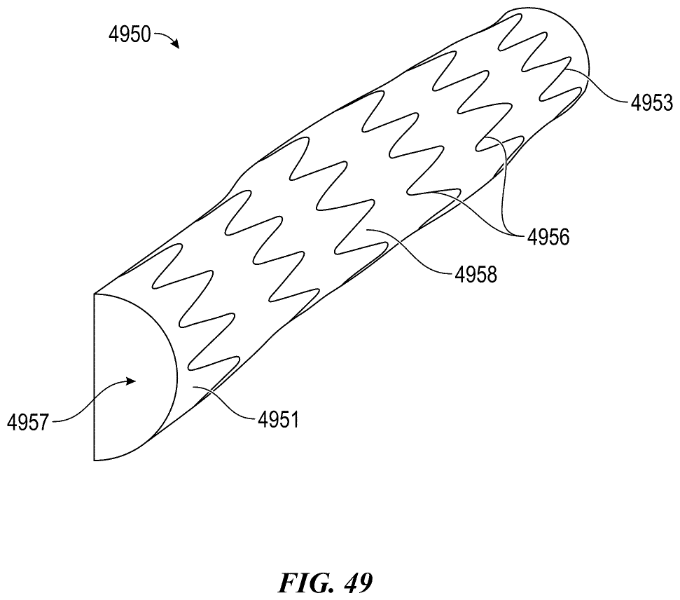

The primary lumen 4823 is configured (e.g., shaped, sized, and position) to receive a spanning member therein and/or therearound when the base member 4810 is implanted within an aorta. FIG. 49, for example, is an isometric side view of a spanning member 4950 configured to be coupled to the base member 4810 in accordance with embodiments of the present technology. In the illustrated embodiment the spanning member 4950 is a stent graft comprising a graft material 4958 defining a spanning lumen 4957 and extending between a proximal end portion 4951 and a distal end portion 4953.

Referring to FIGS. 48A-49 together, the spanning member 4950 can have a D-like cross-sectional shape that is sized to match the D-like cross-sectional shape of the primary lumen 4823. The spanning member 4950 can be secured to the proximal stents 4826a around and/or within the primary lumen 4823 such that the graft material 4958 of the spanning member 4950 engages and seals to (i) the graft material 4828 around the secondary lumen 4825 (e.g., the septum 4818) and (ii) a wall of the aorta. In this manner, the body 4812 (e.g., the graft material 4828 around the secondary lumen 4825) and the spanning member 4950 coupled in/around the primary lumen 4823 can together provide a complete circumferential seal around the wall of the aorta and can be positioned against/adjacent a diseased portion of the aorta, such as the origin of a dissection and/or aneurysm, to block blood flow into the diseased portion by diverting the blood flow through the base member 4810 and the spanning member 4950 and past the diseased portion.

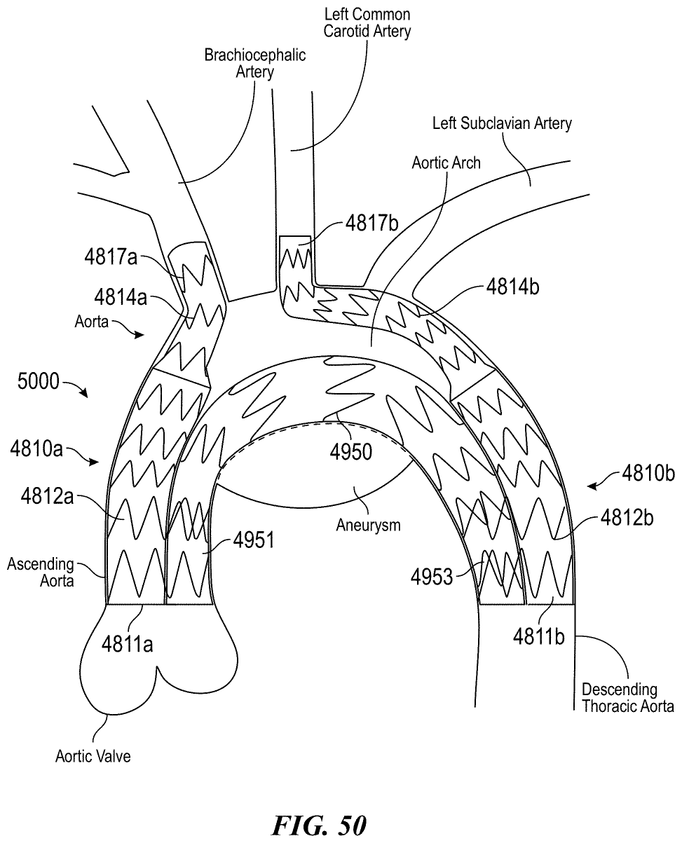

FIG. 50 is a side view of an aortic repair device 4600 comprising a pair of the base members 4810 of FIGS. 48A-48H (identified individually as a proximal base member 4810a and a distal base member 4810b) and the spanning member 4950 of FIG. 49 implanted within an aorta in accordance with embodiments of the present technology. The various components of the proximal base member 4810a are referenced with a trailing “a” (e.g., body 4812a), and the various components of the distal base member 4810b are referenced with a trailing “b” (e.g., body 4812b). In some embodiments, the aorta can include an aneurysm, dissection, and/or other diseased portion as described in detail above with reference to FIGS. 1A-2B.

In the illustrated embodiment, the body 4812a of the proximal base member 4810a is implanted within the proximal aorta (e.g., the ascending aorta and/or the aortic arch) with (i) the proximal end portion 4811a positioned proximate to the aortic valve and (ii) the secondary leg 4814a extending from the body 4812a to the brachiocephalic artery where the distal end portion 4817a is positioned. The body 4812b of the distal base member 4810b is implanted within the descending thoracic aorta (and/or partially within the aortic arch) with (i) the end portion 4811b positioned distalmost within the aorta and (ii) the secondary leg 4814b extending from the body 4812b to the left common carotid artery where the end portion 4817b is positioned. Accordingly, the distal base member 4810b can be oriented opposite the proximal base member 4810a. The spanning member 4950 can extend between the base members 4810 with the proximal end portion 4951 coupled to the body 4812a of the proximal base member 4810a and the distal end portion 4953 coupled to the body 4812b of the distal base member 4810b.

The body 4812a of the proximal base member 4810a and the proximal end portion 4951 of the spanning member 4950 can together engage and seal against a proximal portion of the aorta (e.g., a diseased portion of the ascending aorta including a tear). The body 4812b of the distal base member 4810b and the distal end portion 4953 of the spanning member 4950 can together engage and seal against a distal portion of the aorta (e.g., a portion of the descending thoracic aorta). Accordingly, the proximal base member 4810a can direct blood flow from the aortic valve (i) through the secondary leg 4814a into the brachiocephalic artery to perfuse the brachiocephalic artery and (ii) through the spanning member 4950 and past the distal base member 4810b into the aorta to perfuse the aorta. In the illustrated embodiment, the spanning member 4950 routes blood flow past a diseased portion of the aortic arch, such as the illustrated aneurysm. In some embodiments, blood can flow in retrograde from the aorta through the secondary leg 4814b to perfuse the left common carotid artery. In some embodiments, a bypass can be surgically placed between the brachiocephalic artery and the left subclavian artery and/or between the left common carotid artery and the left subclavian artery to perfuse the left subclavian artery.

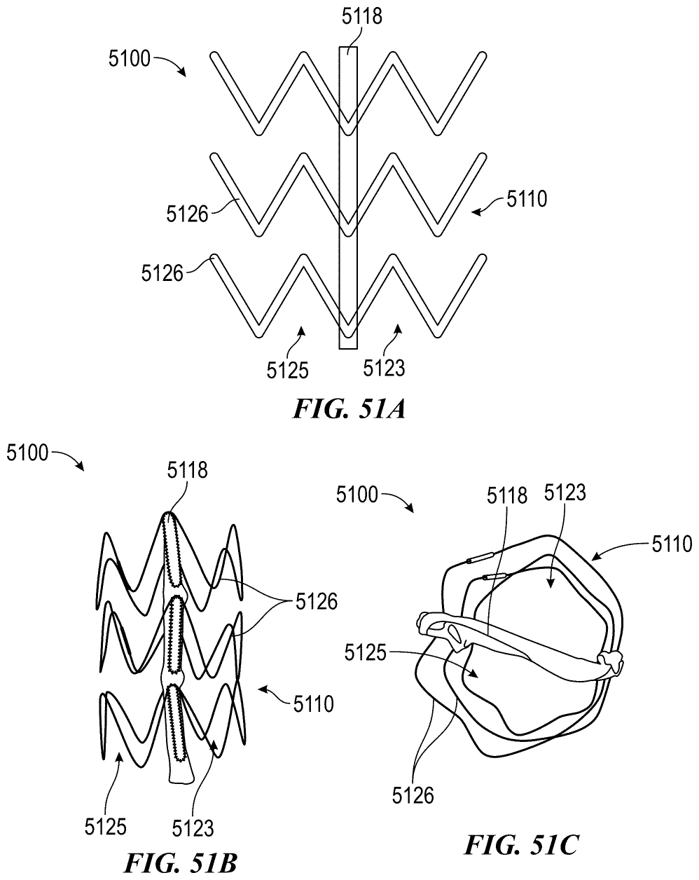

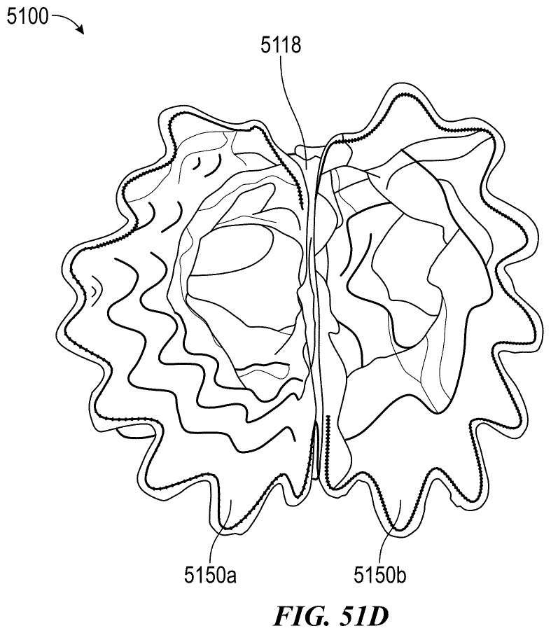

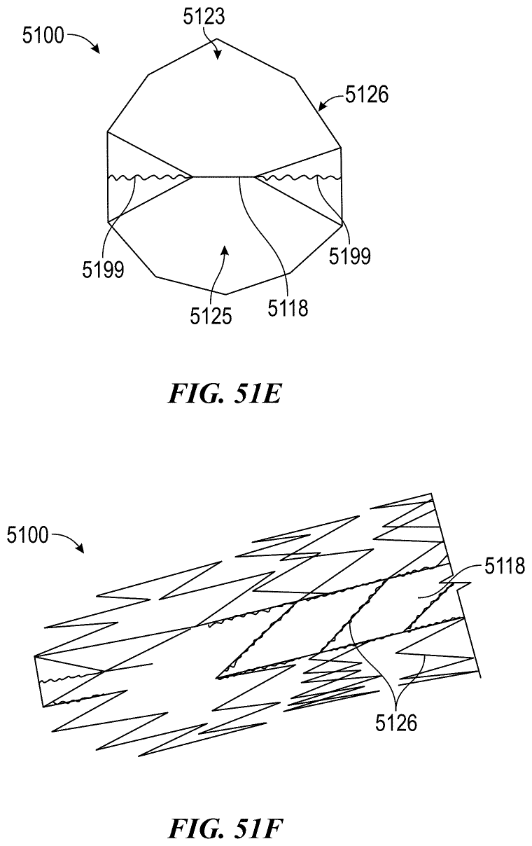

FIGS. 51A-51C are a side view, a perspective side view, and a perspective end view, respectively, of an aortic repair device 5100 in accordance with embodiments of the present technology. In the illustrated embodiment, the aortic repair device 5100 includes a base member 5110 comprising a stent graft including a septum 5118 attached to a plurality of stents 5126. In the illustrated embodiment, the stents 5126 are generally bare (e.g., not covered in a graft material) and each have a generally circular or ring-like shape with a V-pattern and are at least partially coaxially aligned. The septum 5118 interconnects the stents 5126 and divides the base member 5110 into a primary lumen 5123 and a secondary lumen 5125. In some embodiments, the septum 5118 extends along a diameter of each of the stents 5126 such that the primary and secondary lumens 5123, 5125 have the same size and a D-like cross-sectional shape. In other embodiments, one or more of the stents 5126 are offset relative to the septum 5118 such that the primary and secondary lumens 5123, 5125 have different sizes.

FIGS. 51E and 51F are a distally-facing end view and a side view, respectively, of the aortic repair device 5100 in accordance with additional embodiments of the present technology. Referring to FIGS. 51E and 51F together, in the illustrated embodiment the septum 5118 includes wedge-shaped sections 5199 to provide a more oval cross-sectional shape to the primary and secondary lumens 5123, 5125 and to help prevent the septum 5118 from flopping into one lumen or the other. In some embodiments, the ends of the wedge-shaped sections 5199 are sewn shut to prevent blood flow through the wedge-shaped sections 5199. In order to hold the septum 5118 with the wedge-shaped ends open appropriately, the stents 5126 can be shaped to provide longitudinal struts at the apices of the wedge-shaped sections 5199 to which the septum 5118 can be sewn, as shown in FIG. 51F.

The primary and secondary lumens 5123, 5125 are each configured (e.g., shaped, sized, and positioned) to receive a spanning member therein and/or therearound when the base member 5110 is implanted within an aorta. FIG. 51D, for example, is a distally-facing perspective end view of the aortic repair device 5100 of FIGS. 51A-51C including a first spanning member 5150a and a second spanning member 5150b coupled to the base member 5110 in accordance with embodiments of the present technology. The spanning members 5150a-b can each comprise an expandable stent graft sized and shaped such that, when the spanning members 5150a-b are coupled to/within the primary and secondary lumens 5123, 5125, respectively, and the aortic repair device 5100 is implanted within an aorta, the spanning members 5150 engage and seal against (i) the septum 5118, (ii) one another, and/or (iii) the wall of the aorta.

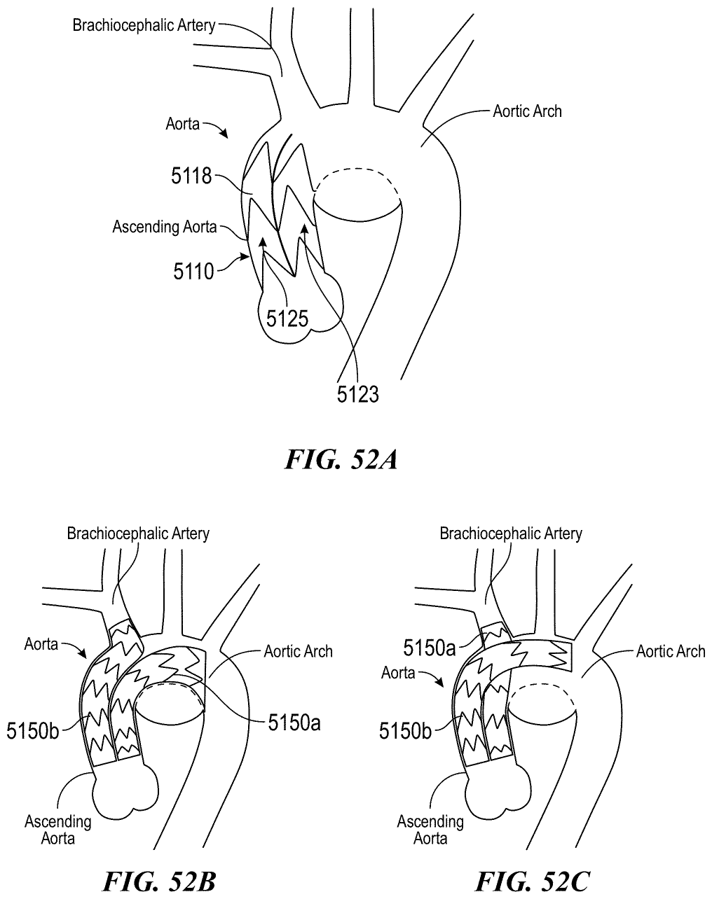

More specifically, FIGS. 52A-52C are side views of the aortic repair device 5100 of FIG. 51D during different stages of implantation within an aorta in accordance with embodiments of the present technology. Referring first to FIG. 52A, the base member 5110 is initially implanted within the proximal aorta (e.g., the ascending aorta and/or the aortic arch). The base member 5110 can be delivered to the aorta in a collapsed configuration within a catheter system (not shown). In some aspects of the present technology, omitting graft material from around the primary and secondary lumens 5123, 5125 by including only the septum 5118 can reduce the overall delivery profile of the base member 5110-potentially allowing the base member 5110 to be loaded into and delivered through a relatively smaller catheter and/or delivered along a narrower intravascular delivery pathway.

Referring to FIGS. 52B and 52C together, next the spanning members 5150a-b can be delivered to and coupled to the base member 5110 over and/or within the primary and secondary lumens 5123, 5125 (FIG. 52A). The spanning members 5150a-b (e.g., proximal end portions thereof) can engage and seal against (i) the septum 5118 (FIG. 52A), (ii) one another, and/or (iii) the wall of the aorta to divert blood flow through the spanning members 5150a-b and past a diseased portion of the aorta. As shown in FIG. 52A, in some embodiments the spanning members 5150a-b can be arranged parallel to one another within the aorta such with the first spanning member 5150a routing blood flow farther through the aorta and the second spanning member 5150b routing blood flow from the aorta to a branch vessel, such as the brachiocephalic artery. In other embodiments, as shown in FIG. 52C, the spanning members 5150a-b can be arranged to cross one another within the aorta such with the first spanning member 5150a routing blood flow from the aorta to a branch vessel, such as the brachiocephalic artery, and the second spanning member 5150b routing blood flow farther through the aorta.

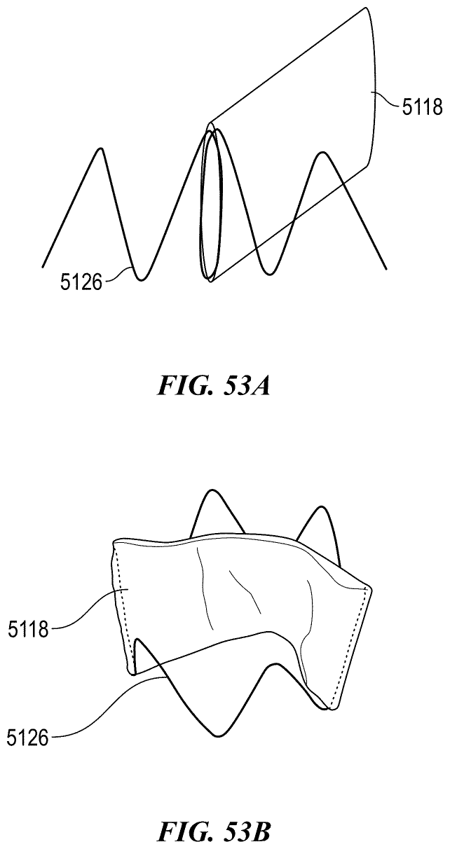

Referring to FIGS. 51A-52C together, in some embodiments the septum 5118 is configured to promote sealing between the spanning members 5150 and inhibit leaks. FIGS. 53A and 53B are an enlarged side view and a perspective side view, respectively, of the septum 5118 and one of the stents 5126 of the base member 5110 of FIGS. 51A-51C in accordance with embodiments of the present technology. Referring to FIGS. 53A and 53B together, the septum 5118 can comprise a single sheet of graft material that is attached to the stent 5126 in a sideways loop. The loop can provide an increased septum thickness that facilitates sealing and conformance with the spanning members 5150a-b (FIG. 51D). In some embodiments, an expandable foam can be placed inside the sideways loop of graft material of the stent 5126 to cause it to further expand and seal against the spanning members 5150a-b.

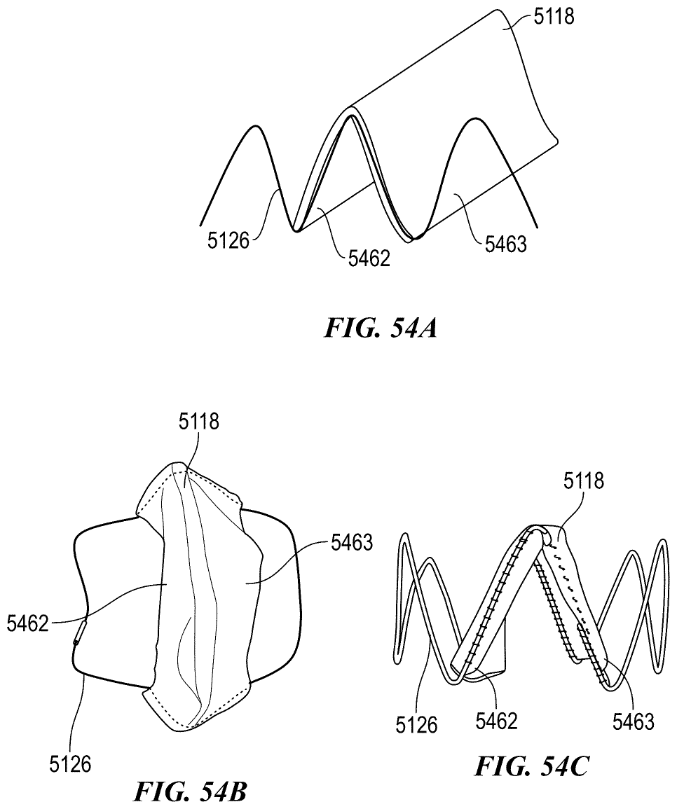

Similarly, FIGS. 54A-54C are an enlarged side view, a perspective top view, and a perspective side view, respectively, of the septum 5118 and one of the stents 5126 of the base member 5110 of FIGS. 51A-51C in accordance with additional embodiments of the present technology. Referring to FIGS. 54A-54C together, the septum 5118 can comprise a single sheet of graft material that is attached to the stent 5126 along the V-pattern of the stent 5126 such that the septum 5118 has a corresponding folded or V-shape. Accordingly, the septum 5118 can have a first end portion 5462 (e.g., a first free end portion) and a second end portion 5463 (e.g., a second free end portion) that may conform (e.g., mold, pinch, flex) inward against the spanning members 5150a-b (FIG. 51D) to facilitate sealing and conformance with the spanning members 5150a-b.

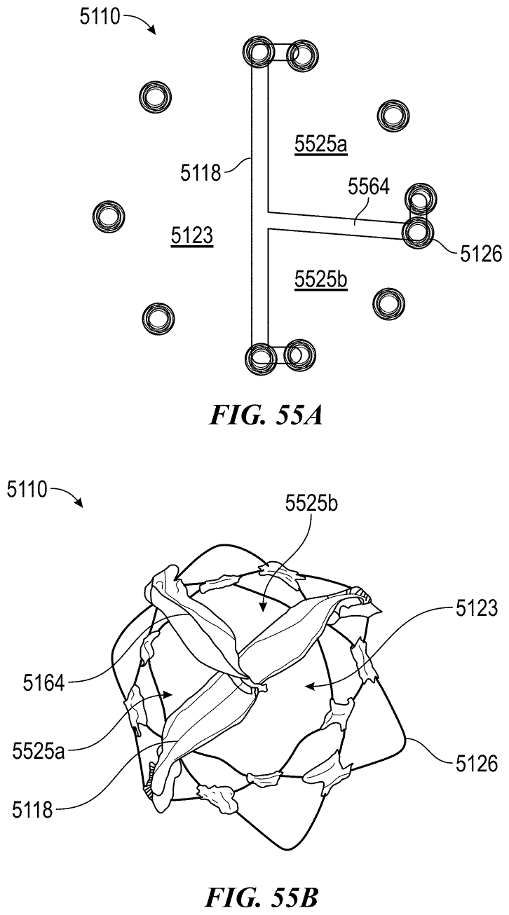

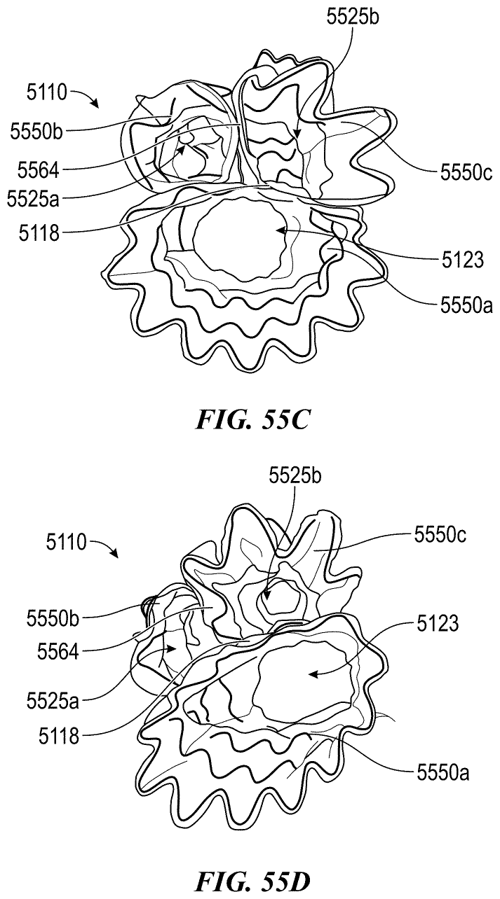

Referring again to FIGS. 51A-52C together, in some embodiments the base member 5110 can include multiple septa that define more than two lumens within the base member 5110 that can each receive a corresponding spanning member. FIGS. 55A and 55B, for example, are a cross-sectional view and a perspective end view, respectively, of the base member 5110 in accordance with additional embodiments of the present technology. Referring to FIGS. 55A and 55B together, in the illustrated embodiment the base member 5110 further includes a second septum 5564 extending between the first septum 5118 and/one or more of the stents 5126. Accordingly, the first and second septa 5118, 5564 can define the primary lumen 5123, a first secondary lumen 5525a, and a second secondary lumen 5525b that can, for example, each receive a different spanning member for perfusing a different branch vessel or portion of the aorta. In other embodiments, the base member 5110 can include any number of lumens, and/or the first and second septa 5118, 5564 can be positioned differently (e.g., offset) such that the lumens have different relative shapes and sizes.

FIGS. 55C and 55D, for example, are perspective end views of the base member 5110 including a first spanning member 5550a, a second spanning member 5550b, and a third spanning member 5550c, coupled to the base member 5110 in accordance with embodiments of the present technology. The base member 5110 is in a relaxed configuration in FIG. 55C and in a radially-compressed position in FIG. 55D. Referring to FIGS. 55C and 55D together, the primary lumen 5123 receives the first spanning member 5550a therein, the first secondary lumen 5525a receives the second spanning member 5550b therein, and the second secondary lumen 5525b receives the third spanning member 5550c therein. As shown in FIG. 55D, the spanning members 5550a-c can conform to one another and sealingly engage the first and second septa 5118, 5564 to inhibit openings (e.g., gutters, leak paths) therebetween even when radially compressed.

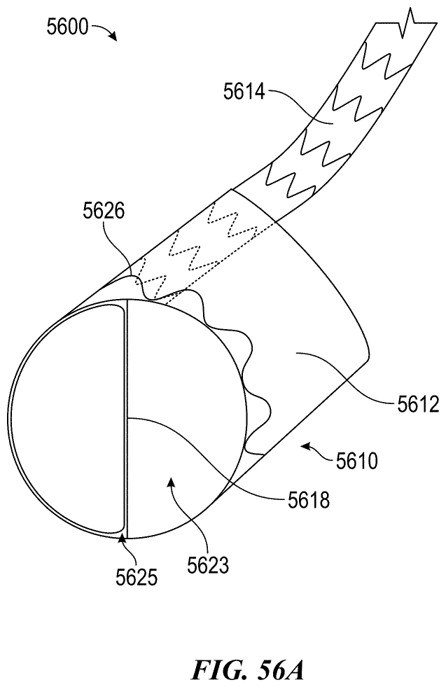

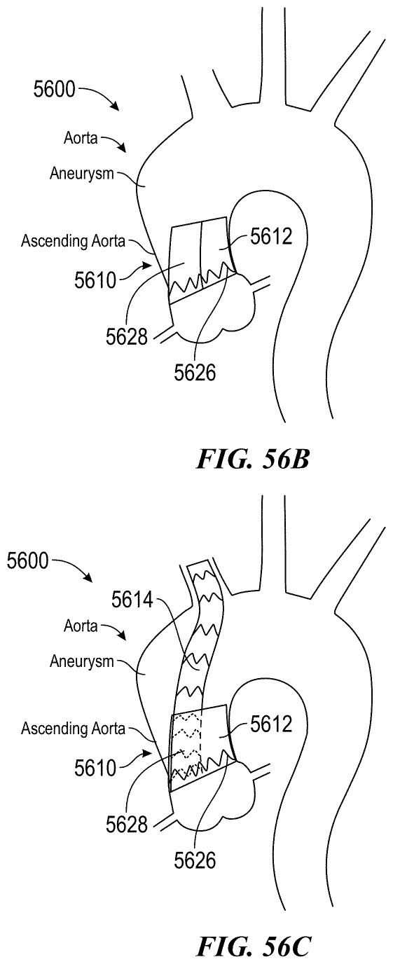

FIG. 56A is an enlarged isometric end view of an aortic repair device 5600 in accordance with embodiments of the present technology. In the illustrated embodiment, the aortic repair device 5600 includes a base member 5610 having a body 5612 including a septum 5618 defining a primary lumen 5623 and a secondary lumen 5625. The aortic repair device 5600 further includes a separate leg member 5614 coupled to the body 5612 within the secondary lumen 5625. In some embodiments, the body 5612 is a stent graft including a proximal fixation stent 5626 and a graft material 5628 attached to and extending distally from the proximal fixation stent 5626. That is, the body 5612 does not include multiple stents spaced apart along its length but only the proximal fixation stent 5626.

FIGS. 56B and 56C are side views of the aortic repair device 5600 of FIG. 56A during different stages of implantation within an aorta in accordance with embodiments of the present technology. Referring first to FIG. 56B, the base member 5610 is initially implanted within the proximal aorta (e.g., the ascending aorta, the aortic arch, and/or the aortic root) with the proximal fixation stent 5626 at least temporarily securing the position of the base member 5610. The base member 5610 can be delivered to the aorta in a collapsed configuration within a catheter system (not shown). In some aspects of the present technology, including only the proximal fixation stent 5626 rather than multiple stents along the body 5612 can reduce the overall delivery profile of the base member 5610-potentially allowing the base member 5610 to be loaded into and delivered through a relatively smaller catheter and/or delivered along a narrower intravascular delivery pathway.

Referring to FIG. 56C, next the leg member 5614 can coupled within the secondary lumen 5625 and can sealingly engage the body 5612. In some embodiments, the radial outward force of the leg member 5614 and the proximal fixation stent 5626 can drive the graft material 5628 to engage and seal the aorta. Accordingly, the aortic repair device 5600 can route blood flow past a diseased portion of the aorta (such as an ascending aortic aneurysm) to one or more branch vessels and the distal aorta.

### V. SELECTED EMBODIMENTS OF AORTIC REPAIR DEVICES HAVING MULTIPLE COMPONENTS, SUCH AS FOR FULL AORTIC ARCH TREATMENT

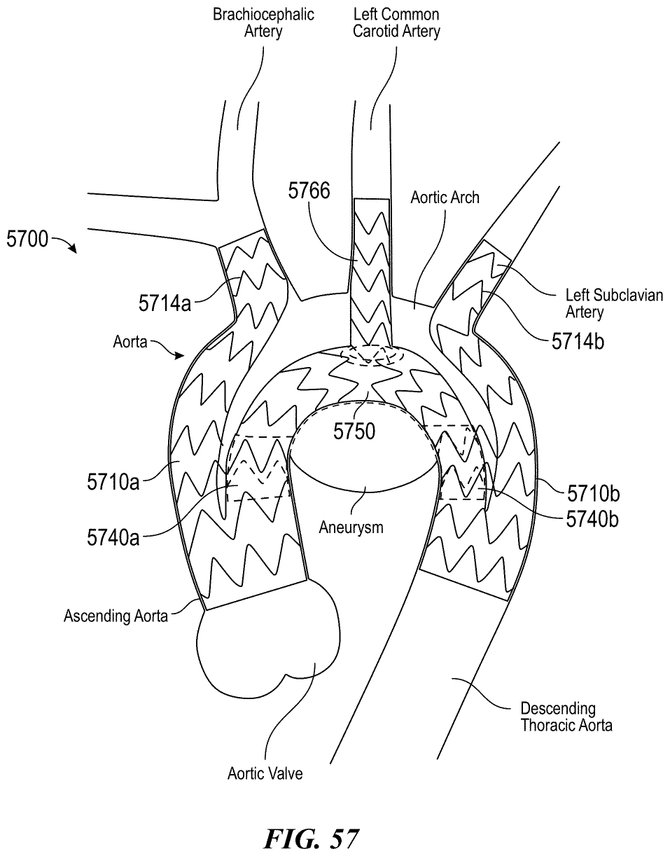

Embodiments of the present technology—including one or more the disclosed base members, spanning members, and/or other implants—can be combined to provide arrangements of aortic repair devices for treating all or substantially all of the entire proximal aorta (e.g., including the ascending aorta and the aortic arch). That is, the aortic repair devices of the present technology can effectively reconstruct a portion of the proximal aorta and/or multiple aortic branch vessels. FIGS. 57-65B are certain example combinations of the disclosed technology for full arch treatment. However, one of ordinary skill in the art will appreciate that any of the devices and components described herein can be substituted in the exemplary combinations and that other combinations are possible.

FIG. 57, for example, is a side view of an aortic repair device 5700 implanted within an aorta in accordance with embodiments of the present technology. In the illustrated embodiment, the aortic repair device 5700 includes a first base member 5710a implanted at least partially within the ascending aorta, a second base member 5710b implanted at least partially within the descending thoracic aorta, and a spanning member 5750 extending between and coupling the first and second base members 5710a-b. For example, the first base member 5710a can include a primary leg 5740a that sealing engages and secures a proximal portion of the spanning member 5750 (e.g., as described in detail above with reference to FIGS. 43A-46), and the second base member 5710b can similarly include a primary leg 5740b that sealing engages and secures a distal portion of the spanning member 5750. Thus, the spanning member 5750 can route blood flow distally through the aorta past a diseased portion of the aorta, such as an aortic arch aneurysm. The first base member 5710a can further include a secondary leg 5714a extending into a first branch artery (e.g., the brachiocephalic artery) for perfusing the first branch artery, and the second base member 5710b can include a secondary leg 5714b extending into a second branch artery (e.g., the left subclavian artery) for perfusing the second branch artery.

In the illustrated embodiment, the aortic repair device 5700 further includes a branch perfusion member 5766 coupling the spanning member 5750 to a third branch artery (e.g., the left common carotid artery) for perfusing the third branch artery. In some embodiments, the spanning member 5750 can be fenestrated (either before implantation or in situ after implantation) and the branch prefusion member 5766 coupled thereto to provide a flow path to the third branch artery. In other embodiments, the third branch artery can be perfused via bypass or via a spanning member connected to one or both of the base members 5710a-b.

Notably, in the illustrated embodiment, the second base member 5710b is implanted in a reversed or flipped orientation relative to the first base member 5710a and is substantially similar to the first base member 5710. Accordingly, the “distal” portions of the first base member 5710a correspond to the “proximal portions” of the second base member 5710b and vice versa. Therefore, one of ordinary skill in the art will understand that the use of the terms “proximal” and “distal” is based on the orientation of the base members 5710a-b and that “distal” components of one of the base members 5710a-b can correspond to the “proximal” components of the other one of the base members 5710a-b and vice versa.

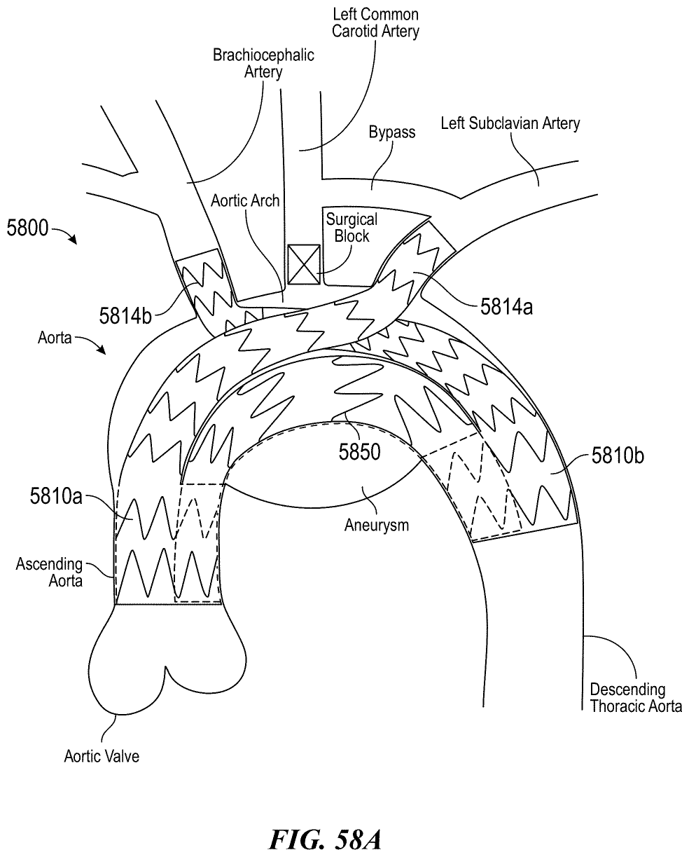

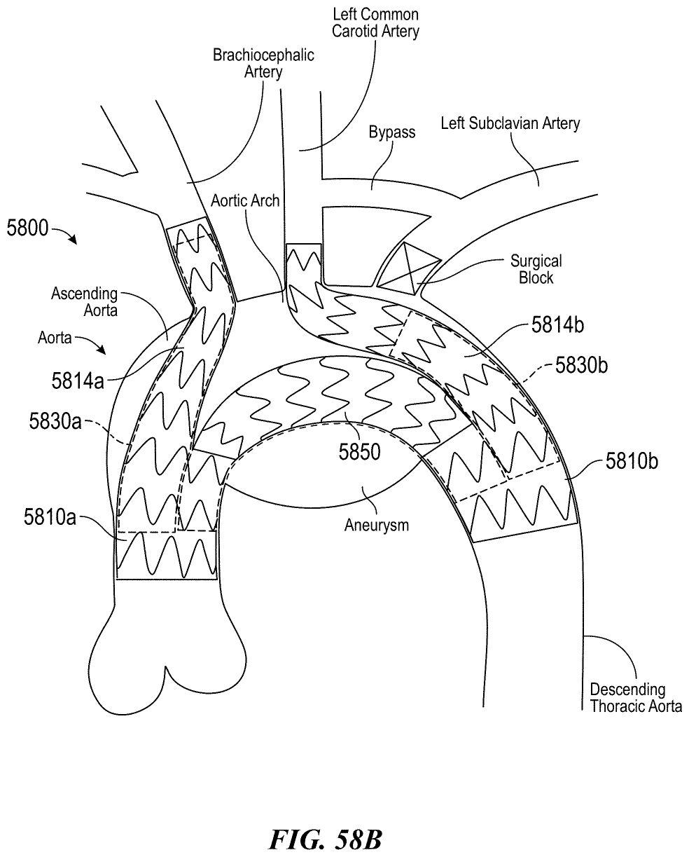

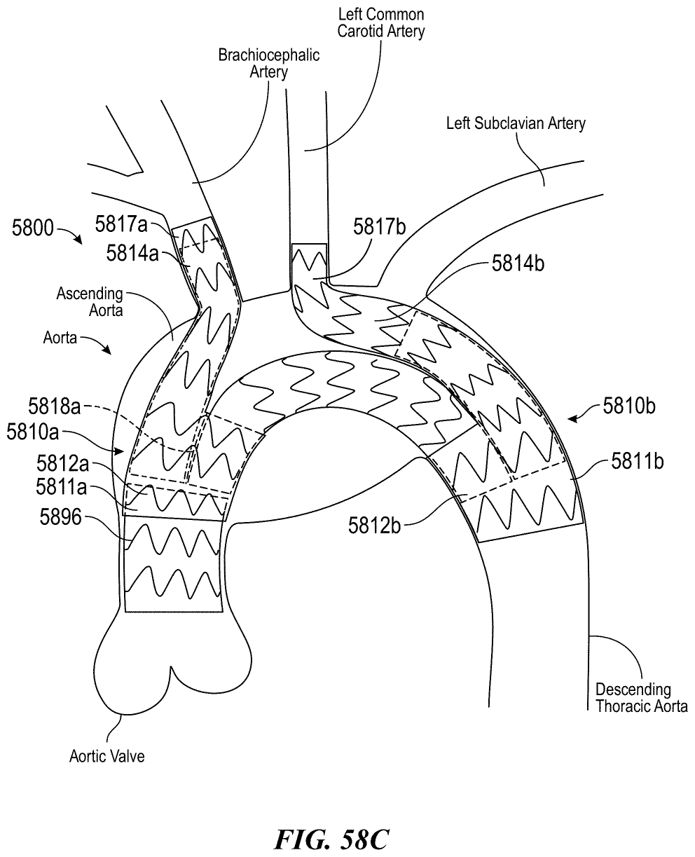

FIG. 58A is a side view of an aortic repair device 5800 implanted within an aorta in accordance with additional embodiments of the present technology. In the illustrated embodiment, the aortic repair device 5800 includes a first base member 5810a implanted at least partially within the ascending aorta, a second base member 5810b implanted at least partially within the descending thoracic aorta, and a spanning member 5850 extending between and coupling the first and second base members 5810a-b. For example, the first base member 5810a can include a primary internal lumen defined by a septum (e.g., as described in detail about with reference to FIGS. 3A-42D) that sealingly engages and secures a proximal portion of the spanning member 5850, and the second base member 5810b can similarly include a primary internal lumen defined by a septum that sealing engages and secures a distal portion of the spanning member 5850. Thus, the spanning member 5850 can route blood flow distally through the aorta past a diseased portion of the aorta, such as an aortic arch aneurysm. The first base member 5810a can further include a leg 5814a extending into a first branch artery (e.g., the left subclavian artery) for perfusing the first branch artery, and the second base member 5810b can include a leg 5814b extending into a second branch artery (e.g., the brachiocephalic artery) for perfusing the second branch artery. In the illustrated embodiment, a third branch artery (e.g., the left common carotid artery) includes a surgical block near the aorta and is perfused via a bypass between the first and third branch arteries. In some aspects of the present technology, the crisscross or overlapping arrangement of the legs 5814a-b can be easier to achieve during delivery than an embodiment in which the legs 5814a-b perfuse the nearest supra-aortic branch artery due to the take-off angles of the branch arteries off the aorta and/or the required delivery pathways.

FIG. 58B is a side view of the aortic repair device 5800 of FIG. 58A implanted within an aorta in a different configuration in accordance with embodiments of the present technology. In the illustrated embodiment, the first base member 5810a is implanted at least partially within the ascending aorta, the second base member 5810b is implanted at least partially within the descending thoracic aorta, and the spanning member 5850 extends between and couples the first and second base members 5810a-b. However, in the illustrated embodiment the leg 5814a of the first base member 5810a extends into the brachiocephalic artery for perfusing the brachiocephalic artery, and the leg 5814b of the second base member 5810b extends into the left common carotid artery for perfusing the left common carotid artery. In some embodiments, a surgical block is created in the left subclavian artery near the aorta and can be perfused via a bypass between the left common carotid artery and the left subclavian artery. In some embodiments, a first stent 5830a can be implanted at least partially within the first leg 5814a to maintain the lumen therethrough, and a second stent 5830b can be implanted at least partially within the second leg 5814b to maintain the lumen therethrough.

FIG. 58C is a side view of the aortic repair device 5800 of FIG. 58B implanted within an aorta and including an additional extending member 5896 in accordance with embodiments of the present technology. In the illustrated embodiment, the extending member 5896 (which can also be referred to as an extender, a cuff, an anchor, a seal, and/or the like) is coupled to and/or positioned within a proximal end portion 5811a of the first base member 5810a. More specifically, the extending member 5896 can be a stent graft or other device and can be at least partially positioned within a body 5812a of the first base member 5810a to extend proximally from the body 5810a toward the aortic valve. For example, the first base member 5810a can include a septum 5818a that extends only partially through the first body 5818a (e.g., as described in detail above with reference to FIGS. 5A-8H) such that the extending member 5896 conforms to the full internal circumference of the body 5810a at and proximate to the proximal end portion 5811a of the first body 5810a.

The extending member 5896 can sealingly engage (i) the body 5812a within the lumen (e.g., a primary lumen) of the body 5812a and (ii) the aorta proximal to the first base member 5810 to define a continuous blood flow path through the extending member 5896 and the first base member 5810a. The extending member 5896 can be implanted during the same procedure as the first base member 5810a or a separate (e.g., later) procedure, and is configured to extend the sealing engagement of the aortic repair device 5800 within the aorta. Alternatively or additionally, the extending member 5896 can help anchor the first base member 5810a within the aorta. In some such embodiments, the extending member 5896 can be a bare stent that does not sealingly engage the aorta. In some embodiments, the extending member 5896—or an additional one or more of the extending members 5896—can be coupled to a distal end portion 5811b of a body 5812b of the second base member 5810b, to an end portion 5817a of the leg 5814a of the first base member 5810a within the brachiocephalic artery (or another branch vessel), and/or to an end portion 5817b of the leg 5814b of the second base member 5810b within the left common carotid artery (or another branch vessel) to provide extended sealing and/or anchoring.

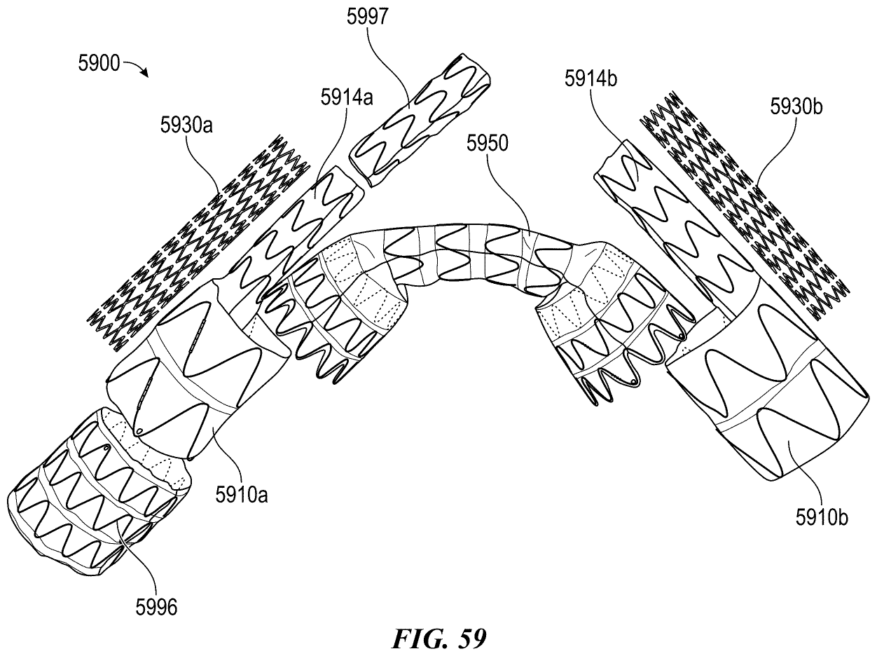

FIG. 59 is a side view of an aortic repair device 5900 in accordance with embodiments of the present technology. In the illustrated embodiment, the aortic repair device 5900 includes a (i) first base member 5910a having a leg 5914a and configured to be implanted at least partially within the ascending aorta, and (ii) a second base member 5910b having a leg 5914b configured to be implanted at least partially within the descending thoracic aorta. The aortic repair device 5900 further includes a spanning member 5950 configured to extend between and couple the first and second base members 5910a-b. In the illustrated embodiment, the spanning member 5950 has enlarged side portions and a reduced diameter middle portion as described in detail above with reference to FIG. 21. In other embodiments, the spanning member 5950 can have other configurations described herein. For example, the spanning member 5950 can have a generally constant diameter as described in detail above with reference to FIGS. 13A-14B. The aortic repair device 5900 further includes (i) a first stent 5930a configured to optionally be implanted within the leg 5914a of the first base member 5910a to help maintain the shape and patency of a lumen therethrough, and (ii) a second stent 5930b configured to optionally be implanted within the leg 5914b of the second base member 5910b to help maintain the shape and patency of a lumen therethrough.

In some embodiments, the aortic repair device 5900 further includes an extending member 5996 configured to be coupled to the first base member 5910a to provide extended sealing and/or anchoring within the ascending aorta. The aortic repair device 5900 can further include an additional extending member (not shown) configured to be coupled to the second base member 5910b to provide extended sealing and/or anchoring within the descending aorta. In the illustrated embodiment, the aortic repair device 5900 further includes an extender stent graft 5997 configured to be coupled to the leg 5914a of the first base member 5910a to, for example, provide extended sealing and/or anchoring within the aorta and/or a branch artery such as the brachiocephalic artery. The aortic repair device 5900 can further include an additional extender stent graft (not shown) configured to be coupled to the leg 5914b of the second base member 5910b to provide extended sealing and/or anchoring within the aorta and/or a branch artery such as the left common carotid artery.

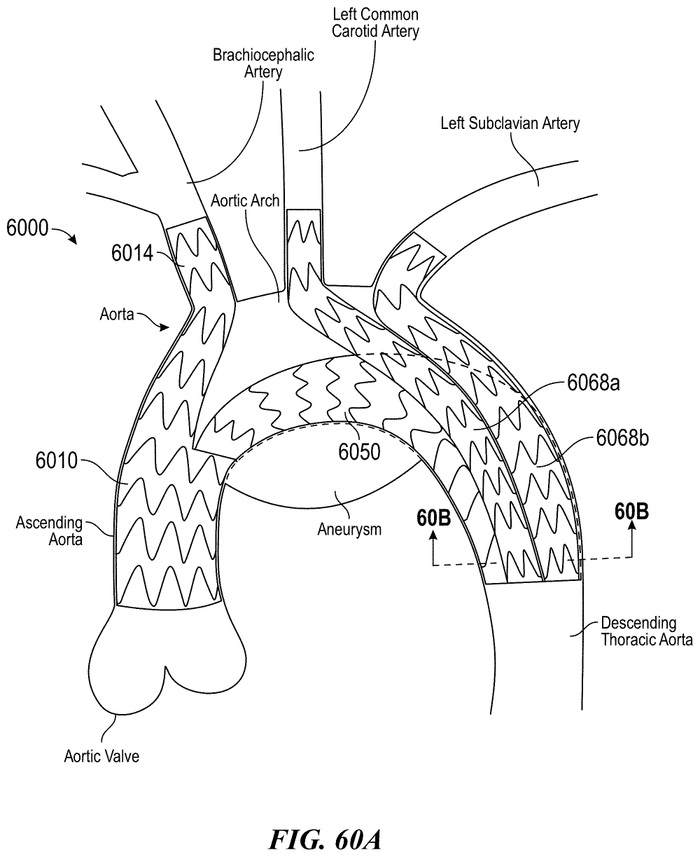

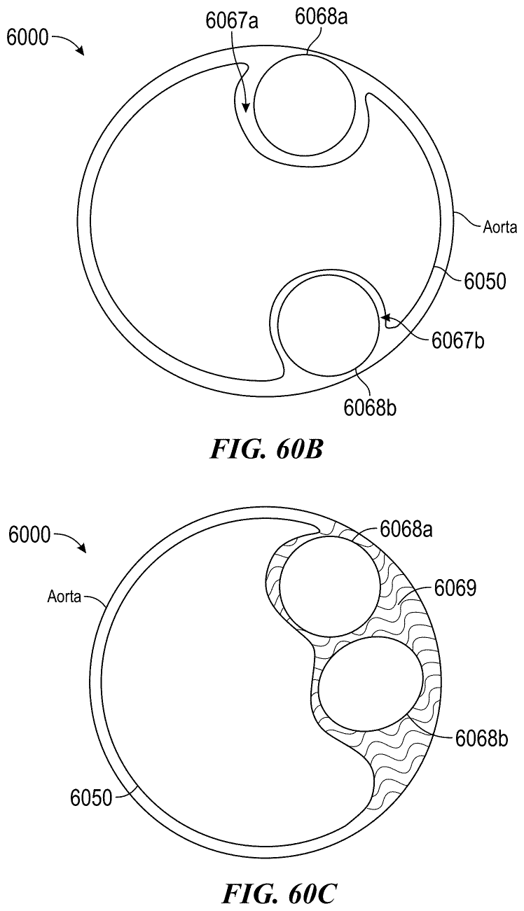

FIG. 60A is a side view of an aortic repair device 6000 implanted within an aorta in accordance with additional embodiments of the present technology. In the illustrated embodiment, the aortic repair device 6000 includes a base member 6010 implanted at least partially within the ascending aorta, and a spanning member 6050 coupled to the base member 6010 and extending past the aortic arch into the descending aorta. The base member 6010 can include a primary internal lumen defined by a septum (e.g., as described in detail about with reference to FIGS. 3A-38B) that sealing engages and secures a proximal portion of the spanning member 6050. Thus, the spanning member 6050 can route blood flow distally through the aorta past a diseased portion of the aorta, such as an aortic arch aneurysm. The base member 6010 can further include a leg 6014 extending into a first branch artery (e.g., the brachiocephalic artery) for perfusing the first branch artery.

In the illustrated embodiment, the aortic repair device 6000 further includes a first parallel member 6068a and a second parallel member 6068b extending from the aorta to second and third branch arteries, respectively (e.g., the left common carotid artery and the left subclavian artery). The first and second parallel members 6068a-b can comprise expandable stent grafts and can be at least partially arranged in parallel with the spanning member 6050 (e.g., extend next to one another). In some embodiments, the spanning member 6050 and the first and second parallel members 6068a-b can conform to one another and the wall of the aorta to circumferentially seal the aorta such that blood does not substantially flow therebetween and is contained within the spanning member 6050 and the first and second parallel member 6068a-b. Accordingly, blood can flow in retrograde from the distal outlet of the spanning member 6050 and into and through the first and second parallel member 6068a-b to the left subclavian artery and the left common carotid artery to perfuse these vessels. In some aspects of the present technology, intravascularly delivering and arranging the spanning member 6050 and the first and second parallel member 6068a-b in the parallel arrangement can be relatively easier than docking/attaching these components to a distal base member

FIGS. 60B and 60C are cross-sectional views of the aortic repair device 6000 and the aorta taken along the line 60B-60B in FIG. 60A in accordance with embodiments of the present technology. FIG. 60B illustrates a first configuration of the spanning member 6050 and the first and second parallel member 6068a-b to inhibit leaks (e.g., gutter leaks) therebetween. In the illustrated embodiment, the first and second parallel member 6068a-b each have a circular-cross sectional shape and the spanning member 6050 has a general circular cross-sectional including a first channel (e.g., cut-out, depression) 6067a and a second channel 6067b shaped to receive the first and second parallel member 6068a-b, respectively, therein. Accordingly, the spanning member 6050 and the first and second parallel member 6068a-b can together define a generally circular cross-sectional shape for engaging and sealing the aorta.

FIG. 60C illustrates a second configuration of the spanning member 6050 and the first and second parallel members 6068a-b to inhibit leaks therebetween. In the illustrated embodiment, the first and second parallel members 6068a-b each have a circular-cross sectional shape and the spanning members 6050 is formed to be more compliant/conformant than the first and second parallel members 6068a-b such that that the spanning member 6050 can conform around the more rigid first and second parallel members 6068a-b. In some embodiments, the aortic repair device 6000 can further include a filler material 6069 at least partially around/between the spanning member 6050 and the first and second parallel members 6068a-b that seals the interfaces therebetween. The filler material 6069 can be a foam, hydrogel, and/or other suitable material non-permeable to blood.

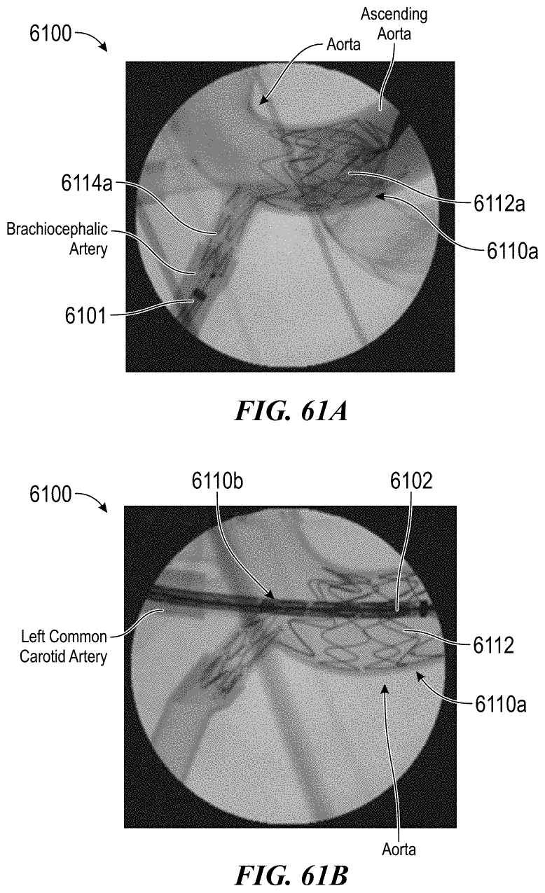

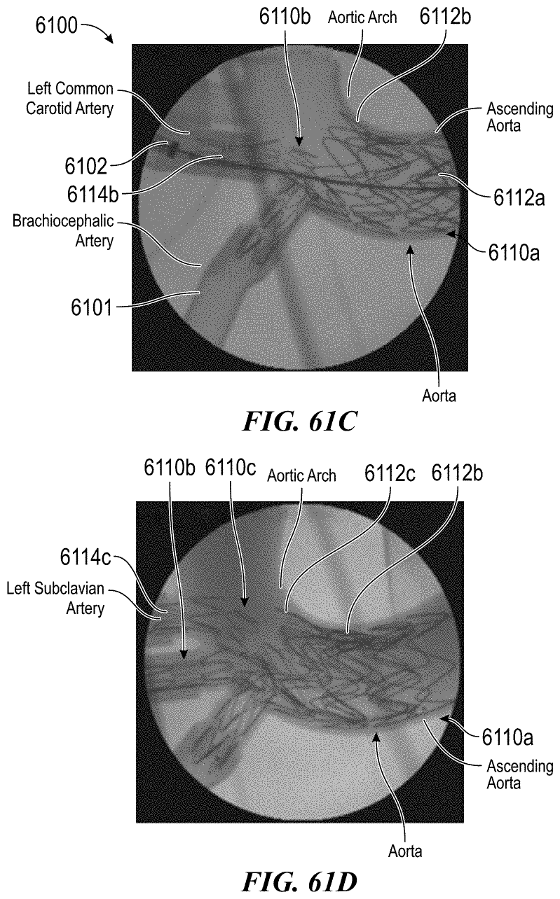

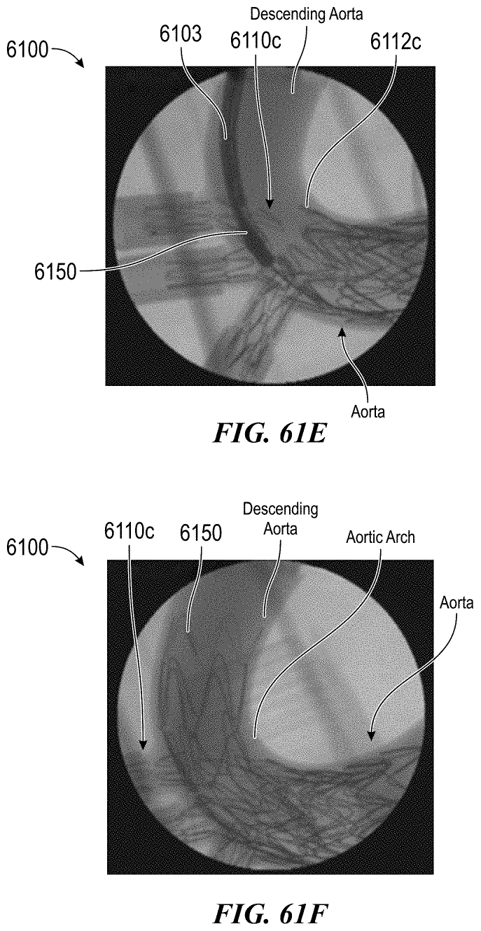

In some embodiments, multiple base members of an aortic repair device can be nested together to treat all or substantially all of the entire proximal aorta (e.g., including the ascending aorta and the aortic arch). FIGS. 61A-61F, for example, are side views (e.g., fluoroscopic images) of various stages of a procedure to implant an aortic repair device 6100 within the aorta of a patient in accordance with embodiments of the present technology.

Referring first to FIG. 61A, a first base member 6110a can be implanted within the ascending aorta via, for example, a first catheter 6101. In some embodiments, the first catheter 6101 can be advanced to the ascending aorta through the brachiocephalic artery and/or via a transapical approach. The first base member 6110a can include a body 6112a positioned within the aorta, a leg 6114a extending from the body 6112a into a first branch artery such as the brachiocephalic artery, and a septum (e.g., as described in detail about with reference to FIGS. 3A-38B) extending fully or partially through the body 6112a and defining (i) a primary lumen for blood flow through the body 6112a to the aorta and (ii) a secondary lumen for blood flow through the body 6112a to the leg 6114a and the first branch artery.

Referring next to FIG. 61B, a second catheter 6102 containing (e.g., housing, constraining) a second base member 6110b can be advanced toward the first base member 6110a and into the primary lumen of the body 6112a thereof. In some embodiments, the second catheter 6102 can be advanced through a second branch artery, such as the left common carotid artery, and into the aorta (e.g., via a transcarotid approach).

As shown in FIG. 61C, the second catheter 6102 can be withdrawn relative to the second base member 6110b to deploy the second base member 6110b within the first base member 6110a within the aorta. More specifically, a body 6112b of the second base member 6110b can be deployed at least partially within the primary lumen of the first base member 6110a and a leg 6114b of the second base member 6110b can be deployed within the second branch artery (e.g., the left common carotid artery). The body 6112b of the second base member 6110b can include a septum extending fully or partially through the body 6112b and defining (i) a primary lumen for blood flow through the body 6112b to the aorta and (ii) a secondary lumen for blood flow through the body 6112b to the leg 6114b and the second branch artery. The body 6112a and the septum of the first base member 6110a can sealingly engage and secure a proximal portion of the body 6112b of the second base member 6110b. In some embodiments, the second base member 6110b has a smaller diameter than that of the first base member 6110a. In some embodiments, the body 6112b of the second base member 6110b extends distally from the body 6112a of the first base member 6110a to extend a sealing zone of the aortic repair device 6100 within the aorta along the ascending aorta and/or the aortic arch.

Referring next to FIG. 61D, a third base member 6110c can be delivered to via a third catheter (not shown) and implanted within the second base member 6110b within the aorta via, for example, a third branch artery such as the left subclavian artery. More specifically, a body 6112c of the third base member 6110c can be deployed at least partially within the primary lumen of the second base member 6110b and a leg 6114c of the third base member 6110c can be deployed within the third branch artery (e.g., the left subclavian artery). The body 6112c of the second base member 6110c can include a septum extending fully or partially through the body 6112c and defining (i) a primary lumen for blood flow through the body 6112c to the aorta and (ii) a secondary lumen for blood flow through the body 6112c to the leg 6114c and the third branch artery. The body 6112b and the septum of the second base member 6110b can sealingly engage and secure a proximal portion of the body 6112c of the third base member 6110c. In some embodiments, the third base member 6110c has a smaller diameter than that of the first base member 6110a and the second base member 6110b. In some embodiments, the body 6112c of the third base member 6110c extends distally from the body 6112b of the second base member 6110b to extend a sealing zone of the aortic repair device 6100 within the aorta along the ascending aorta and/or the aortic arch.

Referring next to FIG. 61E, a fourth catheter 6103 containing (e.g., housing, constraining) a spanning member 6150 can be advanced toward the third base member 6110c and into the primary lumen of the body 6112c thereof. In some embodiments, the fourth catheter 6103 can be advanced through the descending aorta.

As shown in FIG. 61F, the fourth catheter 6103 (FIG. 61E) can be withdrawn relative to the spanning member 6150 to deploy the spanning member 6150 within the third base member 6110c within the aorta. The body 6112c and the septum of the third base member 6110c can sealingly engage and secure a proximal portion of the spanning member 6150. The spanning member 6150 can extend along the aortic arch and into the descending aorta to route blood flow distally through the aorta (e.g., to the descending thoracic aorta) past a diseased portion of the aorta, such as an aortic arch aneurysm. Referring to FIGS. 61A-61F together, the aortic repair device 6100 can provide for a full arch treatment in which (i) the leg 6114a of the first base member 6110a directs blood flow to a first branch artery (e.g., the brachiocephalic artery), (ii) the leg 6114b of the second base member 6110b directs blood flow to a second branch artery (e.g., the left common carotid artery), (iii) the leg 6114c of the third base member 6110c directs blood flow to a third branch artery (e.g., the left subclavian artery), (iv) the primary lumens of the first through third base members 6110a-c direct blood flow to the spanning member 6150, and (v) the spanning member 6150 directs blood flow into the descending aorta.

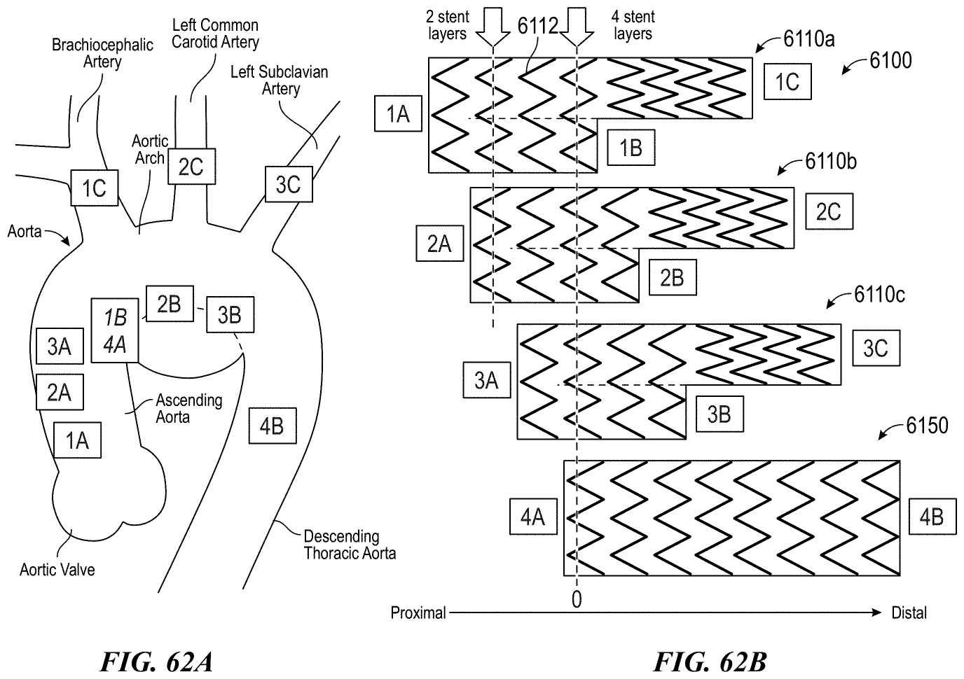

FIGS. 62A and 62B provide a schematic view of the aortic repair device 6100 of FIGS. 61A-61F implanted within the aorta in accordance with embodiments of the present technology. Specifically, FIG. 62B is an exploded view of the various components of the aortic repair device 6100 illustrating their relative overlap in a proximal to distal direction, and FIG. 62A is a side view of the aorta illustrating locations where the various components shown in FIG. 62A are implanted within the aorta. For example, as shown in FIG. 62B, the first base member 6110a has a proximal end portion or opening labeled as “1A”, a distal end portion defining an opening or exit to the primary lumen labeled as “1B,” and a leg defining a distal opening or exit to the secondary lumen labeled as “1C.” These components of the first base member 6110a are configured to be implanted within the aorta shown in FIG. 62A at the corresponding locations of the reference labels shown in FIG. 62A. For example, the proximal opening of the body 6112 (labeled “1A” in FIG. 62B) is configured to be implanted in the ascending aorta as shown by reference label “1A” in FIG. 62A, the distal opening of the body 6112 (labeled “1B” in FIG. 62B) is configured to be implanted distal from the proximal opening with the ascending aorta and/or proximate the aortic arch as shown by reference label “1B” in FIG. 62A, and the distal opening of the leg (labeled “1C” in FIG. 62B) is configured to be implanted within the brachiocephalic artery as shown by reference label “1C” in FIG. 62A. The various portions of the second base member 6110b, the third base member 6110c, and the spanning member 6150 are similarly labeled in FIGS. 61A and 61B. Accordingly, the reference labels shown in FIG. 62A illustrate the relative positioning and/or overlap of the corresponding portions of the base members 6110a-c and the spanning member 6150 when the aortic repair device 6100 is implanted within the aorta.

In some embodiments, referring to FIGS. 61A and 61B together, when the aortic repair device 6100 is implanted within the aorta, at least a portion of each of the base members 6110a-c and the spanning member 6150 can at least partially overlap (e.g., be nested with each other) such as along a line O shown in FIG. 62B. Accordingly, the aortic repair device 6100 can have four layers of stents along this region, which can make the aortic repair device 6100 relatively stiff. In some embodiments, the stents of one or more of the base members 6110a-c and/or the spanning member 6150 can be configured for increased flexibility and/or reduced thickness along such an overlap region to reduce the total material of the aortic repair device 6100 and/or to reduce the stiffness of the aortic repair device 6100.

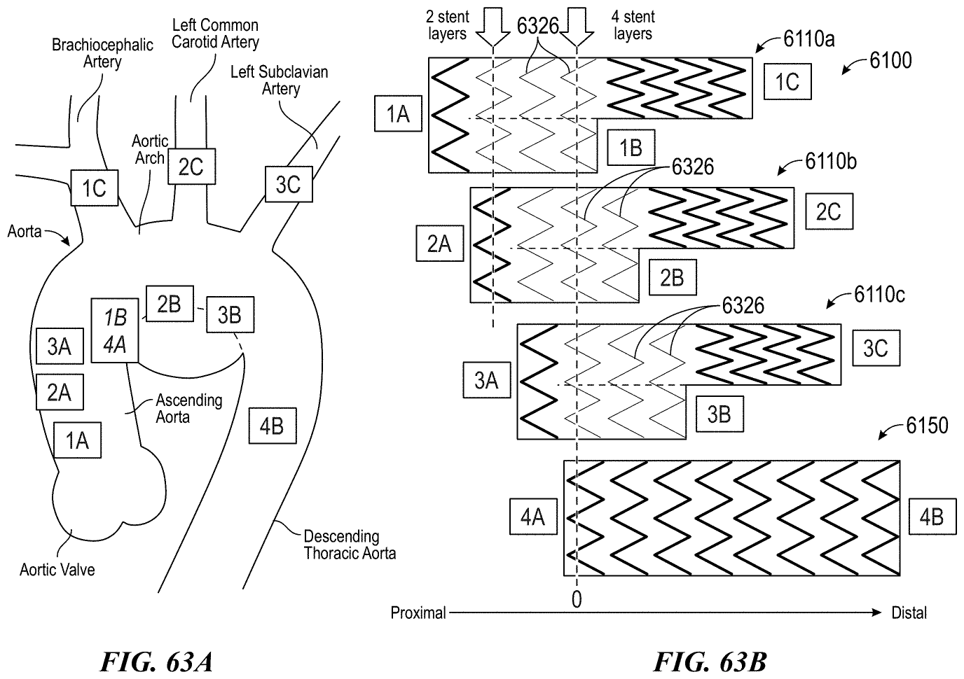

For example, FIGS. 63A and 63B provide a schematic view of the aortic repair device 6100 of FIGS. 61A-61F implanted within the aorta in accordance with additional embodiments of the present technology. The various positionings indicated by the reference labels (e.g., “1A,” “1B,” etc.) can be the same as those shown in and described in detail with reference to FIGS. 61A-62B. In the illustrated embodiment, the base members 6110a-c each have stents 6326 along the body thereof that can have a reduced diameter compared to those shown in FIGS. 62A and 62B. In some aspects of the present technology, the reduced diameter stents 6326 can reduce the total material (e.g., metal) of the aortic repair device 6100 and/or increase the relative flexibility of the aortic repair device 6100, while also providing enough radial strength for deploying and opening the base members 6100a-c. In some embodiments, the spanning member 6150 can similarly include reduced diameter stents, such as in the overlap region indicated by the line O.

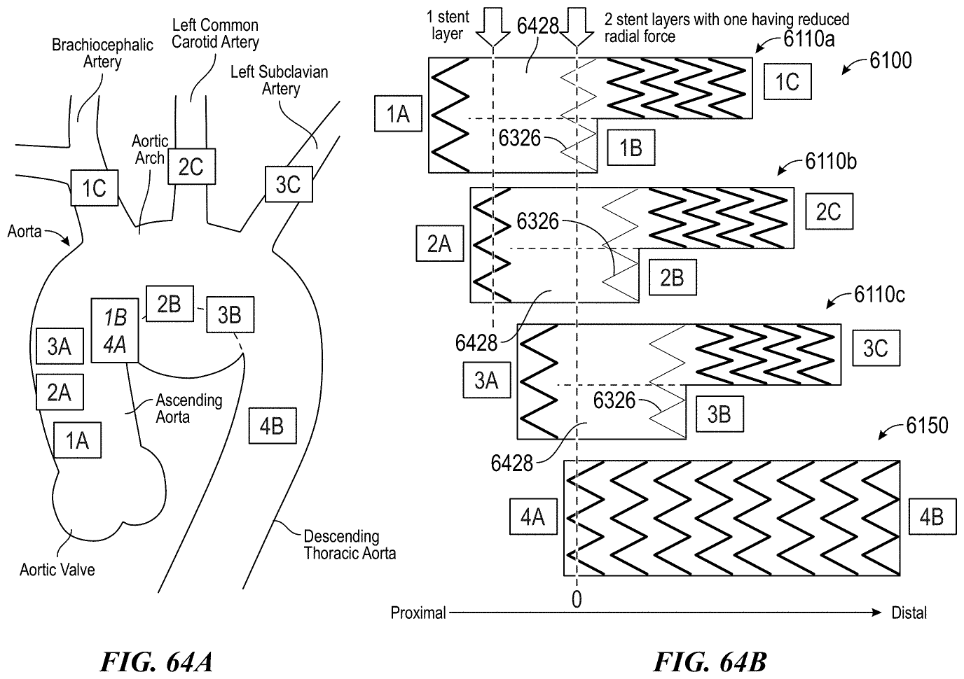

Similarly, FIGS. 64A and 64B provide a schematic view of the aortic repair device 6100 of FIGS. 61A-61F implanted within the aorta in accordance with additional embodiments of the present technology. The various positionings indicated by the reference labels (e.g., “1A,” “1B,” etc.) can be the same as those shown in and described in detail with reference to FIGS. 61A-62B. In the illustrated embodiment, the base members 6110a-c each have a single reduced diameter stent 6326 at the distal end portion, and further include a section 6428 of unsupported graft material in which stents have been omitted compared to the embodiments shown in FIGS. 62A-63B. In some aspects of the present technology, omitting the stents in the section 6428 of graft material can reduce the total material (e.g., metal) of the aortic repair device 6100 and/or increase the relative flexibility of the aortic repair device 6100, while the remaining stents provide enough radial strength for deploying and opening the base members 6100a-c. Additionally, as shown in FIG. 64B, the sections 6428 of graft material can be selected such that the aortic repair device 6100 has at least two stent layers in each section of the aortic repair device 6100, such as in the overlap region indicated by the line O. In some embodiments, the spanning member 6150 can similarly include reduced diameter stents and/or an unsupported section of graft material, such as in the overlap region indicated by the line O.

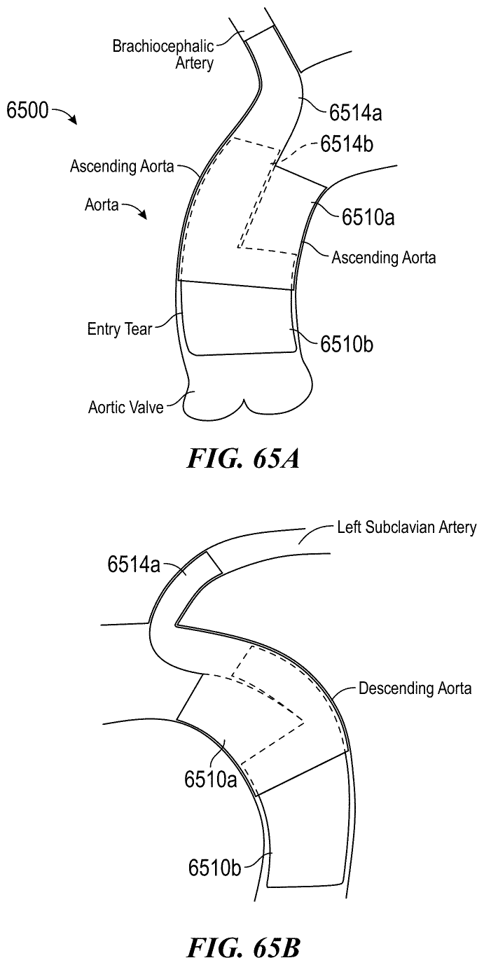

FIG. 65A is a side view of an aortic repair device 6500 implanted within an ascending aorta in accordance with embodiments of the present technology. In the illustrated embodiment, the aortic repair device 6500 includes a first base member 6510a implanted at least partially within the ascending aorta and a second base member 6510b coupled to the first base member 6510a and extending proximally from the first base member 6510a. The base members 6510a-b can each include a primary internal lumen defined by a septum (e.g., as described in detail about with reference to FIGS. 3A-42D), and a first leg 6514a and a second leg 6514b, respectively. The base members 6510a-b can conform and seal against the aorta. At least a portion of the second base member 6510b can extend into and seal against at least a portion of the first base member 6510a. For example, the second leg 6514b can extend at least partially into the first leg 6514a or the secondary lumen coupled thereto. The first leg 6514a can extend into a first branch artery (e.g., the brachiocephalic artery) for perfusing the first branch artery. Therefore, the second base member 6510b can route blood into the first base member 6510a, which routes the blood into the first branch artery via the first leg 6514a and distally through the aorta. In some aspects of the present technology, the first base member 6510a can be deployed before the first base member 6510a, and the second base member 6510b can then be deployed at least partially within the first base member 6510a to extend the sealing range of the aortic repair device 6500. In additional aspects of the present technology, deploying the second base member 6510b at least partially within the first base member 6510a can improve deployment accuracy and control of the second base member 6510b.

Similarly, FIG. 65B is a side view of an aortic repair device 6500 implanted within the descending aorta in accordance with additional embodiments of the present technology. In the illustrated embodiment, the second base member 6510b can similarly improve the sealing range of aortic repair device 6500 within the descending aorta while also having improved deployment and accuracy and control from being deployed within the first base member 6510a. The first base member 6510a can be positioned to perfuse a second branch artery (e.g., the left subclavian artery) via the first arm 6514a. In some embodiments, a spanning member can be coupled between the aortic repair devices 6500 shown in FIGS. 65A and 65B to fully treat the aortic arch.

### VI. SELECTED ADDITIONAL EMBODIMENTS OF AORTIC REPAIR DEVICES HAVING REDUCED DELIVERY PROFILES

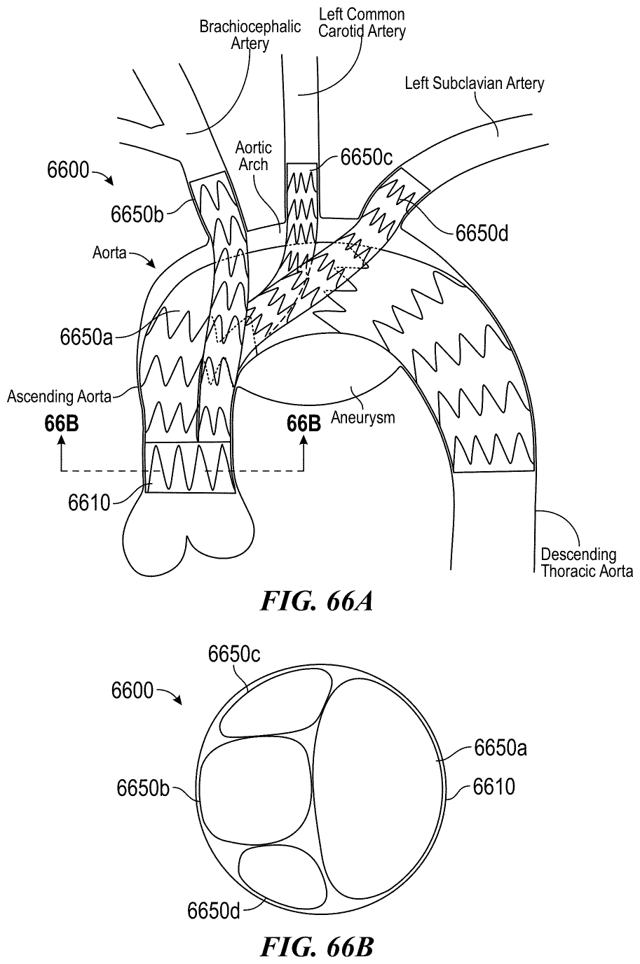

FIGS. 66A-69B illustrate additional embodiments of aortic repair devices including a (i) dock or base member configured to be implanted within the aorta and (ii) multiple spanning members configured to “dock” to the base member to provide different blood flow paths to the distal aorta and/or the branch arteries. The various aortic repair devices can include some features that are at least generally similar in structure and function, or identical in structure and function, to one another and/or the corresponding features of the aortic repair devices described in detail above with reference to FIGS. 3A-65B, and can operate in a generally similar or identical manner to one another and/or the aortic repair devices described with reference to FIGS. 3A-65B.

FIG. 66A is a side view of an aortic repair device 6600 implanted within an aorta in accordance with additional embodiments of the present technology. FIG. 66B is a cross-sectional view of the aortic repair device 6600 taken along the line 66B-66B in FIG. 66A in accordance with additional embodiments of the present technology. Referring to FIGS. 66A and 66B together, the aortic repair device 6600 includes a dock or base member 6610 implanted at least partially within the ascending aorta, and a plurality of spanning members 6650 (identified individually as first through fourth spanning members 6650a-6650d, respectively) coupled to (e.g., docked to) the base member 6610. The base member 6610 and the spanning members 6650 can each comprise an expandable stent graft. In the illustrated embodiment, each of the spanning members 6650 includes a proximal end portion positioned within the base member 6610.

In some embodiments, the base member 6610 defines a plurality of lumens (e.g., separated by one or more septa of graft material) each configured to receive and secure a corresponding one of the spanning members 6650. In some embodiments, the base member 6610 comprises a circular ring defining a single lumen and the spanning members 6650 are positioned therein in a parallel arrangement. In some embodiments, one or more of the spanning members 6650 can be integrally formed with the base member 6610 (e.g., can comprise an integral “leg”). For example, in the illustrated embodiment the second spanning member 6650b is integrally formed with the base member 6610.

In the illustrated embodiment, an outer surface of the base member 6610 engages and seals with the wall of the aorta to circumferentially seal the aorta. The spanning members 6650 can engage and/or conform to one another and an inner surface of the base member 6610 such that blood does not substantially flow therebetween and is contained within the spanning members 6650. Accordingly, blood can flow in antegrade through each of the spanning members 6650. In the illustrated embodiment, the first spanning member 6650a has a larger cross-sectional area than each of the second through fourth spanning members 6650b-d and routes blood flow distally through the aorta (e.g., to the descending thoracic aorta) past a diseased portion of the aorta, such as an aortic arch aneurysm. The second through fourth spanning members 6650b-d extend from the base member 6610 to the brachiocephalic artery, the left common carotid artery, and the left subclavian artery, respectively, and route blood flow thereto to perfuse those vessels. In some aspects of the present technology, the aortic repair device 6600 is configured to perfuse the distal aorta and each of the supra-aortic branch vessels with antegrade blood flow-which can improve the volumetric flow rates through these vessels and help ensure sufficient perfusion.

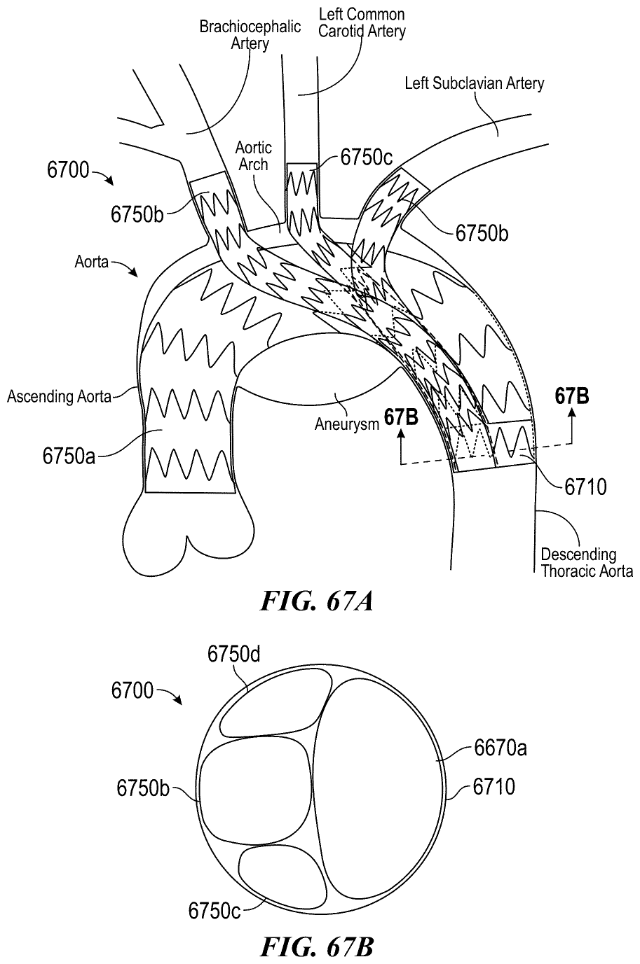

FIG. 67A is a side view of an aortic repair device 6700 implanted within an aorta in accordance with additional embodiments of the present technology. FIG. 67B is a cross-sectional view of the aortic repair device 6700 taken along the line 67B-67B in FIG. 67A in accordance with additional embodiments of the present technology. Referring to FIGS. 67A and 67B together, the aortic repair device 6700 includes a dock or base member 6710 implanted at least partially within the descending thoracic aorta, and a plurality of spanning members 6750 (identified individually as first through fourth spanning members 6750a-6750d, respectively) coupled to (e.g., docked to) the base member 6710. Each of the spanning members 6750 can include a distal end portion positioned within the base member 6710. The base member 6710 can include one or more lumens for receiving the distal end portions of the spanning members 6750, and/or one or more of the spanning members 6750 can be integrally formed with the base member 6710. For example, in the illustrated embodiment the second spanning member 6750b is integrally formed with the base member 6710.

In the illustrated embodiment, an outer surface of the base member 6710 engages and seals with the wall of the aorta to circumferentially seal the aorta. The distal end portions of the spanning members 6750 can engage and/or conform to one another and an inner surface of the base member 6710 such that blood does not substantially flow therebetween and is contained within the spanning member spanning members 6750. In the illustrated embodiment, the first spanning member 6750a has a larger cross-sectional area than each of the second through fourth spanning members 6750b-d and has a proximal end portion positioned in the ascending aorta. The first spanning member 6750a can engage and seal against the wall of the ascending aorta such that blood flows in antegrade therethrough toward the base member 6710. Accordingly, the first spanning member 6750a can route blood flow distally through the aorta (e.g., to the descending thoracic aorta) past a diseased portion of the aorta, such as an aortic arch aneurysm. Blood can then flow in retrograde into and through the second through fourth spanning members 6750b-d, which extend from the base member 6710 to the brachiocephalic artery, the left common carotid artery, and the left subclavian artery, respectively, and route blood flow thereto to perfuse those vessels. In some aspects of the present technology, the base member 6710 and/or the spanning members 6750 can be delivered via a transfemoral approach because base member 6710 is not positioned in the proximal aorta and thus the spanning members 6750 need not be “docked to” the base member 6710 within the proximal aorta.

FIG. 60A is a side view of an aortic repair device 6000 implanted within an aorta in accordance with additional embodiments of the present technology. FIG. 60B is a cross-sectional view of the aortic repair device 6000 taken along the line 60B-60B in FIG. 60A in accordance with additional embodiments of the present technology. Referring to FIGS. 60A and 60B together, the aortic repair device 6000 includes a dock or base member 6010 implanted at least partially within the ascending aorta, and a plurality of spanning members 6050 (identified individually as first through third spanning members 6050a-6050c, respectively) coupled to (e.g., docked to) the base member 6010. Each of the spanning members 6050 can include a proximal end portion positioned within the base member 6010.

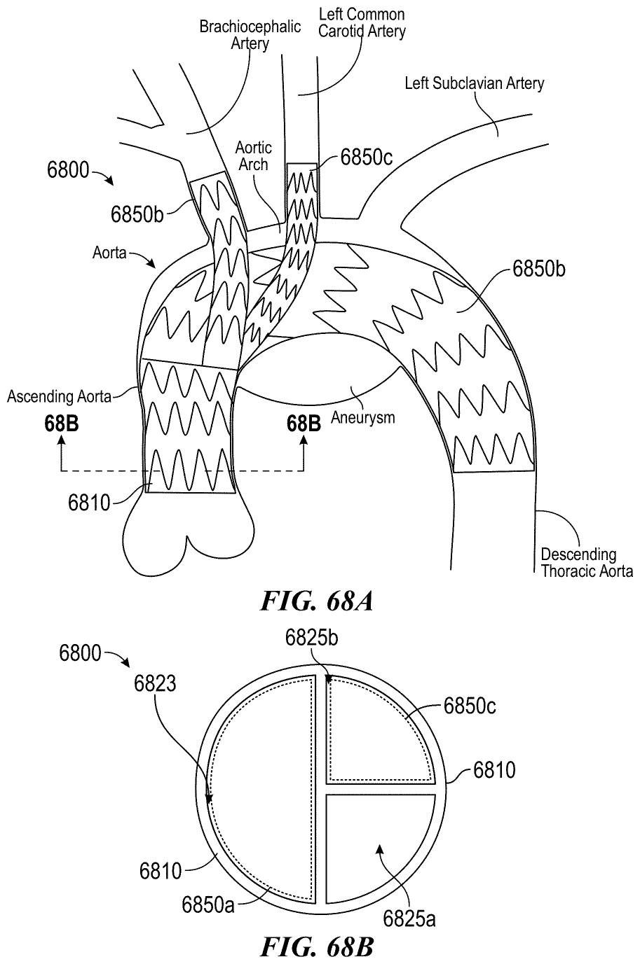

In the illustrated embodiment, the base member 6810 defines a primary lumen 6823, a first secondary lumen 6825a, and second secondary lumen 6825b. In some embodiments, as shown in FIG. 68B, the base member 6810 includes one or more fabric septa extending therethrough that define the primary lumen 6823, the first secondary lumen 6825a, and/or second secondary lumen 6825b. In the illustrated embodiment, the second spanning member 6850b is integrally formed with the base member 6810 such that the first secondary lumen 6825a extends continuously therethrough. The first spanning member 6850a includes a proximal end portion at least partially positioned within the primary lumen 6823 and configured (e.g., shaped, sized) to conform and seal against a circumference thereof. Likewise, the third spanning member 6850c includes a proximal end portion at least partially positioned within the second secondary lumen 6825b and configured (e.g., shaped, sized) to conform and seal against a circumference thereof. Accordingly, the spanning members 6850 can engage and/or conform to the lumens 6823, 6825a, 6825b and the circumference of the base member 6810 such that blood does not substantially flow therebetween and is contained within the spanning members 6850. An outer surface of the base member 6810 engages and seals with the wall of the aorta to circumferentially seal the aorta. Accordingly, blood can flow in antegrade through each of the spanning members 6850.

In the illustrated embodiment, the first spanning member 6850a has a larger cross-sectional area than the second and third spanning members 6850b-c and routes blood flow distally through the aorta (e.g., to the descending thoracic aorta) past a diseased portion of the aorta, such as an aortic arch aneurysm. The second and third spanning members 6850b-c extend from the base member 6810 to the brachiocephalic artery and the left common carotid artery, respectively, and route blood flow thereto to perfuse those vessels. In some embodiments, the left subclavian artery can be perfused via a surgical bypass or another implant coupled to one or more of the components of the aortic repair device 6800.

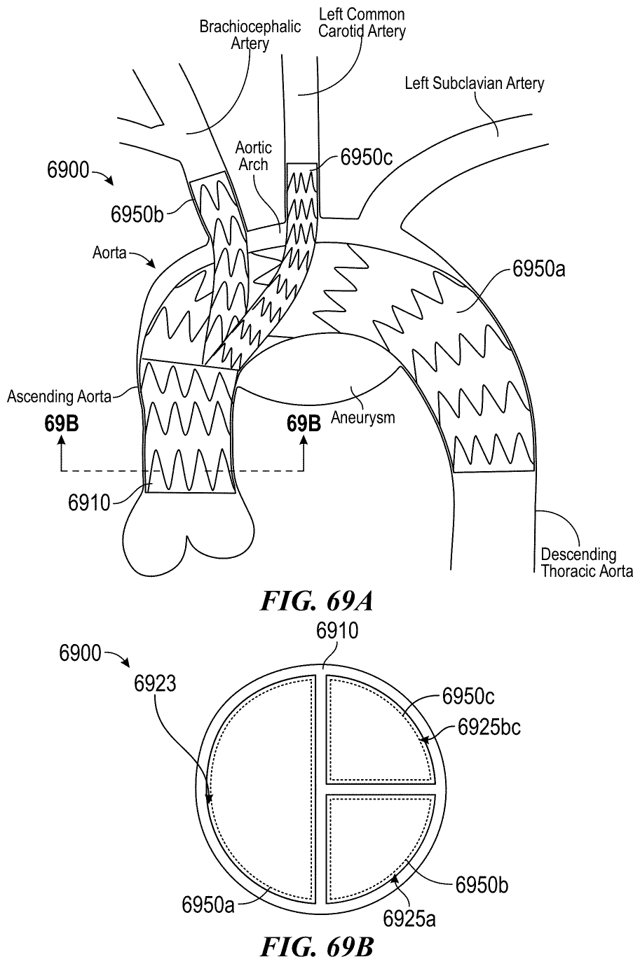

FIG. 69A is a side view of an aortic repair device 6900 implanted within an aorta in accordance with additional embodiments of the present technology. FIG. 69B is a cross-sectional view of the aortic repair device 6900 taken along the line 69B-69B in FIG. 69A in accordance with additional embodiments of the present technology. Referring to FIGS. 69A and 69B together, the aortic repair device 6900 includes a dock or base member 6910 implanted at least partially within the ascending aorta, and a plurality of spanning members 6950 (identified individually as first through third spanning members 6950a-6950c, respectively) coupled to (e.g., docked to) the base member 6910. Each of the spanning members 6950 can include a proximal end portion positioned within the base member 6910.

In the illustrated embodiment, the base member 6910 defines a primary lumen 6923, a first secondary lumen 6925a, and second secondary lumen 6925b. In the illustrated embodiment, each of the spanning members 6950 are separate components that can be modularly coupled to the base member 6910 (e.g., none of the spanning members 6950 are integrally formed with the base member 6910). The first through third spanning members 6950a-c each include a proximal end portion at least partially positioned within the primary lumen 6923, the first secondary lumen 6925a, and the second secondary lumen 6925b, respectively, and configured (e.g., shaped, sized) to conform and seal against a circumference thereof. Accordingly, the spanning members 6950 can engage and/or conform to the lumens 6923, 6925a, 6925b and the circumference of the base member 6910 such that blood does not substantially flow therebetween and is contained within the spanning members 6950. An outer surface of the base member 6910 engages and seals with the wall of the aorta to circumferentially seal the aorta. Accordingly, blood can flow in antegrade through each of the spanning members 6950. In some aspects of the present technology, forming the spanning members 6950 as separate components not integrally attached to the base member 6910 can reduce the profile of the base member 6910-thereby allowing the base member 6910 to be deployed through a smaller delivery catheter and/or a narrower delivery path (e.g., via transcarotid rather than transfemoral approach).

In the illustrated embodiment, the first spanning member 6950a has a larger cross-sectional area than the second and third spanning members 6950b-c and routes blood flow distally through the aorta (e.g., to the descending thoracic aorta) past a diseased portion of the aorta, such as an aortic arch aneurysm. The second and third spanning members 6950b-c extend from the base member 6910 to the brachiocephalic artery and the left common carotid artery, respectively, and route blood flow thereto to perfuse those vessels. In some embodiments, the left subclavian artery can be perfused via a surgical bypass or another implant coupled to one or more of the components of the aortic repair device 6900.

### VII. SELECTED ADDITIONAL EMBODIMENTS OF ANCHORING FEATURES FOR SECURING AORTIC REPAIR DEVICES

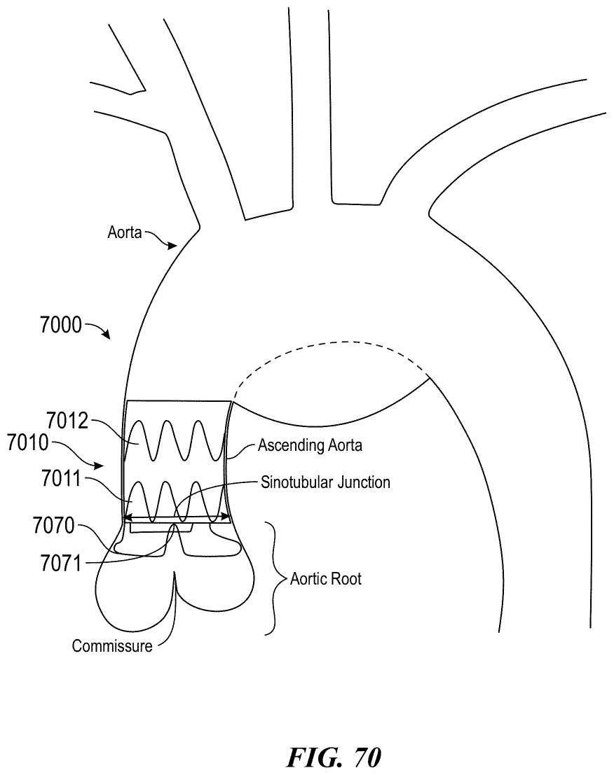

FIGS. 70-74B illustrate additional embodiments of aortic repair devices including anchoring features for inhibiting proximal migration of aortic repair devices through the aorta. The illustrated aortic repair devices can include some features that are at least generally similar in structure and function, or identical in structure and function, to one another and/or and the corresponding features of the aortic repair devices described in detail above with reference to FIGS. 3A-61B, and can operate in a generally similar or identical manner to one another and/or the aortic repair devices described with reference to FIGS. 3A-69B. Similarly, any of the anchoring or other features described in detail with reference to FIGS. 70-74B can be incorporated into the aortic repair devices of FIGS. 3A-61B.

FIG. 70 is a side view of an aortic repair device 7000 implanted within an aorta in accordance with embodiments of the present technology. In the illustrated embodiment, the aortic repair device 7000 includes a dock or base member 7010 implanted at least partially within the ascending aorta. The base member 7010 can comprise an expandable stent graft configured to expand and seal around a diseased portion of the aorta and defining one or more lumens for modularly receiving one or more spanning members for routing blood flow distally through the aorta and/or to one or more supra-aortic branch vessels. More specifically, the base member 7010 includes a tubular body 7012 configured to expand within and seal against the aorta.

In the illustrated embodiment, the base member 7010 further includes an anchor 7070 (e.g., a root anchor, a proximal anchor) extending proximally from a proximal end portion 7011 of the body 7012. In some embodiments, the anchor 7070 comprises a wire form that has a “hoop-like” shape with a larger expanded diameter than the body 7012. The anchor 7070 can extend at least partially into the aortic root to anchor the body 7012 in place within the proximal aorta. The aortic root is typically bulbous and has a maximum diameter that is larger than a healthy ascending aorta. The sinotubular junction is the junction between the distal end of the aortic root and the proximal end of the ascending aorta and is known to have “tougher” tissue as compared to the tissue on either side of it (e.g., like a fibrous ring). Accordingly, in some aspects of the present technology the anchor 7070 can at least partially engage the sinotubular junction to provide a sturdy anchor point for the base member 7010 that inhibits or even prevents proximal migration of the body 7012. In some embodiments, the anchor 7070 can include openings or arches 7071 around the commissures of the aortic valve to provide spaces for the commissures to slot into so that the function of the aortic valve is not comprised.

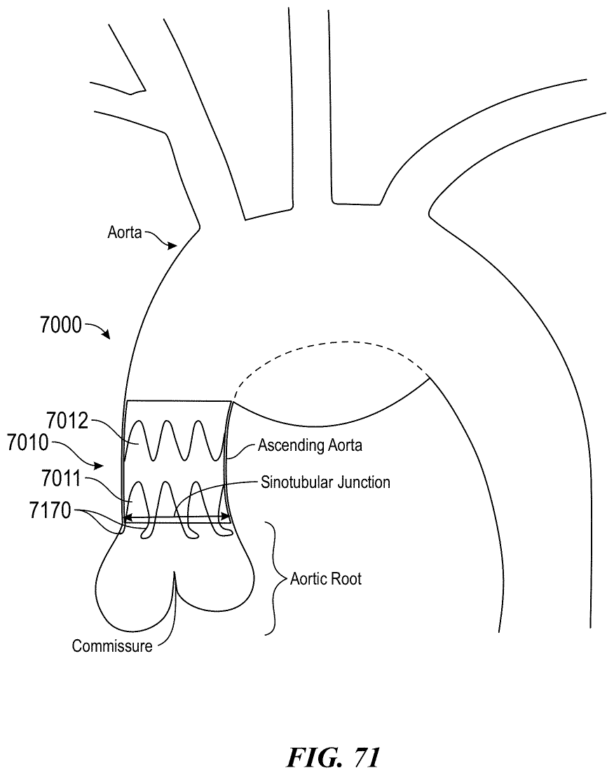

In the illustrated embodiment, the anchor 7070 comprises a plurality of elongate hoop-like shapes spaced circumferentially about the body 7012. In other embodiments, the anchor 7070 can have other shapes. For example, FIG. 71 is a side view of the aortic repair device 7000 implanted within the aorta and including an anchor 7170 in accordance with additional embodiments of the present technology. In the illustrated embodiment, the anchor 7170 is a wire form including a plurality of small, flared loops that engage the sinotubular junction. As shown in FIG. 71, these flared wire loops may be extensions of wire loops which form the tubular shape of the body 7010, thereby giving the flared wire loops additional strength to engage the sinotubular junction.

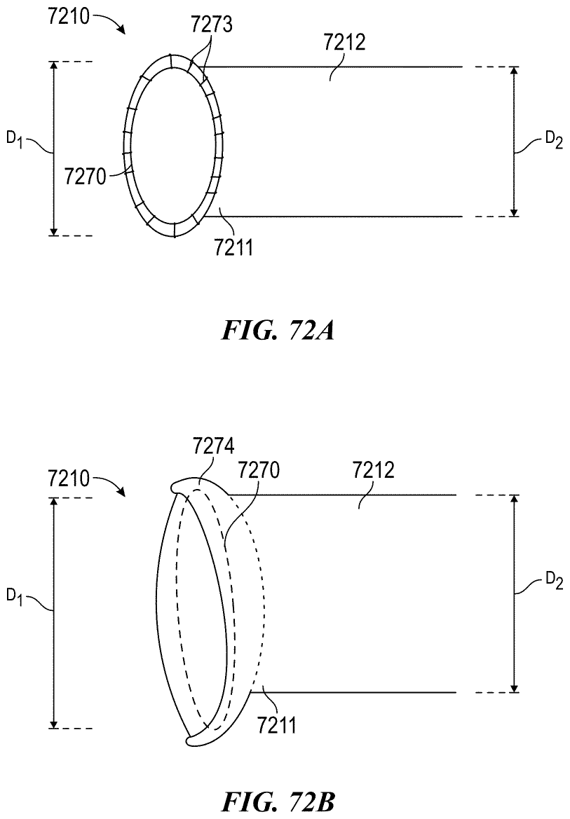

FIGS. 72A and 72B are enlarged isometric side views of base member 7210 of an aortic repair device in accordance with embodiments of the present technology. Referring to FIGS. 72A and 72B together, the base member 7210 includes a tubular body 7212 having a proximal end portion 7211, and an anchor 7270 coupled to the proximal end portion 7211 of the body 7212. In the illustrated embodiment, the anchor 7270 is a circular member, such as a ring stent formed of nitinol wire, having a diameter D₁ greater than a diameter D₂ of the body 7212. The anchor 7270 can be attached to the proximal end portion 7211 of the body 7212 by being directly attached to an edge thereof (e.g., via stitches 7273) as shown in FIG. 72A, or by being positioned in a pocket 7274 as shown in FIG. 72B. The body 7212 can comprise an expandable graft material or stent graft configured to expand and seal around a diseased portion of the aorta and defining one or more lumens for modularly receiving one or more spanning members for routing blood flow distally through the aorta and/or to one or more supra-aortic branch vessels. The anchor 7270 is configured (e.g., sized and shaped) to engage the sinotubular junction when the base member 7210 is implanted within an aorta to anchor the body 7212 and inhibit or even prevent distal migration of the body 7212. In some aspects of the present technology, the base member 7210 can have a windsock-shape when implanted within the aorta.

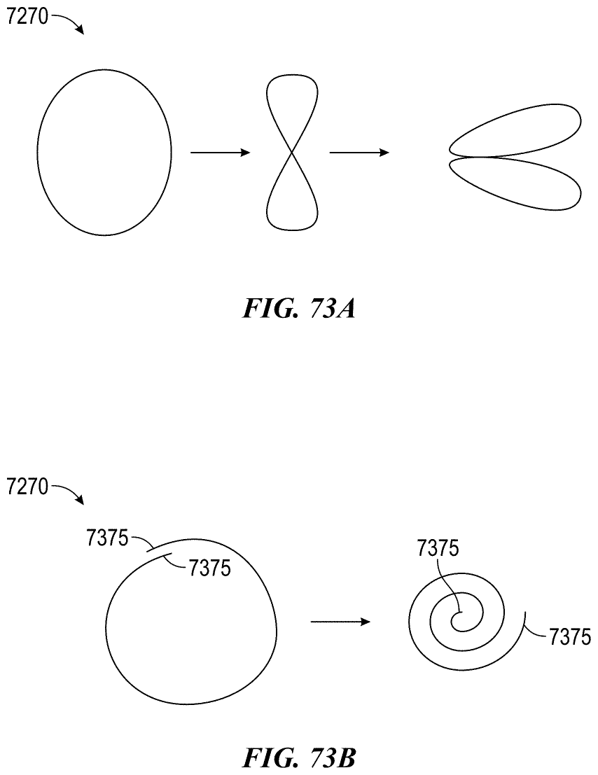

The anchor 7270 can comprise a continuous ring or an open ring (e.g., including a pair of free ends, a slot therethrough) and can be collapsed for loading into a delivery catheter system. For example, FIG. 73A includes end views illustrating the sequential collapse of the anchor 7270 for loading into a delivery catheter system when the anchor 7270 comprises a closed ring in accordance with embodiments of the present technology. As shown, the anchor 7270 can be folded into a figure-eight shape to collapse the anchor 7270 to a smaller delivery profile. Similarly, FIG. 73B includes end views illustrating the sequential collapse of the anchor 7270 for loading into a delivery catheter system when the anchor 7270 comprises an open ring in accordance with embodiments of the present technology. As shown, the anchor 7270 can include a pair of opposing free ends 7375 that can be moved relative to one another to coil the anchor 7270 into a smaller delivery profile.

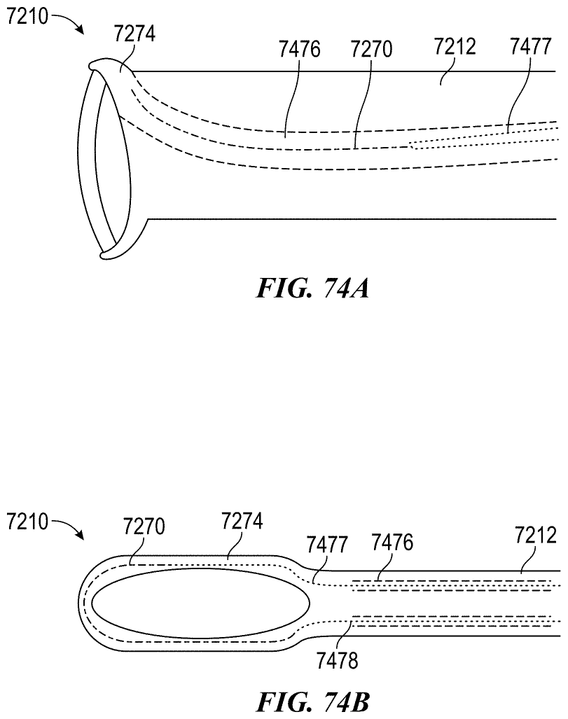

FIGS. 74A and 74B are enlarged isometric side views of the base member 7210 of FIG. 72B in accordance with embodiments of the present technology. Referring to FIGS. 74A and 74B together, in some embodiments the anchor 7270 comprises an open ring that is positioned within the pocket 7274 by being advanced through a channel 7476 in the body 7212 (FIG. 74A) and around the pocket 7274 (FIG. 74B) while in an elongated state. In some embodiments, a pushing element 7477 and/or a pulling element 7478 can be attached to a proximal and distal end of the anchor, respectively, and used to advance the anchor 7270 through the channel 7476 and around the pocket 7274. The anchor 7270 can be delivered to the pocket 7274 before or after the body 7212 is implanted within an aorta.

Additional means of anchoring to the aorta wall could be used with the base members of the devices described in FIGS. 70-74B, or the base members described in FIGS. 3-69B, or any of the other spanning members, parallel members, or other elements described herein. Anchors which pierce the aortic wall, such as the Heli-FX device manufactured by Medtronic, could additionally be used. The proximal end of any of the base members described herein, including the flared ends of the base members described in FIGS. 70-74B, may be particularly reinforced or otherwise designed to accommodate piercing anchors. Adhesives or other materials which promote ingrowth could be applied to the members, or delivered separately. Stents with protruding elements which can engage the aorta wall, such as those described in International Patent Application Publication No. PCT/US2021/024020, titled “EXPANDABLE DEVICES,” and filed Mar. 24, 2020, which is incorporated herein by reference in its entirety, could also be used at the ends of the base members to improve engagement and permanent fixation with the vessel wall.

### VIII. SELECTED ADDITIONAL EMBODIMENTS OF AORTIC REPAIR DEVICES INCLUDING SPIRAL STENTS

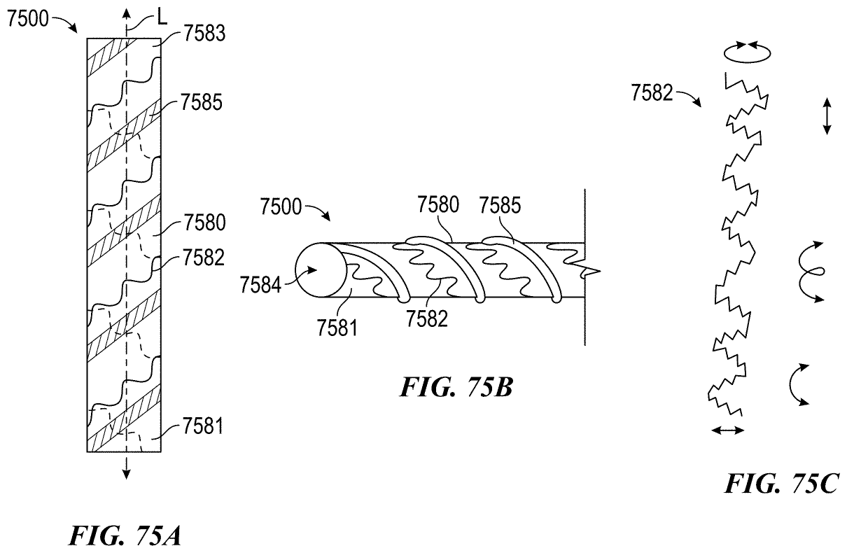

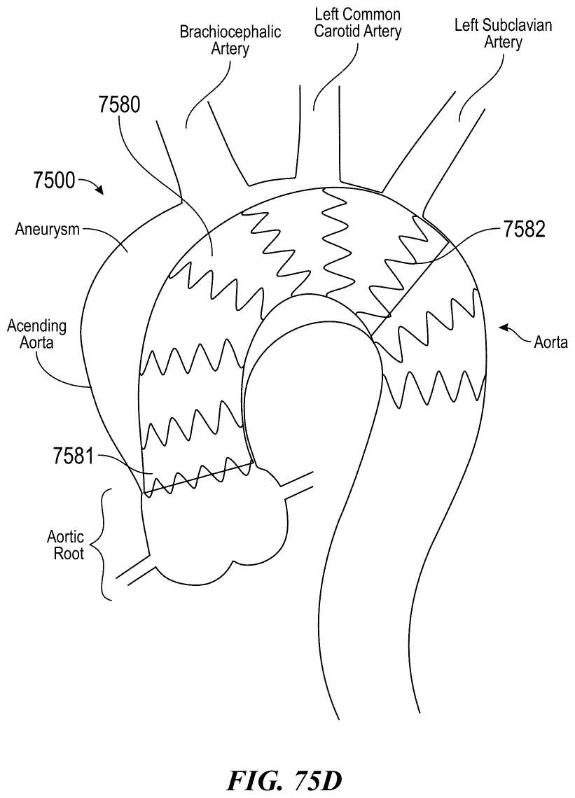

FIGS. 75A-81B illustrate additional embodiments of aortic repair devices including spiral stents. The illustrated aortic repair devices can include some features that are at least generally similar in structure and function, or identical in structure and function, to one another and/or and the corresponding features of the aortic repair devices described in detail above with reference to FIGS. 3A-74B, and can operate in a generally similar or identical manner to one another and/or the aortic repair devices described with reference to FIGS. 3A-74B. Similarly, any of the stents or other features described in detail with reference to FIGS. 75A-81B can be incorporated into the aortic repair devices of FIGS. 3A-74B.

FIGS. 75A and 75B are a side view and enlarged isometric side view, respectively, of an aortic repair device 7500 in accordance with embodiments of the present technology. Referring to FIGS. 75A and 75B together, the aortic repair device 7500 can have a generally tubular shape extending along a longitudinal axis L (FIG. 75A), and including a proximal end portion 7581 and a distal end portion 7583 and defining a lumen 7584. In the illustrated embodiment, the aortic repair device 7500 includes a graft material 7580 coupled to a stent 7582. In some embodiments, the graft material 7580 can include at least one pleat 7585 (e.g., ridge) extending in a spiral or other pattern at least partially between the proximal and distal end portions 7581, 7583. In some aspects of the present technology, the pleat 7585 can increase the flexibility and/or strength for the aortic repair device 7500 along its length.

FIG. 75C is an isometric side view of the stent 7582 in accordance with embodiments of the present technology. In the illustrated embodiment, the stent 7582 comprises a single integral member that spirals along and around the longitudinal axis L (FIG. 75A). Additionally, the stent 7582 includes a Z-like or V-like pattern along its length. The stent 7582 can be referred to as a “Z-stent,” a “spiral stent,” a “Z-spiral stent,” and/or the like. In some embodiments, the stent 7582 may include features to enhance radiopacity, such as a platinum core and/or radiopaque markers attached thereto.

FIG. 75D is a side view of the aortic repair device 7500 of FIGS. 75A-75C implanted within an aorta in accordance with embodiments of the present technology. In the illustrated embodiment, the proximal end portion 7581 of the aortic repair device 7500 is implanted within the proximal aorta (e.g., the ascending aorta, the aortic root) and the distal end portion 7583 is implanted within the aortic arch and/or the descending thoracic aorta. Accordingly, the aortic repair device 7500 can route blood can flow through the lumen 7584 (FIG. 75B) and past a diseased portion of the aorta, such as an ascending aortic aneurysm. The supra-aortic branch arteries (e.g., the brachiocephalic artery, the left common carotid artery, and the left subclavian artery) can be perfused via retrograde flow, other implants (not shown), and/or surgical bypasses. For example, the aortic repair device 7500 can be fenestrated in place and then one or more branch perfusion members can be fluidly connected to the fenestrations. In some embodiments, a spiral direction of the stent 7582 and the graft material 7580 can mimic the chiral blood flow through the aorta. In some aspects of the present technology, the spiral shape of the stent 7582 provides for radial, circumferential (e.g., twisting), and longitudinal support of the graft material 7580 and flexibility within the aorta during motion caused by the cardiac cycle (e.g., as opposed to separate ring stents that may rely upon a graft material for longitudinal and circumferential flexibility).

Referring to FIGS. 75A-75D together, in some embodiments the graft material 7580 and the stent 7582 can coextend (e.g., as shown in FIG. 75A) while, in other embodiments, the stent 7582 can extend farther distally than the graft material 7580 (e.g., as shown in FIG. 75D) or vice versa. Further, the graft material 7580 can be fixedly attached to the stent material 7582 during manufacturing and prior to delivery to the aorta, or can be separate from the stent material 7582 such that the graft material 7580 and the stent 7582 can be delivered separately to the aorta.

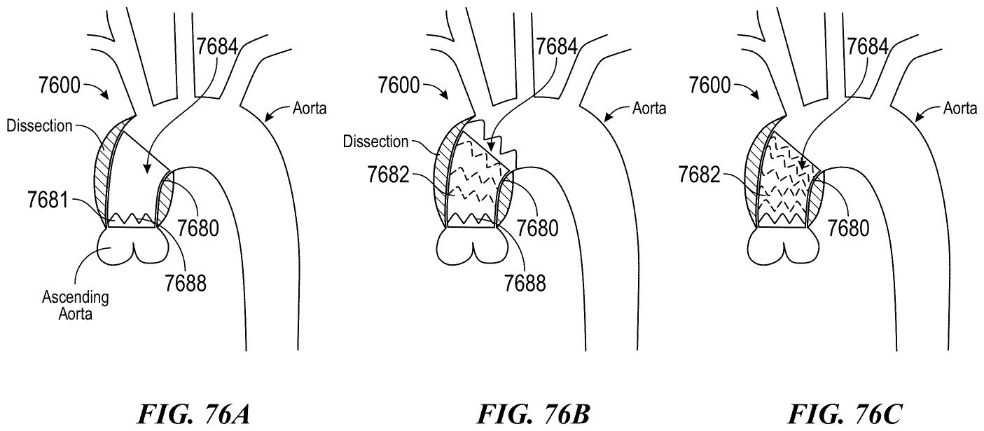

For example, FIGS. 76A-76C are side views of an aortic repair device 7600 during different stages of implantation within an aorta in accordance with embodiments of the present technology. Referring to FIGS. 76A-76C together, the aortic repair device 7600 includes a graft material 7680 and a Z-stent 7682 configured to be positioned within and expand against the graft material 7680.

Referring to FIG. 76A, the graft material 7680 can be delivered first—before the Z-stent 7682—to the proximal aorta (e.g., the ascending aorta). In the illustrated embodiment, the graft material 7680 is implanted adjacent to a diseased portion of the aorta, such as a dissection. In some embodiments, the aortic repair device 7600 includes an anchor 7688 attached to a proximal end portion 7681 of the graft material 7680. The anchor 7688 can include a metallic framing (e.g., an expandable stent) and can at least temporarily maintain (i) the fixation of the graft material 7680 within the aorta, (ii) the sealing of the graft material 7680 within the aorta, and/or (iii) the patency of a lumen 7684 of the graft material 7680.

Next, referring to FIG. 76B, the Z-stent 7682 can be advanced to and expanded at least partially within the lumen 7684 of the graft material 7680. When expanded, the Z-stent 7682 can exert a radial outward force against the graft material 7680 such that the graft material 7680 seals with the wall of the aorta to divert blood flow through the lumen 7684 past the diseased portion of the aorta. In some embodiments, the graft material 7680 can be secured to the anchor 7688 and/or other anchoring features attached to the graft material 7680.

In some embodiments, a total length of the Z-stent 7682 can be modified to vary the pitch of the spiral shape to vary the radial-outward force provided by the Z-stent 7682. For example, increasing the length of the Z-stent 7682 can decrease the radial-outward force exerted by the Z-stent 7682, while decreasing the length of the Z-stent 7682 can increase the radial-outward force exerted by the Z-stent 7682. FIG. 76C, for example, illustrates the Z-stent 7682 having a smaller total length and positioned entirely within the lumen 7684 of the graft material 7680 to provide a greater radial-outward force than the arrangement of the Z-stent 7682 shown in FIG. 76C. The length of the Z-stent 7682 can be determined and varied intraoperatively using procedural imaging until a desired radial force is achieved.

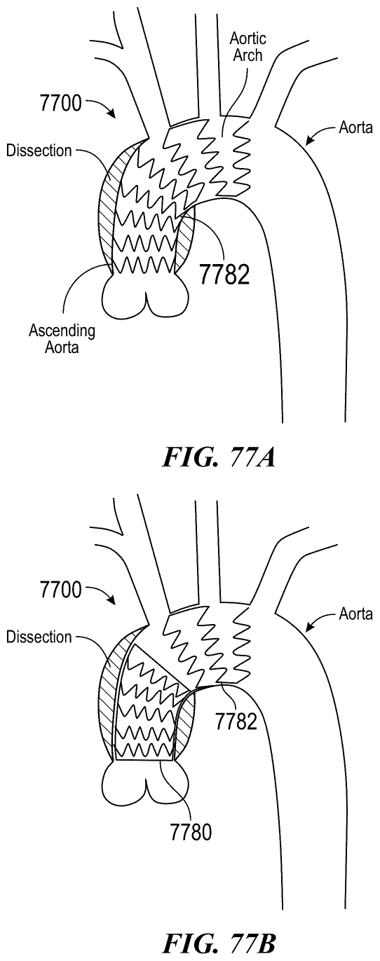

FIGS. 77A and 77B are side views of an aortic repair device 7700 during different stages of implantation within an aorta in accordance with embodiments of the present technology. Referring to FIGS. 77A and 77B together, the aortic repair device 7700 includes a graft material 7780 and a Z-stent 7782 configured to be positioned within and/or around the graft material 7780. Referring to FIG. 77A, the Z-stent 7782 can be delivered first—before the graft material 7780—to the proximal aorta (e.g., the ascending aorta and/or the aortic arch). In the illustrated embodiment, the Z-stent 7782 is implanted adjacent to a diseased portion of the aorta, such as a dissection. Next, as shown in FIG. 77B, the graft material 7780 can be advanced to and coupled to at least a portion of the Z-stent 7782 (e.g., targeted anchor locations), such as a portion adjacent the diseased portion of the aorta. The graft material 7780 can include anchoring features (not shown) for interlocking with the Z-stent 7782. After deployment, the graft material 7780 seals with the wall of the aorta to divert blood flow through aortic repair device 7700 past the diseased portion of the aorta.

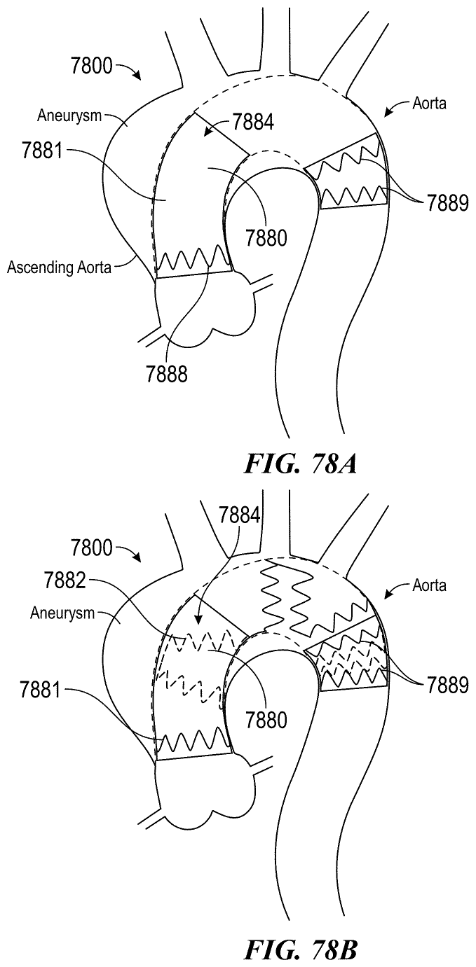

FIGS. 78A and 78B are side views of an aortic repair device 7800 during different stages of implantation within an aorta in accordance with embodiments of the present technology. Referring to FIGS. 78A and 78B together, the aortic repair device 7800 includes a graft material 7880 and a Z-stent 7882 configured to be positioned within and expand against the graft material 7880.

Referring to FIG. 78A, the graft material 7880 can be delivered first—before the Z-stent 7882—to the proximal aorta (e.g., the ascending aorta). In the illustrated embodiment, the graft material 7880 is implanted adjacent to a diseased portion of the aorta, such as an aneurysm. In the illustrated embodiment, the aortic repair device 7800 includes (i) a proximal anchor 7888 attached to a proximal end portion 7881 of the graft material 7880 and (ii) a distal anchor 7889 implanted distal of the proximal anchor 7888 (e.g., within the aortic arch and/or the descending thoracic aorta). The proximal and distal anchors 7888, 7889 can each include a metallic framing (e.g., an expandable stent, mesh, and/or other structure). In the illustrated embodiment, the graft material 7880 is not connected to the distal anchor 7889 and extends only partially distally theretoward. The proximal anchor 7888 can at least temporarily maintain the position of the graft material 5880.

Next, referring to FIG. 78B, the Z-stent 7882 can be advanced to and expanded at least partially within lumen 7884 of the graft material 7880. In some embodiments, the graft material 7880 is secured to the proximal and distal anchors 7888, 7889 within the aorta. When expanded, the Z-stent 7882 can exert a radial outward force against the graft material 7880 such that the graft material 7880 seals with the wall of the aorta to divert blood flow through the lumen 7884 past the diseased portion of the aorta.

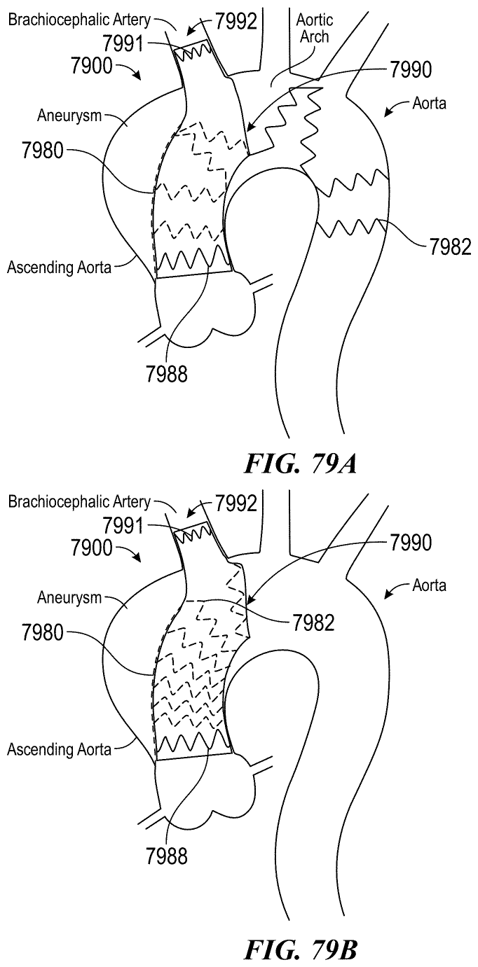

FIGS. 79A and 79B are side views of an aortic repair device 7900 implanted within an aorta in accordance with embodiments of the present technology. Referring to FIGS. 79A and 79B together, the aortic repair device 7900 can include a graft material 7980 having a proximal end portion 7981 attached to a proximal anchor 7988 and implanted at least partially within the proximal aorta. A Z-stent 7982 can be positioned at least partially within the graft material 7980 and secured to the proximal anchor 7988. More specifically, the Z-stent 7982 is elongated and extends distally of the graft material 7980 in FIG. 79A, while the Z-stent 7982 is compressed and contained substantially entirely within the graft material 7980 in FIG. 79B to provide a greater radial-outward force. In the illustrated embodiment, the graft material 7980 includes (i) a primary outlet 7990 positioned to direct blood flow distally into the aorta to perfuse the aorta and (ii) a secondary outlet 7992 positioned to direct blood flow into a branch vessel, such as the brachiocephalic artery, to perfuse the branch vessel. The secondary outlet 7992 can include a branch anchor 7991, such as a stent, coupled thereto for securing the secondary outlet 7992 within the brachiocephalic artery.

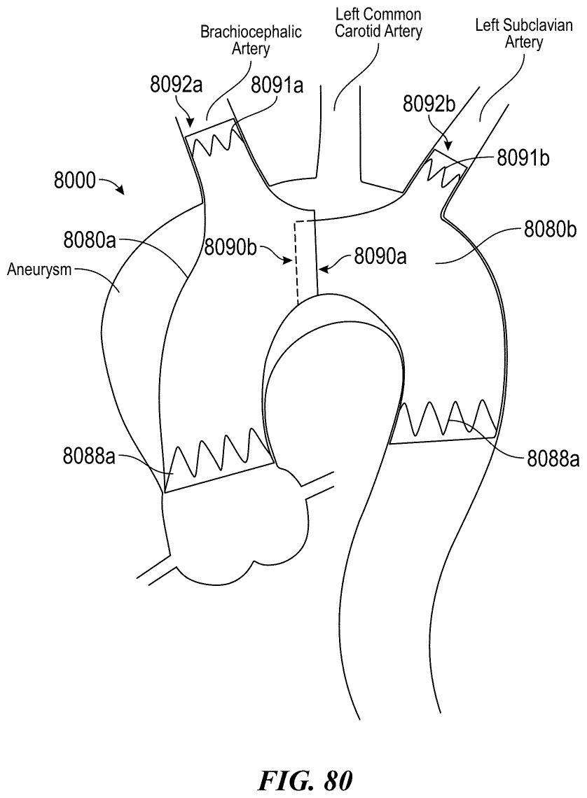

In some embodiments, multiple sections of branched grafts can be positioned within an aorta to provide a full aortic arch treatment. For example, FIG. 80 is a side view of an aortic repair device 8000 implanted within an aorta in accordance with embodiments of the present technology. In illustrated embodiment, the aortic repair device 8000 includes a first branched graft 8080a and a second branched graft 8080b implanted within the aorta. More specifically, the first branched graft 8080a is implanted within the proximal aorta adjacent to a diseased portion of the aorta, such as an aneurysm, and includes a primary outlet 8090a positioned to direct blood flow distally into the aorta, and a secondary outlet 8092a positioned to direct blood flow into a branch artery, such as the brachiocephalic artery. The first branched graft 8080a can be secured to a proximal anchor 8088a and/or a branch anchor 8091a. Similarly, the second branched graft 8080b can be implanted distal of the first branched graft 8080a within the aorta and includes a primary inlet 8090b, and a secondary outlet 8092b positioned to direct blood flow into a branch artery, such as the left subclavian artery. The second branched graft 8080b can be secured to a distal anchor 8088b and/or a branch anchor 8091b.

In the illustrated embodiment, the primary outlet 8090a of the first branched graft 8080a at least partially overlaps and seals against the primary inlet 8090b of the second branched graft 8080b. Accordingly, the aortic repair device 8000 is configured to route blood flow through the first and second branched grafts 8080a-b to the brachiocephalic artery, the left subclavian artery, and distally within the aorta. In some embodiments, a Z-stent (not shown) can be deployed within one or both of the first and second branched grafts 8080a-b to secure and help seal the aortic repair device 8000 within the aorta.

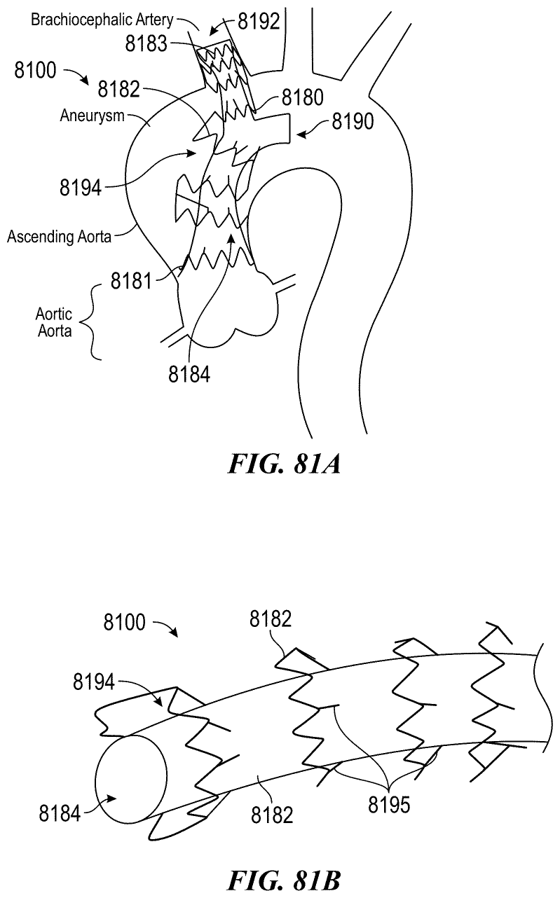

FIG. 81A is a side view of an aortic repair device 8100 implanted within an aorta in accordance with embodiments of the present technology. In the illustrated embodiment, the aortic repair device 8100 includes a Z-stent 8182 and a graft material 8180 coupled to the Z-stent 8182. The Z-stent can have a proximal end portion 8181 positioned in the proximal aorta (e.g., in the aortic root and/or the ascending aorta) and distal end portion 8183 positioned within a branch vessel, such as the brachiocephalic artery. In other embodiments, the distal end portion 8183 can be positioned within the aorta or another branch vessel. The graft material 8180 can have (i) primary outlet 8190 positioned to direct blood flow distally into the aorta to perfuse the aorta and (ii) a secondary outlet 8192 positioned to direct blood flow into a branch vessel, such as the brachiocephalic artery, to perfuse the branch vessel.

The Z-stent 8182 defines a lumen 8194, and the graft material 8180 is attached to the Z-stent 8182 within the lumen 8194 and itself defines a flow lumen 8184. More specifically, FIG. 81B is an enlarged isometric side view of a portion of the aortic repair device 8100 in accordance with embodiments of the present technology. As shown, the graft material 8180 can be supported within the lumen 8194 by connecting members 8195 located at select points along the stent 8182. For example, referring to FIGS. 81A and 81B together, the connecting members 8195 can attach to the graft material 8180 at and/or near the proximal and distal end portions 8181, 8183 to maintain the patency of the flow lumen 8184 and such that the graft material 8180 seals against the aortic wall at the proximal and distal end portions 8181, 8183 to direct blood flow substantially entirely through the lumen 8184. In some embodiments, the flow lumen 5184 can have a substantially constant diameter between the proximal and distal end portions 8181, 8183. In some aspects of the present technology, the Z-stent 8182 is oversized relative to the aorta such that motion caused by the cardiac cycle and across tortuous anatomies does not substantially impact the patency of the flow lumen 8184. In some embodiments, the graft material 8180 is reinforced through specific weave density for additional structural support between connecting members 8195. In some aspects of the present technology, positioning the graft material 8180 within the lumen 8194 of the Z-stent 8182 can reduce the total amount of graft material of the device 8100 as compared to, for example, embodiments in which the graft material 8180 is coupled directly to the outside or inside surface of a stent. This can help reduce the overall delivery profile of the device 8100.

### IX. SELECTED EMBODIMENTS OF METHODS OF FABRICATING AORTIC REPAIR DEVICES

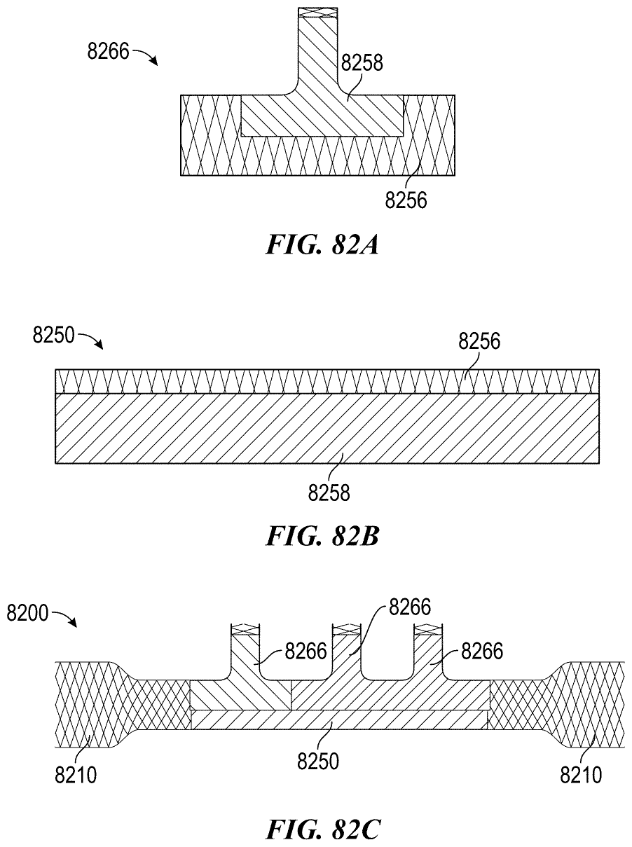

FIGS. 82A-85B illustrate various methods of fabricating aortic repair devices. The illustrated methods can be used to fabricate any of the aortic repair devices or other features described in detail with reference to FIGS. 3A-81B.

In many of the embodiments described above, different components of an aortic repair device comprise separate stent grafts that can be modularly coupled (e.g., mated, docked) together such that the graft material seals a region of a diseased aorta. In some embodiments, rather than covering the circumference of each stent graft with a graft material, graft material can be omitted from some components along regions of the components where the components are configured to contact another stent graft (e.g., reducing redundant graft material). As described above, graft material is the primary element of an aortic repair device that contributes to the volume of the device and therefore its ability to be compressed into a smaller-diameter catheter for delivery. Accordingly, decreasing the amount of graft material in this manner can reduce the delivery profile of the aortic repair device-potentially allowing the aortic repair device to be loaded into and delivered through a relatively smaller catheter and/or delivered along a narrower intravascular delivery pathway.

More specifically, for example, FIGS. 82A and 82B are side views of a branch perfusion member 8266 and a spanning member 8250, respectively, in accordance with embodiments of the present technology. Referring to FIGS. 82A and 82B together, the branch perfusion member 8266 and the spanning member 8250 each comprise a graft material 8258 attached to a stent structure 8256 and extending only partially about the stent structure 8256.

FIG. 82C is a side view of a portion of a modularly assembled aortic repair device 8200 in accordance with embodiments of the present technology. In the illustrated embodiment, the aortic repair device 8200 includes multiple ones of the connecting members 8266 (FIG. 82A) coupled to the spanning member 8250 (FIG. 82B) and to a pair of base members 8210. Referring to FIGS. 82A-82C together, the graft material 8258 can be positioned on each of the components such that the assembled aortic repair device 8200 is entirely (or to a selected amount) covered by the graft material 8258. As noted above, this can reduce the overall profile of the aortic repair device 8200. In additional aspects of the present technology, the partially-covered components can be more easily delivered into the aorta because the open portions of the stent structure 8256 allow blood flow therethrough-reducing forces on the components during delivery.

In many of the embodiments described above, an aortic repair device includes multiple ring stents spaced apart from one another that can be completely bare or only partially covered by a graft material. In some embodiments, it can be advantageous to secure some or all of such stents together to increase the strength and/or flexibility of the aortic repair device and/or to inhibit entanglement of the stents.

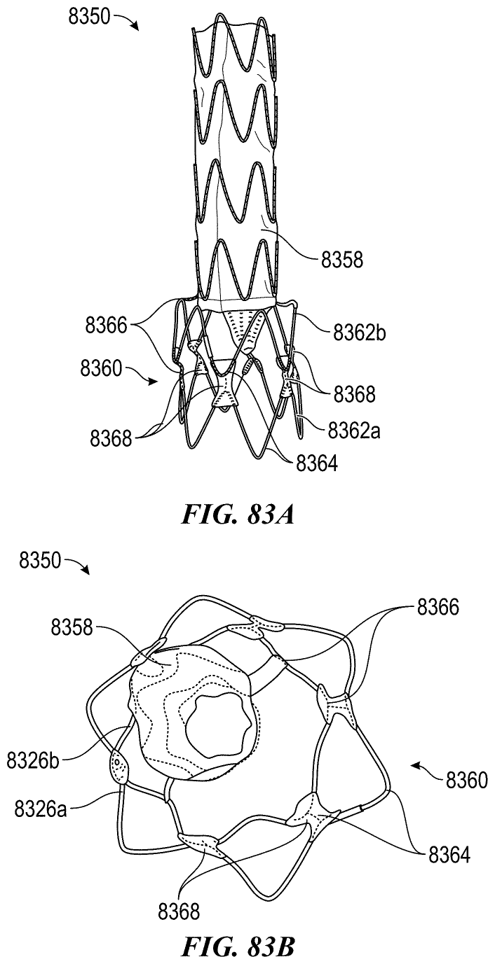

More specifically, for example, FIGS. 83A and 83B are a perspective side view and a distally-facing perspective end view, respectively, of a spanning member 8350 of an aortic repair device in accordance with embodiments of the present technology. Referring to FIGS. 83A and 83B together, the spanning member 8350 can include a proximal anchor 8360 including multiple bare ring stents 8362 (including an individually identified proximal stent 8362a and a distal stent 8362b) that are spaced apart from one another and that are not covered by a graft material 8358 (e.g., as described in detail above with reference to FIGS. 48A-55D). In the illustrated embodiment, the stents 8362 each have a V-like pattern including proximal apices 8364 and distal apices 8366. The spanning member 8350 further includes connectors 8368, such as fabric pieces having a bowtie-like shape, extending between and coupling the distal apices 8366 of the proximal stent 8362a to the proximal apices 8364 of the distal stent 8362b. In some aspects of the present technology, the connectors 8368 allow the stents 8362 to flex without becoming entangled.

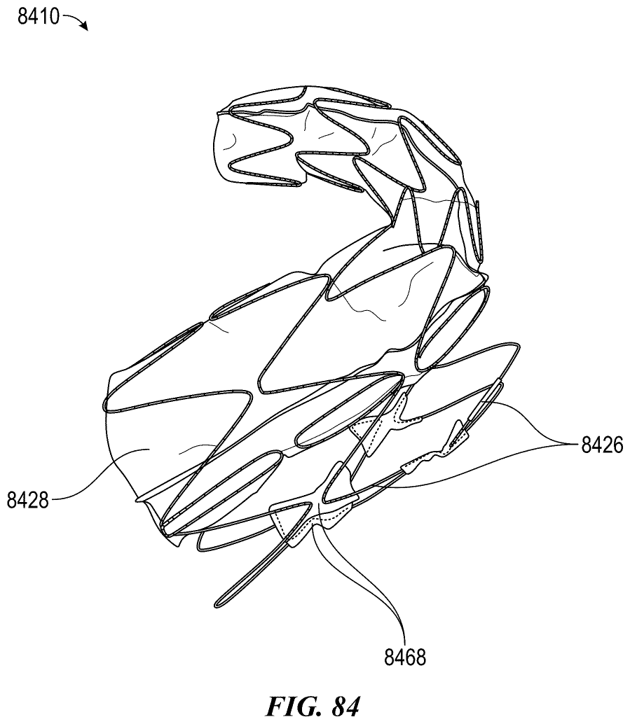

FIG. 84 is a perspective side view of a base member 8410 of an aortic repair device in accordance with embodiments of the present technology. The base member 8410 can include a plurality of proximal stents 8426 that are spaced apart from another and that are partially exposed from a graft material 8428 (e.g., as described in detail above with reference to FIGS. 35-37B). In the illustrated embodiment, adjacent ends of the stents 8426 are connected together via connectors 5848 in the manner described in detail with reference to FIGS. 55A and 55B.

FIGS. 85A and 85B are a perspective side view and a distally-facing perspective end view, respectively, of a base member 8510 of an aortic repair device in accordance with embodiments of the present technology. Referring to FIGS. 85A and 85B together, the base member 8510 can include multiple bare ring stents 8526 that are spaced apart from one another and connected via a septum 8518 (e.g., as described in detail above with reference to FIGS. 29A-33). In the illustrated embodiment, the stents 8526 each have a V-like pattern including proximal apices 8564 and distal apices 8566. The base member 8510 further includes connectors 8568, such as elongate fabric strips, extending between and coupling at least some of (i) the distal apices 8566 of adjacent ends of the stents 8526 and (ii) the proximal apices 8564 of adjacent ends of the stents 8526. In the illustrated embodiment, the connectors 8568 are positioned on only one side of the septum 8518 while, in other embodiments, the base member 8510 can include any number of the connectors 8568. In some aspects of the present technology, the connectors 8568 allow the stents 8526 to flex without becoming entangled.

### X. ADDITIONAL EXAMPLES

The following examples are illustrative of several embodiments of the present technology:
1. An aortic repair device, comprising:
a main body having a first end portion and a second end portion, the main body comprising a frame coupled to a cover that defines a fluid conduit, wherein the first end portion of the main body defines a first fluid opening;
a septum extending through the main body from the second end portion of the main body toward the first end portion of the main body, wherein the septum divides the fluid conduit into a primary lumen having a first cross-sectional area and a secondary lumen having a second cross-sectional area less than the first cross-sectional area, and wherein the second end portion of the main body defines a second fluid opening for the primary lumen; and
a leg extending from the second end portion of the main body, wherein the leg defines a leg lumen fluidly coupled to the secondary lumen, and wherein the leg has an end portion defining a third fluid opening for the secondary lumen.
2. The aortic repair device of example 1 wherein:
the main body is configured to be implanted within an ascending portion of the thoracic aorta,
the cover comprises a graft material coupled to the stents,
the aortic repair device further comprises a plurality of secondary lumen stents positioned within the secondary lumen,
the secondary lumen extends along an axis,
the secondary lumen stents are configured to expand to an expanded state, and
at least one of the secondary lumen stents has a generally D-shape or bowed-D shape about the axis in the expanded state.
3. The aortic repair device of example 1 wherein:
the main body is configured to be implanted within a descending portion of the thoracic aorta,
the cover comprises a graft material coupled to the stents,
the aortic repair device further comprises a plurality of secondary lumen stents positioned within the secondary lumen,
the secondary lumen extends along an axis,
the secondary lumen stents are configured to expand to an expanded state, and
at least one of the secondary lumen stents has a generally D-shape or bowed-D shape about the axis in the expanded state.
4. The aortic repair device of any one of examples 1-3 wherein the septum extends from the second end portion of the main body and terminates a distance from the first end portion of the main body.
5. The aortic repair device of any one of examples 1-3 wherein the septum extends from the second end portion of the main body entirely to the first end portion of the main body.
6. The aortic repair device of any one of examples 1-5 wherein:
the frame of the main body comprises a plurality of stents;
the cover comprises a graft material coupled to the stents; and
the aortic repair device further comprises a stent structure positioned within the secondary lumen.
7. The aortic repair device of example 6 wherein the stent structure is a tubular stent.
8. The aortic repair device of example 6 or example 7 wherein the stent structure comprises a plurality of secondary lumen stents.
9. The aortic repair device of example 8 wherein the secondary lumen extends along an axis, wherein the secondary lumen stents are configured to expand to an expanded state, and wherein at least one of the secondary lumen stents has a generally circular shape about the axis in the expanded state.
10. The aortic repair device of example 8 wherein the secondary lumen extends along an axis, wherein the secondary lumen stents are configured to expand to an expanded state, and wherein at least one of the secondary lumen stents has a generally D-shape about the axis in the expanded state.
11. The aortic repair device of example 8 or example 9 wherein the secondary lumen extends along an axis, wherein the secondary lumen stents are configured to expand to an expanded state, and wherein at least one of the secondary lumen stents has a bowed-D shape about the axis in the expanded state.
12. The aortic repair device of any one of examples 8-11 wherein the secondary lumen extends along an axis, wherein the secondary lumen stents are configured to expand to an expanded state, and wherein, in the expanded state:
at least one of the secondary lumen stents has a first curved portion extending along the main body within the secondary lumen and a second curved portion extending along the septum within the secondary lumen,
the first curved portion has a first radius of curvature, and
the second curved portion has a second radius of curvature different from the first radius of curvature.
13. The aortic repair device of example 12 wherein the first radius of curvature is less than the second radius of curvature.
14. The aortic repair device of any one of examples 8-13 wherein the secondary lumen stents extend only partially about the secondary lumen.
15. The aortic repair device of any one of examples 1-14 wherein:
the main body extends along an axis and has a first side portion and a second side portion, the first side portion being proximate to the secondary lumen and the second side portion being proximate to the primary lumen; and
the frame comprises a plurality of stents, the stents each have an amplitude along the axis, and the amplitude of at least one of the stents decreases in a direction from the first side portion toward the second side portion.
16. The aortic repair device of any one of examples 1-15 wherein:
the main body extends along an axis and has a first side portion and a second side portion, the first side portion being proximate to the secondary lumen and the second side portion being proximate to the primary lumen; and
the aortic repair device further comprises a tension member positioned at the second side portion and configured to tension the second side portion to have a curved shape.
17. The aortic repair device of example 16 wherein the frame of main body comprises a plurality of stents, wherein the cover comprises a graft material coupled to the stents, and wherein the tension member comprises a plurality of elastic fibers secured between corresponding ones of the stents.
18. The aortic repair device of any one of examples 1-17 wherein the main body extends along a first axis and the leg extends along a second axis non-parallel to the first axis.
19. The aortic repair device of any one of examples 1-8 wherein the main body has a diameter of between about 26-54 millimeters, and wherein the leg has a diameter of between about 10-22 millimeters.
20. The aortic repair device of any one of examples 1, 2, or 4-19 wherein the main body is configured to be positioned in an ascending portion of the thoracic aorta such that the first fluid opening receives blood flow from the ascending portion of the thoracic aorta and the second fluid opening discharges the blood flow into the thoracic aorta, and wherein at least a portion of the leg is configured to be positioned within a branch vessel branching from the thoracic aorta.
21. The aortic repair device of any one of examples 1, 3, or 4-19 wherein the main body is configured to be positioned in a descending portion of the thoracic aorta such that the second fluid opening receives blood flow from the thoracic aorta and the first fluid opening discharges the blood flow into the descending portion of the thoracic aorta, and wherein at least a portion of the leg is configured to be positioned within a branch vessel branching from the thoracic aorta.
22. A method of repairing a thoracic aorta, the method comprising:
intravascularly delivering an aortic repair device to a target site in the thoracic aorta;
positioning a main body of the aortic repair device such that an exterior surface of the main body sealingly contacts a wall of the thoracic aorta, a first fluid opening of the main body is positioned to receive blood flow from the thoracic aorta, and a second fluid opening of the main body is positioned to discharge a first portion of the blood flow into the thoracic aorta, wherein the aortic repair device comprises a septum extending at least partially
   between the first fluid opening and the second fluid opening of the main body, the septum dividing the main body into a primary lumen and a secondary lumen, and
   wherein the first fluid opening of the main body is fluidly coupled to the primary lumen to receive the first portion of the blood flow therethrough; and
positioning at least a portion of a leg of the aortic repair device within a branch vessel branching from the thoracic aorta such that a third fluid opening of the leg is positioned within the branch vessel to discharge a second portion of the blood flow into the branch vessel,
   wherein the third fluid opening is fluidly coupled to the secondary lumen to receive the second portion of the blood flow therethrough.
23. The method of example 22 wherein the main body has a length extending between the first fluid opening and the second fluid opening, and wherein positioning the main body of the aortic repair device includes positioning the main body to sealingly contact the wall of the thoracic aorta along substantially the entire length of the main body.
24. The method of example 22 or example 23 wherein the wall of the thoracic aorta is a wall of an ascending portion of the thoracic aorta, wherein the leg extends from the main body proximate to the second fluid opening, and wherein the septum extends from the second fluid opening at least partially toward the first fluid opening.
25. The method of example 24 wherein the branch vessel is a brachiocephalic artery.
26. The method of example 24 wherein the branch vessel is a left subclavian artery.
27. The method of example 24 wherein the branch vessel is a left common carotid artery.
28. The method of example 22 or example 23 wherein the wall of the thoracic aorta is a wall of a descending portion of the thoracic aorta, wherein the leg extends from the main body proximate to the first fluid opening, and wherein the septum extends from the first fluid opening at least partially toward the first fluid opening.
29. The method of example 28 wherein the branch vessel is a left subclavian artery.
30. The method of example 28 wherein the branch vessel is a brachiocephalic artery.
31. The method of example 28 wherein the branch vessel is a left common carotid artery.
32. The method of any one of examples 22-32, further comprising expanding a stent structure at least partially within the secondary lumen.
33. The method of example 32 wherein expanding the stent structure includes deploying a tubular stent at least partially within the secondary lumen.
34. The method of example 32 or example 33 wherein expanding the stent structure includes permitting a plurality of stents within the secondary lumen to self-expand.
35. The method of example 34 wherein the stents each have a generally D-shape or bowed-D shape.
36. The method of any one of examples 32-35, further comprising maintaining a cross-sectional area of the secondary lumen with the stent structure.
37. The method of any one of examples 32-36, further comprising: positioning a portion of a spanning member of the aortic repair device within a portion of the primary lumen of the main body; and maintaining a cross-sectional area of the secondary lumen with the stent structure when the portion of the spanning member is positioned within the secondary lumen.
38. The method of example 37 wherein the wall of the thoracic aorta is a wall of an ascending portion of the thoracic aorta, wherein the leg extends from the main body proximate to the second fluid opening, and wherein positioning the portion of the spanning member within the portion of the primary lumen of the main body includes inserting the portion of the spanning member through the second fluid opening.
39. The method of example 37 wherein the wall of the thoracic aorta is a wall of a descending portion of the thoracic aorta, wherein the leg extends from the main body proximate to the first fluid opening, and wherein positioning the portion of the spanning member within the portion of the primary lumen of the main body includes inserting the portion of the spanning member through the first fluid opening.
40. The method of any one of examples 22-39 wherein positioning the main body of the aortic repair device includes positioning the main body adjacent to an aortic aneurysm.
41. The method of any one of examples 22-40 wherein positioning the main body of the aortic repair device includes positioning the main body adjacent to an aortic dissection.
42. An aortic repair device, comprising:
a base member including—
   a main body having a first end portion and a second end portion, the main body comprising a frame coupled to a cover that defines a fluid conduit, wherein the first end portion of the main body defines a first body fluid opening;
   a septum extending through the main body from the second end portion of the main body toward the first end portion of the main body, wherein the septum divides the fluid conduit into a primary lumen having a first cross-sectional area and a secondary lumen having a second cross-sectional area less than the first cross-sectional area, and wherein the second end portion of the main body defines a second body fluid opening for the primary lumen; and
   a leg extending from the second end portion of the main body, wherein the leg defines a leg lumen fluidly coupled to the secondary lumen, and wherein the leg has an end portion defining a leg fluid opening for the secondary lumen; and
a spanning member having a first end portion defining a first spanning fluid opening and a second end portion defining a second spanning fluid opening, wherein the spanning member defines a spanning lumen extending between the first spanning fluid opening and the second spanning fluid opening, and wherein the first end portion is configured to (a) extend through the second body fluid opening, (b) be secured within the primary lumen, and (c) sealingly engage the septum and the main body within the primary lumen to define a continuous flow path through the primary lumen, the first spanning fluid opening, the spanning lumen, and the second spanning fluid opening.
43. The aortic repair device of example 42 wherein the base member comprises a first stent-graft, and wherein the spanning member comprises a second stent-graft.
44. The aortic repair device of example 42 or example 43 wherein the frame of the main body comprises a plurality of stents, wherein the cover comprises a graft material coupled to the stents, and wherein the base member further comprises a stent structure positioned within the secondary lumen to maintain the second cross-sectional area when the first end portion of the spanning member is secured within the primary lumen.
45. The aortic repair device of example 44 wherein the stent structure is a tubular stent.
46. The aortic repair device of example 44 wherein the stent structure comprises a plurality of secondary lumen stents.
47. The aortic repair device of example 46 wherein the secondary lumen extends along an axis, wherein the secondary lumen stents are configured to an expand to an expanded state, and wherein at least one of the secondary lumen stents has a generally circular shape about the axis in the expanded state.
48. The aortic repair device of example 46 or example 47 wherein the secondary lumen extends along an axis, wherein the secondary lumen stents are configured to an expand to an expanded state, and wherein at least one of the secondary lumen stents has a generally D-shape about the axis in the expanded state.
49. The aortic repair device of example 46 or example 47 wherein the secondary lumen extends along an axis, wherein the secondary lumen stents are configured to an expand to an expanded state, and wherein at least one of the secondary lumen stents has a generally bowed-D shape about the axis in the expanded state.
50. The aortic repair device of any one of examples 42-49 wherein the spanning member has a scalloped pattern at the first end portion.
51. The aortic repair device of any one of examples 42-50 wherein the spanning member has a middle portion between the first end portion and the second end portion, wherein the first end portion has a first diameter, and wherein the middle portion has a second diameter less than the first diameter.
52. The aortic repair device of any one of examples 42-51 wherein the spanning member extends along an axis, wherein the spanning member has a middle portion between the first end portion and the second end portion, wherein the spanning member comprises a graft material coupled to a plurality of stents, wherein the stents extend circumferentially about and have a periodic shape about the axis, wherein a first one of the stents at the first end portion has a first amplitude, and wherein a second of the stents at the middle portion has a second amplitude greater than the first amplitude.
53. The aortic repair device of any one of examples 42-52 wherein the spanning member extends along an axis, wherein the spanning member has a middle portion between the first end portion and the second end portion, wherein the spanning member comprises a graft material coupled to a plurality of stents, wherein the stents extend circumferentially about and have a periodic shape about the axis, wherein a first one of the stents at the first end portion has a first frequency, and wherein a second of the stents at the middle portion has a second frequency smaller than the first frequency.
54. The aortic repair device of any one of examples 42-53 wherein the spanning member extends along an axis, wherein the spanning member has a middle portion between the first end portion and the second end portion, wherein the spanning member comprises a graft material coupled to a plurality of stents, wherein the stents extend circumferentially about and have a periodic shape about the axis, wherein a first one of the stents at the first end portion has a first frequency and a first amplitude, wherein a second of the stents at the middle portion has a second frequency and a second amplitude, wherein the first frequency is greater than the second frequency, and wherein the first amplitude is smaller than the second amplitude.
55. The aortic repair device of any one of examples 42-54 wherein:
the main body is configured to be positioned in an ascending portion of the thoracic aorta such that the first body fluid opening receives blood flow from the ascending portion of the thoracic aorta,
at least a portion of the leg is configured to be positioned within a branch vessel branching from the thoracic aorta such that the leg (a) receives a first portion of the blood flow from the first body fluid opening through the secondary lumen and (b) routes the first portion of the blood flow for discharge through the leg fluid opening to the branch vessel, and
the spanning member is configured to be positioned within the thoracic aorta to span an arched portion of the thoracic aorta such that (a) the first spanning inlet receives a second portion of the blood flow from the first body fluid opening through the primary lumen and (b) the spanning lumen routes the second portion of the blood flow to the second spanning outlet for discharge through the second spanning fluid opening to the thoracic aorta.
56. The aortic repair device of any one of examples 42-54 wherein:
the spanning member is configured to be positioned within the thoracic aorta to span an arch of the thoracic aorta such that (a) the second spanning fluid opening receives blood flow from the thoracic aorta and (b) the spanning lumen routes the blood flow to the first spanning fluid opening,
the main body is configured to be positioned in a descending portion of the thoracic aorta such that (a) the second body fluid opening receives the blood flow from the first spanning fluid opening and (b) the primary lumen routes the blood flow to the first body fluid opening for discharge to the descending thoracic aorta, and
at least a portion of the leg is configured to be positioned within a branch vessel branching from the thoracic aorta.
57. The aortic repair device of example 56 wherein:
at least a portion of the leg is configured to be positioned within a branch vessel branching from the thoracic aorta,
the first body fluid opening of the main body is further configured to receive retrograde blood flow from the descending thoracic aorta and/or a portion of the blood flow from the primary lumen, and
the secondary lumen is configured to route the retrograde blood flow and/or the portion of the blood flow from the primary lumen to the leg for discharge through the leg fluid opening to the branch vessel.
58. A method of repairing a thoracic aorta, the method comprising:
intravascularly delivering an aortic repair device to a target site in the thoracic aorta;
positioning a main body of a base member of the aortic repair device such that an exterior surface of the main body sealingly contacts a wall of the thoracic aorta, wherein the main body comprises a first body fluid opening, a second body fluid opening, and a septum extending from the second body fluid opening at least partially toward the first body fluid opening, the septum dividing the main body into a primary lumen and a secondary lumen, and
   wherein the second body fluid opening of the main body is fluidly coupled to the primary lumen;
positioning at least a portion of a leg of the base member within a branch vessel branching from the thoracic aorta such that a leg fluid opening of the leg is positioned within the branch vessel,
   wherein the leg fluid opening is fluidly coupled to the secondary lumen, and
   wherein the leg extends from the main body proximate to the second body fluid opening;
positioning a spanning member within the thoracic aorta, wherein the spanning member has a first end portion defining a first spanning fluid opening and a second end portion defining a second spanning fluid opening, and wherein the spanning member defines a spanning lumen extending between the first spanning fluid opening and the second spanning fluid opening;
positioning the first end portion of the spanning member within the primary lumen through the second body fluid opening; and
sealingly engaging the first end portion of the spanning member with the septum and the main body within the primary lumen to define a continuous flow path through the spanning lumen and the primary lumen.
59. The method of example 58 wherein the wall of the thoracic aorta is a wall of an ascending portion of the thoracic aorta, and wherein positioning the main body of the base member includes positioning the first body fluid opening to receive blood flow from the ascending portion of the thoracic aorta such that (a) the primary lumen receives a first portion of the blood flow and routes the first portion of the blood flow toward the second body fluid opening and (b) the secondary lumen receives a second portion of the blood flow and routes the second portion of the blood flow to the leg fluid opening.
60. The method of example 59 wherein positioning the first end portion of the spanning member within the primary lumen includes positioning the spanning member to (a) receive the first portion of the blood flow from the secondary lumen of the main body and (b) discharge the first portion of the blood flow through the second spanning fluid opening into the thoracic aorta.
61. The method of example 60 wherein positioning the spanning member within the thoracic aorta includes positioning the spanning member to span an arched portion of the thoracic aorta, and wherein positioning the first end portion of the spanning member within the primary lumen includes positioning the spanning member to discharge the first portion of the blood flow into a descending portion of the thoracic aorta.
62. The method of example 58 wherein the wall of the thoracic aorta is a wall of a descending portion of the thoracic aorta, wherein positioning the spanning member within the thoracic aorta includes positioning the spanning member such that (a) the second spanning fluid opening receives blood flow from the thoracic aorta and (b) the spanning lumen routes the blood flow to the primary lumen of the main body through the first spanning fluid opening.
63. The method of example 62 wherein positioning the main body of the base member includes positioning the first body fluid opening to discharge the blood flow to the descending portion of the thoracic aorta.
64. The method of example 63 wherein positioning the main body of the base member includes positioning (a) the first body fluid opening to receive retrograde blood flow from the descending portion of the thoracic aorta and (b) the secondary lumen to route the retrograde blood flow to the leg for discharge through the leg fluid opening to the branch vessel.
65. The method of example 63 or example 64 wherein the septum extends from the second body fluid opening of the main body and terminates a distance from the first body fluid opening of the main body, and wherein positioning the main body of the base member includes positioning the secondary lumen to (a) receive a portion of the blood flow along the distance and (b) route the portion of the blood flow to the leg for discharge through the leg fluid opening to the branch vessel.
66. The method of any one of examples 62-65 wherein positioning the spanning member within the thoracic aorta includes positioning the spanning member to span an arched portion of the thoracic aorta such that the second spanning fluid opening receives the blood from an ascending portion of the thoracic aorta.
67. The method of any one of examples 58-66 wherein the main body has a length extending between the first body fluid opening and the second body fluid opening, and wherein positioning the main body of the base member includes positioning the main body to sealingly contact the wall of the thoracic aorta along substantially the entire length of the main body.
68. The method of any one of examples 58-67 wherein positioning the spanning member within the thoracic aorta includes positioning the spanning member to span an arched portion of the thoracic aorta.
69. The method of any one of examples 58-68, further comprising expanding a stent structure at least partially within the secondary lumen to inhibit compression of the septum by the spanning member and maintain a cross-sectional area of the secondary lumen.
70. The method of example 69 wherein expanding the stent structure includes deploying a tubular stent at least partially within the secondary lumen.
71. The method of example 69 or example 70 wherein expanding the stent structure includes permitting a plurality of stents within the secondary lumen to self-expand.
72. The method of example 71 wherein the stents each have a generally D-shape or generally bowed-D shape.

### XI. CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. An aortic repair device, comprising:
a main body having a first end portion and a second end portion, the main body comprising a frame coupled to a cover that defines a fluid conduit, wherein the first end portion of the main body defines a first fluid opening;
a septum extending through the main body from the second end portion of the main body toward the first end portion of the main body, wherein the septum divides the fluid conduit into a primary lumen having a first cross-sectional area and a secondary lumen having a second cross-sectional area less than the first cross-sectional area, and wherein the second end portion of the main body defines a second fluid opening for the primary lumen; and
a leg extending from the second end portion of the main body, wherein the leg defines a leg lumen fluidly coupled to the secondary lumen, and wherein the leg has an end portion defining a third fluid opening for the secondary lumen.

2. The aortic repair device of claim 1 wherein:
the main body is configured to be implanted within an ascending portion of the thoracic aorta,
the cover comprises a graft material coupled to the stents,
the aortic repair device further comprises a plurality of secondary lumen stents positioned within the secondary lumen,
the secondary lumen extends along an axis,
the secondary lumen stents are configured to expand to an expanded state, and
at least one of the secondary lumen stents has a generally D-shape or bowed-D shape about the axis in the expanded state.

3. The aortic repair device of claim 1 wherein:
the main body is configured to be implanted within a descending portion of the thoracic aorta,
the cover comprises a graft material coupled to the stents,
the aortic repair device further comprises a plurality of secondary lumen stents positioned within the secondary lumen,
the secondary lumen extends along an axis,
the secondary lumen stents are configured to expand to an expanded state, and
at least one of the secondary lumen stents has a generally D-shape or bowed-D shape about the axis in the expanded state.

4. The aortic repair device of claim 1 wherein the septum extends from the second end portion of the main body and terminates a distance from the first end portion of the main body.

5. The aortic repair device of claim 1 wherein the septum extends from the second end portion of the main body entirely to the first end portion of the main body.

6. The aortic repair device of claim 1 wherein:
the frame of the main body comprises a plurality of stents;
the cover comprises a graft material coupled to the stents; and
the aortic repair device further comprises a stent structure positioned within the secondary lumen.

7. The aortic repair device of claim 6 wherein the stent structure is a tubular stent.

8. The aortic repair device of claim 6 wherein the stent structure comprises a plurality of secondary lumen stents.

9. The aortic repair device of claim 8 wherein the secondary lumen extends along an axis, wherein the secondary lumen stents are configured to expand to an expanded state, and wherein at least one of the secondary lumen stents has a generally circular shape about the axis in the expanded state.

10. The aortic repair device of claim 8 wherein the secondary lumen extends along an axis, wherein the secondary lumen stents are configured to expand to an expanded state, and wherein at least one of the secondary lumen stents has a generally D-shape about the axis in the expanded state.

11. The aortic repair device of claim 8 wherein the secondary lumen extends along an axis, wherein the secondary lumen stents are configured to expand to an expanded state, and wherein at least one of the secondary lumen stents has a bowed-D shape about the axis in the expanded state.

12. The aortic repair device of claim 8 wherein the secondary lumen extends along an axis, wherein the secondary lumen stents are configured to expand to an expanded state, and wherein, in the expanded state:
at least one of the secondary lumen stents has a first curved portion extending along the main body within the secondary lumen and a second curved portion extending along the septum within the secondary lumen,
the first curved portion has a first radius of curvature, and
the second curved portion has a second radius of curvature different from the first radius of curvature.

13. The aortic repair device of claim 12 wherein the first radius of curvature is less than the second radius of curvature.

14. The aortic repair device of claim 8 wherein the secondary lumen stents extend only partially about the secondary lumen.

15. The aortic repair device of claim 1 wherein:
the main body extends along an axis and has a first side portion and a second side portion, the first side portion being proximate to the secondary lumen and the second side portion being proximate to the primary lumen; and
the frame comprises a plurality of stents, the stents each have an amplitude along the axis, and the amplitude of at least one of the stents decreases in a direction from the first side portion toward the second side portion.

16. The aortic repair device of claim 1 wherein:
the main body extends along an axis and has a first side portion and a second side portion, the first side portion being proximate to the secondary lumen and the second side portion being proximate to the primary lumen; and
the aortic repair device further comprises a tension member positioned at the second side portion and configured to tension the second side portion to have a curved shape.

17. The aortic repair device of claim 16 wherein the frame of main body comprises a plurality of stents, wherein the cover comprises a graft material coupled to the stents, and wherein the tension member comprises a plurality of elastic fibers secured between corresponding ones of the stents.

18. The aortic repair device of claim 1 wherein the main body extends along a first axis and the leg extends along a second axis non-parallel to the first axis.

19. The aortic repair device of claim 1 wherein the main body has a diameter of between about 26-54 millimeters, and wherein the leg has a diameter of between about 10-22 millimeters.

20. The aortic repair device of claim 1 wherein the main body is configured to be positioned in an ascending portion of the thoracic aorta such that the first fluid opening receives blood flow from the ascending portion of the thoracic aorta and the second fluid opening discharges the blood flow into the thoracic aorta, and wherein at least a portion of the leg is configured to be positioned within a branch vessel branching from the thoracic aorta.

21. The aortic repair device of claim 1 wherein the main body is configured to be positioned in a descending portion of the thoracic aorta such that the second fluid opening receives blood flow from the thoracic aorta and the first fluid opening discharges the blood flow into the descending portion of the thoracic aorta, and wherein at least a portion of the leg is configured to be positioned within a branch vessel branching from the thoracic aorta.

22. A method of repairing a thoracic aorta, the method comprising:
intravascularly delivering an aortic repair device to a target site in the thoracic aorta;
positioning a main body of the aortic repair device such that an exterior surface of the main body sealingly contacts a wall of the thoracic aorta, a first fluid opening of the main body is positioned to receive blood flow from the thoracic aorta, and a second fluid opening of the main body is positioned to discharge a first portion of the blood flow into the thoracic aorta,
wherein the aortic repair device comprises a septum extending at least partially between the first fluid opening and the second fluid opening of the main body, the septum dividing the main body into a primary lumen and a secondary lumen, and
wherein the first fluid opening of the main body is fluidly coupled to the primary lumen to receive the first portion of the blood flow therethrough; and
positioning at least a portion of a leg of the aortic repair device within a branch vessel branching from the thoracic aorta such that a third fluid opening of the leg is positioned within the branch vessel to discharge a second portion of the blood flow into the branch vessel,
wherein the third fluid opening is fluidly coupled to the secondary lumen to receive the second portion of the blood flow therethrough.

23. The method of claim 22 wherein the main body has a length extending between the first fluid opening and the second fluid opening, and wherein positioning the main body of the aortic repair device includes positioning the main body to sealingly contact the wall of the thoracic aorta along substantially the entire length of the main body.

24. The method of claim 22 wherein the wall of the thoracic aorta is a wall of an ascending portion of the thoracic aorta, wherein the leg extends from the main body proximate to the second fluid opening, and wherein the septum extends from the second fluid opening at least partially toward the first fluid opening.

25. The method of claim 24 wherein the branch vessel is a brachiocephalic artery.

26. The method of claim 24 wherein the branch vessel is a left subclavian artery.

27. The method of claim 24 wherein the branch vessel is a left common carotid artery.

28. The method of claim 22 wherein the wall of the thoracic aorta is a wall of a descending portion of the thoracic aorta, wherein the leg extends from the main body proximate to the first fluid opening, and wherein the septum extends from the first fluid opening at least partially toward the first fluid opening.

29. The method of claim 28 wherein the branch vessel is a left subclavian artery.

30. The method of claim 28 wherein the branch vessel is a brachiocephalic artery.

31. The method of claim 28 wherein the branch vessel is a left common carotid artery.

32. The method of claim 22, further comprising expanding a stent structure at least partially within the secondary lumen.

33. The method of claim 32 wherein expanding the stent structure includes deploying a tubular stent at least partially within the secondary lumen.

34. The method of claim 32 wherein expanding the stent structure includes permitting a plurality of stents within the secondary lumen to self-expand.

35. The method of claim 34 wherein the stents each have a generally D-shape or bowed-D shape.

36. The method of claim 32, further comprising maintaining a cross-sectional area of the secondary lumen with the stent structure.

37. The method of claim 32, further comprising:
positioning a portion of a spanning member of the aortic repair device within a portion of the primary lumen of the main body; and
maintaining a cross-sectional area of the secondary lumen with the stent structure when the portion of the spanning member is positioned within the secondary lumen.

38. The method of claim 37 wherein the wall of the thoracic aorta is a wall of an ascending portion of the thoracic aorta, wherein the leg extends from the main body proximate to the second fluid opening, and wherein positioning the portion of the spanning member within the portion of the primary lumen of the main body includes inserting the portion of the spanning member through the second fluid opening.

39. The method of claim 37 wherein the wall of the thoracic aorta is a wall of a descending portion of the thoracic aorta, wherein the leg extends from the main body proximate to the first fluid opening, and wherein positioning the portion of the spanning member within the portion of the primary lumen of the main body includes inserting the portion of the spanning member through the first fluid opening.

40. The method of claim 22 wherein positioning the main body of the aortic repair device includes positioning the main body adjacent to an aortic aneurysm.

41. The method of claim 22 wherein positioning the main body of the aortic repair device includes positioning the main body adjacent to an aortic dissection.

42. An aortic repair device, comprising:
a base member including—
a main body having a first end portion and a second end portion, the main body comprising a frame coupled to a cover that defines a fluid conduit, wherein the first end portion of the main body defines a first body fluid opening;
a septum extending through the main body from the second end portion of the main body toward the first end portion of the main body, wherein the septum divides the fluid conduit into a primary lumen having a first cross-sectional area and a secondary lumen having a second cross-sectional area less than the first cross-sectional area, and wherein the second end portion of the main body defines a second body fluid opening for the primary lumen; and
a leg extending from the second end portion of the main body, wherein the leg defines a leg lumen fluidly coupled to the secondary lumen, and wherein the leg has an end portion defining a leg fluid opening for the secondary lumen; and
a spanning member having a first end portion defining a first spanning fluid opening and a second end portion defining a second spanning fluid opening, wherein the spanning member defines a spanning lumen extending between the first spanning fluid opening and the second spanning fluid opening, and wherein the first end portion is configured to (a) extend through the second body fluid opening, (b) be secured within the primary lumen, and (c) sealingly engage the septum and the main body within the primary lumen to define a continuous flow path through the primary lumen, the first spanning fluid opening, the spanning lumen, and the second spanning fluid opening.

43. The aortic repair device of claim 42 wherein the base member comprises a first stent-graft, and wherein the spanning member comprises a second stent-graft.

44. The aortic repair device of claim 42 wherein the frame of the main body comprises a plurality of stents, wherein the cover comprises a graft material coupled to the stents, and wherein the base member further comprises a stent structure positioned within the secondary lumen to maintain the second cross-sectional area when the first end portion of the spanning member is secured within the primary lumen.

45. The aortic repair device of claim 44 wherein the stent structure is a tubular stent.

46. The aortic repair device of claim 44 wherein the stent structure comprises a plurality of secondary lumen stents.

47. The aortic repair device of claim 46 wherein the secondary lumen extends along an axis, wherein the secondary lumen stents are configured to an expand to an expanded state, and wherein at least one of the secondary lumen stents has a generally circular shape about the axis in the expanded state.

48. The aortic repair device of claim 46 wherein the secondary lumen extends along an axis, wherein the secondary lumen stents are configured to an expand to an expanded state, and wherein at least one of the secondary lumen stents has a generally D-shape about the axis in the expanded state.

49. The aortic repair device of claim 46 wherein the secondary lumen extends along an axis, wherein the secondary lumen stents are configured to an expand to an expanded state, and wherein at least one of the secondary lumen stents has a generally bowed-D shape about the axis in the expanded state.

50. The aortic repair device of claim 42 wherein the spanning member has a scalloped pattern at the first end portion.

51. The aortic repair device of claim 42 wherein the spanning member has a middle portion between the first end portion and the second end portion, wherein the first end portion has a first diameter, and wherein the middle portion has a second diameter less than the first diameter.

52. The aortic repair device of claim 42 wherein the spanning member extends along an axis, wherein the spanning member has a middle portion between the first end portion and the second end portion, wherein the spanning member comprises a graft material coupled to a plurality of stents, wherein the stents extend circumferentially about and have a periodic shape about the axis, wherein a first one of the stents at the first end portion has a first amplitude, and wherein a second of the stents at the middle portion has a second amplitude greater than the first amplitude.

53. The aortic repair device of claim 42 wherein the spanning member extends along an axis, wherein the spanning member has a middle portion between the first end portion and the second end portion, wherein the spanning member comprises a graft material coupled to a plurality of stents, wherein the stents extend circumferentially about and have a periodic shape about the axis, wherein a first one of the stents at the first end portion has a first frequency, and wherein a second of the stents at the middle portion has a second frequency smaller than the first frequency.

54. The aortic repair device of claim 42 wherein the spanning member extends along an axis, wherein the spanning member has a middle portion between the first end portion and the second end portion, wherein the spanning member comprises a graft material coupled to a plurality of stents, wherein the stents extend circumferentially about and have a periodic shape about the axis, wherein a first one of the stents at the first end portion has a first frequency and a first amplitude, wherein a second of the stents at the middle portion has a second frequency and a second amplitude, wherein the first frequency is greater than the second frequency, and wherein the first amplitude is smaller than the second amplitude.

55. The aortic repair device of claim 42 wherein:
the main body is configured to be positioned in an ascending portion of the thoracic aorta such that the first body fluid opening receives blood flow from the ascending portion of the thoracic aorta,
at least a portion of the leg is configured to be positioned within a branch vessel branching from the thoracic aorta such that the leg (a) receives a first portion of the blood flow from the first body fluid opening through the secondary lumen and (b) routes the first portion of the blood flow for discharge through the leg fluid opening to the branch vessel, and
the spanning member is configured to be positioned within the thoracic aorta to span an arched portion of the thoracic aorta such that (a) the first spanning inlet receives a second portion of the blood flow from the first body fluid opening through the primary lumen and (b) the spanning lumen routes the second portion of the blood flow to the second spanning outlet for discharge through the second spanning fluid opening to the thoracic aorta.

56. The aortic repair device of claim 42 wherein:
the spanning member is configured to be positioned within the thoracic aorta to span an arch of the thoracic aorta such that (a) the second spanning fluid opening receives blood flow from the thoracic aorta and (b) the spanning lumen routes the blood flow to the first spanning fluid opening,
the main body is configured to be positioned in a descending portion of the thoracic aorta such that (a) the second body fluid opening receives the blood flow from the first spanning fluid opening and (b) the primary lumen routes the blood flow to the first body fluid opening for discharge to the descending thoracic aorta, and
at least a portion of the leg is configured to be positioned within a branch vessel branching from the thoracic aorta.

57. The aortic repair device of claim 56 wherein:
at least a portion of the leg is configured to be positioned within a branch vessel branching from the thoracic aorta,
the first body fluid opening of the main body is further configured to receive retrograde blood flow from the descending thoracic aorta and/or a portion of the blood flow from the primary lumen, and
the secondary lumen is configured to route the retrograde blood flow and/or the portion of the blood flow from the primary lumen to the leg for discharge through the leg fluid opening to the branch vessel.

58. A method of repairing a thoracic aorta, the method comprising:
intravascularly delivering an aortic repair device to a target site in the thoracic aorta;
positioning a main body of a base member of the aortic repair device such that an exterior surface of the main body sealingly contacts a wall of the thoracic aorta, wherein the main body comprises a first body fluid opening, a second body fluid opening, and a septum extending from the second body fluid opening at least partially toward the first body fluid opening, the septum dividing the main body into a primary lumen and a secondary lumen, and
wherein the second body fluid opening of the main body is fluidly coupled to the primary lumen;
positioning at least a portion of a leg of the base member within a branch vessel branching from the thoracic aorta such that a leg fluid opening of the leg is positioned within the branch vessel,
wherein the leg fluid opening is fluidly coupled to the secondary lumen, and
wherein the leg extends from the main body proximate to the second body fluid opening;
positioning a spanning member within the thoracic aorta, wherein the spanning member has a first end portion defining a first spanning fluid opening and a second end portion defining a second spanning fluid opening, and wherein the spanning member defines a spanning lumen extending between the first spanning fluid opening and the second spanning fluid opening;
positioning the first end portion of the spanning member within the primary lumen through the second body fluid opening; and
sealingly engaging the first end portion of the spanning member with the septum and the main body within the primary lumen to define a continuous flow path through the spanning lumen and the primary lumen.

59. The method of claim 58 wherein the wall of the thoracic aorta is a wall of an ascending portion of the thoracic aorta, and wherein positioning the main body of the base member includes positioning the first body fluid opening to receive blood flow from the ascending portion of the thoracic aorta such that (a) the primary lumen receives a first portion of the blood flow and routes the first portion of the blood flow toward the second body fluid opening and (b) the secondary lumen receives a second portion of the blood flow and routes the second portion of the blood flow to the leg fluid opening.

60. The method of claim 59 wherein positioning the first end portion of the spanning member within the primary lumen includes positioning the spanning member to (a) receive the first portion of the blood flow from the secondary lumen of the main body and (b) discharge the first portion of the blood flow through the second spanning fluid opening into the thoracic aorta.

61. The method of claim 60 wherein positioning the spanning member within the thoracic aorta includes positioning the spanning member to span an arched portion of the thoracic aorta, and wherein positioning the first end portion of the spanning member within the primary lumen includes positioning the spanning member to discharge the first portion of the blood flow into a descending portion of the thoracic aorta.

62. The method of claim 58 wherein the wall of the thoracic aorta is a wall of a descending portion of the thoracic aorta, wherein positioning the spanning member within the thoracic aorta includes positioning the spanning member such that (a) the second spanning fluid opening receives blood flow from the thoracic aorta and (b) the spanning lumen routes the blood flow to the primary lumen of the main body through the first spanning fluid opening.

63. The method of claim 62 wherein positioning the main body of the base member includes positioning the first body fluid opening to discharge the blood flow to the descending portion of the thoracic aorta.

64. The method of claim 63 wherein positioning the main body of the base member includes positioning (a) the first body fluid opening to receive retrograde blood flow from the descending portion of the thoracic aorta and (b) the secondary lumen to route the retrograde blood flow to the leg for discharge through the leg fluid opening to the branch vessel.

65. The method of claim 63 wherein the septum extends from the second body fluid opening of the main body and terminates a distance from the first body fluid opening of the main body, and wherein positioning the main body of the base member includes positioning the secondary lumen to (a) receive a portion of the blood flow along the distance and (b) route the portion of the blood flow to the leg for discharge through the leg fluid opening to the branch vessel.

66. The method of claim 62 wherein positioning the spanning member within the thoracic aorta includes positioning the spanning member to span an arched portion of the thoracic aorta such that the second spanning fluid opening receives the blood from an ascending portion of the thoracic aorta.

67. The method of claim 58 wherein the main body has a length extending between the first body fluid opening and the second body fluid opening, and wherein positioning the main body of the base member includes positioning the main body to sealingly contact the wall of the thoracic aorta along substantially the entire length of the main body.

68. The method of claim 58 wherein positioning the spanning member within the thoracic aorta includes positioning the spanning member to span an arched portion of the thoracic aorta.

69. The method of claim 58, further comprising expanding a stent structure at least partially within the secondary lumen to inhibit compression of the septum by the spanning member and maintain a cross-sectional area of the secondary lumen.

70. The method of claim 69 wherein expanding the stent structure includes deploying a tubular stent at least partially within the secondary lumen.

71. The method of claim 69 wherein expanding the stent structure includes permitting a plurality of stents within the secondary lumen to self-expand.

72. The method of claim 71 wherein the stents each have a generally D-shape or generally bowed-D shape.
